(19) United States
(12) Patent Application Publication
(10) Pub. No.: US 2025/0075256 A1
(43) Pub. Date: Mar. 6, 2025

(54) PORTABLE, LOW-COST PATHOGEN DETECTION AND STRAIN IDENTIFICATION PLATFORM

(71) Applicants: ARIZONA BOARD OF REGENTS ON BEHALF OF ARIZONA STATE UNIVERSITY, Scottsdale, AZ (US); PRESIDENT AND FELLOWS OF HARVARD COLLEGE, Cambridge, MA (US); MASSACHUSETTS INSTITUTE OF TECHNOLOGY, Cambridge, MA (US)

(72) Inventors: Alexander Green, Boston, MA (US); Dana Braff, Cambridge, MA (US); Melissa Takahashi, Cambridge, MA (US); Keith Pardee, Toronto (CA); James J. Collins, Cambridge, MA (US); Guillaume Lambert, Ithaca, NY (US); Thomas Ferrante, Cambridge, MA (US)

(21) Appl. No.: 18/830,513

(22) Filed: Sep. 10, 2024

Related U.S. Application Data
(62) Division of application No. 17/849,250, filed on Jun. 24, 2022, now Pat. No. 12,116,623.
Application No. 17/849,250 is a continuation of application No. 16/303,937, filed on Nov. 21, 2018, now Pat. No. 11,371,081.
(60) Provisional application No. 62/341,221, filed on May 25, 2016.
(60) Provisional application No. 62/403,778, filed on Oct. 4, 2016.

(51) Int. Cl. C12Q 1/6827; C12Q 1/6816; C12Q 1/70

(52) U.S. Cl. CPC C12Q 1/6827; C12Q 1/6816; C12Q 1/701

(57) ABSTRACT

Methods for detecting the presence of a pathogen infection are described. In particular, this document provides a method of detecting target nucleic acids, such as pathogen-specific RNA, in a biological sample obtained from a subject, where the method comprises using one or more toehold switch sensors and an isothermal amplification step to detect the target nucleic acid. Methods specific for detecting and identify the presence of a virus such as Zika virus are also provided.

### CROSS-REFERENCE TO RELATED APPLICATIONS

This application is a divisional of U.S. application Ser. No. 17/849,250, filed Jun. 24, 2022, which is a continuation of U.S. application Ser. No. 16/303,937, filed Nov. 21, 2018, granted as U.S. Pat. No. 11,371,081 on Jun. 28, 2022, which is the U.S. National Phase of PCT/US2017/034545 filed May 25, 2017, which claims the benefit of and priority to U.S. Provisional Patent Application No. 62/341,221, filed on May 25, 2016, and U.S. Provisional Patent Application No. 62/403,778, filed on Oct. 4, 2016, each of which is incorporated by reference in its entirety.

### STATEMENT REGARDING FEDERALLY SPONSORED RESEARCH OR DEVELOPMENT

Not applicable.

### SEQUENCE LISTING

The content of the electronic sequence listing (112624.01472.xml; Size: 1,041,039 bytes; and Date of Creation: Sep. 6, 2024) is herein incorporated by reference in its entirety.

### BACKGROUND

Synthetic biology is an emerging discipline that has great potential to respond to global pandemics. The increasing ability of synthetic biologists to repurpose and engineer natural biological components for practical applications has led to new opportunities for molecular diagnostics.

In the case of viral outbreaks, standard serological approaches such as antibody detection have limited diagnostic value due to cross-reactivity in patients that have previously been infected by other flaviviruses circulating in the region. As a result, accurate diagnosis requires nucleic acid-based detection methods, such as PCR and isothermal nucleic acid amplification. However, such techniques are expensive, require technical expertise to run and interpret, and use equipment that is incompatible with use in remote and low-resource locations where surveillance and containment are critically needed. Accordingly, there remains a need in the art for improved methods and devices for rapid detection of target nucleic acids, including pathogen-specific nucleic acids for infection detection and for accurate strain identification.

### BRIEF SUMMARY

In a first aspect, provided herein is a method of detecting a target nucleic acid in a sample. In some cases, the method comprises or consists essentially of the steps of: (a) obtaining nucleic acid from a biological sample obtained from a subject; (b) amplifying the nucleic acid using isothermal amplification; (c) contacting the amplified nucleic acid to a toehold switch, wherein the toehold switch encodes a reporter protein and comprises one or more single-stranded toehold sequence domains that are complementary to a target nucleic acid or the reverse complement thereof, wherein the contacting occurs under conditions that allow translation of the coding domain in the presence of the target nucleic acid but not in the absence of the target nucleic acid, and detecting the reporter protein as an indicator that the target nucleic acid is present in the amplified nucleic acid of the subject; and (d) identifying the target nucleic acid as containing a target protospacer adjacent motif (PAM), wherein identifying comprises: (i) amplifying nucleic acid obtained from the biological sample using a reverse primer designed to append the trigger sequence of one or more toehold switch sequence domains; (ii) contacting the amplified nucleic acid of (i) to CRISPR/Cas under conditions that allow for sequence-specific cleavage of the contacted nucleic acid by CRISPR/Cas when the target PAM is present in the amplified nucleic acid; and (iii) detecting activation of the toehold switch, wherein activation does not occur in the event of CRISPR/Cas-mediated sequence-specific cleavage, thereby indicating the presence of the target PAM. The toehold switch can comprise one or more single-stranded toehold sequence domains, a fully or partially double-stranded stem domain comprising an initiation codon, a loop domain comprising a ribosome binding site, and a coding domain. The toehold and stem domains can be complementary in sequence to a naturally occurring RNA. The loop domain can be complementary in sequence to a non-naturally occurring RNA. The target nucleic acid can be an RNA specific to a pathogen. The pathogen is selected from the group consisting of a virus, bacterium, fungus, and parasite. In some cases, the pathogen is a virus. The virus can be Zika virus. The virus can an American Zika variant (GenBank: KU312312). The virus strain can be an African Zika variant (GenBank: KF268950). The toehold switch can comprise an E. coli lacZ gene encoding β-galactosidase. Detecting activation of the one or more toehold switch sensors can comprise performing a LacZ-based colorimetric assay. Isothermal amplification can be selected from the group consisting of NASBA (nucleic acid sequence-based amplification), loop-mediated isothermal amplification (LAMP), recombinase polymerase amplification (RPA), and helicase-dependent amplification (HDA). The biological sample can be selected from the group consisting of blood, serum, urine, saliva, tissue, cell, and organ, or a fraction or portion thereof.

In another aspect, provided herein is a method of detecting a target nucleic acid in a sample. In some cases, the method comprises or consists essentially of: (a) obtaining RNA from a biological sample obtained from a subject; (b) amplifying the RNA using isothermal amplification; (c) contacting the amplified RNA to a toehold switch, wherein the toehold switch encodes a reporter protein and comprises one or more single-stranded toehold sequence domains that are complementary to a target RNA or the reverse complement thereof, wherein the contacting occurs under conditions that allow translation of the coding domain in the presence of the target RNA but not in the absence of the target RNA, and detecting the reporter protein as an indicator that the target RNA is present in the amplified RNA of the subject; and (d) identifying the target RNA as containing a target protospacer adjacent motif (PAM), wherein identifying comprises: (i) amplifying RNA obtained from the biological sample using a reverse primer designed to append the trigger sequence of one or more toehold switch sequence domains; (ii) contacting the amplified RNA of (i) to CRISPR/Cas under conditions that allow for sequence-specific cleavage of the contacted RNA by CRISPR/Cas when the target PAM is present in the amplified RNA; and (iii) detecting activation of the toehold switch, wherein activation does not occur in the event of CRISPR/Cas-mediated sequence-specific cleavage, thereby indicating the presence of the target nucleic acid. The toehold switch can comprise one or more single-stranded toehold sequence domains, a fully or partially double-stranded stem domain comprising an initiation codon, a loop domain comprising a ribosome binding site, and a coding domain. The toehold and stem domains can be complementary in sequence to a naturally occurring RNA. The loop domain can be complementary in sequence to a non-naturally occurring RNA. The target nucleic acid can be an RNA specific to a pathogen. The pathogen can be selected from the group consisting of a virus, bacterium, fungus, and parasite. In some cases, the pathogen is a virus. The virus can be Zika virus. The virus can be an American Zika variant (GenBank: KU312312). The virus can be an African Zika variant (GenBank: KF268950). The toehold switch can comprise an E. coli lacZ gene encoding B-galactosidase. Detecting activation of the one or more toehold switch sensors can comprise performing a LacZ-based colorimetric assay. Isothermal amplification can be selected from the group consisting of NASBA (nucleic acid sequence-based amplification), loop-mediated isothermal amplification (LAMP), recombinase polymerase amplification (RPA), and helicase-dependent amplification (HDA). The biological sample is selected from the group consisting of blood, serum, urine, saliva, tissue, cell, and organ, or a fraction or portion thereof.

In a further aspect, provided herein is a method of detecting presence of virus in a sample. The method can comprise or consist essentially of the steps of: (a) obtaining RNA from a biological sample obtained from a subject; (b) amplifying the RNA using isothermal amplification; (c) contacting the amplified RNA to a toehold switch, wherein the toehold switch encodes a reporter protein and comprises one or more single-stranded toehold sequence domains that are complementary to an endogenous virus RNA sequence or the reverse complement thereof, wherein the contacting occurs under conditions that allow translation of the coding domain in the presence of the endogenous virus RNA but not in the absence of the endogenous virus RNA, and detecting the reporter protein as an indicator that the endogenous virus RNA is present in the amplified RNA of the subject. The virus can be Zika virus. The toehold switch can comprise one or more Zika genome-specific single-stranded toehold sequence domains, a thermodynamically stable double-stranded stem domain, a loop domain comprising a ribosome binding site, and a coding domain. The loop domain can be complementary in sequence to a naturally occurring RNA. The loop domain can be complementary in sequence to a non-naturally occurring RNA. The loop domain can be 11 nucleotides or 12 nucleotides. The toehold switch can comprise an E. coli lacZ gene encoding β-galactosidase. Isothermal amplification can be selected from the group consisting of NASBA (nucleic acid sequence-based amplification), loop-mediated isothermal amplification (LAMP), recombinase polymerase amplification (RPA), and helicase-dependent amplification (HDA).

In another aspect, provided herein is a device for identifying a pathogen, comprising a preserved paper test article, wherein a methods described herein is performed using the preserved paper test article. The paper test article can be preserved by freeze-drying.

In another aspect, provided herein is a kit for detecting a pathogen, comprising one or more of a device as described herein and an electronic optical reader.

In a further aspect, provided herein is a method of genotyping a nucleic acid molecule. The method can comprise or consist essentially of contacting the nucleic acid molecule with: a programmable nuclease; and a sgRNA, wherein the combination of the nuclease and sgRNA can specifically bind to at least one sequence variant of the nucleic acid molecule; and detecting the presence or absence of a cut in the nucleic acid molecule generated by the nuclease. In some cases, the method further comprises a first step of performing reverse transcription on a RNA molecule and performing 2nd strand DNA synthesis with a toehold primer to generate the nucleic acid molecule; and wherein the detecting step comprises: transcribing an RNA from the nucleic acid molecule after contacting it with the nuclease, using a primer which initiates transcription from a location distal of the sequence variation site with respect to the location of the toehold primer sequence; and contacting a sensor with the RNA resulting from step a) and detecting the presence or absence of sensor activation; wherein the sensor is activated if the nuclease is not able to cut the nucleic acid molecule in step a). The presence of a cut can indicate that the nucleic acid molecule has a sequence variant to which the sgRNA and nuclease can specifically bind. The presence of a cut can indicate that the nucleic acid molecule has a sequence variant to which the nuclease specifically binds. The programmable nuclease can be Cas. The sequence variant can occur at a PAM site. The nucleic acid molecule can be of human, animal, prokaryotic, eukaryotic, pathogenic, or synthetic origin. The nucleic acid molecule can be of viral origin. The viral nucleic acid molecule can be a Zika virus nucleic acid molecule. The sequence variant being detected can differentiate at least one of the African, American, and Asian Zika strains from the others. The sequence variant can be selected from Table 2. The sequence variant being detected can differentiate the African and American Zika virus strains. The sequence variant can be the SNP occurring at site 7330 of the African (GenBank: KF268950) and American (GenBank: KU312312) Zika strains. The sgRNA can have the sequence of SEQ ID NO: 1.

In another aspect, provided herein is a composition comprising a sgRNA which can specifically bind to a sequence flanking at least one sequence variant selected from Table 2, wherein the sequence variation occurs at a CRISPR/Cas PAM binding site. The sgRNA can comprise SEQ ID NO: 1. The sgRNA can be selected from Table 2.

In a further aspect, provided herein is a composition comprising a CRISPR/Cas nuclease and a sgRNA that specifically binds to a sequence flanking at least one sequence variant occurring in a population. The population can be a viral population. The viral population can be a Zika virus population. The variant can be selected from Table 2. The sgRNA can be selected from Table 2. The sgRNA can comprise SEQ ID NO: 1.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### BRIEF DESCRIPTION OF THE DRAWINGS

The present invention will be better understood and features, aspects, and advantages other than those set forth above will become apparent when consideration is given to the following detailed description thereof. Such detailed description makes reference to the following drawings, wherein:

FIG. 1 presents an exemplary workflow for rapid prototyping of paper-based, biomolecular sensors. Using sequence information from online databases, primers for isothermal RNA amplification and toehold switch-based RNA sensors were designed in silico using purpose-built algorithms. Once synthesized, the resulting sequence-specific toehold sensors can be assembled and validated in less than 7 hours (hrs.). In under a day, validated sensors can be embedded into paper and freeze-dried along with a cell-free transcription and translation system to be deployed in the field as stable diagnostics. For the diagnostic test, extracted RNA is isothermally amplified via NASBA and used to rehydrate the freeze-dried paper sensors. The detection of the appropriate trigger RNA is indicated by a color change in the paper disc from yellow to purple.

FIGS. 2A-2D demonstrate rapid prototyping of 48 paper-based RNA toehold sensors for Zika virus detection. (A) Series A toehold switch sensor schematic. The sensor design from Green et al., Cell 159:925-939 (2014) was modified with a shortened 11-nucleotide (nt) loop sequence to reduce leakage of output gene expression. (B) Series B toehold switch sensor schematic. Based on the same Zika genomic region as the A series, these sensors include a 12-nt loop and lack the refolding domain. These modifications were made to further reduce LacZ reporter leakage in the OFF state. (C) Maximum fold change in the rate of LacZ production for the Series A Zika virus RNA sensors during the first 90 minutes (min) at 37° C. Fold change of LacZ production rate is determined from the slope of absorbance at 570 nm over time (sensor alone versus sensor with 3,000 nM RNA trigger). Sensors are ordered according to fold change. (D) Maximum fold change in the rate of LacZ production for the Series B Zika virus RNA sensors during the first 90 min at 37° C. Error bars represent SD from three replicates. Inset: average LacZ absorbance of the OFF states at 60 min indicates an overall reduction in LacZ reporter leakage for the Series B sensors. Error bars represent SD across the 24 sensors.

FIGS. 3A-3C demonstrate detection of femtomolar (fM) concentrations of Zika virus RNA fragments. (A) Sensitivity of six of the best performing Series A and B sensors without RNA amplification. Fold change is calculated from absorbance (570 nm) after 30 minutes at 37° C. Error bars represent SD from three replicates. (B) A schematic of NASBA (nucleic acid sequence based amplification)-mediated RNA amplification. (C) Zika RNA fragments diluted in water or 7% human serum were amplified using NASBA with input concentrations ranging from 30 pM down to 3 fM. A 1:7 dilution of the NASBA reaction in water was then used to rehydrate freeze-dried, paper-based reactions containing sensors 27B and 32B. Fold change is calculated as described in (A) after 30 minutes at 37° C.

FIGS. 4A-4F demonstrate sensor specificity and sensitivity. (A) Linear response of sensors 27B, 31B and 32B to corresponding RNA trigger at 0 nM, 3 nM, 30 nM and 300 nM. Each point represents the mean of triplicate data taken at 60 min. (B) Orthogonality of sensors 27B, 7A and 32B to treatments of 3000 nM of trigger RNA from each of the three sensors. The absorbance output (570 nm) of the sensors at each time point was converted to a ratio of the maximum absorbance of respective sensor at the 90 min time point and plotted as a heat map. Yellow indicates no sensor activation and purple indicates maximum sensor activation. (C) Reproducibility of NASBA reactions. Samples of Zika RNA in water or 7% human serum were amplified in three independent 2 hr. NASBA reactions. Each NASBA reaction was diluted 1:7 in water and used to rehydrate three freeze-dried, paper-based reactions containing sensor 27B for a total of nine replicates. Fold change was calculated from absorbance (570 nm) after 30 minutes at 37° C. Error bars represent SD from nine replicates for the 3 pM sample and three replicates for the 0 pM sample. (D) Effect of NASBA reaction time on sensitivity. Samples of Zika RNA in 7% human serum were amplified in NASBA reactions for 30, 60, and 90 minutes. Diluted NASBA reactions (1:7) were tested with sensor 32B. Fold change was calculated as above. Error bars represent SD of three replicates. (E) NASBA with freeze-dried reagents. Samples of Zika RNA in 7% human serum were amplified by NASBA reagents in the standard formulation and by reagents freeze-dried in-house. Fold change and error bars were calculated as above after 60 minutes. (F) Removing the 65° C. step from NASBA protocol. Samples of Zika RNA in 7% human serum incubated at 95° C. for two minutes, mimicking viral lysis, and then amplified by NASBA according to the standard procedure without the 65° C. step. Fold change and error bars were calculated as above after 60 minutes.

FIGS. 5A-5C present sequence alignments and RNA extraction optimization data. (A and B) Sequence alignments of Zika virus and Dengue virus genomic regions targeted by sensors (A) 27B (SEQ ID NOS 777 and 778, respectively) and (B) 32B (SEQ ID NOS 779 and 780, respectively). Red boxes indicates sequences targeted by the respective toehold switches, red and blue boxes indicate the NASBA-amplified regions, and the remaining sequence indicates natural flanking RNA sequences from each virus. The entire Zika 32 sequence shown here was cloned into lentivirus to make proxy Zika virus. (C) Effect of boiling time on RNA extraction. Lentivirus was packaged with the Zika virus RNA fragment corresponding to sensor 32B. Virus was diluted to 10 and 3 fM target RNA in 7% human serum. Twenty-five μL of virus was heated to 95° C. for 1 and 2 minutes. One μL was then used to initiate NASBA-mediated RNA amplification. A 1:7 dilution of 2 hours NASBA reactions in water was then used to rehydrate freeze-dried, paper-based reactions. Fold change was calculated from absorbance (570 nm) after 60 minutes at 37° C. Error bars represent SD of three replicates.

FIGS. 6A-6D present data collected during development of a field-ready diagnostic platform. (A) Sequence specificity of Zika virus sensors 27B and 32B. Sensors were challenged with 3,000 nM of RNA corresponding to target sequences from the Zika virus or the homologous region of the Dengue virus. Fold change is calculated from absorbance (570 nm) at 60 minutes after rehydration and incubation of freeze dried, paper-based reactions at 37° C. Error bars represent SD from three replicates. (B) Zika virus sensors 27B and 32B were tested for specificity using NASBA reaction products derived from 300 fM input RNA corresponding to target genomic regions of the Zika or Dengue viruses in 7% human serum. Fold change was calculated as in (A). (C) Using the portable electronic reader, time-course data were collected for Zika virus sensor 32B in the presence of RNA amplified from 1 fM or 3 fM inputs of trigger RNA in 7% human serum. To increase sensitivity, NASBA reactions were run for 2.5 hours. Graphs plot the relative absorbance of 570 nm wavelength light compared to background, which was collected every minute from freeze-dried, cell-free reactions embedded into paper. (D) Incorporating viral sample processing into the diagnostic workflow. Lentivirus was packaged with Zika RNA or homologous Dengue RNA fragments targeted by sensor 32B. Three femtomolar of virus was spiked into 7% human serum and heated to 95° C. for 2 minutes to extract viral RNA. The boiled lysate was used to initiate NASBA-mediated RNA amplification. A 1:7 dilution of the 2 hours NASBA reaction in water was then used to rehydrate freeze-dried paper-based reactions. Time-course data were collected on the portable electronic reader as in (C).

FIGS. 7A-7C present an exemplary portable electronic optical reader. (A) Line drawings used to cut the housing for the electronic reader from black acrylic using a laser cutter. (B) Image of the 16-reaction reader from the front. Chip containing paper-based sensors slides into the slot illuminated by the green light. Reader dimensions: 106 mm wide×116 mm deep×96 mm high. (C) Components and circuit design used to assemble the electronic optical reader.

FIGS. 8A-8E illustrate an exemplary protocol for strain differentiation at single-base resolution. (A) Schematic representation of NASBA-CRISPR Cleavage (NASBACC)-genotyping following a positive Zika diagnosis. A synthetic trigger sequence is appended to a NASBA-amplified RNA fragment through reverse transcription. The presence of a strain-specific PAM leads to the production of either truncated or full-length trigger RNA, which differentially activates a toehold switch (sensor H) (Pardee et al., 2014). (B) The probability that a non-biased single nucleotide polymorphism (SNP) between two strains can be discriminated by CRISPR/Cas9 is 48% (Table S4). Hence, genetic drift between the American and African or Asian strains, while relatively small (14.4% and 4.9% sequence dissimilarity, respectively), has created hundreds of strain-specific PAM sites. (C) A SNP between African (GenBank: KF268950) and American (GenBank: KU312312) strains at site 7330 (SEQ ID NOS 782 and 781, respectively) disrupts an existing PAM site, allowing for Cas9-mediated DNA cleavage only in the American strain. (D) Sensor 32B can distinguish between Dengue and Zika RNA sequences but cannot discriminate between American and African Zika strains. Paper discs containing sensor 32B were rehydrated with 300 nM trigger RNA corresponding to sequences from American-Zika, African-Zika, or Dengue. Colorimetric outputs: a purple color indicates the activation of LacZ expression from the toehold switch, and a yellow color indicates the toehold switch remained inactive. (E) NASBACC can discriminate between American- and African-lineages of Zika virus. Paper discs containing sensor H were rehydrated with a 1:10 dilution of NASBACC reactions initiated with 0.05 μl of a 300 nM RNA sample. In this case, an inactive toehold switch leads to a positive identification of the American Zika strain.

FIGS. 9A-9B present data from a CRISPR nuclease assay using fresh and freeze-dried reactions. (A) Sequence information and location of the gRNA used to target the lacZ gene (SEQ ID NOS 783-788, respectively, in order of appearance). Each sequence was selected for maximum activity using the Doench et al. scoring algorithm (Doench et al., 2014). (B) Gel showing the length of supercoiled versus cut DNA following the in vitro digestion of a lacZ-containing plasmid for fresh and freeze-dried reactions. Note that the activity of some gRNA/Cas9 combinations is improved under freeze-dried conditions.

FIGS. 10A-10D are graphs data from assays validating diagnostic workflow on live Zika virus samples. (A) Specificity of sensor 32B against purified genomic RNA. Sensor 32B was tested for specificity using NASBA reaction products performed on 30 fM RNA purified from Zika virus and three different Dengue virus serotypes. Fold change is calculated from absorbance (570 nm) at 60 minutes (min) after rehydration and incubation of freeze-dried, paper-based reactions at 37° C. Error bars represent SD from three replicates. (B) Detection of live Zika virus. Ten femtomolar (fM) of laboratory-cultured Zika virus was spiked into human serum (7%), heated to 95° C. for 2 min, and used to initiate NASBA-mediated RNA amplification. A 1:7 dilution of the 3 hour (hr.) NASBA reaction in water was then used to rehydrate freeze-dried, paper-based reactions. Time-course data were collected on the portable electronic reader. Graph plots the relative absorbance of 570 nm wavelength light compared to background. Error bars represent SD from three replicates. (C and D) Detection of Zika virus in viremic rhesus macaque plasma using sensors 27B and 32B. Plasma containing 2.8 fM of Zika virus was diluted 1:10 in nuclease free water, heated to 95° C. for 2 minutes, and used to initiate NASBA-mediated RNA amplification. 3 hr. NASBA reactions were monitored on the portable electronic reader as in (B).

FIG. 11 depicts a schematic of NASBA-CRISPR Cleavage (NASBACC).

FIG. 12 demonstrates that Cas9 without guide RNA does not interfere with NASBA.

FIG. 13 demonstrates that Cas9 with a gRNA targeting a site lacking a PAM site does not interfere with NASBA.

FIG. 14 depicts a graph of the effect of primer concentration on NASBACC.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

All publications, including but not limited to patents and patent applications, cited in this specification are herein incorporated by reference as though set forth in their entirety in the present application.

The methods and compositions provided herein are based at least in part on the Inventors' development of a diagnostic platform utilizing engineered biomolecular, nucleic acid-based sensors and CRISPR-based technology that permits rapid, specific, and low-cost detection of viral nucleic acids at clinically relevant concentrations. In particular, the inventors developed engineered biomolecular sensors for the specific detection of pathogen genomes such as viral RNA genomes.

Without being bound to any particular theory or mechanism of action, it is believed that the inventors addressed limitations in the practical deployment of nucleic acid-based molecular diagnostics by combining isothermal RNA amplification with toehold switch sensors on a freeze-dried, paper-based platform. By automating the amplification primer and sensor design process using in silico algorithms, the methods described herein provide clinically relevant sensitivity, discriminating between pathogen genotypes with single-base resolution.

Accordingly, in a first aspect, provided herein is a method of detecting a target nucleic acid in a biological sample obtained from a subject. As described herein, the method comprises or consists essentially of (a) obtaining nucleic acid (e.g., DNA, RNA) from a biological sample containing or suspected of containing a target nucleotide sequence; (b) amplifying the nucleic acid using a primer designed to hybridize to the target nucleotide sequence; (c) contacting the amplified nucleic acid to a toehold switch, where the riboregulator encodes a reporter protein and comprises one or more toehold sequence domains that are complementary to the target nucleotide sequence, where the contacting occurs under conditions that allow translation of the coding domain in the presence of the target nucleic acid but not in the absence of the target nucleic acid, and detecting the reporter protein as an indicator that the target nucleic acid is present in the amplified nucleic acid of the subject.

In certain embodiments, the target nucleotide sequence is a nucleic acid from a pathogen, where the biological sample contains or is suspected of containing the pathogen. Accordingly, the methods provided herein are useful to detect any pathogen or infectious agent. Pathogens and infectious agents may comprise viruses, (e.g., single stranded RNA viruses, single stranded DNA viruses, Zika virus, HIV, hepatitis A, B, and C virus, HSV, CMV EBV, HPV), parasites (e.g., protozoan and metazoan pathogens such as Plasmodia species, Leishmania species, Schistosoma species, Trypanosoma species), bacteria (e.g., Mycobacteria, in particular, M. tuberculosis, Salmonella, Streptococci, E. coli, Staphylococci), fungi (e.g., Candida species, Aspergillus species), Pneumocystis carinii, and prions. In certain embodiments, the pathogen is a virus, and the methods can be used to detect any virus. In other embodiments, the pathogens that are detected are bacteria, fungi, or parasites. An advantage of the methods and systems described herein is that they can be applied for the detection and identification of essentially any nucleic acid-containing organism. Accordingly, the pathogen or infectious agent can be virtually any pathogen or infectious agent for which genetic information (e.g., gene sequences) is available. In other cases, the target nucleic acid is human in origin. In such cases, the methods can be employed to detect one or more target nucleic acids in a biological sample such as a biological sample obtained for forensic analysis, for genotyping, and the like.

In such cases, the methods provided herein can further comprise identifying the pathogen detected in the biological sample. For example, the method can further comprise (i) amplifying RNA obtained from the biological sample; (ii) contacting the amplified RNA of (i) to a nuclease under conditions that allow for sequence-specific cleavage of the contacted RNA by the nuclease when a pathogen strain-specific protospacer adjacent motif (PAM) is present; and (iii) detecting activation of a toehold switch, where activation does not occur in the event of nuclease-mediated sequence-specific cleavage, thereby indicating the presence of the pathogen strain-specific PAM. In other cases, DNA is obtained from the biological sample and amplified as described above.

Other target nucleotide sequences include, without limitation, DNA or RNA sequences that can identify a species (e.g., ribosomal RNAs or DNAs); DNA or RNA sequences that are associated with a particular genetic condition (e.g., where the target comprises a single nucleotide polymorphism (SNP) for which PAM identification is advantageous, including, without limitation, BRCA1/BRCA2 mutations, cystic fibrosis, Duchenne muscular dystrophy, hemochromatosis); DNA or RNA sequences for identifying a particular person with high certainty (e.g., identifying a suspect in a criminal investigation; identifying a “high value target” in a military operation).

For forensic applications, the target nucleotide sequence can be a DNA or RNA sequence associated with one or more particular identifiable features (e.g., skin color, hair color, eye color). In such cases, a biological sample can be assayed to detect a target nucleic acid of an unknown subject or for comparison to samples from known individuals. For applications related to pathogen detection, detection of particular RNA sequences is advantageous for determining, for example, the life cycle stage of a pathogen associated with an infection. By way of example, particular target nucleic acids can be detected to detect the presence of malaria parasite

Plasmodium falciparum and to determine whether the parasite is in a life cycle phase in which it can reproduce and, thus, transmit infection. Other applications for which the methods provided herein include, without limitation, profiling species in an environment (e.g., water); profiling species in an human or animal microbiome; food safety applications (e.g., detecting the presence of a pathogenic species, determining or confirming food source/origin such as type of animal or crop plant); obtaining patient expression profiles (e.g., detecting expression of a gene or panel of genes (e.g., biomarkers) to monitor the patient's response to a therapeutic regimen, to select a therapeutic regimen suitable for the patient, or to detect exposure of the patient to a toxin or environmental agent that affects expression of the gene or panel of genes; and molecular encryption applications such as marking certain products (e.g., high value products) using nucleic acid barcodes.

The nucleic acid molecule can be, e.g., an RNA, a DNA, an mRNA, and/or a genomic nucleic acid. In some embodiments of any of the aspects, the nucleic acid molecule can be human, animal, prokaryotic, eukaryotic, or pathogenic in origin. In some embodiments of any of the aspects, the nucleic acid molecule can be of viral origin. Nucleic acids and/or other moieties of the invention may be isolated. As used herein, “isolated” means separate from at least some of the components with which it is usually associated whether it is derived from a naturally occurring source or made synthetically, in whole or in part.

Nucleic acids and/or other moieties of the invention may be purified. As used herein, purified means separate from the majority of other compounds or entities. A compound or moiety may be partially purified or substantially purified. Purity may be denoted by a weight by weight measure and may be determined using a variety of analytical techniques such as but not limited to mass spectrometry, HPLC, etc.

Biological samples appropriate for use according to the methods provided herein include, without limitation, blood, serum, urine, saliva, tissues, cells, and organs, or portions thereof.

Since the methods of the present invention provide single-base discrimination, the methods are particularly suited to distinguishing between genomes of a pathogen strain (e.g., to distinguish between pathogen strains) and/or identifying the presence of nucleic acids specific to a particular pathogen. As described herein, the methods incorporate isothermal RNA amplification and the sequence-specific nuclease activity of a CRISPR/Cas system. “Clustered Regularly Interspaced Palindromic Repeats (CRISPRs)/CRISPR associated (Cas)” systems have been employed for targeted genome editing applications across many species. CRISPR systems belong to different classes, with different repeat patterns, sets of genes, and species ranges. The number of Cas genes at a given CRISPR locus can vary between species. The terms “Cas gene” and “CRISPR-associated (Cas) gene” are used interchangeably herein. A comprehensive review of the Cas protein family is presented in Haft et al. (2005) Computational Biology, PLoS Comput. Biol. 1:e60 (doi:10.1371/journal.pcbi.0010060). At least 41 CRISPR-associated (Cas) gene families have been described.

Without being bound to any particular theory or mechanism of action, Cas enzymes recognize a strain-specific protospacer adjacent motif (PAM) sequence. In one embodiment, in the case of the enzyme Cas9 the PAM sequence is NGG, where N can be any DNA base. Thus, a single base mutation, such as one that changes the sequence AGG to AAG, abolishes the PAM site and prevents Cas nuclease-based cleavage. As used herein, the term “protospacer” refers to the portion of a crRNA (or sgRNA) that is complementary to the genomic DNA target sequence. Generally, protospacers are usually 20 nucleotides in length. Referring to FIG. 8, the methods provided herein can employ pathogen strain-specific “NGG” protospacer adjacent motif (PAM) sequences and isothermal RNA amplification using primers having specificity to the toehold switch domain. In such cases, the amplified DNA will undergo Cas-mediated cleavage only if the appropriate strain-specific PAM sequence is present. The truncated RNA, generated through transcription of the cleaved DNA product, is unable to activate the toehold switch. In the absence of the PAM sequence, the full-length RNA product containing the toehold switch domain is generated, allowing for nucleic acid-based sensor activation. Trigger RNA is only amplified from DNA that is not cut by Cas, thereby allowing for strain-specific detection using the toehold switch. With respect to distinguishing between Zika virus strains, analysis of the sequences of the American Zika variant (GenBank: KU312312) and an African Zika variant (GenBank: KF268950) revealed over 600 sites at which a PAM site was present in one strain and not the other. Since both viruses have genomes of ˜10.5 kb in length, PAM sites that can be used to identify viruses in a strain-specific manner occur approximately every 17 bases within the genomes of the two closely related strains and thus provide considerable opportunities for strain identification according to the methods provided herein.

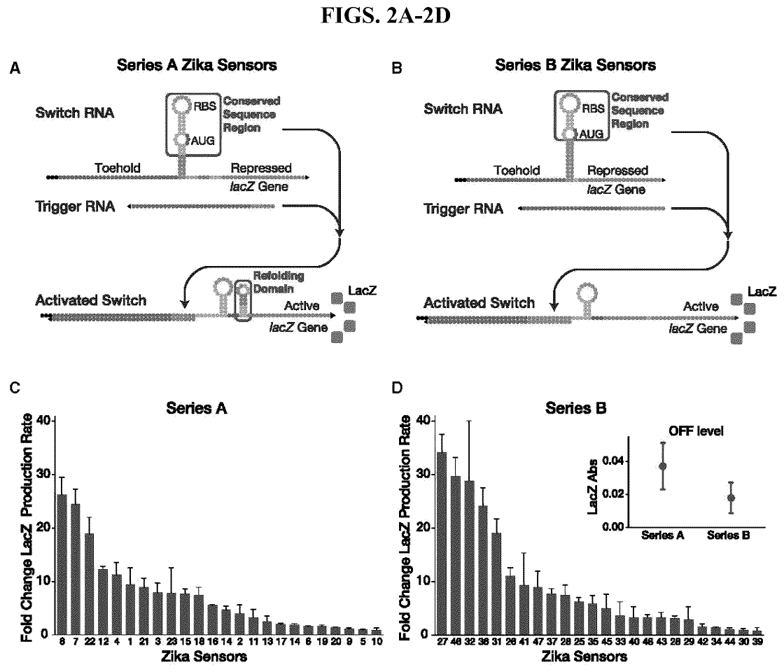

As used herein, the term “toehold switch” generally refers to a nucleic acid-based regulator of gene expression, configured to repress or activate translation of an open reading frame and thus production of a protein. Toehold switches, which are a type of prokaryotic riboregulator, activate gene expression in response to cognate RNAs with essentially arbitrary sequences. Gene regulation is achieved through the presence of a regulatory nucleic acid element (the cis-repressive RNA or crRNA) within the 5′ untranslated region (5′ UTR) of an mRNA molecule. The cis-repressive nucleic acid element (crRNA) forms a hairpin structure comprising a stem domain and a loop domain through complementary base pairing. The hairpin structure blocks access to the mRNA transcript by the ribosome, thereby preventing translation. In some embodiments, the stem domain of the hairpin structure sequesters the ribosome binding site (RBS). In some embodiments, including for example embodiments involving eukaryotic cells, the stem domain of the hairpin structure is positioned upstream of the start (or initiation) codon, within the 5′ UTR of an mRNA. In some cases, riboregulators comprise synthetic (engineered) molecules. In other cases, toehold switches comprise endogenous, naturally occurring RNAs or regions thereof. See, for example, U.S. 2015/0275203. The stem domain can be as small as 12 bps, but in some cases will be longer than 12 bps, including 13, 14, 15, 16, 17, 18, 19, 20, or more base pairs in length. In some cases, the loop domain is complementary to a naturally occurring RNA. In other cases, the loop domain is complementary to a non-naturally occurring RNA. The toehold domain can be 5, 6, 7, 8, 9, 10, 11, 12, 13, 14, 15, 16, 17, 18, 19, 20, or more nucleotides in length. Referring to FIGS. 2A and 2B, an exemplary toehold switch domain comprises an 11-nt or 12-nt loop domain.

The toehold switch further comprises a fully or partially double-stranded stem domain comprising an initiation codon, a loop domain comprising a ribosome binding site (RBS), and a coding domain. The unpaired region upstream of the RBS in a toehold switch can be shortened or lengthened to modulate protein output and, in turn, device dynamic range. In some cases, the toehold and stem domains are complementary in sequence to a naturally occurring RNA. In other cases, the sequence detected can also be the complement of the naturally occurring RNA. For example, after isothermal amplification, it is possible to transcribe the antisense of the RNA rather than the sense.

The toehold switch can further comprise a thermodynamically stable double-stranded stem domain, a loop domain comprising a ribosome binding site, and a coding domain. In some cases, the loop domain is complementary in sequence to a naturally occurring Zika virus RNA. In other cases, the loop domain is complementary in sequence to a non-naturally occurring RNA. Preferably, the loop domain is 11 nucleotides or 12 nucleotides in length. In some cases, the length of loop domains can be increased or decreased, for example, to alter reaction thermodynamics.

As shown in FIGS. 2A and 2B, the toehold switch can be operably linked to a reporter element (e.g., an E. coli lacZ reporter element encoding β-galactosidase) that is 3′ to the hairpin structure. As used herein, the term “operably linked” refers to a relationship between two nucleic acid sequences wherein the production or expression of one of the nucleic acid sequences is controlled by, regulated by, modulated by, etc., the other nucleic acid sequence. Reporter proteins appropriate for the methods provided herein include, without limitation, enzymatic reporters (e.g., β-galactosidase, alkaline phosphatase, DHFR, CAT), fluorescent or chemiluminescent reporters (e.g., GFP variants, mCherry, luciferase, e.g., luciferase derived from the firefly (Photinus pyralis) or the sea pansy (Renilla reniformis) and mutants thereof), etc.

Any isothermal amplification protocol can be used according to the methods provided herein. Exemplary types of isothermal amplification include, without limitation, nucleic acid sequence-based amplification (NASBA), loop-mediated isothermal amplification (LAMP), strand displacement amplification (SDA), helicase-dependent amplification (HDA), nicking enzyme amplification reaction (NEAR), signal mediated amplification of RNA technology (SMART), rolling circle amplification (RCA), isothermal multiple displacement amplification (IMDA), single primer isothermal amplification (SPIA), recombinase polymerase amplification (RPA), and polymerase spiral reaction (PSR, available at nature.com/articles/srep12723 on the World Wide Web). In some cases, a forward primer is used to introduce a T7 promoter site into the resulting DNA template to enable transcription of amplified RNA products via T7 RNA polymerase. In other cases, a reverse primer is used to add a trigger sequence of a toehold sequence domain.

As used herein, “nuclease” refers to an enzyme capable of cleaving the phosphodiester bonds between the nucleotide subunits of nucleic acids. Nucleases can be site-specific, i.e. site-specific nucleases cleave DNA bonds only after specifically binding to a particular sequence. Therefore, nucleases specific for a given target can be readily selected by one of skill in the art. Nucleases often cleave both strands of dsDNA molecule within several bases of each other, resulting in a double-stranded break (DSB). Exemplary nucleases include, but are not limited to Cas9; Cas13; meganucleases; TALENs; zinc finger nucleases; FokI cleavage domain; RNA-guided engineered nucleases; Cas-derived nucleases; homing endonucleases (e.g. I-AniI, I-CreI, and I-SceI) and the like. In some embodiments of any of the aspects, the nuclease is an endonuclease. As used herein, “endonuclease” refers to an enzyme capable of cleaving the phosphodiester bonds between the nucleotide subunits of nucleic acids within a polynucleotide, e.g., cleaving a phosphodiester bond that is not either the 5′ or 3′ most bond present in the polynucleotide. In other embodiments of any of the aspects, the nuclease is a meganuclease. As used herein, “meganuclease” refers to endonucleases, which have a large recognition sequence (e.g., dsDNA sequences of 12-40 bp). Due to the size of the recognition sequences, meganucleases are particularly specific. Meganuclease specificity can be engineered. In some embodiments of any of the aspects, the meganuclease can be a LAGLIDADG homing endonuclease (SEQ ID NO: 2).

In some embodiments, the nuclease can be an engineered nuclease. As used herein, the terms “engineered” and “genetically engineered” are used interchangeably and refer to the aspect of having been manipulated by the hand of man. For example, a nuclease is considered to be “engineered” when the sequence of the nuclease is manipulated by the hand of man to differ from the sequence of the nuclease as it exists in nature. As is common practice and is understood by those in the art, progeny and copies of an engineered polynucleotide and/or polypeptide are typically still referred to as “engineered” even though the actual manipulation was performed on a prior entity. Methods of engineering nucleases to achieve a desired sequence specificity are known in the art and are described, e.g., in Kim and Kim. Nature Reviews Genetics 2014 15:321-334; Kim et al. Genome Res. 2012 22:1327-1333; Belhaj et al. Plant Methods 2013 9:39; Urnov et al. Nat Rev Genet 2010 11:636-646; Bogdanove et al. Science 2011 333:1843-6; Jinek et al. Science 2012 337:816-821; Silva et al. Curr Gene Ther 2011 11:11-27; Ran et al. Cell 2013 154:1380-9; Carlson et al. PNAS 212 109:17382-7, Guerts et al. Science 2009 325:433-3; Takasu et al. Insect Biochem Mol Biol 2010 40:759-765; and Watanabe et al. Nat. Commun. 2012 3; each of which is incorporated by reference herein in its entirety.

In some embodiments, the nuclease is a programmable nuclease. As used herein “programmable nuclease” refers to a nuclease that has been engineered to create a double-stranded break (DSB) or nick at a nucleic acid sequence that the native nuclease would not act upon, e.g. the sequence specificity of the nuclease has been altered. As described herein, programmable nucleases can be used to genotype a nucleic acid and/or determine the sequence of a nucleic acid. In particular, programmable nucleases can differentiate between point mutations or SNPs, e.g., SNPs that occur in a PAM site. In one aspect of any of the embodiments, described herein is a method of genotyping a nucleic acid molecule, the method comprising: a) contacting the nucleic acid molecule with: a programmable nuclease; and a single guide (“sgRNA”) which can specifically bind to at least one sequence variant of the nucleic acid molecule; and b) detecting the presence or absence of a cut in the nucleic acid molecule generated by the nuclease. In one aspect of any of the embodiments, described herein is a method of genotyping a nucleic acid molecule, the method comprising: a) contacting the nucleic acid molecule with: a programmable nuclease; and a sgRNA wherein the combination of the nuclease and sgRNA can specifically bind to at least one sequence variant of the nucleic acid molecule; and b) detecting the presence or absence of a cut in the nucleic acid molecule generated by the nuclease. In some embodiments of any of the aspects, the presence of a cut indicates that the nucleic acid molecule has a sequence variant for which the sgRNA is specific. In some embodiments of any of the aspects, the presence of a cut indicates that the nucleic acid molecule has a sequence variant to which the nuclease specifically binds. In some embodiments of any of the aspects, the presence of a cut indicates that the nucleic acid molecule has a sequence variant for which the sgRNA is specific and has a sequence variant to which the nuclease specifically binds. In some embodiments of any of the aspects, the presence of a cut indicates that the nucleic acid molecule has a sequence variant to which the sgRNA and nuclease can specifically bind.

By way of non-limiting example, the programmable nuclease can be Cas9; Cas13, a Cas nickase mutant; TALEN; ZFNs; Cpf1; and/or SaCas9. In some embodiments of any of the aspects, the programmable nuclease is Cas9. In some embodiments, the programmable nuclease is Cas9. In some embodiments of any of the aspects, the programmable nuclease is S. pyogenes Cas9 or a variant thereof, e.g., New England Biolabs #M0386 (Ipswich, MA). When Cas9 nuclease (or Cas9-derived nuclease) is selected for use, the nuclease will generate a cut and/or nick where the guide RNA hybridizes to the nucleic acid molecule.

In order for a Cas nuclease to recognize and cleave a target nucleic acid molecule, a CRISPR targeting RNA (“crRNA”) and trans-activating crRNA (“tracrRNA”) must be present. crRNAs hybridize with tracrRNA to form a hybrid guide RNA (“gRNA”) which then associates with the Cas9 nuclease. Alternatively, the gRNA can be provided as a single contiguous RNA, and forms a single guide RNA (“sgRNA”). Once the sgRNA is complexed with Cas, the complex can bind to a target nucleic acid molecule. The sgRNA binds specifically to a complementary target sequence via a target-specific sequence in the crRNA portion (e.g., the spacer sequence), while Cas itself binds to a protospacer adjacent motif (CRISPR/Cas protospacer-adjacent motif; PAM). The Cas nuclease then mediates cleavage of the target nucleic acid to create a double-stranded break within the sequence bound by the sgRNA. Different Cas enzymes have different PAM recognition sequences. For example, S. pyogenes Cas9 requires a NGG PAM sequence while other CRISPR/Cas systems have been described in other prokaryotic species, which recognize a different PAM sequence (e.g., CCN, TCN, TTC, AWG, CC, NNAGNN, NGG, NGGNG).

In some embodiments of any of the aspects, the sgRNA is provided as a single continuous nucleic acid molecule. In some embodiments of any of the aspects, a hybrid gRNA is provided as a set of hybridized molecules, e.g., a crRNA and tracrRNA.

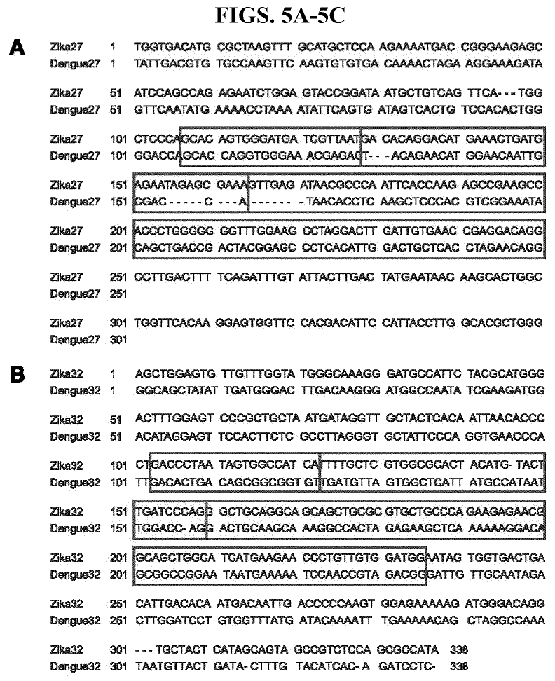

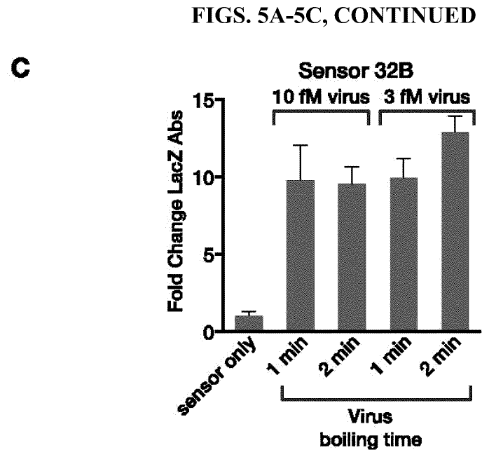

In embodiments in which the nuclease to which the amplified DNA or RNA is contacted is a Cas nuclease, a method of detecting a viral nucleic acid comprises or consists essentially of: (a) obtaining DNA or RNA from a biological sample obtained from a subject; (b) amplifying the DNA or RNA using a primer designed to append a trigger sequence of one or more toehold sequence domains; (c) contacting the amplified DNA or RNA to a toehold switch, where the riboregulator encodes a reporter protein and comprises one or more toehold sequence domains, where the contacting occurs under conditions that allow translation of the coding domain in the presence of the endogenous virus DNA or RNA but not in the absence of the endogenous virus DNA or RNA, and detecting the reporter protein as an indicator that the endogenous virus DNA or RNA is present in the amplified DNA or RNA of the subject; and (d) identifying the strain of virus, where identifying comprises: (i) amplifying DNA or RNA from the biological sample; (ii) contacting the amplified DNA or RNA of (i) to Cas (e.g., Cas9, Cas13) under conditions that allow for sequence-specific cleavage of the contacted RNA by Cas (e.g., Cas9, Cas13) when a virus strain-specific protospacer adjacent motif (PAM) is present; and (iii) detecting activation of the toehold switch, where activation does not occur in the event of Cas-mediated sequence-specific cleavage, thereby indicating the presence of the virus strain-specific PAM. For example, the methods provided herein can be used to distinguish between viral strains, e.g., where one strain comprises a PAM site while the second strain comprises a SNP that eliminates the PAM site, such an American Zika variant (GenBank: KU312312) and an African Zika variant (GenBank: KF268950), and also between other flavivirus strains. See, FIGS. 5A-5B. In such cases the toehold switch comprises one or more Zika genome-specific single-stranded toehold sequence domains. Exemplary sequences of toehold switches suitable for use for Zika RNA detection are provided in Table 8.

In some cases, the one or more toehold sequence domains are complementary to an endogenous virus DNA or RNA sequence. In such cases, where the toehold switch recognizes an endogenous RNA sequence, there is no requirement for a primer that appends a toehold sequence domain.

With respect to the amplification step, the target sequence for a toehold switch is in some cases added via an amplification primer for the NASBACC process. In other cases, a toehold switch that detects an endogenous pathogen DNA or RNA sequence is used.

In another aspect, provided herein is a method of detecting Zika virus in a sample. The methods can comprises, or consist essentially of, (a) obtaining RNA from a biological sample obtained from a subject; (b) amplifying the RNA using isothermal amplification; and (c) contacting the amplified RNA to a riboregulator, wherein the riboregulator encodes a reporter protein and comprises one or more toehold domains that is complementary to a Zika virus RNA, wherein the contacting occurs under conditions that allow translation of the coding domain in the presence of the Zika virus RNA but not in the absence of the Zika virus RNA, and detecting the reporter protein as an indicator that the Zika virus RNA is present in the amplified RNA of the subject.

In some cases, it may be advantageous to adapt the methods described herein for high-throughput, reproducible, and rapid detection, for example in a clinical setting. When riboregulator output is coupled to a reporter element, such as a LacZ reporter element, the riboregulator acts as a genetically encodable sensor and detectable probe for endogenous DNA or RNA (e.g., endogenous pathogen DNA, endogenous pathogen RNA) in a sample. For example, such toehold switches can be provided in a device configured for rapid, reproducible detection in a clinical setting. In some cases, the device comprises a preserved paper test article, upon which any step(s) of the method provided herein can be performed. In preferred embodiments, the paper test article is preserved by freeze-drying. The reporter element can be a reporter protein, e.g., a polypeptide with an easily assayed enzymatic activity or detectable signal that is naturally absent from the host cell. Exemplary but non-limiting reporter proteins include lacZ, catalase, xylE, GFP, RFP, YFP, CFP, neomycin phosphotransferase, luciferase, mCherry, and derivatives or variants thereof. In some embodiments of any of the aspects, the reporter protein is suitable for use in a colorimetric assay. Examples of genes encoding fluorescent proteins that may be used in accordance with the invention include, without limitation, those proteins provided in U.S. Patent Application No. 2012/0003630 (see Table 59 therein), incorporated herein by reference.

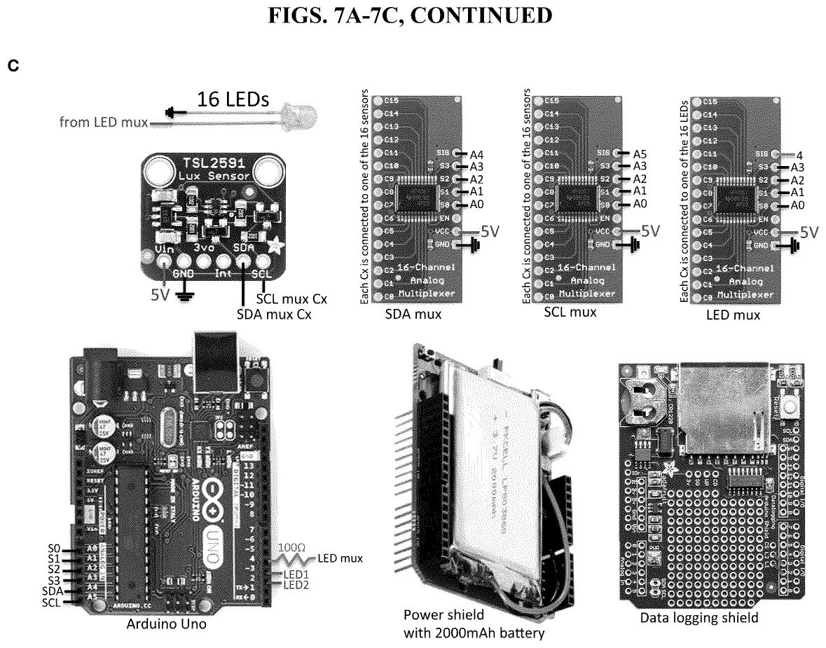

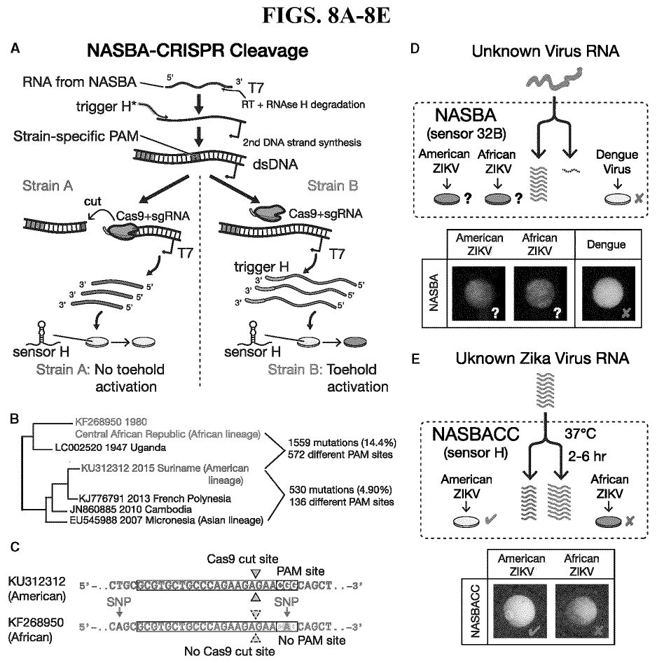

In some cases, the device is used with a portable electronic reader. In this manner, the electronic reader serves as companion technology that provides robust and quantitative measurements of device outputs. As shown in FIGS. 7A-7C, an exemplary electronic reader comprises readily available consumer components, open-source code, and laser-cut acrylic housing, and is powered by a rechargeable lithium ion battery. The electronic reader can further comprise an onboard data storage unit. In some cases, to achieve sensitive detection of toehold switch signal output, an acrylic chip that holds the freeze-dried, paper-based reactions is placed into the reader between an LED light source (570 nm) and electronic sensors. Using onboard electronics, samples can be read at a rate of 29 reads per minute. Accordingly, the portable electronic reader provides low-noise measurements of changes associated with the reporter element including changes in light transmission due to LacZ-mediated color change.

As used herein, “sequence variations” can refer to substitutions, insertions, deletions, duplications, and/or rearrangements. Sequence variations of a locus occurring in a population are referred to as alleles. Sequence variations can be present in (and therefore, detected in) the gDNA and/or mRNA of a gene. In some embodiments of any of the aspects, the sequence variation is a point mutation, e.g. a single nucleotide polymorphism (SNP). As used herein, a “point mutation” refers to the identity of the nucleotide present at a site of a mutation in the mutant copy of a genomic locus (including insertions and deletions), i.e., it refers to an alteration in the sequence of a nucleotide at a single base position from the wild type sequence. A SNP (single nucleotide polymorphism) is one type of point mutation that occurs at the same genomic locus between different individual subjects or entities in a population or different strains in a species. SNPs can be allelic. At least four alleles of a SNP locus are possible, although SNPs that vary only between two nucleotides at the target site are not uncommon.

In some embodiments of any of the aspects, the target nucleic acid is a Zika virus nucleic acid molecule, e.g., a Zika virus genomic molecule or a molecule transcribed from the Zika virus genome.

The methods described herein can permit identification of the species of virus present in a sample (e.g., a sample obtained from a subject), and/or permit identification of the strain of a virus present in a sample based upon sequence variations found between species and/or strains. Such information can be used to direct treatment, e.g., different strains of Zika virus are known to cause different symptoms and secondary conditions at varying frequencies. In some embodiments of any of the aspects, the sequence variant being detected differentiates at least one of the African, American, and Asian Zika strains from the others. Exemplary sequence variants that differentiate these strains are provided in Table 5.

In certain embodiments, provided herein is a method for genotyping a nucleic acid molecule. The method can comprise or consist essentially of contacting the nucleic acid molecule with a programmable nuclease and a sgRNA, where the combination of the nuclease and sgRNA can specifically bind to at least one sequence variant of the nucleic acid molecule; and detecting the presence or absence of a cut in the nucleic acid molecule generated by the nuclease. In some cases, the method further comprises a first step of performing reverse transcription on a RNA molecule and performing 2nd strand DNA synthesis with a toehold primer to generate the nucleic acid molecule. In such cases, the detecting step comprises: (i) transcribing an RNA from the nucleic acid molecule after contacting it with the nuclease, using a primer which initiates transcription from a location distal of the sequence variation site with respect to the location of the toehold primer sequence; and (ii) contacting a sensor with the RNA resulting from step (a) and detecting the presence or absence of sensor activation; wherein the sensor is activated if the nuclease is not able to cut the nucleic acid molecule in step (a). As used herein, the term “toehold primer” refers to an oligonucleotide primer configured to add a detectable tag or label sequence, where the tag or label sequence is detectable by a downstream nucleic acid sensor.

Primers and sgRNAs can readily be designed for a given variant according to the principles described herein. Cas9 selectively cleaves DNA only in the presence of an NGG protospacer adjacent motif (PAM). As demonstrated herein, e.g. in Example 1, numerous strain-specific PAM sites exist. The reverse transcription primer is designed to specifically bind near the selected PAM site such that reverse transcription proceeds towards the PAM site. The sgRNA and/or guide RNA is then designed to specifically bind to a sequence located between the PAM site and the sequence to which the reverse transcription primer specifically binds. Tools for designing primers and sgRNAs are known in the art. For example, a primer sequence can be selected to have a desired T_{M} (melting temperature) using any of a number of widely available algorithms (e.g., OLIGO™ (Molecular Biology Insights Inc. Colorado) primer design software and VENTRO NTI™ (Invitrogen, Inc. California) primer design software and programs available on the internet, including Primer3 and Oligo Calculator). Algorithms are also widely available for sgRNA design (e.g., several online tools (e.g., The Broad Institute's sgRNA Design tool, CRISPR Design or CHOPCHOP, which are available on the internet). Methods of making primers and other nucleic acid sequences (e.g., oligonucleotides, sgRNAs) are well known in the art, and numerous commercial sources offer oligonucleotide synthesis services suitable for providing molecules according to the methods and compositions described herein, e.g. INVITROGEN™ Custom DNA Oligos; Life Technologies; Grand Island, NY or custom DNA Oligos from IDT; Coralville, IA).

In some embodiments of any of the aspects, the sequence variant being detected differentiates the African and American Zika virus strains. In some embodiments of any of the aspects, the sequence variant is the SNP occurring at site 7330 of the African (GenBank: KF268950) and American (GenBank: KU312312) Zika strains. In some embodiments of any of the aspects, the sgRNA has the sequence of SEQ ID NO: 1. In some embodiments of any of the aspects, the method differentiates the African and American Zika virus strains by detecting the presence or absence of the SNP occurring at site 7330 of the African (GenBank: KF268950) and American (GenBank: KU312312) Zika strains, and the sgRNA has the sequence of SEQ ID NO: 1.

### Articles of Manufacture

In another aspect, the present invention provides articles of manufacture useful for detecting a virus or identifying a virus strain. In preferred embodiments, the article of manufacture is a kit for detecting a virus, where the kit comprises a plurality of preserved paper test articles and an electronic optical reader. Optionally, a kit can further include instructions for performing the virus detection and/or strain identification methods provided herein.

In some aspects of any of the embodiments, described herein is a composition comprising a Cas nuclease and a sgRNA which can specifically bind to at least one sequence variant occurring in a population. In some aspects of any of the embodiments, described herein is a composition comprising a Cas9 nuclease and a sgRNA which can specifically bind to at least one sequence variant occurring in a population, wherein the sequence variation occurs at the Cas9 PAM binding site.

In some aspects of any of the embodiments, described herein is a composition comprising a Cas nuclease and a sgRNA which can specifically bind to at least one sequence variant occurring in a viral population. In some aspects of any of the embodiments, described herein is a composition comprising a Cas9 nuclease and a sgRNA which can specifically bind to at least one sequence variant occurring in a viral population, wherein the sequence variation occurs at the Cas9 PAM binding site.

In some aspects of any of the embodiments, described herein is a composition comprising a Cas nuclease and a sgRNA which can specifically bind to at least one sequence variant occurring in a Zika virus population. In some aspects of any of the embodiments, described herein is a composition comprising a Cas9 nuclease and a sgRNA which can specifically bind to at least one sequence variant occurring in a Zika virus population, wherein the sequence variation occurs at the Cas9 PAM binding site.

In some aspects of any of the embodiments, described herein is a composition comprising a Cas nuclease and a sgRNA which can specifically bind to at least one sequence variant selected from Table 10. In some aspects of any of the embodiments, described herein is a composition comprising a Cas9 nuclease and a sgRNA which can specifically bind to at least one sequence variant selected from Table 5, wherein the sequence variation occurs at the Cas9 PAM binding site.

In some aspects of any of the embodiments, described herein is a composition comprising a Cas9 nuclease and a sgRNA comprising SEQ ID NO: 1. In some aspects of any of the embodiments, described herein is a composition comprising a Cas9 nuclease and a sgRNA comprising SEQ ID NO: 1, wherein the sequence variation occurs at the Cas9 PAM binding site. In some embodiments of any of the aspects, the sgRNA consists of SEQ ID NO: 1.

In some aspects of any of the embodiments, described herein is a sgRNA which can specifically bind to a sequence flanking at least one sequence variant selected from Table 5, wherein the sequence variation occurs at a Cas9 PAM binding site. In some aspects of any of the embodiments, described herein is a composition a sgRNA comprising SEQ ID NO:1. In some aspects of any of the embodiments, described herein is a composition a sgRNA consisting of SEQ ID NO:1.

Methods for sgRNA selection and design are described elsewhere herein. In some embodiments of any of the aspects, a sgRNA which can specifically bind to a sequence flanking a given sequence variant can comprise a 20 nt sequence complementary to a sequence found from 1-30 nucleotides from the sequence variation. In some embodiments of any of the aspects, a sgRNA which can specifically bind to a sequence flanking a given sequence variant can comprise a 20 nt sequence complementary to a sequence found from 1-25 nucleotides from the sequence variation. In some embodiments of any of the aspects, a sgRNA which can specifically bind to a sequence flanking a given sequence variant can comprise a 20 nt sequence complementary to a sequence found from 1-20 nucleotides from the sequence variation.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, “about” means within 5% of a stated concentration range or within 5% of a stated time frame.

It should also be understood that, unless clearly indicated to the contrary, in any methods claimed herein that include more than one step or act, the order of the steps or acts of the method is not necessarily limited to the order in which the steps or acts of the method are recited.

Having now described the invention, the same will be illustrated with reference to certain examples, which are included herein for illustration purposes only, and which are not intended to be limiting of the invention.

### EXAMPLES

Reference is now made to the following examples, which together with the above descriptions illustrate the invention in a non-limiting fashion.

### Example 1: Rapid, Low-Cost Detection of Zika Virus Using Programmable Biomolecular Components

### Materials and Methods

In Silico Sensor Design and DNA Synthesis: A set of 48 toehold switch sensors and corresponding NASBA primers were generated using an integrated in silico design algorithm.

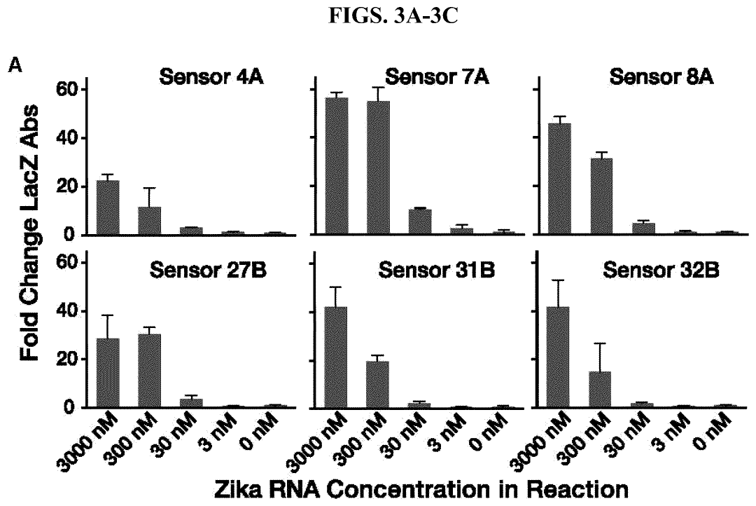

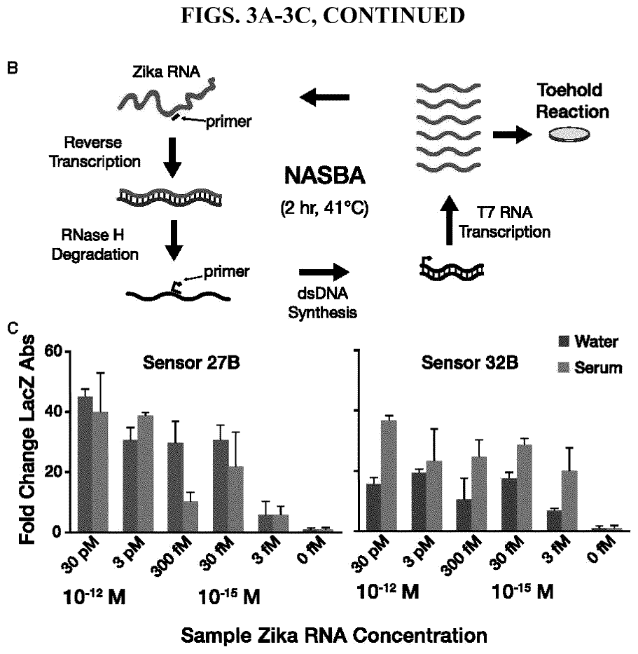

DNA Sensor Assembly: Toehold switch constructs were amplified from DNA templates (Integrated DNA Technologies) and ligated to the lacZ reporter gene via PCR. Plasmids were constructed for characterization of the top six toehold switches (FIG. 3A). The DNA templates were amplified using PCR and inserted into pET system parent plasmids (EMD Millipore) using Gibson assembly (Gibson et al., 2009) with 30 bp overlap regions. Plasmids for sensors 27B and 32B are available through Addgene (plasmid numbers: 75006-75011).

Cell-Free Reactions: Details of RNA sensor validation are described in Pardee et al. (2014). Briefly, amplified sensor DNA was column purified and tested on paper discs (2 mm) containing freeze-dried, cell-free reactions (NEB, PURExpress) in the presence or absence of trigger RNA coding for a complementary region of the Zika virus genome (128-178 nts). The cell-free reactions consisted of: NEB Solution A (40%) and B (30%), chlorophenol red-b-D-galactopyranoside (Sigma, 0.6 mg/ml), RNase inhibitor (Roche, 03335402001; 0.5%), and linear DNA constructs encoding the toehold sensors (0.33 nM). The paper discs (Whatman, 1442-042) were blocked in 5% BSA overnight prior to use. Trigger RNA was produced using T7 RNAP-based transcription (Epicenter ASF3257) from linear DNA templates. Paper-based reactions (1.8 μl) were incubated at 37° C. using either our companion electronic reader inside a humidified chamber or a plate reader (BioTek Neo). For the in-house reader, paper discs were placed into 2 mm holes in a removable acrylic chip; for the plate reader, paper discs were placed into black, clear bottom 384-well plates (Corning 3544).

NASBA: For NASBA reactions, the trigger elements (128-178 nts) were extended by 100 nts on the 5′ and 3′ ends with the relevant Zika genome sequence to provide suitable template RNAs. RNA amplicons were spiked into 7% human serum (Sigma H4522) where indicated. Reaction Buffer (Life Sciences NECB-24; 33.5%), Nucleotide Mix (Life Sciences NECN-24; 16.5%), RNase inhibitor (Roche, 03335402001; 0.5%), 12.5 mM of each NASBA primer (2%), nuclease free water (2.5%), and RNA amplicon (20%) were assembled at 4° C. and incubated at 65° C. for 2 min, followed by a 10 min incubation at 41° C. Enzyme Mix (Life Sciences NEC-1-24; 25%) was then added to the reaction (for a final volume of 5 μL), and the mixture was incubated at 41° C. for 2 hr. unless noted otherwise. For output reads with paper-based toeholds, the NASBA reactions were diluted 1:7 in water. See Table 2 for primer sequences.

Lentivirus Preparation and Processing: HEK293FT cells (Life Technologies, R70007) used for virus packaging were cultured in DMEM supplemented with 10% FBS, 1% penicillin-streptomycin, and 4 mM GlutaMAX (ThermoFisher Scientific). 12 hr. prior to transfection, 6.5×10⁶ cells were seeded in a 10 cm dish. 7.5 mg psPAX2, 2.5 mg pMD2.G, and 10 mg pSB700 modified to include a Zika or Dengue RNA fragment were transfected using the HeBS-CaCl₂ method. Media was changed 12 hr. post-transfection. 27 hr. after changing media, viral supernatant was harvested and filtered using a 0.45 mm syringe filter. Viral supernatant was then purified with ViraBind Lentivirus Purification Kit (Cell Biolabs VPK-104) and buffer exchanged into 1×PBS with Lenti-X Concentrator (Clontech, 631231). Viral RNA concentration was quantified using QuickTiter Lentivirus Quantification Kit (VPK-112). Virus samples were spiked into 7% human serum at a final volume of 25 μl. Samples were heated to 95° C. for 1 and 2 min and used as input to NASBA.

Zika Virus Preparation and Processing: 100 μl of Zika virus isolate (MR 766) was utilized for infection of 106 Vero cells in 4 ml of media (DMEM supplemented with 2% fetal calf serum [FCS] and penicillin-streptomycin). The supernatant was removed after 2 hr. of incubation at 37° C. and replaced with fresh media (DMEM, 10% FCS) for 48 hr. of infection. Cell debris was removed by centrifugation at 1,500 rcf for 10 min, and aliquots of the virus were stored at −80° C. until use. The virus was buffer exchanged into 1×PBS with Lenti-X Concentrator (Clontech, 631231). Viral RNA concentrations were determined from virus purified with the QIAamp Viral RNA Mini Kit (QIAGEN 52904) and confirmed with qRT-PCR. The titer of the Zika virus used was 6.7×10⁷ infectious units per milliliter (Lambeth et al., 2005). Virus samples were spiked into 7% human serum at a final volume of 30 μl. Samples were heated to 95° C. for 2 min and used as input to NASBA. NASBA primers were re-designed to accommodate the MR 766 strain sequence.

Dengue Orthogonality: Genomic RNA from three Dengue serotypes was purified using the QIAamp Viral RNA Mini Kit (QIAGEN 52904). Dengue 1 (GenBank: KM204119), Dengue 2 (GenBank: KM204118), Dengue 4 (GenBank: AF326573). NASBA reactions using the sensor 32B primer set were performed on 30 pM RNA for 2 hr. NASBA reactions were diluted 1:7 in water and used to rehydrate freeze-dried, paper-based reactions containing sensor 32B.

Electric Optical Reader: The portable device consists of four layers housed within a laser-cut acrylic box fastened together with metal screws and mounting brackets (FIG. 7; McMaster-Carr, 8505K14, 98164A061; Digi-Key, 36-621-ND). The top layer holds a multiplexer (Sparkfun, BOB-09056), solderable breadboard (Sparkfun, PRT-12702), friction lock connectors (Digi-Key, A31001-ND, A19473-ND) and 16 LEDs (Digi-Key, 754-1262-ND). The LEDs have a very narrow viewing angle and an emission of 570 nm to match the absorbance maximum of the chlorophenol red product from the LacZ reaction. The LEDs were placed in close proximity to the chip in the middle layer, which holds 16 paper disks within 2 mm apertures. The apertures prevented transmission of stray light and were coaxial with the LEDs in the top layer and the array of 16 TSL2591 sensors (Adafruit, 1980) in the third layer below, which also contained two solderable breadboards and connectors as above. The bottom layer contains the Arduino Uno with an attached Power Shield (Adafruit, 2708) connected to a rechargeable 2,000 mAh lithium ion battery (Adafruit, 2011) on which a datalogging shield (Adafruit, 1141) was stacked with connectors (Digi-Key, A30954-ND, A19476) and a 4 GB SD/MicroSD Card (Adafruit, 102). To prevent crosstalk between reads, reactions were read in series by sequentially activating each LED and sensor pair. The read frequency and pattern of the reader can be easily adjusted by modifying and uploading alternative sketches to the Arduino. The raw data (which is the median of 29 100 ms, 4283 gain reads per minute) was saved to the SD card along with the date and time of the run, integration time and gain settings. The data were processed with the MATLAB script and graphed in Prism. A diagram of the circuit and an overview of the laser cut parts can be found in FIG. 7, and laser cutting patterns, the Arduino sketch, and MATLAB script are in Appendix A.

Calculation of Fold Change: The calculation of fold change for plate reader data was done by first subtracting the background absorbance measured from paper-based reactions that did not contain sensor DNA or trigger RNA. These normalized values were smoothed to reduce measurement noise using a three-point average of the time point and the data collected 10 min before and after. The minimum value of each well was then adjusted to zero. For data presented in FIGS. 3, 6, and 10, fold change was calculated from these zero adjusted values by dividing the wells at each time point by the average signal from the corresponding sensor-alone control wells. For our initial sensor screen (FIG. 2), we used a more sensitive measure of fold change based on the difference in the rate of color change between control and RNA trigger wells. This was done by calculating the rate of change in normalized absorbance (570 nm) values using slope; where, at each 10 min time point, the rate was calculated using Sn=(Tₙ₊₁−Tₙ)/10, where T is the normalized data at a time point (Tₙ) and the time point 10 min later (Tₙ₊₁), and Sn is the slope reported for Tₙ. Fold change was then calculated as above. MATLAB script to analyze data collected on a plate reader is provided in Appendix A.

NASBA-CRISPR Cleavage (NASBACC): Reactions were performed in a 5 μl volume containing (NASBA buffer), 1 μl of a 250 nM Cas9 nuclease (NEB, M0386), and 250 nM purified gRNA (GeneArt precision gRNA synthesis kit, ThermoFisher Scientific, A29377) mix, 3 nM NASBACC primers, and 0.4 units of RNase inhibitor (NEB, M0314). The forward NASBACC primer is composed of the reverse complement of the trigger H sequence (5′-GTT TGA ATG AAT TGT AGG CTT GTT ATA GTT ATG TTT-3′ (SEQ ID NO: 3)) and the forward binding sequence of the (region 32) NASBA primers. The reverse NASBACC primer contains the T7 promoter sequence (5′-CTA ATA CGA CTC ACT ATA GG-3′ (SEQ ID NO: 4)) followed by the reverse binding sequence of the (region 32) NASBA primers. The assembled reaction was incubated at 37° C. for 2 to 6 hours. For toehold activation assay on freeze-dried paper, NASBACC reactions were diluted 1:10 in nuclease-free water.

Zika Virus Stock Production for Macaque Infection: ZIKV strain H/PF/2013 (GenBank accession number: KJ776791), originally isolated from a 51-year-old female in France returning from French Polynesia with a single round of amplification on Vero cells, was obtained from Xavier de Lamballerie (European Virus Archive, Marseille France). Virus stocks were prepared by inoculation onto a confluent monolayer of C6/36 mosquito cells. A single harvest of virus with a titer of 1.26×10⁶ PFU/ml for the Asian-lineage (equivalent to 1.43×10⁹ vRNA copies/ml) was used.

Viremic Plasma Processing: All Indian-origin rhesus macaque monkeys from which plasma was isolated were cared for by the staff at the Wisconsin National Primate Research Center (WNPRC) in accordance with the regulations and guidelines outlined in the Animal Welfare Act and the Guide for the Care and Use of Laboratory Animals and the recommendations of the Weatherall report. This study was approved by the University of Wisconsin-Madison Graduate School Institutional Animal Care and Use Committee (Animal Care and Use Protocol Number G005401). For all procedures (i.e., physical examination, virus inoculation, blood and swab collection), animals were anesthetized with an intramuscular dose of ketamine (10 ml/kg). Blood samples were obtained using a vacutainer system or needle and syringe from the femoral or saphenous vein. For processing, plasma was diluted 1:10 in nuclease free water, heated to 95° C. for 2 min, and immediately added to a NASBA reaction. NASBA was run for 3 hr.

Zika Virus Challenge of Macaques, Plasma Collection, and Processing: The virus stock was thawed, diluted in PBS to the appropriate concentration for each challenge, and loaded into a 1 ml syringe that was kept on ice until challenge. Animals were anesthetized as described above, and 1 ml of inocula was administered subcutaneously over the cranial dorsum. At the conclusion of the procedure, animals were closely monitored by veterinary and animal care staff for adverse reactions and signs of disease. Fresh plasma and PBMC were isolated from EDTA-treated whole blood by Ficoll density centrifugation at 1860 rcf for 30 min. The plasma layer was collected and centrifuged for an additional 8 min at 670 rcf to remove residual cells. The supernatant plasma was then filtered over a 0.45 μm syringe filter. Collected plasma was diluted 1:10 in nuclease free water. Diluted samples were heated to 95° C. for two minutes and immediately added to a NASBA reaction as described above. NASBA was run for three hours.

qRT-PCR to Determine Macaque Plasma Viral Loads: Viral RNA was extracted from 300 μl of plasma using the Viral Total Nucleic Acid Purification Kit (Promega) on a Maxwell 16 MDx instrument. Viral RNA was quantified by qRT-PCR using the primers and probe designed by Lanciotti et al. (2008). The RT-PCR was performed using the SuperScript III Platinum one-step quantitative RT-PCR system (Invitrogen) on the LightCycler 480 instrument (Roche Diagnostics). Primers and probe were used at final concentrations of 600 nm and 100 nm, respectively, along with 150 ng random primers (Promega). Cycling conditions were as follows: 37° C. for 15 min, 50° C. for 30 min, and 95° C. for 2 min, followed by 50 cycles of 95° C. for 15 seconds and 60° C. for 1 min. Virus concentration was determined by interpolation onto an internal standard curve composed of seven 10-fold serial dilutions of a synthetic ZIKV RNA fragment based on the Asian lineage.

### Results

In Silico Toehold Switch Design: Toehold switch sensors are programmable synthetic riboregulators that control the translation of a gene via the binding of a trans-acting trigger RNA. The switches contain a hairpin structure that blocks gene translation in cis by sequestration of the ribosome binding site (RBS) and start codon. Upon a switch binding to a complementary trigger RNA, sequestration of the RBS and start codon is relieved, activating gene translation (FIGS. 2A-2B) (Green et al., 2014). To allow for colorimetric detection of trigger RNA sequences, the sensors can be designed to regulate translation of the enzyme LacZ, which mediates a color change by converting a yellow substrate (chlorophenol red-b-D-galactopyranoside) to a purple product (chlorophenol red).

Toehold switch sensors for sequence-based detection of Zika virus were generated using an expanded version of the previously developed in silico design algorithm (Green et al., 2014). The modified algorithm screened the genome of the Zika strain prevalent in the Americas (Genbank: KU312312) for regions compatible with RNA amplification and toehold switch activation. The selected Zika genome regions were then computationally filtered to eliminate potential homology to the human transcriptome and to a panel of related viruses, including Dengue and Chikungunya. A total of 24 unique regions of the Zika genome compatible with downstream sensing efforts were identified.

Two toehold switches, each utilizing a different design scheme, were designed for each region, resulting in a total of 48 sensors. The first design scheme, termed the A series, utilizes a modification to the original toehold switch (Green et al., 2014) that reduces the size of the loop domain from 18 nts to 11 nts (FIG. 2A) to discourage loop-mediated docking of the ribosome and therefore reduce leakage in the OFF state. The second design scheme, termed the B series, features a 12-nt loop and incorporates a more thermodynamically stable stem in order to lower OFF state gene expression (FIG. 2B).

Rapid In Vitro Sensor Assembly and Screening: In vitro assembly and initial screening of all 48 sensors took place in a 7 hr. time period, with low costs associated with sensor development (DNA input $20 USD/sensor) and testing ($0.10-$1/test). All 48 sensors and 24 targeted genomic regions were assembled in-house using in vitro protocols. Toehold switches were constructed by ligating the sensors (˜130 nt) to a LacZ reporter element in a single 2 hr. PCR-based step. Sensor performance screening to assess each sensor against its respective trigger RNA element (Zika genome fragment) was completed using low volume, cell-free transcription and translation reactions on paper. We found that 25 (52%) of the 48 sensors produce a fold change of five or greater in the presence of the appropriate trigger element (128-178 nucleotide regions of the Zika genome; FIGS. 2C, 2D). The top-ranked sensors exhibited activation as high as 34-fold over sensor alone (sensor 27B) and were activated in as quickly as 20 minutes after incubation at 37° C. (sensors 7A and 8A). For all sensors, maximum fold change occurred within the first 90 min. Averaging the LacZ output from sensors not exposed to trigger RNA confirmed that the low background design of the series B toehold switch sensors successfully reduced signal leakage (FIG. 2D, inset).

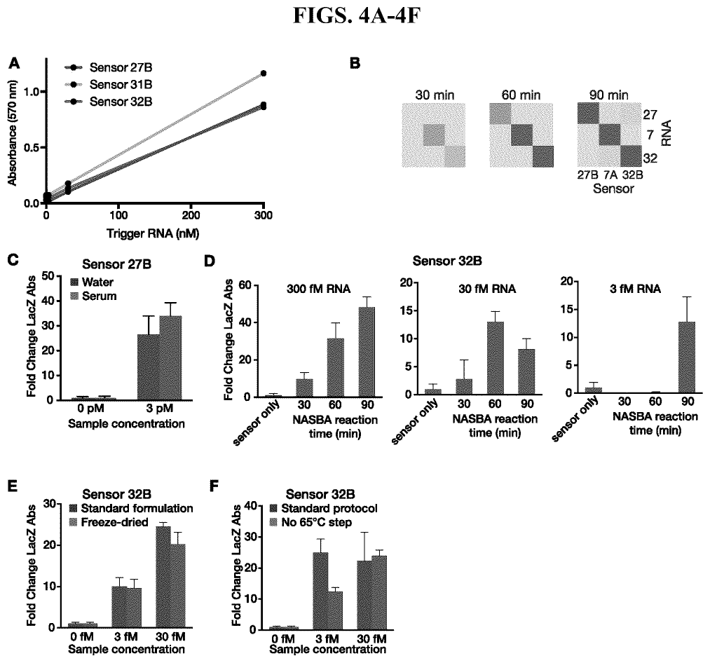

Assessing and Improving Zika Virus Sensor Sensitivity: We selected top performing sensors from both the A and B series for trigger RNA titration experiments and found that all chosen sensors were activated with as little as 30 nM of trigger RNA (FIG. 3A). The sensors displayed a linear response to RNA concentration, providing semi-quantitative information on input trigger RNA values (FIG. 4A). Additionally, our top three sensors were highly orthogonal to each other when challenged with a high dose of trigger RNA from off-target Zika sequences (3,000 nM) (FIG. 4B).

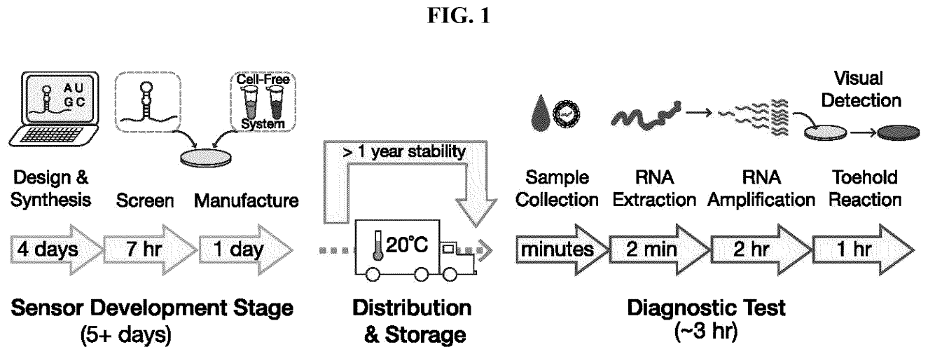

Though the sensors displayed specificity for their respective Zika RNA trigger, they were unable to detect clinically relevant RNA concentrations. Zika viral loads have been documented as high as 202×10⁶ copies/ml (365 fM) in urine (Gourinat et al., 2015). However, viral loads in saliva and serum are reportedly even lower, with 3×10⁶ copies/ml (4.9 fM) (Barzon et al., 2016) documented in patient saliva and 2.5×10⁶ copies/ml (4.1 fM) (Zika Experimental Science Team, 2016) and 7.2×10⁵ copies/ml (1.2 fM) (Lanciotti et al., 2008) in primate and patient serum, respectively. Accordingly, to increase the sensitivity of our diagnostic platform, we incorporated an isothermal RNA amplification technique known as NASBA (nucleic acid sequence-based amplification) into our workflow (FIG. 1).

NASBA is a promising candidate for use with our diagnostic scheme because it is known to be extremely sensitive and has a proven track record in field-based diagnostic applications (Cordray and Richards-Kortum, 2012). The amplification process begins with reverse transcription of a target RNA that is mediated by a sequence-specific reverse primer to create an RNA/DNA duplex. RNase H then degrades the RNA template, allowing a forward primer containing the T7 promoter to bind and initiate elongation of the complementary strand, generating a double-stranded DNA product. T7-mediated transcription of the DNA template then creates copies of the target RNA sequence. Importantly, each new target RNA can be detected by the toehold switch sensors and also serve as starting material for further amplification cycles. NASBA requires an initial heating step (65° C.), followed by isothermal amplification at 41° C. (FIG. 3B) (Guatelli et al., 1990).

NASBA was performed on trigger RNA corresponding to Zika genomic regions for sensors 27B and 32B. Trigger RNAs were spiked into either water or human serum (7%) to more closely mimic clinical samples. NASBA reactions were run for 2 hr. and then applied to freeze-dried, paper-based sensors. We saw detection with Zika sensors from NASBA reactions initiated with as little as 3 fM of trigger RNA (FIG. 3C), a value within the range of reported patient viral loads. Zika sensor detection of NASBA-amplified trigger RNA proved to be reliable on samples spiked into either serum or water (FIG. 4C). Additionally, for reactions initialized with high concentrations of trigger RNA (>300 fM), NASBA reaction times could be reduced to as little as 30 minutes (FIG. 4D). NASBA reagents are compatible with freeze-drying (FIG. 4E) and could therefore be easily deployed and utilized alongside our paper-based sensors. We also demonstrated that NASBA can be run in the absence of the initial heating step (65° C.) (FIG. 4F), further reducing the technical and power requirements for deployment.

Field-Ready Diagnostic Platform: To move our experiments toward conditions more representative of those found in clinics worldwide, we focused on three key efforts: (1) testing sensor specificity against related viruses that share clinical symptoms, partial homology, and geographic range with Zika virus; (2) building a second-generation portable, battery-powered reader to provide lab-quality results in low resource environments; and (3) developing a low-cost and tractable method for viral RNA extraction.

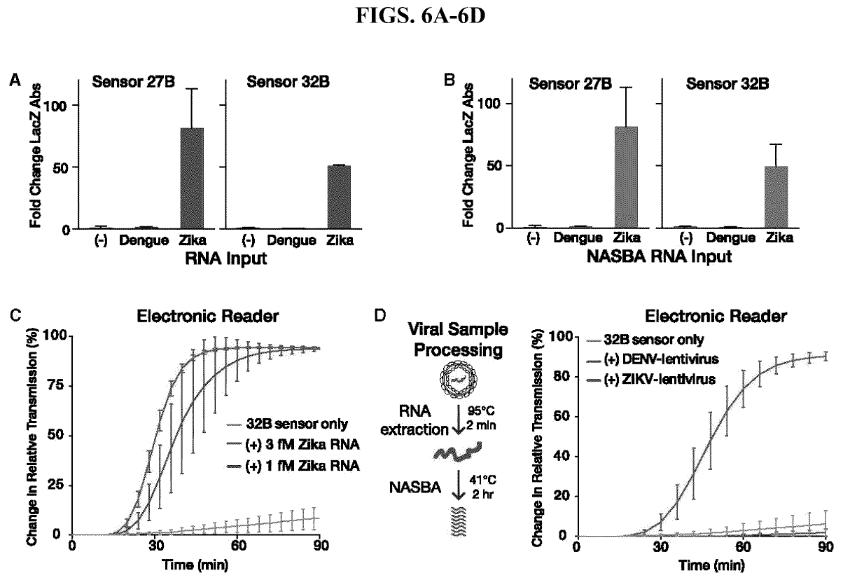

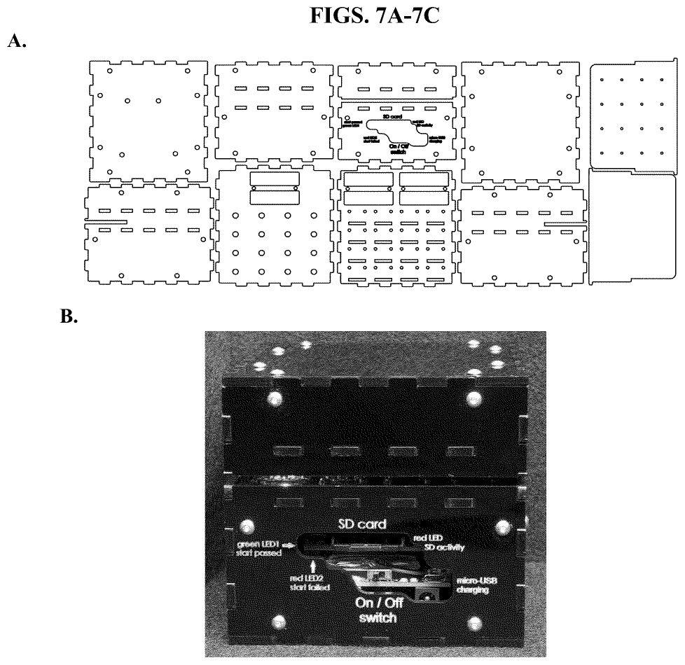

Although our sensor design algorithm screened for Zika genomic sequences that are mostly distinct from those of related viruses, the targeted Zika sequences do share substantial similarity (51%-59%) with their Dengue virus counterparts (FIGS. 5A-5B). To test the Zika sensors for possible cross-reactivity, we exposed the sensors to regions of the Dengue genome that share a degree of homology with regions targeted in the Zika genome. Sensors 27B and 32B were treated with high concentrations of RNA amplicons (3,000 nM) from either Zika or Dengue genomic regions. As seen in FIG. 6A, Dengue RNA sequences failed to activate the toehold switch sensors. We also tested our NASBA primer sets for specificity to their targeted Zika sequences by applying the NASBA-mediated amplification and paper-based detection scheme to 300 fM inputs of the Dengue and Zika RNA in human serum (7%). Again, no response to the Dengue RNA sequences was observed, demonstrating robust sequence specificity in our amplification and detection scheme (FIG. 6B).

As part of our efforts to advance the paper-based sensor platform toward field-ready diagnostics, we designed a second generation portable electronic reader to serve as an accessible, low-cost companion technology that provides robust and quantitative measurements of sensor outputs. The electronic reader was assembled using readily available consumer components, open-source code, and laser-cut acrylic housing, with a total cost of just under $250 (FIG. 7 and Table 3). The reader is powered by a lithium ion battery (18.5 hr.) that can be re-charged via micro USB and houses onboard data storage (4 GB) to resolve the need for an attached laptop during diagnostic reads (Pardee et al., 2014). To achieve sensitive detection of toehold switch signal output, an acrylic chip that holds the freeze-dried, paper-based reactions is placed into the reader between an LED light source (570 nm) and electronic sensors (FIG. 7B). Using onboard electronics, each sample is read 29 times per minute, providing low-noise measurements of changes in light transmission due to LacZ-mediated color change.

To demonstrate the utility of the companion reader, we monitored detection of 1 fM and 3 fM of Zika RNA amplicons that had been amplified in NASBA reactions for 2.5 hr. The reader detected significant signal from both samples, which are within the reported range of Zika virus in patient serum (1.2 fM) and urine (365 fM) (Gourinat et al., 2015; Lanciotti et al., 2008), after just over 20 min (FIG. 6C).

Our next challenge was to develop a technique to release RNA from the viral capsid using simple methodology compatible with low-resource environments. To this end, we tested the efficacy of boiling viral samples to break down the capsid. For initial development, we engineered lentivirus, which is also an RNA virus, to encapsulate the regions of either the Zika or Dengue genomes that correspond to the sensor 32B target sequence (FIG. 5B). These proxy Zika and Dengue viruses were spiked into human serum (7%) at a final concentration of 3 fM and heated to 95° C. for either 1 or 2 min. The resulting lysates were then immediately used to initiate NASBA reactions, in order to simulate what might be recovered from a patient sample. Boiling the viral samples for one minute was sufficient to release detectable amounts of RNA in our amplification and toehold switch detection scheme (FIG. 5C). NASBA reactions from 2 min boiled samples were also monitored for sensor activation on the portable electronic reader. We detected strong sensor activation in less than 30 minutes from 3 fM of lentivirus carrying Zika RNA. We were also able to demonstrate clear discrimination between lentiviruses containing Zika and Dengue RNA sequences (FIG. 6D).

NASBA-CRISPR Cleavage Assay to Discriminate Between Zika Strains: During epidemic outbreaks, it is often valuable to monitor pathogen lineage and geographic spread. In some cases, genetic variants maybe responsible for different clinical manifestations of infection. For example, the Zika strain found in Brazil has been uniquely connected with higher incidences of fetal microcephaly and Guillain-Barre syndrome (Calvet et al., 2016; Mlakar et al., 2016). To allow for strain-specific detection and tracking, we developed an assay that provides single-base discrimination in a manner that is compatible with our freeze-dried sensor platform. Our assay, which we term NASBA-CRISPR Cleavage (NASBACC), leverages the sequence-specific nuclease activity of CRISPR/Cas9 to discriminate between viral lineages (FIG. 8A). To do this, NASBACC exploits the ability of Cas9 to selectively cleave DNA only in the presence of an NGG protospacer adjacent motif (PAM). Since any non-biased mutation has a 48% probability of either creating a new PAM site or destroying an existing one (Table 4), there are many strain-specific PAM sites that can be used for lineage discrimination (FIGS. 8B-8C). In the NASBACC detection scheme, RNA sequences undergo NASBA amplification utilizing a reverse primer designed to append the trigger sequence of a synthetic toehold switch (sensor H, FIG. 8A) (Pardee et al., 2014). In the presence of the appropriate PAM sequence and guide RNA target site, the double-stranded DNA that is synthesized as part of the NASBA reaction undergoes Cas9-mediated cleavage, resulting in a truncated RNA product that is unable to activate the sensor H toehold switch. In the absence of the PAM sequence, the full-length RNA product containing the sensor H trigger sequence is generated, allowing for sensor H activation. Trigger RNA is only amplified from DNA that is not cut by Cas9, thereby allowing for strain-specific detection using toehold sensor H.

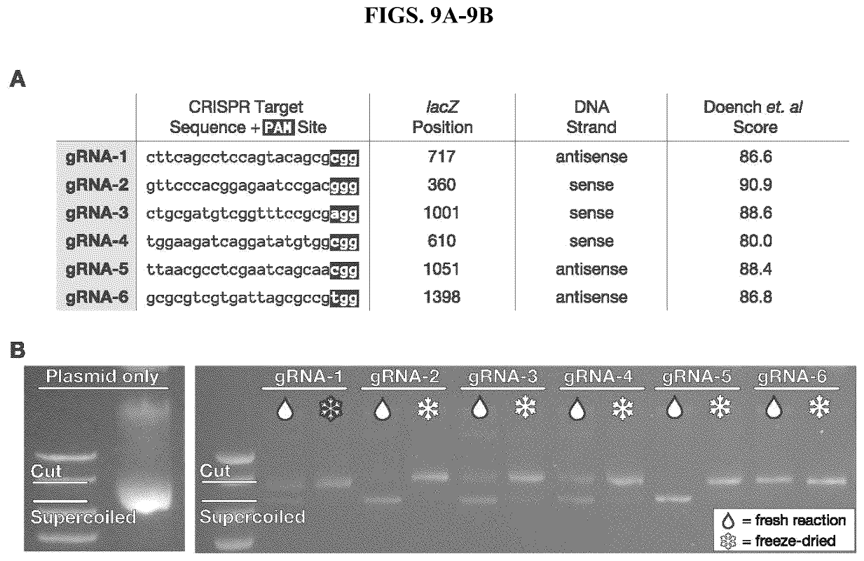

Using the paper-based system, sensor 32B was able to distinguish between Zika and Dengue RNA sequences. However, this sensor could not discriminate between the African (GenBank: KF268950) and American (GenBank: KU312312) Zika variants (FIG. 8D), a feature that may be useful in certain diagnostic applications. To address this, we applied our NASBACC detection scheme to discriminate between the African and American Zika strains. Due to a single-base difference in the trigger regions of these two strains, a PAM site only exists in the American-lineage sequence (FIG. 8C). Thus, only the American strain sequence was cleaved by Cas9, which led to amplification of truncated RNA that did not activate the sensor H toehold switch (FIG. 8E). Conversely, the African strain sequence does not contain the PAM site and was not cleaved by Cas9, which resulted in amplification of full-length RNA that activated the sensor H toehold switch. Incorporating NASBACC into our diagnostic workflow can provide precise genotypic information within a few hours. As with the other biomolecular elements of this workflow, Cas9 is compatible with lyophilization and could be used in the field (FIG. 9).

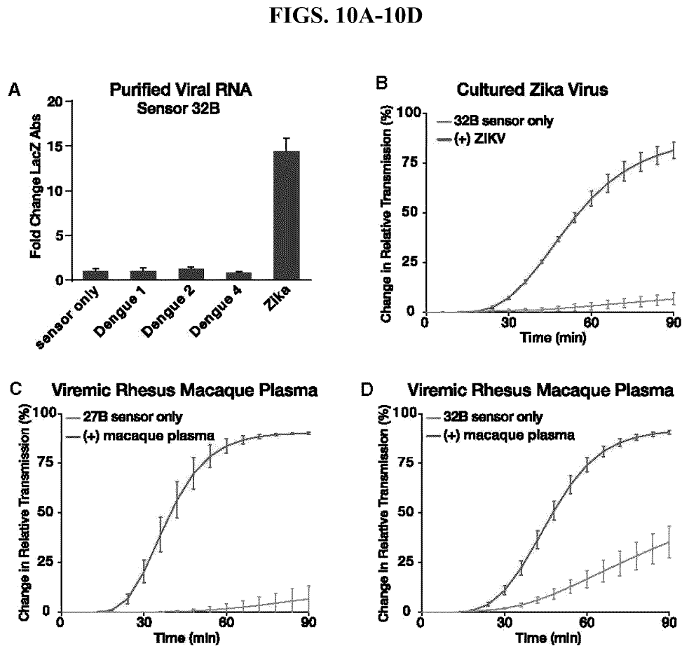

Diagnostic Workflow Validation with Active Zika Virus: We next sought to validate our sensor platform with live Zika virus. First, we verified that our amplification and detection scheme could successfully detect full-length genomic RNA purified from Zika virus (Uganda strain MR 766) (FIG. 10A). We designed new NASBA primers to accommodate sequence differences between the Uganda Zika strain (GenBank: AY632535) and the American Zika strain (GenBank: KU312312) that our sensors and primers had originally been designed to detect. Computational analysis suggested that Uganda-lineage Zika RNA would activate sensor 32B despite two base mismatches in the toehold region, and this was confirmed experimentally (FIG. 10A). We also demonstrated sensor orthogonality to full length genomic Dengue RNA isolated from three different Dengue serotypes using these methods (FIG. 10A).

Once we confirmed that the sensors behaved as expected on full-length genomic RNA, we sought to validate the sample preparation scheme and diagnostic workflow from start to finish. Active Zika virus was cultured in the laboratory and spiked into human serum (7%) at a final concentration of 10 fM, to mimic a clinical sample. The viral sample was then heated to 95° C. for 2 min, and the resulting lysate was subjected to NASBA amplification for three hours. Sensor activation from the NASBA-amplified viral sample was monitored on the portable electronic reader. We successfully detected activation of sensor 32B from a diagnostic workflow initiated with live Zika virus (FIG. 10B).

For the final validation of our system, we acquired and tested plasma samples from a viremic macaque infected with Zika virus (GenBank: KJ776791) (Zika Experimental Science Team, 2016). The macaque was found to have a plasma viral load of 1.7×10⁶ copies/ml (2.8 fM) by a standard qRT-PCR protocol, which was within the detection limits of our platform as tested on synthetic RNA amplicons (FIG. 6C). The viremic plasma was diluted 1:10 in water to reduce known inhibitory effects of plasma on downstream reactions and was then taken through our sample processing and diagnostic workflow. The sample was heated to 95° C. for 2 min and then amplified via NASBA for 3 hr. Paper-based reactions were monitored on the portable electronic reader and showed strong activation with both sensors 27B and 32B in less than 30 min (FIGS. 10C-10D).

In Silico Strategy for Toehold Switch Sensor and NASBA Primer Design. An integrated in silico strategy was developed for generating optimal NASBA primers and toehold switches for detection of Zika. Mirroring the procedure used for running the paper-based diagnostic assay, a set of optimal primers was initially generated for the NASBA reaction and then a series of toehold switch designs screened for activity on the RNA transcripts produced by NASBA was developed.

Identification of Optimal NASBA Primers for Zika Amplification. A set of potential primer pairs with favorable characteristics for NASBA reactions as described by Deiman et al. (Deiman et al., 2002) was generated. The Zika genome was analyzed to identify all potential forward and reverse priming sites that had the following characteristics:
GC content between 40-60%
Template hybridization regions of 20- to 24-nts and with DNA melting temperatures above 41° C.
No consecutive runs of four or more nucleotides
An A base at the final 3′ nucleotide
Minimal DNA primer internal secondary structure, including the T7 promoter region
Minimal DNA primer dimer formation probability
Higher GC content in the 6-nts at the 5′ end of the primer that hybridized to the template
Higher AT content in the 6-nts at the 3′ end of the primer

All subsequences from the Zika genome that did not satisfy requirements 1 to 4 were immediately eliminated from consideration. The remaining primers were then analyzed for characteristics 5-8 and the deviation from optimal 50% GC content, with each parameter converted into a numerical score. The forward NASBA primers, which append the T7 promoter site to NASBA DNA intermediate, were modeled with this T7 promoter site present. Accordingly, the prefix sequence AATTCTAATACGACTCACTATAGGGAGAAGG (SEQ ID NO: 5) (T7 promoter sequence underlined) was appended to the 5′ end of each forward primer. The resulting scores were combined and used to estimate the overall favorability of every potential primer in the Zika genome. Following this initial screen, the top 2% of all the potential primers were then compared to the rest of the Zika genome to determine the longest contiguous region of the primer that matched more than one site in the genome. This comparison provided a coarse check of primer specificity in advance of more detailed primer specificity screening conducted later in the design process. The length of this contiguous region for each primer was then incorporated into a final score for each primer.

The first stage of screening resulted in a set of forward and reverse primers to provide optimal characteristics for binding to the target genome; however, it did not consider other important effects on NASBA efficiency, namely the length and secondary structure of the amplicon produced by NASBA. To consider these amplicon-related effects, the binding sites for each primer on the genome were identified and, if more than one primer acted at the same site, the primer awarded the highest favorability score was selected. A set of potential forward and reverse primer pairs was constructed based on the recommended amplicon lengths for successful NASBA reactions. Although NASBA is known to work best with amplicons having lengths between approximately 120-nts to 250-nts (Deiman et al., 2002), this rapid screening approach initially employs synthetic DNA strands as templates for transcribing the target RNA. Since the length of these DNA oligos (IDT Ultramers) is currently limited to 200-nts including the T7 promoter site, primer pairs were instead examined for amplicons ranging from 120- to 176-nts in length. After applying all the above constraints on primer and amplicon sequences, a set of 4351 potential NASBA primer pairs remained. The RNA amplicons generated for each of these primers pairs were then assessed for their secondary structure. NASBA reactions are known to be more effective when applied to templates having low secondary structure. Consequently, the degree of amplicon single-strandedness was examined using the NUPACK ensemble defect function (Zadeh et al., 2011a, 2011b).

Next, each of the potential NASBA primers were then coarsely screened for sequence similarities with 11 viruses known to be closely related to Zika: Dengue virus 1, Dengue virus 2, Dengue virus 3, Dengue virus 4, West Nile virus, St. Louis encephalitis virus, yellow fever virus, Powassan virus, Semliki Forest virus, O'nyong'nyong virus, and Chikungunya virus. Sequence similarities were estimated by determining the maximum contiguous sequence in the primer that was found in any of the 11 related viruses. More stringent specificity screening was carried out in later stages as described below.

After all the above screening procedures, the final NASBA primer pairs were sorted by quality after taking into account the favorability scores of each primer, the secondary structure of the amplicon, and the potential for non-specific binding with other viruses.

Identification of Optimal Toehold Switches for NASBA Products. Out of the 4351 NASBA amplicons, the top 1025 were selected as potential targets for toehold switches. The in silico design process for these toehold switches followed closely that previously used for designing mRNA sensors in vivo (Green et al., 2014) and on paper (Pardee et al., 2014). Briefly, toehold switch mRNA sensors were designed that hybridized to the NASBA amplicon at 1-nt increments. This sliding window encompassed the internal region of the NASBA product outside of the primer binding sites. This internal region was selected to avoid any potential for sensor activation or competitive binding by residual NASBA primers. The resulting toehold switches were analyzed for secondary structure and toehold availability, and screened to eliminate any sensors with unwanted in-frame stop codons in the output gene sequence. The target transcript itself was again assayed for single-strandedness and availability of the sensor binding sites. The above factors were incorporated into a sensor design score as described previously (Green et al., 2014). The highest scoring toehold switch sensor for each amplicon was then passed on to the final selection stage.

Final Design Selection Process. After the above screening and design stages, the set of 1025 NASBA primer pairs was assembled with corresponding optimized toehold switch sensors. The top overall designs were selected by combining the favorability scores obtained from the NASBA and toehold switch evaluation steps. The primer and sensor sequences from these top designs were then tested for specificity against the human transcriptome and the same panel of closely related viral genomes listed above using NCBI/Primer-BLAST. Moreover, they were screened for specificity within the Zika genome itself. Ultimately, the top 24 designs that survived the Primer-BLAST specificity stage were selected for testing using our rapid in vitro screening approach.

Modifying the Toehold Switch Sensor Design for Decreased Signal Leakage. Detailed studies of the toehold switch design parameters (Green et al., 2014) and thermodynamic considerations suggested two simple strategies for decreasing leakage from the toehold switches: reducing the size of the loop containing the ribosome binding site (RBS) in the sensor, and further stabilization of the sensor stem. Both these strategies were applied in the Zika-specific toehold switches.

The ON and OFF state signals from the toehold switches increase as the size of the loop in the switch RNA increases. This effect is likely due to two factors: increased accessibility of the RBS to the ribosome, which promotes translation in the presence or absence of the target RNA; and entropic effects that discourage stem formation as the loop becomes longer. Conversely, decreasing the size of the loop is associated with lower leakage, albeit with a decrease in ON state activity. Stabilizing the switch RNA stem by adding additional base pairs or by eliminating the downstream refolding domain (Green et al., 2014) increases the free energy required to unwind the sensor stem and thus encourages decreased signal leakage.

In accordance with above factors, two different types of toehold switches were tested aiming to lower leakage. The first type of sensor, referred to as the A series, are nearly identical to those previously used for mRNA sensing (Green et al., 2014; Pardee et al., 2014), except their loop domain has been reduced from 18-nts to 11-nts. These A sensors retained the downstream refolding domain to encourage sensor triggering and they all have the same sequence at the top of the sensor stem-loop (GUUAUAGUUAUGAACAGAGGAGACAUAACAUGAAC (SEQ ID NO: 6)) as illustrated in FIG. 3A.

The second type of sensor, referred to as the B series, possesses a stem that has been lengthened by one base pair overall and a loop region that is only 12-nts. Importantly, the B sensors also lack the downstream refolding domain to further stabilize the OFF state. The parental toehold switch for the B sensors exhibited extremely low leakage in preliminary measurements in paper-based reactions and provided a sizeable ON/OFF ratio of ˜600-fold regulating GFP expression in E. coli. These B sensors all featured the same conserved sequence (GGACUUUAGAACAGAGGAGAUAAAGAUG (SEQ ID NO: 7)) at the top of their stem-loops as illustrated in FIG. 2B.

### Considerations of Sequence Information in Design of the Biomolecular Diagnostics

Evolutionary drift. At the time we began our experiments, few complete genomes for the Zika virus had been reported. In fact, the first complete genome of the strain circulating in the Americas was only published on January 7 in The Lancet (Enfissi et al., 2016). However, previous comparisons of Zika strains do provide information on the degree of evolutionary drift for the virus. Haddow et al. found that a Zika strain isolated in Malaysia in 1966 differed by only 4.3% in nucleotide sequence from isolates obtained Micronesia and Cambodia in 2007 and 2010, respectively (Haddow et al., 2012). Authors also found ≤11.7% nucleotide sequence difference between African and Asian virus lineages, which they argued provided sufficient conserved sequence for genetic tests for both lineages. More recent studies have shown the rate of mutation of Zika is ˜10-3 nucleotide changes/site/year, which is relatively high among flaviviruses, but a manageable rate for our diagnostic assay (Faria et al., 2016).

Specificity of the NASBA/toehold switch isothermal assay. Since the experiments are performed at mostly 41° C. and 37° C., melting-temperature-dependent tuning of primer specificity is not possible in our assays. The benefit of this temperature limitation is that our NASBA and toehold switch detection schemes are able to tolerate mismatches and compensate for variability in the sequence of the target RNA molecules. The binding between the toehold switch 32B, our highest performing sensor, and RNAs from homologous regions in Zika strains isolated from Africa (Uganda, Nigeria, Senegal) and Asia (Malaysia, Cambodia, French Polynesia) were analyzed. All these strains are predicted to fully activate the toehold sensors even with up to 4-nt (11%) mismatches.

Compensating for evolutionary drift. The above analysis is borne out in data showing that sensor 32B can detect the target sequence from both the American and African strains of the virus (FIG. 8E). This flexibility in sequence detection is balanced with the three combined layers of specificity in our biomolecular approach. This includes the extensive in silico screening of NASBA primers and toehold switch sequences to limit cross-reactivity with off-target sequences and the single-base discrimination of NASBACC. The net result is a programmable platform that can be manufactured to produce diagnostics with both high sequence specificity and the capacity to manage sequence diversity as pathogens evolve. It is also worth mentioning that, in addition to increasing specificity, NASBACC can be used to remove non-specific sequences from samples as well as aid in the discovery mutations in the target regions.

Integration with signature erosion analysis tools. Software such as BioVelocity (Sozhamannan et al., 2015) and TOPSI (Vijaya Satya et al., 2010) are adept at determining conserved sequence regions across multiple genomes and eliminating those shared with other pathogens or humans. Using these valuable tools, a set of specific sites for NASBA priming and toehold switch binding can be generated and then subjected to the same screening by toehold secondary structure and NASBA primer characteristics described in this work.

Freeze-dried NASBA. For freeze-dried NASBA experiments, Enzyme Mix was lyophilized separately from the other components. The solution containing reaction buffer, nucleotide mix, RNase inhibitor, and primers was reconstituted in 15% DMSO, while the Enzyme Mix was reconstituted in nuclease-free water.

Freeze-dried CRISPR/Cas9 nuclease assay. Reactions were performed in a 30 j.tl volume of 1× Cas9 Nuclease Reaction Buffer (NEB #M0386S) containing 30 nM of guide RNA (gRNA), 30 nM of Cas9 Nuclease (S. pyogenes, NEB #M0386S), and 3 nM of substrate DNA (pAG_TS1_KS001 plasmid). Six gRNA sequences targeting the lacZ gene (Doench et al., 2014) inserted into plasmid pAG_TS1_KS001 were used for the CRISPR/Cas9 freeze-dried assay. All components except substrate DNA were first combined in a 27 μl reaction volume and incubated for 10 minutes at 25° C. to allow Cas9+gRNA to form duplexes. For fresh reactions, 3 μl of a 30 nM solution of substrate DNA was added to the solution. For freeze-dried reactions, the 27 μl solution was lyophilized overnight and reconstituted with 30 j.tl of a solution containing 3 nM of substrate DNA. After the addition of substrate DNA, the solution was incubated for 1 h at 37 C and run on a 1% agarose gel for fragment analysis.

Care and use of macaques at the Wisconsin National Primate Research Center. All Indian-origin rhesus macaque monkeys from which plasma was isolated were cared for by the staff at the Wisconsin National Primate Research Center (WNPRC) in accordance with the regulations and guidelines outlined in the Animal Welfare Act and the Guide for the Care and Use of Laboratory Animals and the recommendations of the Weatherall report. This study was approved by the University of Wisconsin-Madison Graduate School Institutional Animal Care and Use Committee (Animal Care and Use Protocol Number G005401). For all procedures (i.e., physical examination, virus inoculation, blood and swab collection), animals were anesthetized with an intramuscular dose of ketamine (10 ml/kg). Blood samples were obtained using a vacutainer system or needle and syringe from the femoral or saphenous vein.

Zika virus stock production for macaque infection. ZIKV strain H/PF/2013 (GenBank accession number: KJ776791), originally isolated from a 51-year-old female in France returning from French Polynesia with a single round of amplification on Vero cells, was obtained from Xavier de Lamballerie (European Virus Archive, Marseille France). Virus stocks were prepared by inoculation onto a confluent monolayer of C6/36 mosquito cells. A single harvest of virus with a titer of 1.26×10⁶ PFU/ml for the Asian-lineage (equivalent to 1.43×10⁹ vRNA copies/ml) was used.

Zika virus challenge of macaques, plasma collection and processing. The virus stock was thawed, diluted in PBS to the appropriate concentration for each challenge, and loaded into a 1 ml syringe that was kept on ice until challenge. Animals were anesthetized as described above, and 1 ml of inocula was administered subcutaneously over the cranial dorsum. At the conclusion of the procedure, animals were closely monitored by veterinary and animal care staff for adverse reactions and signs of disease. Fresh plasma and PBMC were isolated from EDTA-treated whole blood by Ficoll density centrifugation at 1860 rcf for 30 min. The plasma layer was collected and centrifuged for an additional 8 min at 670 rcf to remove residual cells. The supernatant plasma was then filtered over a 0.45 μm syringe filter. Collected plasma was diluted 1:10 in nuclease free water. Diluted samples were heated to 95 C for two minutes and immediately added to a NASBA reaction as described above. NASBA was run for three hours.

### Example 2

Cas9 interferes with T7-mediated RNA production. RNA can be produced in vitro from a DNA template containing the T7 promoter sequence (5′-TAATACGACTCACTATAGGG-3′ (SEQ ID NO: 768)). If the DNA template has viable CRISPR-Cas9 target sites, addition of Cas9+gRNA complex to the in vitro RNA production reaction leads to the production of truncated RNA product.

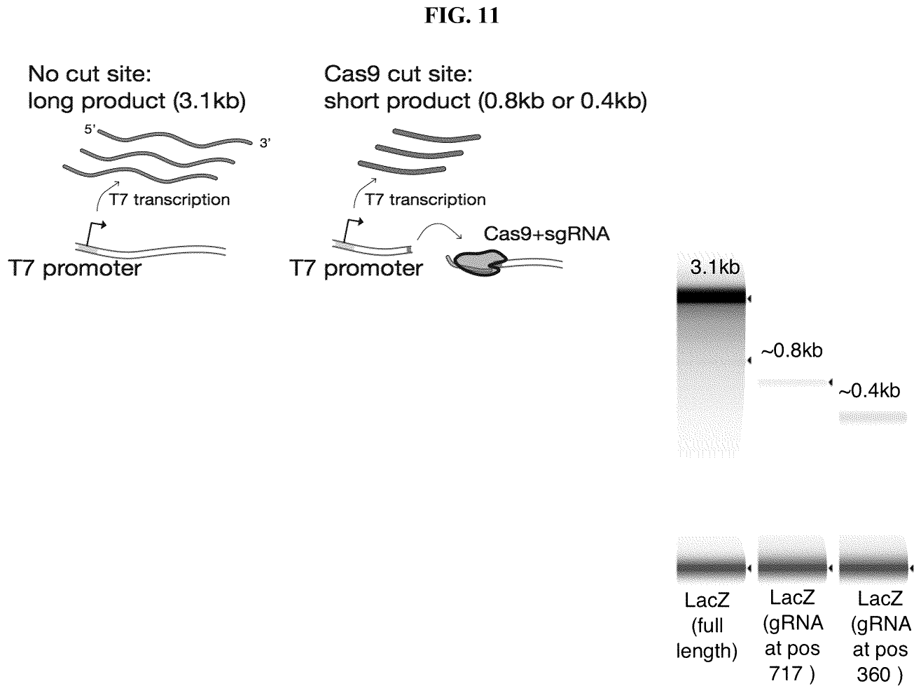

In FIG. 11, RNA was produced from a DNA template using NEB's HiScribe™ T7 Quick High Yield RNA Synthesis Kit. In lane 1, Cas9 without a gRNA was added to the reaction. In lanes 2 and 3, Cas9+gRNA that target the LacZ gene at positions 717 (gRNA: cttcagcctccagtacageg (SEQ ID NO: 769)) and 360 (gRNA: gttcccacggagaatccgac (SEQ ID NO: 770)) was added to the reaction. Truncated RNA products were produced in lanes 2&3, and full-length RNA only in lane 1. Traces were generated using a BioAnalyzer (Agilent).

Cas9 can be freeze-dried and remain active. It was next tested whether guide RNA targeting the LacZ sequence contained in the LacZ gene would remain active after being freeze-dried. In particular, a reaction containing Cas9+gRNA complex in Cas9 buffer (NEB #m0386) was freeze dried and re-hydrated with a solution containing 3 nM of template DNA. The template DNA is a supercoiled plasmid containing the LacZ gene.

In FIGS. 9A-9B, the sequence of 6 gRNA is listed and the gel image resulting nuclease reaction is shown. Nuclease activity was maintained for all gRNA target sequences, and some gRNA (#1, 2, 3, 4, 5) showed increase activity following the freeze-drying process).

Cas9 without guide RNA does not interfere with NASBA. The presence of Cas9 nuclease alone does not interfere with NASBA reactions. The non-reactivity of NASBA reactions to Cas9 nucleases was demonstrated by adding Cas9 nuclease and Cas9 buffer to a NASBA reaction. Since the stability of thermal Cas9 has not been demonstrated above 37° C., the NASBA reaction was performed at 37° C.

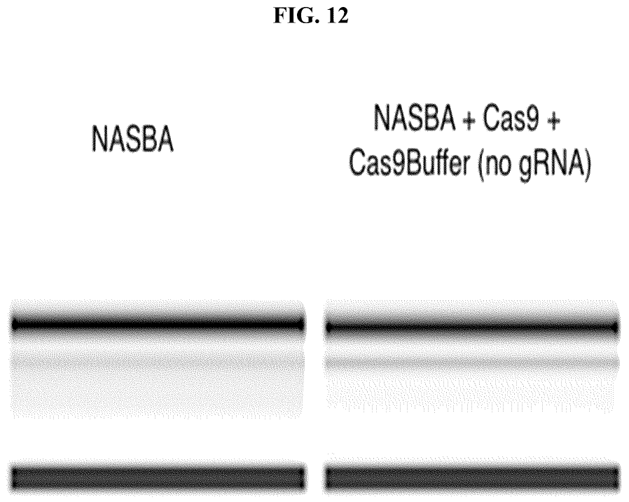

FIG. 12 shows the RNA product from a NASBA reaction (left) and a NASBA reaction with added Cas9, Cas9 buffer, but with no guide RNA (right). Both RNA produces are of the same quality/length. Traces were generated using a BioAnalyzer (Agilent).

Cas9 with a gRNA targeting a site lacking a PAM site does not interfere with NASBA. A key aspect of this invention is that a single base mutation affecting a PAM site will present Cas9 from binding/cleaving template DNA. To demonstrate this, a guide RNA with the 20-bp spacer sequence neg4-noGG (neg4-noGG TTTCAAGAATGGAAAACATC (SEQ ID NO: 771)) was designed to be homologous a region of the American strain (GenBank: KU3 12312) of Zika RNA, but at a location that lacks a PAM site (ATG): neg4-noNGG, Loc=2621 of GenBank: KU312312, sequence:

(SEQ ID NO: 772)
gcgggatctcctctgTTTCAAGAATGGAAAACATCATGtggagatcagtag
aa.

For comparison, another guide RNA sequence with the spacer sequence pos4 (pos4: GATCTCCTCTGTTTCAAGAA (SEQ ID NO: 773)) was designed near a PAM site (TGG) to cut the template DNA and interfere with NASBA amplification: pos4, Loc=2610 of GenBank: KU312312, sequence:

(SEQ ID NO: 774)
agatggtatctgcggGATCTCCTCTGTTTCAAGAATGGaaaacatcatgtg
ga.

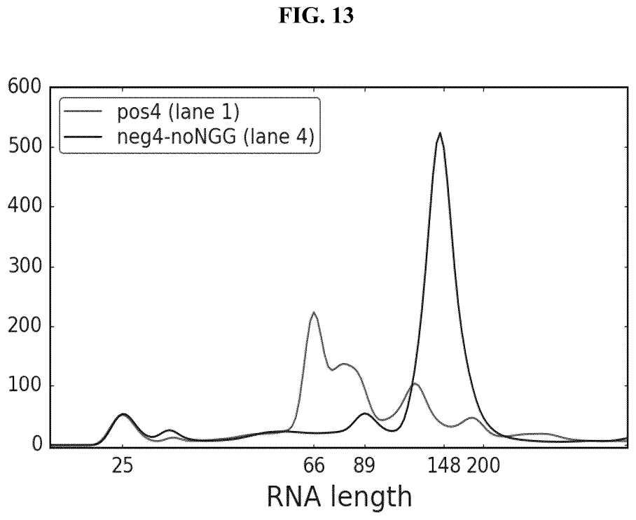

FIG. 13 depicts the resulting RNA tract of the NASBA amplification reaction as quantified on a BioAnalyzer. The “pos4” gRNA cuts the intermediate template DNA and results in a shorter RNA product compared with the “neg4-noNGG” gRNA (66 bp vs. 148 bp).

Cas9 with a gRNA that targets reverse-transcribed RNA interferes with NASBA. This is demonstrated in FIGS. 8A-8E.

Cas9 can interfere with NASBA amplification when using low reverse transcription (RT) primers concentration of 3 nM or less. The amount of template DNA that is generated during the reverse-transcription step of a NASBA reaction has to remain low. Using the conditions detailed above herein (250 nM final concentration of Cas9+gRNA), it was found that the amount of reverse transcription primer present in the NASBA has to be 3 nM or lower in order to efficiently cleave any intermediate template DNA generated during a NASBA reaction. A final concentration of 1 nM is preferable.

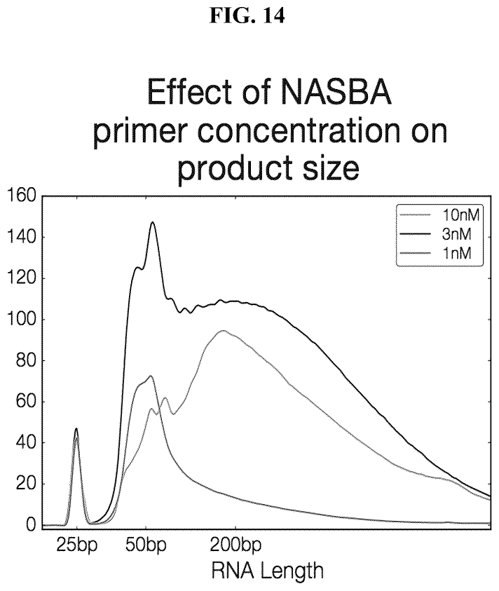

FIG. 14 depicts the RNA product generated from three reactions containing 1 nM, 3 nM, or 10 nM of NASBA primers. The guide RNA with the spacer sequence TGGAGTCCCGCTGCTAATGA (SEQ ID NO: 775) was designed to target the African strain (GenBank: KF268950) of the Zika virus. The expected size following a successful Cas9 cleavage is 50 base pairs (bp). The full-length, uncut RNA is 200 bp.

8gRNA, Loc=7208 of (GenBank: KF268950)

Sequence:
(SEQ ID NO: 776)
ttatgcatgggacttTGGAGTCCCGCTGCTAATGATGGgttgctactca
caat

Table showing the PAM site creation/annihilation following a random mutation (Table 4). This table shows all the possible outcomes of a random mutation, where a PAM site can be created or annihilated.

Algorithm to detect diverging PAM sites between two strains. List of PAM sites that differ between the American-African strains (572, FIGS. 8A-8E): see Table 5. Each gRNA sequence in Table 5 provides the 30-nt region immediately 5′ to the PAM sequence in the last column. To generate the full sgRNA sequence used, the 20 bases adjacent to the 3′ end of each gRNA sequence column are identified, and the following sequence is appended to the 3′ end of that 20-base sequence (an example is shown in paragraph 000145): 5′-

(SEQ ID NO: 789)
5′-GTTTTAGAGCTAGAAATAGCAAGTTAAAATAAGGCTAGTCCGTTATCA
ACTTGAAAAAGTGGCACCGAGTCGGTGCTTTT-3′.

sgRNA sequence: Region 8 (8gRNA)+crRNA/tracrRNA

(SEQ ID NO: 1)
TGGAGTCCCGCTGCTAATGAGTTTTAGAGCTAGAAATAGCAAGTTAAAATA
AGGCTAGTCCGTTATCAACTTGAAAAAGTGGCACCGAGTCGGTGCTTTT.

The present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

TABLE 2
Sequences of NASBA Primers Used for Zika RNA Amplification

| Toehold Switch | | NASBA Primers for Zika Virus from | | | |
|---|---|---|---|---|---|
| Sensor | | the Americas (KU312312) | | | |
| A Series | B series | Forward | SEQ ID NO: | Reverse | SEQ ID NO: |
| 1A | 25B | AATTCTAATACGACT | 8 | GCCCA | 32 |
| | | CACTATAGGGAGAAG | | GCTAA | |
| | | GCAGTGATCTAGGCT | | AGACT | |
| | | ACTGGA | | TGGGT | |
| | | | | ATGA | |
| 2A | 26B | AATTCTAATACGACT | 9 | CATCC | 33 |
| | | CACTATAGGGAGAAG | | AGTGA | |
| | | GGTGCCAGAGTTGTG | | TCCTC | |
| | | TGTACA | | GTTCA | |
| 3A | 27B | AATTCTAATACGACT | 10 | CCTGT | 34 |
| | | CACTATAGGGAGAAG | | CCTCG | |
| | | GGCACAGTGGGATGA | | GTTCA | |
| | | TCGTTA | | CAATC | |
| | | | | AA | |
| 4A | 28B | AATTCTAATACGACT | 11 | AATCT | 35 |
| | | CACTATAGGGAGAAG | | CTGTG | |
| | | GCGGGATCTCCTCTG | | GACCT | |
| | | TTTCAA | | CTCCA | |
| 5A | 29B | AATTCTAATACGACT | 12 | GAGGC | 36 |
| | | CACTATAGGGAGAAG | | CAACA | |
| | | GCCATCACTGGGTCT | | ATTCC | |
| | | CATCAA | | GACAC | |
| | | | | TA | |
| 6A | 30B | AATTCTAATACGACT | 13 | CTGTC | 37 |
| | | CACTATAGGGAGAAG | | CTCGG | |
| | | GAATGCTGTCAGTTC | | TTCAC | |
| | | ATGGCTCCCA | | AATCA | |
| 7A | 31B | AATTCTAATACGACT | 14 | CCAGA | 38 |
| | | CACTATAGGGAGAAG | | ACCTT | |
| | | GATGGTCTCTTCCTG | | GGATC | |
| | | GTTGTGGA | | GTTCA | |
| 8A | 32B | AATTCTAATACGACT | 15 | CCATC | 39 |
| | | CACTATAGGGAGAAG | | CACAA | |
| | | GGACCCTAATAGTGG | | CAGGG | |
| | | CCATCA | | TTCTT | |
| | | | | CA | |
| 9A | 33B | AATTCTAATACGACT | 16 | CAGCC | 40 |
| | | CACTATAGGGAGAAG | | CTGGG | |
| | | GGTTTGGTATGGGCA | | ATCAA | |
| | | AAGGGA | | GTACA | |
| | | | | TGTA | |
| 10A | 34B | AATTCTAATACGACT | 17 | GCCCA | 41 |
| | | CACTATAGGGAGAAG | | TACCA | |
| | | GGCCATCTATGCTGC | | AACAA | |
| | | CTTGACAA | | CACTC | |
| | | | | CA | |
| 11A | 35B | AATTCTAATACGACT | 18 | GCTTA | 42 |
| | | CACTATAGGGAGAAG | | GCCAG | |
| | | GGTGATTCTGCTCAT | | GTCAC | |
| | | GGTGCA | | TCATT | |
| | | | | GA | |
| 12A | 36B | AATTCTAATACGACT | 19 | AAGTT | 43 |
| | | CACTATAGGGAGAAG | | GGCGC | |
| | | GGTTTGTTCCAAGCG | | CCATC | |
| | | TGAGGA | | TCTGA | |
| | | | | AA | |
| 13A | 37B | AATTCTAATACGACT | 20 | GTTCT | 44 |
| | | CACTATAGGGAGAAG | | TTCCT | |
| | | GAGATCAACCACTGC | | GGGCC | |
| | | AAGCGGAA | | TTATC | |
| | | | | TCCA | |
| 14A | 38B | AATTCTAATACGACT | 21 | CCTTC | 45 |
| | | CACTATAGGGAGAAG | | TTGAC | |
| | | GAGTGGTTCCACGAC | | TCCCT | |
| | | ATTCCA | | AGAAC | |
| | | | | CA | |
| 15A | 39B | AATTCTAATACGACT | 22 | ATCTC | 46 |
| | | CACTATAGGGAGAAG | | TCTGT | |
| | | GTGGACGCCAGAGTT | | CATGT | |
| | | TGTTCAGA | | GTCCT | |
| | | | | GGCA | |
| 16A | 40B | AATTCTAATACGACT | 23 | TCTGA | 47 |
| | | CACTATAGGGAGAAG | | ACAAA | |
| | | GGCCGGAATAACCTA | | CTCTG | |
| | | CACAGA | | GCGTC | |
| | | | | CA | |
| 17A | 41B | AATTCTAATACGACT | 24 | GCCCA | 48 |
| | | CACTATAGGGAGAAG | | GCTAA | |
| | | GGGCTACTGGATTGA | | AGACT | |
| | | GAGTGAGA | | TGGGT | |
| | | | | ATGA | |
| 18A | 42B | AATTCTAATACGACT | 25 | TGTTC | 49 |
| | | CACTATAGGGAGAAG | | TCTCC | |
| | | GCAGCCAGAATTGCA | | AACCA | |
| | | TGTGTCCTCA | | TCCGA | |
| 19A | 43B | AATTCTAATACGACT | 26 | GCTCT | 50 |
| | | CACTATAGGGAGAAG | | GCAAC | |
| | | GGGAGCGGACAAGTT | | CAGTT | |
| | | GTCACTTA | | AGTCA | |
| 20A | 44B | AATTCTAATACGACT | 27 | ATCTT | 51 |
| | | CACTATAGGGAGAAG | | CCCAG | |
| | | GGTGCTCGGTGGACT | | GCTTG | |
| | | TCTCAAAGAA | | CTTGA | |
| 21A | 45B | AATTCTAATACGACT | 28 | TGCCA | 52 |
| | | CACTATAGGGAGAAG | | TTCGT | |
| | | GAGTGGTGCAACTCA | | TTGAG | |
| | | TTCGGA | | CCTAT | |
| | | | | CCCA | |
| 22A | 46B | AATTCTAATACGACT | 29 | GCAGT | 53 |
| | | CACTATAGGGAGAAG | | GGTTG | |
| | | GCAATACCAGAGAGG | | ATCTC | |
| | | GCTACA | | AGAGA | |
| 23A | 47B | AATTCTAATACGACT | 30 | CGCAG | 54 |
| | | CACTATAGGGAGAAG | | GTCAA | |
| | | GAGTAGGTCTTCTGG | | TGTCC | |
| | | GCTTGA | | ATTGA | |
| | | | | GA | |
| 24A | 48B | AATTCTAATACGACT | 31 | CCACT | 55 |
| | | CACTATAGGGAGAAG | | CTTGT | |
| | | GGCTCAAACGAATGG | | GTGTC | |
| | | CAGTCA | | CTTCC | |
| | | | | TA | |

| Toehold Switch | | | |
|---|---|---|---|
| Sensor | | NASBA Primers for MR 766 Zika Virus (AY632535) | |
| A Series | B series | Forward | Reverse |
| 1A | 25B | | |
| 2A | 26B | | |
| 3A | 27B | AATTCTAATACGACT | CCTGTCCTCGGTTCA |
| | | CACTATAGGGAGAAG | CAATCAA |
| | | GGCACAGTGGGATGA | (SEQ ID NO: 58) |
| | | TCGTTA | |
| | | (SEQ ID NO: 56) | |
| 4A | 28B | | |
| 5A | 29B | | |
| 6A | 30B | | |
| 7A | 31B | | |
| 8A | 32B | AATTCTAATACGACT | CCATCCACAACGGGA |
| | | CACTATAGGGAGAAG | TTCTTCA |
| | | GACTCTGATAGTAGC | (SEQ ID NO: 59) |
| | | TATCA (SEQ | |
| | | ID NO: 57) | |
| 9A | 33B | | |
| 10A | 34B | | |
| 11A | 35B | | |
| 12A | 36B | | |
| 13A | 37B | | |
| 14A | 38B | | |
| 15A | 39B | | |
| 16A | 40B | | |
| 17A | 41B | | |
| 18A | 42B | | |
| 19A | 43B | | |
| 20A | 44B | | |
| 21A | 45B | | |
| 22A | 46B | | |
| 23A | 47B | | |
| 24A | 48B | | |

TABLE 3
Exemplary Materials for Portable Electronic Reader
total $: 244.238

| | | | total | | |
|---|---|---|---|---|---|
| Vendor | quantity | price | price | item # | Description |
| Adafruit | 1 | 24.95 | 24.95 | 50 | Arduino Uno R3 (Atmega328 - assembled) |
| Adafruit | 1 | 19.95 | 19.95 | 2078 | Adafruit PowerBoost 500 Shield - |
| | | | | | Rechargeable 5 V Power Shield |
| Adafruit | 1 | 12.5 | 12.5 | 2011 | Lithium Ion Battery - 3.7 v 2000 mAh |
| Adafruit | 1 | 19.95 | 19.95 | 1141 | Adafruit Assembled Data Logging shield |
| | | | | | for Arduino |
| Adafruit | 1 | 7.95 | 7.95 | 102 | SD/MicroSD Memory Card (4 GB SDHC) |
| Adafruit | 16 | 6.26 | 100.16 | 1980 | Adafruit TSL2591 High Dynamic Range |
| | | | | | Digital Light Sensor |
| Sparkfun | 3 | 2.8 | 8.4 | PRT-12702 | SparkFun Solderable Breadboard - Mini |
| Digi-Key | 2 | 0.49 | 0.98 | A19473-ND | CONN HEADER VERT 9POS.100 TIN |
| Digi-Key | 2 | 0.643 | 1.286 | A19476-ND | CONN HEADER VERT 13POS.100 TIN |
| Digi-Key | 2 | 1.21 | 2.42 | A31001-ND | CONN RECEPT 9POS 28AWG MTA100 |
| Digi-Key | 2 | 1.93 | 3.86 | A30954-ND | CONN RECEPT 13POS 28AWG MTA100 |
| Digi-Key | 20 | 0.2246 | 4.492 | 36-621-ND | BRACKET RT ANG MOUNT 4-40 STEEL, |
| | | | | | for reader assembly |
| McMaster-Carr | 1 | 2.36 | 2.36 | 90760A005 | Zinc Plated Steel Narrow Hex Nut |
| | | | | | 4-40 Thread Size, 3/16″ Wide, |
| | | | | | 1/16″ High, for reader assembly |
| McMaster-Carr | 1 | 6.74 | 6.74 | 8505K11 | Black Acrylic, for reader |
| McMaster-Carr | 2 | 8.04 | 16.08 | 92095A453 | M2 screws, for attaching sensors |
| McMaster-Carr | 1 | 1.04 | 1.04 | 90592A004 | M2 nuts, for attaching sensors |
| Inventables | 1 | 4.84 | 4.84 | 23876-35 | Clear acrylic, for bottom half of cassette |
| Inventables | 1 | 6.28 | 6.28 | 24112-04 | Black acrylic, for top half of cassette |

TABLE 4
Probabilities of PAM Site Disruption From Single Point Mutations
A. Summary of the effect of single point mutations
12 PAM sites
4 double PAM sites
32 PAM site created
12 double PAM site created
32 PAM site destroyed
12 double PAM site destroyed
4 PAM site inverted
B. Detailed list of all single point mutations on every 3-bp sequences.

| | Last | | | | | | | | | Last | | | | | | | |
|---|---|---|---|---|---|---|---|---|---|---|---|---|---|---|---|---|---|
| First | A | | C | | G | | T | | First | A | | C | | G | | T | |
| | Center letter: A | | | | | | | | | Center letter: C | | | | | | | |
| A | AAA | ACA | AAC | ACC | AAG | ACG | AAT | ACT | A | ACA | ACA | ACC | AAC | ACG | AAG | ACT | AAT |
| | | AGA | | AGC | | AGG | | AGT | | | AGA | | AGC | | AGG | | AGT |
| | | ATA | | ATC | | ATG | | ATT | | | ATA | | ATC | | ATG | | ATT |
| C | CAA | CCA | CAC | CCC | CAG | CCG | CAT | CCT | C | CCA | CAA | CCC | CAC | CCG | CAG | CCT | CAT |
| | | CGA | | CGC | | CGG | | CGT | | | CGA | | CGC | | CGG | | CGT |
| | | CTA | | CTC | | CTG | | CTT | | | CTA | | CTC | | CTG | | CTT |
| G | GAA | GCA | GAC | GCC | GAG | GCG | GAT | GCT | G | GCA | GAA | GCC | GAC | GCG | GAG | GCT | GAT |
| | | GGA | | GGC | | GGG | | GGT | | | GGA | | GGC | | GGG | | GGT |
| | | GTA | | GTC | | GTG | | GTT | | | GTA | | GTC | | GTG | | GTT |
| T | TAA | TCA | TAC | TCC | TAG | TCG | TAT | TCT | T | TCA | TAA | TCC | TAC | TCG | TAG | TCT | TAT |
| | | TGA | | TGC | | TGG | | TGT | | | TGA | | TGC | | TGG | | TGT |
| | | TTA | | TTC | | TTG | | TTT | | | TTA | | TTC | | TTG | | TTT |
| | Center letter: G | | | | | | | | | Center letter: T | | | | | | | |
| A | AGA | AAA | AGC | AAC | AGG | AAG | AGT | AAT | A | ATA | AAA | ATC | AAC | ATG | AAG | ATT | AAT |
| | | ACA | | ACC | | ACG | | ACT | | | ACA | | ACC | | ACG | | ACT |
| | | ATA | | ATC | | ATG | | ATT | | | AGA | | AGC | | AGG | | AGT |
| C | CGA | CAA | CGC | CAC | CGG | CAG | CGT | CAT | C | CTA | CAA | CTC | CAC | CTG | CAG | CTT | CAT |
| | | CCA | | CCC | | CCG | | CCT | | | CCA | | CCC | | CCG | | CCT |
| | | CTA | | CTC | | CTG | | CTT | | | CGA | | CGC | | CGG | | CGT |
| G | GGA | GAA | GGC | GAC | GGG | GAG | GGT | GAT | G | GTA | GAA | GTC | GAC | GTG | GAG | GTT | GAT |
| | | GCA | | GCC | | GCG | | GCT | | | GCA | | GCC | | GCG | | GCT |
| | | GTA | | GTC | | GTG | | GTT | | | GGA | | GGC | | GGG | | GGT |
| T | TGA | TAA | TGC | TAC | TGG | TAG | TGT | TAT | T | TTA | TAA | TTC | TAC | TTG | TAG | TTT | TAT |
| | | TCA | | TCC | | TCG | | TCT | | | TCA | | TCC | | TCG | | TCT |
| | | TTA | | TTC | | TTG | | TTT | | | TGA | | TGC | | TGG | | TGT |

Each mutation has a 23% probability (44/192) of creating a new PAM site, a 23% probability (44/192) of destroying and existing PAM site, and a 2% probability (4/192) of inverting the orientation of an existing PAM site.
Overall, any given point mutations has a 48% probability (92/192) of disrupting an existing PAM site.

TABLE 5
Suitable sites in the African, Asian,
and American Zika virus genomes for
CRISPR complex cleavage.

| | | | | | |
|---|---|---|---|---|---|
| Target | | | | SEQ ID | PAM- |
| Strain | Position | strand | gRNA-sequence | NO: | site |

American-African NASBACC genotyping

| | | | | | |
|---|---|---|---|---|---|
| African | 302 | −1 | TTTTTCCCCACAGAACCCCATCTGTTGATG | 60 | AGG |
| African | 406 | 1 | ATGTTGAGAATAATCAATGCTAGGAAGGAG | 61 | AGG |
| African | 433 | −1 | TAGTCAGCAGGAGACCAATGATTCCGATGC | 62 | TGG |
| African | 469 | −1 | CACTTCCACGTCTGGTAATCTCCGCTGCCA | 63 | TGG |
| African | 487 | −1 | CCAAGTACATGTAGTATGCACTTCCACGTC | 64 | TGG |
| African | 517 | 1 | CGTGGAAGTGCATACTACATGTACTTGGAC | 65 | AGG |
| African | 547 | −1 | GGCATTTGTTAACTCCCAAGTTGGTAGCAA | 66 | AGG |
| African | 578 | −1 | CACATGTGCCCGAGATCCATGATCTGTACA | 67 | TGG |
| African | 595 | 1 | AACAAATGCCATGTACAGATCATGGATCTC | 68 | GGG |
| African | 839 | −1 | TTCCTAAATATCCAGTTTTCAACCTTGATC | 69 | AGG |
| African | 842 | 1 | AATCAAGAGAATACACGAAGCACCTGATCA | 70 | AGG |
| African | 871 | 1 | AAGGTTGAAAACTGGATATTTAGGAACCCC | 71 | GGG |
| African | 874 | −1 | AGGCAATGGCAACAGCTGCGAGCGCAAACC | 72 | CGG |
| African | 904 | −1 | CTTTTTGGCTCGTCGAGCTTCCCAAAAGCC | 73 | AGG |
| African | 1036 | −1 | AACCTCCATGTTCCAAGACGACATCAACCC | 74 | AGG |
| African | 1075 | −1 | TGTCAACTGTTGGCTTGTCCTGTGCCATAA | 75 | CGG |
| African | 1121 | 1 | CAACAGTTGACATAGAGTTGGTCACGACAA | 76 | CGG |
| African | 1141 | −1 | TTGATGCCTCATAACAGTAGGATCTTACCT | 77 | CGG |
| African | 1243 | −1 | TGTCCACCAATGTTCTTTTGCACACATATT | 78 | GGG |
| African | 1244 | −1 | CTGTCCACCAATGTTCTTTTGCACACATAT | 79 | TGG |
| African | 1262 | 1 | CAGACACCCAATATGTGTGCAAAAGAACAT | 80 | TGG |
| African | 1285 | 1 | AGAACATTGGTGGACAGAGGTTGGGGAAAT | 81 | GGG |
| African | 1330 | −1 | TCCCAGTCATCTTCTTGGAACACGTGAACT | 82 | TGG |
| African | 1478 | 1 | ATGAAACTGACGAAAACAGAGCGAAAGTCG | 83 | AGG |
| African | 1562 | 1 | TAGGACTTGATTGTGAACCAAGGACAGGCC | 84 | TGG |
| African | 1594 | −1 | CTTTGTGCACCAACCAGTGCTTGTTGTTCA | 85 | TGG |
| African | 1643 | −1 | CCGGTATCTGCCCCAGCATGCCAAGGCAAT | 86 | GGG |
| African | 1691 | 1 | CAGATACCGGAACTCCACACTGGAACAACA | 87 | AGG |
| African | 1694 | 1 | ATACCGGAACTCCACACTGGAACAACAAGG | 88 | AGG |
| African | 1723 | −1 | CTAGAACAACGACGGTTTGCCTCTTGGCGT | 89 | GGG |
| African | 1724 | −1 | CCTAGAACAACGACGGTTTGCCTCTTGGCG | 90 | TGG |
| African | 1741 | −1 | CGGCTCCTTCCTGGCTCCCTAGAACAACGA | 91 | CGG |
| African | 1757 | 1 | AGAGGCAAACCGTCGTTGTTCTAGGGAGCC | 92 | AGG |
| African | 1760 | −1 | CCAGCAAGAGCCGTGTGAACGGCTCCTTCC | 93 | TGG |
| African | 1771 | −1 | CCTCCAGAGCTCCAGCAAGAGCCGTGTGAA | 94 | CGG |
| African | 1837 | −1 | TGTCCATTTTTAAGCGGCATTTCAAATGGC | 95 | CGG |
| African | 1853 | −1 | CCCTTCAATCTAAGCTTGTCCATTTTTAAG | 96 | CGG |
| African | 1906 | −1 | CAGCTGGGACCTTGGTAAATGTGAACGCTG | 97 | CGG |
| African | 1922 | 1 | CCTTGTGCACCGCAGCGTTCACATTTACCA | 98 | AGG |
| African | 1993 | −1 | TGTCCACCGCCATCTGGGCTGGGACCTTGC | 99 | AGG |
| African | 2003 | −1 | AGGGTCTGCATGTCCACCGCCATCTGGGCT | 100 | GGG |
| African | 2008 | −1 | GGGTCAGGGTCTGCATGTCCACCGCCATCT | 101 | GGG |
| African | 2032 | −1 | TGGCGGTTATCAGCCTCCCGACTGGGGTCA | 102 | GGG |
| African | 2033 | −1 | TTGGCGGTTATCAGCCTCCCGACTGGGGTC | 103 | AGG |
| African | 2062 | −1 | AATTCTCAGTGCTTTCAGTAATCACAGGGT | 104 | TGG |
| African | 2117 | −1 | CCTATGACAATGTAAGAATCCCCAAATGGT | 105 | GGG |
| African | 2210 | 1 | GGAGTGGTAGCACCATCGGAAAAGCATTTG | 106 | AGG |
| African | 2248 | 1 | GTGAGAGGTGCCAAGAGAATGGCAGTTCTG | 107 | GGG |
| African | 2249 | 1 | TGAGAGGTGCCAAGAGAATGGCAGTTCTGG | 108 | GGG |
| African | 2278 | 1 | GGGGATACAGCCTGGGACTTCGGATCAGTC | 109 | GGG |
| African | 2279 | 1 | GGGATACAGCCTGGGACTTCGGATCAGTCG | 110 | GGG |
| African | 2318 | −1 | AACAGTGATTTGAAAGCTGCTCCAAAAATC | 111 | TGG |
| African | 2378 | −1 | AAACCCAACCACACCAGCAGCGTGCCTATG | 112 | AGG |
| African | 2390 | 1 | GGTTCTCACAGATCCTCATAGGCACGCTGC | 113 | TGG |
| African | 2428 | −1 | CTCCCCCCAGGGCCAAGCATGTGAGGGAGA | 114 | TGG |
| African | 2447 | 1 | ATGGATCCATCTCCCTCACATGCTTGGCCC | 115 | TGG |
| African | 2471 | −1 | CACCCCACGTCAGCAGAGACAGCCGTGGAG | 116 | AGG |
| African | 2474 | 1 | CCCTGGGGGGAGTGATGATCTTCCTCTCCA | 117 | CGG |
| African | 2537 | 1 | ACTTCTCAAAAAGAGAAACGAGATGTGGCA | 118 | CGG |
| African | 2630 | 1 | CCCCCCGCAGATTGGCAGCAGCAGTCAAGC | 119 | AGG |
| African | 2642 | 1 | TGGCAGCAGCAGTCAAGCAGGCTTGGGAAG | 120 | AGG |
| African | 2644 | 1 | GCAGCAGCAGTCAAGCAGGCTTGGGAAGAG | 121 | GGG |
| African | 2668 | −1 | ATTTCCACATGATGTTTTCCATTCTTGAAA | 122 | CGG |
| African | 2699 | 1 | CAAGAATGGAAAACATCATGTGGAAATCAG | 123 | TGG |
| African | 2792 | −1 | AGCTCATTCACAGGCACTGGCAATCTTTGT | 124 | GGG |
| African | 2864 | 1 | AAGCCTGGGGGAAATCATATTTTGTCAGAG | 125 | CGG |
| African | 2878 | −1 | GTGTGTCACCATCGACAACAAAACTGTTGT | 126 | TGG |
| African | 2981 | −1 | TCTTCTCTAACCTTGAGCCAAACACTGGTG | 127 | TGG |
| African | 2986 | −1 | AGTAGTCTTCTCTAACCTTGAGCCAAACAC | 128 | TGG |
| African | 3032 | −1 | TTTCCCTTAACAGCTGTTCCTATGACGGCT | 129 | GGG |
| African | 3070 | −1 | TTTCAATCCAGTAGCCCAGGTCACTGTGGG | 130 | CGG |
| African | 3073 | −1 | CACTTTCAATCCAGTAGCCCAGGTCACTGT | 131 | GGG |
| African | 3074 | −1 | TCACTTTCAATCCAGTAGCCCAGGTCACTG | 132 | TGG |
| African | 3080 | 1 | TTAAGGGAAAGGAGGCCGCCCACAGTGACC | 133 | TGG |
| African | 3083 | −1 | TCATTCTTTTCACTTTCAATCCAGTAGCCC | 134 | AGG |
| African | 3251 | −1 | GCTTGAGTTCTGTAACCCTCTCTAGTGTTG | 135 | TGG |
| African | 3317 | −1 | TGAACCTTGGTGCCTGGACATTCCTCAAAC | 136 | CGG |
| African | 3340 | −1 | TAGTTCCGCATGTCTCCTCCACGTGAACCT | 137 | TGG |
| African | 3542 | 1 | AGAGCAACTTAGTGAGGTCAATGGTGACAG | 138 | CGG |
| African | 3556 | −1 | GCACTCCAAGAGAGAAGTGGTCCATGTGAT | 139 | CGG |
| African | 3668 | 1 | TCATTATGAGCACATCAATGGCAGTGCTGG | 140 | TGG |
| African | 3676 | −1 | AGTCACTCATTAAAAATCCTCCCAGGATCA | 141 | TGG |
| African | 3764 | 1 | CCTTCGCAGAAATGAACACTGGAGGAGATG | 142 | TGG |
| African | 3779 | 1 | ACACTGGAGGAGATGTGGCTCACTTGGCAT | 143 | TGG |
| African | 3811 | −1 | AATTAGCTCTGAAAATAAAGGAGACCAGCA | 144 | AGG |
| African | 3823 | −1 | CACGAGGTGTCCAATTAGCTCTGAAAATAA | 145 | AGG |
| African | 3872 | −1 | GAGATTGCAGTTTGCAAAAGACACGAAGCC | 146 | AGG |
| African | 3929 | −1 | GCCAACCAGGCCAAAGCAAATCCATTGACG | 147 | AGG |
| African | 3976 | −1 | TTGCCAGAGCAATGTTGTCAGTGCGTGGCA | 148 | CGG |
| African | 4058 | 1 | CCCGAGGTACACTGCTCGTGGCATGGAGAG | 149 | CGG |
| African | 4099 | −1 | GGTTCTTCTTCACACTACCTTTCCCTTTCA | 150 | GGG |
| African | 4102 | 1 | TGTGGAGGGTTTATGCTCCTCTCCCTGAAA | 151 | GGG |
| African | 4130 | −1 | GCAGTCAATCCCAAGGCCATGACAAATGGC | 152 | AGG |
| African | 4409 | 1 | TTGAAAGAGCAGGTGACATCACATGGGAAA | 153 | AGG |
| African | 4639 | −1 | TGGTCTCTCCTTTTTTCACTTCTTTGGGAG | 154 | CGG |
| African | 4672 | 1 | AAAGAAGTGAAAAAAGGAGAGACCACAGAT | 155 | GGG |
| African | 4673 | 1 | AAGAAGTGAAAAAAGGAGAGACCACAGATG | 156 | GGG |
| African | 4696 | −1 | CTCCAACCTGTGTTGAACCCAGCAGTCTGC | 157 | GGG |
| African | 4697 | −1 | ACTCCAACCTGTGTTGAACCCAGCAGTCTG | 158 | CGG |
| African | 4703 | 1 | GGGTATACAGAGTGATGACCCGCAGACTGC | 159 | TGG |
| African | 4715 | 1 | TGATGACCCGCAGACTGCTGGGTTCAACAC | 160 | AGG |
| African | 4754 | −1 | GCAGCTCCTTTTGTGACGTGCCACATGGTG | 161 | TGG |
| African | 4786 | 1 | ATGTGGCACGTCACAAAAGGAGCTGCACTG | 162 | AGG |
| African | 4819 | 1 | AGCGGTGAAGGGAGACTTGATCCATACTGG | 163 | GGG |
| African | 4820 | 1 | GCGGTGAAGGGAGACTTGATCCATACTGGG | 164 | GGG |
| African | 4852 | 1 | GATGTCAAGCAGGACTTAGTGTCATACTGT | 165 | GGG |
| African | 4865 | 1 | ACTTAGTGTCATACTGTGGGCCTTGGAAGT | 166 | TGG |
| African | 4924 | 1 | GTGCAGCTCTTGGCAGTACCCCCCGGAGAG | 167 | AGG |
| African | 4925 | 1 | TGCAGCTCTTGGCAGTACCCCCCGGAGAGA | 168 | GGG |
| African | 4933 | −1 | TGAATATTCCAGGCAGAGTCTGAATGTTTC | 169 | TGG |
| African | 5008 | −1 | TGTCTAGGATCGGGGATCCTGAAGTTCCTG | 170 | CGG |
| African | 5026 | −1 | CTATCACTCTTCCGCATTTGTCTAGGATCG | 171 | GGG |
| African | 5027 | −1 | CCTATCACTCTTCCGCATTTGTCTAGGATC | 172 | GGG |
| African | 5111 | 1 | AGAATGGAAGCTATGTTAGTGCCATAACCC | 173 | AGG |
| African | 5126 | 1 | TTAGTGCCATAACCCAGGGAAAGAGGGAGG | 174 | AGG |
| African | 5135 | 1 | TAACCCAGGGAAAGAGGGAGGAGGAGACTC | 175 | CGG |
| African | 5158 | −1 | GGACAGTTAGCTGCTTCTTCTTCAGCATCG | 176 | AGG |
| African | 5186 | 1 | CGATGCTGAAGAAGAAGCAGCTAACTGTCC | 177 | TGG |
| African | 5189 | −1 | CTAGTCTTTCCGGCTCCTGGATGCAGATCC | 178 | AGG |
| African | 5209 | −1 | CTATTTCAGGAAGAACTCTCCTAGTCTTTC | 179 | CGG |
| African | 5276 | 1 | CCATAAAAAAGAGACTCCGCACAGTGATTT | 180 | TGG |
| African | 5330 | 1 | CTGAGATGGAGGAAGCCTTGAGAGGACTTC | 181 | CGG |
| African | 5371 | 1 | ATGACAACAGCAGTTAACGTCACCCACTCT | 182 | GGG |
| African | 5416 | −1 | TAGGGACTCTGATGGGTTGTAATAGGCGTG | 183 | AGG |
| African | 5423 | −1 | TTGTAATTAGGGACTCTGATGGGTTGTAAT | 184 | AGG |
| African | 5434 | −1 | TGATGTAGAGATTGTAATTAGGGACTCTGA | 185 | TGG |
| African | 5489 | −1 | GATATGTATCCTCTTGCAGCTATACTTGAG | 186 | GGG |
| African | 5678 | −1 | CTTGGAACGAACCAAACTGTTTTCCCAGAA | 187 | TGG |
| African | 5680 | 1 | TCAGGCTTTGATTGGGTGACAGACCATTCT | 188 | GGG |
| African | 5737 | −1 | GTATGACCCGCTTTCCAGCCTTTGTCAGAC | 189 | AGG |
| African | 5773 | 1 | AAGGCTGGAAAGCGGGTCATACAACTCAGC | 190 | AGG |
| African | 5875 | 1 | TCAGAGATGGGCGCGAATTTCAAAGCTGAC | 191 | CGG |
| African | 5876 | 1 | CAGAGATGGGCGCGAATTTCAAAGCTGACC | 192 | GGG |
| African | 5941 | 1 | ATACTTGATGGTGAGAGAGTCATCTTGGCT | 193 | GGG |
| African | 6005 | −1 | CCATACATGTACTCATCTCCAGGTTTGTTA | 194 | GGG |
| African | 6055 | −1 | TTGCTTCAAGCCAGTGTGCATGGTCTTCAT | 195 | CGG |
| African | 6136 | 1 | CTCCAGGATGGCCTCATAGCCTCGCTCTAC | 196 | CGG |
| African | 6140 | −1 | TCAATGGCAGCTACCTTATCGGCCTCAGGC | 197 | CGG |
| African | 6152 | 1 | TAGCCTCGCTCTACCGGCCTGAGGCCGATA | 198 | AGG |
| African | 6236 | −1 | GATGCAACCTGATAGGCTAGCCAAACGGGA | 199 | AGG |
| African | 6403 | 1 | AGAGTGCTCAAACCAAGATGGATGGATGCG | 200 | AGG |
| African | 6404 | 1 | GAGTGCTCAAACCAAGATGGATGGATGCGA | 201 | GGG |
| African | 6418 | −1 | ATTCTTTGAACGACTTCAGGGCAGCATGAT | 202 | CGG |
| African | 6457 | −1 | CCATTACTCCTAAAGCCACTCCTCTTTTCC | 203 | CGG |
| African | 6485 | 1 | GGAAAAGAGGAGTGGCTTTAGGAGTAATGG | 204 | AGG |
| African | 6520 | 1 | CTGGGAACATTGCCAGGACACATGACAGAG | 205 | AGG |
| African | 6587 | 1 | TGCGAGCAGAGACTGGAAGCAGGCCTTACA | 206 | AGG |
| African | 6661 | 1 | CTCTTAGGCTTGTTGGGAACAGTTTCGTTG | 207 | GGG |
| African | 6829 | −1 | TGTCCTGGGGAGATCTTTGCTTCTCTGGCT | 208 | CGG |
| African | 6881 | 1 | AGGACAACCAGATGGCAATCATCATCATGG | 209 | TGG |
| African | 6887 | 1 | ACCAGATGGCAATCATCATCATGGTGGCAG | 210 | TGG |
| African | 6989 | 1 | TAATGGGAAGGAGAGAAGAAGGAGTAACTA | 211 | TGG |
| African | 7033 | −1 | TGAGAGTTGTCAGTGCGGCATAGATAGCCC | 212 | AGG |
| African | 7048 | −1 | GGACGGCTGGGGTGATGAGAGTTGTCAGTG | 213 | CGG |
| African | 7082 | 1 | CAACTCTCATCACCCCAGCCGTCCAACACG | 214 | CGG |
| African | 7206 | 1 | AT-GGGACTTTGGAGTCCCGCTGCTAATGA | 215 | TGG |
| African | 7358 | −1 | CAATGTCAGTTACCACTATTCCATCCACAA | 216 | CGG |
| African | 7526 | −1 | ATTTGTTTGGAGAACCTTCCCACAAGGTGG | 217 | AGG |
| African | 7580 | −1 | GGTAACTTCCTCTGAAGATGTTGCACAGTG | 218 | AGG |
| African | 7611 | −1 | GTCACTGTATAAATAAGAGAAGCGCCTGCC | 219 | AGG |
| African | 7656 | −1 | TCTCCCGTTCCACCTCCACGTCTCTTGACT | 220 | AGG |
| African | 7677 | 1 | CTGGCCTAGTCAAGAGACGTGGAGGTGGAA | 221 | CGG |
| African | 7716 | −1 | GAGTAGAACTCCAGGGCCGACATCTGATTC | 222 | AGG |
| African | 7779 | 1 | AAAAGTCAGGCATCACTGAAGTGTGTAGAG | 223 | AGG |
| African | 7817 | −1 | CGCTTCCCCGGGATACAGCATGTCCTCCTG | 224 | TGG |
| African | 7835 | 1 | GGAGTGGCCACAGGAGGACATGCTGTATCC | 225 | CGG |
| African | 7836 | 1 | GAGTGGCCACAGGAGGACATGCTGTATCCC | 226 | GGG |
| African | 7890 | −1 | CTGCCACATCCGAGGTCAACAACCTTTCCA | 227 | TGG |
| African | 7908 | −1 | TAATAGCTCCAACCCCCTCTGCCACATCCG | 228 | AGG |
| African | 7959 | 1 | GCTATTATGCCGCCACCATCCGGAAAGTGC | 229 | AGG |
| African | 7962 | 1 | ATTATGCCGCCACCATCCGGAAAGTGCAGG | 230 | AGG |
| African | 7980 | 1 | GGAAAGTGCAGGAGGTGAAAGGATACACAA | 231 | AGG |
| African | 7994 | −1 | AGCTTTGCACCAGCATGGGTTCTTCATGAC | 232 | CGG |
| African | 8081 | −1 | CTATGTCACACAGCAAGGTATCACACGGCT | 233 | CGG |
| African | 8096 | −1 | TAGATGATGACTCACCTATGTCACACAGCA | 234 | AGG |
| African | 8157 | −1 | TCAAGCCAGTCCCCCACCATAGAGAGCACT | 235 | CGG |
| African | 8193 | 1 | CTATGGTGGGGGACTGGCTTGAGAAAAGAC | 236 | CGG |
| African | 8195 | 1 | ATGGTGGGGGACTGGCTTGAGAAAAGACCG | 237 | GGG |
| African | 8196 | 1 | TGGTGGGGGACTGGCTTGAGAAAAGACCGG | 238 | GGG |
| African | 8211 | 1 | TTGAGAAAAGACCGGGGGCCTTCTGTATAA | 239 | AGG |
| African | 8262 | −1 | CTGACTAATCCTCCCCCATACCTACGTTGC | 240 | AGG |
| African | 8315 | −1 | TTGCTCCAGAGACCCAATACATCTCATGTG | 241 | TGG |
| African | 8391 | −1 | ACTGGCCTCCTGGGACCATCCATGCGTCCC | 242 | AGG |
| African | 8445 | −1 | CAGCTTGCCACAGCTCGTGTGCCCGAGCCG | 243 | AGG |
| African | 8448 | 1 | TGAAATATGAGGAAGATGTGAACCTCGGCT | 244 | CGG |
| African | 8493 | 1 | TGGCAAGCTGTGCTGAAGCTCCCAACATGA | 245 | AGG |
| African | 8585 | −1 | CTTGTGTGGGGGCTTCGTAGCTCCCATGGT | 246 | AGG |
| African | 8588 | 1 | AACCATCCATACAGGACATGGGCCTACCAT | 247 | GGG |
| African | 8630 | −1 | TTGACAGGAGTCTAACAACCCCATTCACGA | 248 | GGG |
| African | 8631 | −1 | TTTGACAGGAGTCTAACAACCCCATTCACG | 249 | AGG |
| African | 8724 | −1 | GTGTCCACTTTTTCTTTGAAGACTCTTTGT | 250 | TGG |
| African | 8756 | −1 | GGCGAGTGCCTTCTTGGGGATCTGGCACCC | 251 | TGG |
| African | 8787 | −1 | CACAGCCAGGAAGAGACCATGTTCATTGCC | 252 | TGG |
| African | 8817 | 1 | CAATGAACATGGTCTCTTCCTGGCTGTGGA | 253 | AGG |
| African | 8823 | 1 | ACATGGTCTCTTCCTGGCTGTGGAAGGAGT | 254 | TGG |
| African | 8825 | 1 | ATGGTCTCTTCCTGGCTGTGGAAGGAGTTG | 255 | GGG |
| African | 8895 | 1 | TCATCAACAAGGTGCGCAGCAATGCAGCAC | 256 | TGG |
| African | 8931 | 1 | CAATATTTGAAGAGGAAAAAGAATGGAAGA | 257 | CGG |
| African | 8939 | −1 | CCCAAAACCTTGGATCATTCACAGCCTCCA | 258 | CGG |
| African | 8940 | 1 | AAGAGGAAAAAGAATGGAAGACGGCCGTGG | 259 | AGG |
| African | 9105 | 1 | AAGGCAGCCGCGCCATCTGGTACATGTGGT | 260 | TGG |
| African | 9116 | −1 | AGAATCCAAGGGCTTCAAACTCCAAGAATC | 261 | TGG |
| African | 9120 | 1 | TCTGGTACATGTGGTTGGGAGCCAGATTCT | 262 | TGG |
| African | 9159 | −1 | CCACCTCCTGAGTTTTCTCTTCCCATCCAA | 263 | TGG |
| African | 9239 | 1 | AGACTTGGATACATTCTAGAAGAAATGAAT | 264 | CGG |
| African | 9240 | 1 | GACTTGGATACATTCTAGAAGAAATGAATC | 265 | GGG |
| African | 9278 | −1 | CAAACTTACTAATGCGGGTGTCCCAGCCAG | 266 | CGG |
| African | 9326 | −1 | TGTGCCCTTCCTCCATTTGGTTGGTAATTA | 267 | AGG |
| African | 9342 | 1 | AGAATGAAGCCTTAATTACCAACCAAATGG | 268 | AGG |
| African | 9424 | 1 | CAAAGTGGTGAAGGTCCTCAGACCAGCTGA | 269 | AGG |
| African | 9459 | 1 | GGAAAACAGTTATGGACATCATTTCAAGAC | 270 | AGG |
| African | 9494 | −1 | CCACTAAGTTGGTAAATGTGTTGAGAGCAT | 271 | AGG |
| African | 9537 | −1 | ATCTCTAACACTTCCTCAGCCTCCATATTC | 272 | CGG |
| African | 9595 | −1 | ATTGCACTGCAACCATCTGGTCACTTTCTC | 273 | TGG |
| African | 9747 | 1 | ATGACATGGGAAAAGTTAGGAAAGACACAC | 274 | AGG |
| African | 9795 | −1 | TGCAGCTTGTTGAAATGGTGGGAGCAGAAC | 275 | GGG |
| African | 9855 | −1 | CGGCCAATCAATTCATCTTGGTGGCGGCAA | 276 | GGG |
| African | 9932 | −1 | GCCACATCTGTGCATATGATTTTGCTAGAC | 277 | AGG |
| African | 10014 | 1 | ACCTTCGACTGATGGCCAATGCTATTTGTT | 278 | CGG |
| African | 10046 | −1 | CCTTTCCGTGGATTGACCAGGTGGTTCTCC | 279 | CGG |
| African | 10092 | 1 | TCCACGGAAAGGGAGAATGGATGACTACTG | 280 | AGG |
| African | 10146 | 1 | GAGTGTGGATTGAGGAGAACGACCATATGG | 281 | AGG |
| African | 10185 | −1 | CACCATAAGTCCTCCCTTTTTCCCAGATAG | 282 | GGG |
| African | 10200 | 1 | GGACAGACATCCCCTATCTGGGAAAAAGGG | 283 | AGG |
| African | 10223 | −1 | CCCAAGTGGTGCGGGGCCTGTGCCCTATAA | 284 | GGG |
| African | 10224 | −1 | GCCCAAGTGGTGCGGGGCCTGTGCCCTATA | 285 | AGG |
| African | 10232 | 1 | GACTTATGGTGTGGATCCCTTATAGGGCAC | 286 | AGG |
| African | 10241 | −1 | TGTCTTTGATGTTCTCAGCCCAAGTGGTGC | 287 | GGG |
| African | 10242 | −1 | GTGTCTTTGATGTTCTCAGCCCAAGTGGTG | 288 | CGG |
| African | 10332 | 1 | AAGAAAAATACATGGACTACTTATCCACCC | 289 | AGG |
| African | 10341 | −1 | CCAGGTGTGGACCCTTCCTCACCCAAGTAG | 290 | CGG |
| African | 10350 | 1 | ACTTATCCACCCAGGTCCGCTACTTGGGTG | 291 | AGG |
| American | 188 | 1 | AACGCGGAGTAGCCCGTGTGAGCCCCTTTG | 292 | GGG |
| American | 229 | 1 | AGGCTGCCAGCCGGACTTCTGCTGGGTCAT | 293 | GGG |
| American | 275 | 1 | TCTTGGCGATTCTAGCCTTTTTGAGATTCA | 294 | CGG |
| American | 368 | 1 | TGGAAATAATAAAGAAGTTCAAGAAAGATC | 295 | TGG |
| American | 452 | −1 | ACCTCCGCTGCCATAGCTGTGGTCAGCAGG | 296 | AGG |
| American | 463 | −1 | CACGTCTAGTGACCTCCGCTGCCATAGCTG | 297 | TGG |
| American | 476 | 1 | GCCTCCTGCTGACCACAGCTATGGCAGCGG | 298 | AGG |
| American | 490 | 1 | ACAGCTATGGCAGCGGAGGTCACTAGACGT | 299 | GGG |
| American | 530 | 1 | ACTATATGTACTTGGACAGAAACGATGCTG | 300 | GGG |
| American | 552 | −1 | TATATAACACTTATTCATCCCCAATGTGGT | 301 | TGG |
| American | 559 | 1 | GGGGAGGCCATATCTTTTCCAACCACATTG | 302 | GGG |
| American | 646 | 1 | ATGAGCTATGAATGCCCTATGCTGGATGAG | 303 | GGG |
| American | 647 | 1 | TGAGCTATGAATGCCCTATGCTGGATGAGG | 304 | GGG |
| American | 719 | −1 | CTAGATCTCCGTGCTTCACCTTTTTTGTGA | 305 | TGG |
| American | 769 | −1 | ACCGCGTTTGCAGCTTCCTAGTGGAATGGG | 306 | AGG |
| American | 772 | −1 | GCGACCGCGTTTGCAGCTTCCTAGTGGAAT | 307 | GGG |
| American | 773 | −1 | TGCGACCGCGTTTGCAGCTTCCTAGTGGAA | 308 | TGG |
| American | 809 | 1 | GGAAGCTGCAAACGCGGTCGCAAACCTGGT | 309 | TGG |
| American | 1000 | 1 | TACAGCATCAGGTGCATAGGAGTCAGCAAT | 310 | AGG |
| American | 1001 | 1 | ACAGCATCAGGTGCATAGGAGTCAGCAATA | 311 | GGG |
| American | 1136 | 1 | AGCTGGTTACAACAACAGTCAGCAACATGG | 312 | CGG |
| American | 1196 | −1 | TCAAGGTAGGCTTCACCTTGTGTTGGGCAG | 313 | CGG |
| American | 1357 | −1 | ACTCCAGATTCTCTGGCTGGATGCTCTTCC | 314 | CGG |
| American | 1387 | 1 | AAGAGCATCCAGCCAGAGAATCTGGAGTAC | 315 | CGG |
| American | 1391 | −1 | TGCTGGGAGCCATGAACTGACAGCATTATC | 316 | CGG |
| American | 1507 | −1 | CTAGGCTTCCAAACCCCCCCAGGGTGGCTT | 317 | CGG |
| American | 1513 | −1 | CAAGTCCTAGGCTTCCAAACCCCCCCAGGG | 318 | TGG |
| American | 1517 | −1 | CAATCAAGTCCTAGGCTTCCAAACCCCCCC | 319 | AGG |
| American | 1519 | 1 | AATTCACCAAGAGCCGAAGCCACCCTGGGG | 320 | GGG |
| American | 1535 | −1 | AGGCCTGTCCTCGGTTCACAATCAAGTCCT | 321 | AGG |
| American | 1619 | 1 | CTATGAATAACAAGCACTGGCTGGTTCACA | 322 | AGG |
| American | 1634 | −1 | GCCCCAGCGTGCCAAGGTAATGGAATGTCG | 323 | TGG |
| American | 1712 | 1 | GGAACAACAAAGAAGCACTGGTAGAGTTCA | 324 | AGG |
| American | 1742 | 1 | AGGACGCACATGCCAAAAGGCAAACTGTCG | 325 | TGG |
| American | 1783 | −1 | CATCCATCTCAGCCTCCAGAGCTCCAGCAA | 326 | GGG |
| American | 1784 | −1 | CCATCCATCTCAGCCTCCAGAGCTCCAGCA | 327 | AGG |
| American | 1817 | 1 | GAGCTCTGGAGGCTGAGATGGATGGTGCAA | 328 | AGG |
| American | 1834 | −1 | CCATTTTCAGGCGACATTTCAAGTGGCCAG | 329 | AGG |
| American | 1856 | −1 | ACGCCCTTCAATCTAAGTTTATCCATTTTC | 330 | AGG |
| American | 1928 | 1 | GTACTGCAGCGTTCACATTCACCAAGATCC | 331 | CGG |
| American | 1945 | 1 | TTCACCAAGATCCCGGCTGAAACACTGCAC | 332 | GGG |
| American | 2068 | −1 | TCTTAGAGTTCTCAGTGCTTTCAGTGATTA | 333 | CGG |
| American | 2174 | −1 | TTTCCAATGGTGCTGCCACTCCTGTGCCAG | 334 | TGG |
| American | 2315 | −1 | AATGATTTGAAAGCTGCTCCAAAGATTTGA | 335 | TGG |
| American | 2482 | −1 | AGTCCACCGAGCACCCCACATCAGCAGAGA | 336 | CGG |
| American | 2519 | 1 | ATGTGGGGTGCTCGGTGGACTTCTCAAAGA | 337 | AGG |
| American | 2638 | −1 | CAGAGGAGATCCCGCAGATACCATCTTCCC | 338 | AGG |
| American | 2723 | 1 | GATCAGTAGAAGGGGAGCTCAACGCAATCC | 339 | TGG |
| American | 2747 | 1 | CAATCCTGGAAGAGAATGGAGTTCAACTGA | 340 | CGG |
| American | 2756 | 1 | AAGAGAATGGAGTTCAACTGACGGTCGTTG | 341 | TGG |
| American | 2806 | −1 | TCCAGCCGTGGGGCAGCTCGTTCACAGGCA | 342 | CGG |
| American | 2834 | 1 | TGCCTGTGAACGAGCTGCCCCACGGCTGGA | 343 | AGG |
| American | 2891 | 1 | GAGCAGCAAAGACAAATAACAGCTTTGTCG | 344 | TGG |
| American | 2921 | −1 | AGAAAGCTGTTCCATGCTCTATGTTTGAGT | 345 | GGG |
| American | 3175 | −1 | TCTCTTCTATTCCATCTGTCCACAATGTGT | 346 | GGG |
| American | 3176 | −1 | CTCTCTTCTATTCCATCTGTCCACAATGTG | 347 | TGG |
| American | 3223 | −1 | TGTGATGGCTGAGTGGCCCAGCTAAAGACT | 348 | TGG |
| American | 3232 | 1 | AGTGATCTGATCATACCCAAGTCTTTAGCT | 349 | GGG |
| American | 3259 | −1 | GCCCTTTCATTTGGGTCCTGTAGCCCTCTC | 350 | TGG |
| American | 3268 | 1 | CTCAGCCATCACAATACCAGAGAGGGCTAC | 351 | AGG |
| American | 3277 | −1 | GCTCTTCACTGTGCCATGGCCCTTTCATTT | 352 | GGG |
| American | 3278 | −1 | AGCTCTTCACTGTGCCATGGCCCTTTCATT | 353 | TGG |
| American | 3332 | −1 | CATGTTTCCTCCACGTGGACCTTAGTGCCT | 354 | GGG |
| American | 3427 | 1 | AGCGGAAGGGTGATCGAGGAATGGTGCTGC | 355 | AGG |
| American | 3428 | 1 | GCGGAAGGGTGATCGAGGAATGGTGCTGCA | 356 | GGG |
| American | 3457 | 1 | AGGGAGTGCACAATGCCCCCACTGTCGTTC | 357 | CGG |
| American | 3458 | 1 | GGGAGTGCACAATGCCCCCACTGTCGTTCC | 358 | GGG |
| American | 3461 | −1 | TCCATTCCATACCAACAGCCATCTTTAGCC | 359 | CGG |
| American | 3577 | −1 | GCACCATGAGCAGAATCACAAGCACTCCAA | 360 | GGG |
| American | 3578 | −1 | TGCACCATGAGCAGAATCACAAGCACTCCA | 361 | AGG |
| American | 3605 | 1 | TTGGAGTGCTTGTGATTCTGCTCATGGTGC | 362 | AGG |
| American | 3707 | −1 | GTGGCACCCATCAAAATTGCAAGCTTAGCC | 363 | AGG |
| American | 3740 | 1 | AGCTTGCAATTTTGATGGGTGCCACCTTCG | 364 | CGG |
| American | 3850 | −1 | ACGAGGCCAAGGCCAGCAGCATGCTTTCAC | 365 | GGG |
| American | 3851 | −1 | CACGAGGCCAAGGCCAGCAGCATGCTTTCA | 366 | CGG |
| American | 3863 | 1 | CTAATTGGACACCCCGTGAAAGCATGCTGC | 367 | TGG |
| American | 3877 | −1 | AGGCGGAGATCGCAGTTTGCAAAAGACACG | 368 | AGG |
| American | 3904 | −1 | TGATGAGAACCATCAGGTCGCCTTCCAAGG | 369 | CGG |
| American | 3905 | 1 | CGTGTCTTTTGCAAACTGCGATCTCCGCCT | 370 | TGG |
| American | 3907 | −1 | CATTGATGAGAACCATCAGGTCGCCTTCCA | 371 | AGG |
| American | 4000 | −1 | CCAGTGGTGTCAGAGCAGCCAGGATTGCCA | 372 | AGG |
| American | 4025 | 1 | CCTTGGCAATCCTGGCTGCTCTGACACCAC | 373 | TGG |
| American | 4030 | 1 | GCAATCCTGGCTGCTCTGACACCACTGGCC | 374 | CGG |
| American | 4031 | 1 | CAATCCTGGCTGCTCTGACACCACTGGCCC | 375 | GGG |
| American | 4075 | 1 | GTGGCGTGGAGAGCAGGCCTTGCTACTTGC | 376 | GGG |
| American | 4076 | 1 | TGGCGTGGAGAGCAGGCCTTGCTACTTGCG | 377 | GGG |
| American | 4148 | −1 | GGGTCGACCAGCCTCACAGCGGTTAGTCCC | 378 | AGG |
| American | 4159 | −1 | CCACGTTGATGGGGTCGACCAGCCTCACAG | 379 | CGG |
| American | 4166 | 1 | TCATGGCCCTGGGACTAACCGCTGTGAGGC | 380 | TGG |
| American | 4322 | 1 | CAGATATAGAGATGGCTGGGCCCATGGCCG | 381 | CGG |
| American | 4324 | −1 | CCACGTAACTGACAATTAGCAGACCGACCG | 382 | CGG |
| American | 4441 | −1 | CACCACTCTCATCTAGCGCCACATCGAGCC | 383 | GGG |
| American | 4442 | −1 | TCACCACTCTCATCTAGCGCCACATCGAGC | 384 | CGG |
| American | 4480 | −1 | CTCTCATGGGGGGACCGTCATCCTCCACCA | 385 | GGG |
| American | 4501 | −1 | GGACCACCTTGAGTATGATCTCTCTCATGG | 386 | GGG |
| American | 4502 | −1 | AGGACCACCTTGAGTATGATCTCTCTCATG | 387 | GGG |
| American | 4643 | 1 | GTGGTGCTCTATGGGATGTGCCTGCTCCCA | 388 | AGG |
| American | 4655 | 1 | GGGATGTGCCTGCTCCCAAGGAAGTAAAAA | 389 | AGG |
| American | 4657 | 1 | GATGTGCCTGCTCCCAAGGAAGTAAAAAAG | 390 | GGG |
| American | 4658 | 1 | ATGTGCCTGCTCCCAAGGAAGTAAAAAAGG | 391 | GGG |
| American | 4741 | 1 | ACACAAGTTGGAGTGGGAGTTATGCAAGAG | 392 | GGG |
| American | 4742 | 1 | CACAAGTTGGAGTGGGAGTTATGCAAGAGG | 393 | GGG |
| American | 4783 | −1 | GATCAAGTCTCCCTTCACCGCTTCTCAGCG | 394 | CGG |
| American | 4838 | 1 | ATCCATACTGGGGAGATGTCAAGCAGGATC | 395 | TGG |
| American | 4879 | −1 | CCAAGAGCTGCACCTCGCTGTGCCCGTCCC | 396 | AGG |
| American | 4882 | 1 | GGTCCATGGAAGCTAGATGCCGCCTGGGAC | 397 | GGG |
| American | 4912 | −1 | GGATGTTCCTCGCTCTCTCTCCGGGGGGCA | 398 | CGG |
| American | 4930 | 1 | CTCTTGGCCGTGCCCCCCGGAGAGAGAGCG | 399 | AGG |
| American | 4943 | −1 | TCCTTTGTCTTAAATATTCCGGGCAGAGTC | 400 | TGG |
| American | 4954 | −1 | CAATGTCCCCATCCTTTGTCTTAAATATTC | 401 | CGG |
| American | 4985 | 1 | TATTTAAGACAAAGGATGGGGACATTGGAG | 402 | CGG |
| American | 5041 | 1 | ACTTCAGGATCTCCTATCCTAGACAAGTGT | 403 | GGG |
| American | 5086 | 1 | CTTTATGGCAATGGGGTCGTGATCAAAAAT | 404 | GGG |
| American | 5116 | 1 | GGGAGTTATGTTAGTGCCATCACCCAAGGG | 405 | AGG |
| American | 5206 | 1 | CTAACTGTCTTAGACTTGCATCCTGGAGCT | 406 | GGG |
| American | 5218 | −1 | CTTCACGGACTATTTCAGGAAGAACTCTCC | 407 | TGG |
| American | 5312 | 1 | CAACCAGGGTTGTCGCTGCTGAAATGGAGG | 408 | AGG |
| American | 5321 | −1 | GTTGTCATATAACGCACTGGAAGCCCTCTA | 409 | AGG |
| American | 5323 | 1 | GTCGCTGCTGAAATGGAGGAGGCCCTTAGA | 410 | GGG |
| American | 5446 | −1 | GGGCCTCATCCATAATATACAGATTATAGT | 411 | TGG |
| American | 5468 | 1 | TCCCCAACTATAATCTGTATATTATGGATG | 412 | AGG |
| American | 5548 | −1 | TTCCTGGTGGCGTGGCGGTCATGAAGATGG | 413 | CGG |
| American | 5563 | −1 | GAAATGCGTCACGGGTTCCTGGTGGCGTGG | 414 | CGG |
| American | 5591 | 1 | CCACGCCACCAGGAACCCGTGACGCATTTC | 415 | CGG |
| American | 5623 | −1 | AGCTCCAGGCTCTCTCTGGGACTTCCACTT | 416 | CGG |
| American | 5669 | 1 | GAGCCTGGAGCTCAGGCTTTGATTGGGTGA | 417 | CGG |
| American | 5710 | 1 | GGAAAAACAGTTTGGTTTGTTCCAAGCGTG | 418 | AGG |
| American | 5801 | −1 | ACAAAGTCCCACTCTTGATGTTTTGTTTTC | 419 | TGG |
| American | 5863 | −1 | TGGAATCTATGACACGGTCAGCTTTAAAGT | 420 | TGG |
| American | 5893 | −1 | CATCAAGTATGACCGGCTTTAGGCATCTCC | 421 | TGG |
| American | 5906 | 1 | GTGTCATAGATTCCAGGAGATGCCTAAAGC | 422 | CGG |
| American | 5968 | −1 | TGCCTATGCGCCCCCTCCTCTGGGCAGCGC | 423 | TGG |
| American | 5978 | −1 | TTGGGATTCCTGCCTATGCGCCCCCTCCTC | 424 | TGG |
| American | 5980 | 1 | CCTGTCACACATGCCAGCGCTGCCCAGAGG | 425 | AGG |
| American | 5981 | 1 | CTGTCACACATGCCAGCGCTGCCCAGAGGA | 426 | GGG |
| American | 6007 | −1 | CTCCATACAGATACTCATCTCCAGGTTTGT | 427 | TGG |
| American | 6037 | 1 | AAACCTGGAGATGAGTATCTGTATGGAGGT | 428 | GGG |
| American | 6095 | −1 | ATGAGGCCATCTTGGAGGTAAATATTGTCA | 429 | AGG |
| American | 6188 | 1 | CAGCCATTGAGGGAGAGTTCAAGCTTAGGA | 430 | CGG |
| American | 6245 | 1 | TCATGAAAAGAGGAGATCTTCCTGTTTGGC | 431 | TGG |
| American | 6280 | −1 | TGCCATCAAAGCACCATCTTCTATCTGTGT | 432 | AGG |
| American | 6341 | 1 | CCAACAACACCATAATGGAAGACAGTGTGC | 433 | CGG |
| American | 6361 | −1 | TCGGTTTGAGCACTCTTTTCTCTCCGTGTC | 434 | TGG |
| American | 6388 | 1 | AGACACGGAGAGAAAAGAGTGCTCAAACCG | 435 | AGG |
| American | 6406 | −1 | ACTTCAGGGCCGCATGATCTGAACAAACTC | 436 | TGG |
| American | 6422 | 1 | GGATGGACGCCAGAGTTTGTTCAGATCATG | 437 | CGG |
| American | 6440 | 1 | GTTCAGATCATGCGGCCCTGAAGTCATTCA | 438 | AGG |
| American | 6548 | −1 | CTTCCAGTCTCTGCCCGCATGAGCACAGCG | 439 | AGG |
| American | 6559 | 1 | GAAGCCATTGACAACCTCGCTGTGCTCATG | 440 | CGG |
| American | 6560 | 1 | AAGCCATTGACAACCTCGCTGTGCTCATGC | 441 | GGG |
| American | 6595 | −1 | TCTCTAGGGTCTCCGGCAATTGGGCCGCCG | 442 | CGG |
| American | 6596 | 1 | AGACTGGAAGCAGGCCTTACAAAGCCGCGG | 443 | CGG |
| American | 6635 | 1 | TGCCGGAGACCCTAGAGACCATTATGCTTT | 444 | TGG |
| American | 6637 | 1 | CCGGAGACCCTAGAGACCATTATGCTTTTG | 445 | GGG |
| American | 6806 | −1 | TCTGGCTCAGGTATGAGCACCACCAGCAAT | 446 | AGG |
| American | 6913 | −1 | TTGTTCTCTCCAACCATCCGAGTTCATTGG | 447 | CGG |
| American | 6916 | −1 | TCTTTGTTCTCTCCAACCATCCGAGTTCAT | 448 | TGG |
| American | 6953 | −1 | CCCTCCTCTCTCCTTCCCATTAGATGGCTT | 449 | AGG |
| American | 6959 | −1 | GTTGCCCCCTCCTCTCTCCTTCCCATTAGA | 450 | TGG |
| American | 6974 | 1 | GTGACCTAAGCCATCTAATGGGAAGGAGAG | 451 | AGG |
| American | 6977 | 1 | ACCTAAGCCATCTAATGGGAAGGAGAGAGG | 452 | AGG |
| American | 6979 | 1 | CTAAGCCATCTAATGGGAAGGAGAGAGGAG | 453 | GGG |
| American | 6980 | 1 | TAAGCCATCTAATGGGAAGGAGAGAGGAGG | 454 | GGG |
| American | 6991 | −1 | GCCGCAGGTCAATGTCCATTGAGAATCCTA | 455 | TGG |
| American | 7016 | −1 | GCATAGATGGCCCAAGCTGAGGCTGGCCGC | 456 | AGG |
| American | 7039 | −1 | GGGTAATGAAAGTTGTCAAGGCAGCATAGA | 457 | TGG |
| American | 7051 | −1 | GTTGGACGGCTGGGGTAATGAAAGTTGTCA | 458 | AGG |
| American | 7096 | −1 | CCATCGCCATTAAGGAGTAGTTGTTGTATG | 459 | AGG |
| American | 7242 | −1 | TGCGCCACGAGCAAAATGATGGCCACTATT | 460 | AGG |
| American | 7253 | −1 | AGTACATGTAGTGCGCCACGAGCAAAATGA | 461 | TGG |
| American | 7289 | 1 | CTCGTGGCGCACTACATGTACTTGATCCCA | 462 | GGG |
| American | 7329 | 1 | CAGCAGCTGCGCGTGCTGCCCAGAAGAGAA | 463 | CGG |
| American | 7356 | −1 | ATGTCAGTCACCACTATTCCATCCACAACA | 464 | GGG |
| American | 7428 | 1 | TTGACCCCCAAGTGGAGAAAAAGATGGGAC | 465 | AGG |
| American | 7466 | −1 | CCCCCCACCCCCAGGCGGTCCGCGACAGTA | 466 | TGG |
| American | 7484 | −1 | TGATCAGGGCCCCAGCCTCCCCCCACCCCC | 467 | AGG |
| American | 7499 | 1 | TCGCGGACCGCCTGGGGGTGGGGGGAGGCT | 468 | GGG |
| American | 7500 | 1 | CGCGGACCGCCTGGGGGTGGGGGGAGGCTG | 469 | GGG |
| American | 7508 | −1 | CCCACAAAGTGGAAGTTGCGGCTGTGATCA | 470 | GGG |
| American | 7509 | −1 | TCCCACAAAGTGGAAGTTGCGGCTGTGATC | 471 | AGG |
| American | 7520 | −1 | TCGGAGAGCCTTCCCACAAAGTGGAAGTTG | 472 | CGG |
| American | 7595 | 1 | TCTACAGCCACTTCACTGTGTAACATTTTT | 473 | AGG |
| American | 7596 | 1 | CTACAGCCACTTCACTGTGTAACATTTTTA | 474 | GGG |
| American | 7653 | 1 | TAATCTACACAGTAACAAGAAACGCTGGCT | 475 | TGG |
| American | 7667 | 1 | ACAAGAAACGCTGGCTTGGTCAAGAGACGT | 476 | GGG |
| American | 7668 | 1 | CAAGAAACGCTGGCTTGGTCAAGAGACGTG | 477 | GGG |
| American | 7689 | 1 | AGAGACGTGGGGGTGGAACAGGAGAGACCC | 478 | TGG |
| American | 7691 | −1 | GGTTCAAGCGGGCCTTCCATTTCTCTCCCA | 479 | GGG |
| American | 7692 | −1 | TGGTTCAAGCGGGCCTTCCATTTCTCTCCC | 480 | AGG |
| American | 7704 | 1 | GAACAGGAGAGACCCTGGGAGAGAAATGGA | 481 | AGG |
| American | 7712 | −1 | AGAACTCCAGGGCCGACATCTGGTTCAAGC | 482 | GGG |
| American | 7713 | −1 | TAGAACTCCAGGGCCGACATCTGGTTCAAG | 483 | CGG |
| American | 7722 | −1 | TTGTAGGAGTAGAACTCCAGGGCCGACATC | 484 | TGG |
| American | 7748 | −1 | TGCACACCTCGGTGATGCCTGACTTTTTGT | 485 | AGG |
| American | 7767 | 1 | TCTACTCCTACAAAAAGTCAGGCATCACCG | 486 | AGG |
| American | 7769 | −1 | GGGCGCGGCGGGCCTCTTCTCTGCACACCT | 487 | CGG |
| American | 7790 | −1 | CCGTTGCCACACCGTCCTTGAGGGCGCGGC | 488 | GGG |
| American | 7791 | −1 | CCCGTTGCCACACCGTCCTTGAGGGCGCGG | 489 | CGG |
| American | 7815 | 1 | CCCGCCGCGCCCTCAAGGACGGTGTGGCAA | 490 | CGG |
| American | 7827 | −1 | CTCAGCTTTGCACTTCCTCGGGACACAGCA | 491 | TGG |
| American | 7868 | 1 | GGAAGTGCAAAGCTGAGATGGTTGGTGGAG | 492 | CGG |
| American | 7869 | 1 | GAAGTGCAAAGCTGAGATGGTTGGTGGAGC | 493 | GGG |
| American | 7992 | −1 | CTTTGCACCAACACGGGTTCTTCATGACCA | 494 | GGG |
| American | 8043 | −1 | ATATGAAAGACGTCCACCCCACTCTTAAGA | 495 | CGG |
| American | 8051 | 1 | TATGGGTGGAACATAGTCCGTCTTAAGAGT | 496 | GGG |
| American | 8052 | 1 | ATGGGTGGAACATAGTCCGTCTTAAGAGTG | 497 | GGG |
| American | 8144 | 1 | TCATCATCTAGTCCTGAAGTGGAAGAAGCA | 498 | CGG |
| American | 8163 | −1 | CTTTTTTCAAGCCAATCCCCCACCATGGAG | 499 | AGG |
| American | 8168 | −1 | CTGGTCTTTTTTCAAGCCAATCCCCCACCA | 500 | TGG |
| American | 8226 | −1 | TCCAGGGTTTCCATCATAGTGCTGGTGTAT | 501 | GGG |
| American | 8253 | −1 | CCTCCCCCATACCTACGCTGCAGTCGCTCC | 502 | AGG |
| American | 8283 | 1 | AGCGACTGCAGCGTAGGTATGGGGGAGGAC | 503 | TGG |
| American | 8306 | −1 | AGACCCAGTACATCTCATGTGTAGAGTTGC | 504 | GGG |
| American | 8307 | −1 | GAGACCCAGTACATCTCATGTGTAGAGTTG | 505 | CGG |
| American | 8357 | −1 | GGAGCTGGCTCGTGGTGGACACACTTTTTA | 506 | TGG |
| American | 8382 | −1 | CTAGGCCCGTCCATGCGCCCCAAGAGGAGC | 507 | TGG |
| American | 8390 | 1 | AGTGTGTCCACCACGAGCCAGCTCCTCTTG | 508 | GGG |
| American | 8402 | 1 | ACGAGCCAGCTCCTCTTGGGGCGCATGGAC | 509 | GGG |
| American | 8430 | 1 | ACGGGCCTAGGAGGCCAGTGAAATATGAGG | 510 | AGG |
| American | 8456 | 1 | GAGGAGGATGTGAATCTCGGCTCTGGCACG | 511 | CGG |
| American | 8457 | 1 | AGGAGGATGTGAATCTCGGCTCTGGCACGC | 512 | GGG |
| American | 8511 | −1 | TCCGCGTGCTCACTGCGGATCCTTTCAATG | 513 | CGG |
| American | 8516 | 1 | AACATGAAGATCATTGGTAACCGCATTGAA | 514 | AGG |
| American | 8535 | 1 | ACCGCATTGAAAGGATCCGCAGTGAGCACG | 515 | CGG |
| American | 8568 | −1 | TAGCTTCCATGGTAAGCCCATGTCCTATAT | 516 | GGG |
| American | 8598 | 1 | ATAGGACATGGGCTTACCATGGAAGCTATG | 517 | AGG |
| American | 8627 | −1 | ACAGGAGCCTGACAACCCCGTTTATTAGAG | 518 | AGG |
| American | 8645 | 1 | TCAGCGTCCTCTCTAATAAACGGGGTTGTC | 519 | AGG |
| American | 8670 | 1 | TTGTCAGGCTCCTGTCAAAACCCTGGGATG | 520 | TGG |
| American | 8699 | −1 | TTTGCTGACCATACGGTGTGGTGTCGGTCA | 521 | TGG |
| American | 8705 | −1 | AAACTCTTTGCTGACCATACGGTGTGGTGT | 522 | CGG |
| American | 8711 | −1 | CCTTGAAAACTCTTTGCTGACCATACGGTG | 523 | TGG |
| American | 8736 | 1 | CCACACCGTATGGTCAGCAAAGAGTTTTCA | 524 | AGG |
| American | 8769 | −1 | ATGCTCATAACCTGACGAGTGCCTTCTTGG | 525 | GGG |
| American | 8897 | 1 | ATCAACAAGGTTCGTAGCAATGCAGCATTA | 526 | GGG |
| American | 8898 | 1 | TCAACAAGGTTCGTAGCAATGCAGCATTAG | 527 | GGG |
| American | 8997 | −1 | TACACACAACTCTGGCACTCTCCTCTCAGG | 528 | TGG |
| American | 9056 | 1 | AACATGATGGGAAAAAGAGAAAAGAAACAA | 529 | GGG |
| American | 9057 | 1 | ACATGATGGGAAAAAGAGAAAAGAAACAAG | 530 | GGG |
| American | 9069 | 1 | AAAGAGAAAAGAAACAAGGGGAATTTGGAA | 531 | AGG |
| American | 9075 | 1 | AAAAGAAACAAGGGGAATTTGGAAAGGCCA | 532 | AGG |
| American | 9077 | −1 | GCCACATATACCAGATGGCGCGGCTGCCCT | 533 | TGG |
| American | 9107 | 1 | GGCAGCCGCGCCATCTGGTATATGTGGCTA | 534 | GGG |
| American | 9108 | 1 | GCAGCCGCGCCATCTGGTATATGTGGCTAG | 535 | GGG |
| American | 9164 | 1 | CTTGGATTCTTGAACGAGGATCACTGGATG | 536 | GGG |
| American | 9198 | 1 | GAGAGAACTCAGGAGGTGGTGTTGAAGGGC | 537 | TGG |
| American | 9228 | −1 | CTTCCTCCTGGTATACGACTCATCTCTTCT | 538 | AGG |
| American | 9254 | 1 | CTAGAAGAGATGAGTCGTATACCAGGAGGA | 539 | AGG |
| American | 9299 | 1 | GACACTGCTGGCTGGGACACCCGCATTAGC | 540 | AGG |
| American | 9353 | 1 | CTAATCACCAACCAAATGGAGAAAGGGCAC | 541 | AGG |
| American | 9354 | 1 | TAATCACCAACCAAATGGAGAAAGGGCACA | 542 | GGG |
| American | 9362 | −1 | GGTATGTGTACTTGATTATGGCCAATGCCA | 543 | AGG |
| American | 9374 | −1 | CCACTTTGTTTTGGTATGTGTACTTGATTA | 544 | TGG |
| American | 9393 | −1 | GCTGGTCTAAGGACCTTTACCACTTTGTTT | 545 | TGG |
| American | 9467 | 1 | GTTATGGACATTATTTCGAGACAAGACCAA | 546 | AGG |
| American | 9587 | 1 | CTAGAGATGCAAGACTTGTGGCTGCTGCGG | 547 | AGG |
| American | 9632 | 1 | ACTAACTGGTTGCAGAGCAACGGATGGGAT | 548 | AGG |
| American | 9738 | 1 | GGTTCTTGAATGATATGGGAAAAGTTAGGA | 549 | AGG |
| American | 9771 | 1 | ACACACAAGAGTGGAAACCCTCAACTGGAT | 550 | GGG |
| American | 9825 | −1 | GGAACCACAATGGACCTCCCGTCCTTGAGA | 551 | TGG |
| American | 9833 | 1 | CACCACTTCAACAAGCTCCATCTCAAGGAC | 552 | GGG |
| American | 9836 | 1 | CACTTCAACAAGCTCCATCTCAAGGACGGG | 553 | AGG |
| American | 9857 | −1 | CCCGGCCAATCAGTTCATCTTGGTGGCGGC | 554 | AGG |
| American | 9881 | 1 | CCCTGCCGCCACCAAGATGAACTGATTGGC | 555 | CGG |
| American | 9882 | 1 | CCTGCCGCCACCAAGATGAACTGATTGGCC | 556 | GGG |
| American | 9903 | 1 | TGATTGGCCGGGCCCGCGTCTCTCCAGGGG | 557 | CGG |
| American | 9921 | −1 | GCATATGATTTTGCTAGGCAAGCAGTCTCC | 558 | CGG |
| American | 9936 | −1 | AGCTGCCACATTTGCGCATATGATTTTGCT | 559 | AGG |
| American | 9969 | −1 | ATCAGTCGGAGGTCCCTTCTGTGGAAATAA | 560 | AGG |
| American | 9980 | 1 | CAAATGTGGCAGCTCCTTTATTTCCACAGA | 561 | AGG |
| American | 9981 | 1 | AAATGTGGCAGCTCCTTTATTTCCACAGAA | 562 | GGG |
| American | 9993 | −1 | ACAGATGAACAAATGGCATTGGCCATCAGT | 563 | CGG |
| American | 10010 | −1 | GAACCCAGTCAACTGGCACAGATGAACAAA | 564 | TGG |
| American | 10091 | −1 | CTCTGTTCCACACCACAAGCATGTCTTCAG | 565 | TGG |
| American | 10160 | −1 | AATAGGGAATGTCTGTCCATTTCGTAACTG | 566 | GGG |
| American | 10161 | −1 | AAATAGGGAATGTCTGTCCATTTCGTAACT | 567 | GGG |
| American | 10250 | −1 | TGTTGACTGTGTTTTTAATGTTCTCAGCCC | 568 | AGG |
| American | 10326 | −1 | TCTTCACCCAAGTAGCGAACTTGGGTGGAT | 569 | AGG |

American-Asian NASBACC genotyping

| | | | | | |
|---|---|---|---|---|---|
| Asian | 469 | −1 | CACTCCCACGTCTAGTGACCTCCACTGCCA | 570 | TGG |
| Asian | 722 | −1 | CTTCTAGATCTCCGTGCTTCACCTTTTTTG | 571 | TGG |
| Asian | 839 | −1 | TTCCTGAATATCCAATTTTCGACTCTAATC | 572 | AGG |
| Asian | 904 | −1 | CTTTTTGGCTCGTTGAACTTCCCAAAAGCC | 573 | AGG |
| Asian | 1075 | −1 | TGTCGACAGCCGGTTTGTCCTGTGCCATTA | 574 | CGG |
| Asian | 1091 | 1 | GTTGTGTTACCGTAATGGCACAGGACAAAC | 575 | CGG |
| Asian | 1172 | 1 | TAAGATCCTATTGCTATGAGGCATCAATAT | 576 | CGG |
| Asian | 1472 | 1 | CAGGACATGAAACTGATGAGAATAGAGCGA | 577 | AGG |
| Asian | 1906 | −1 | CAGCCGGGATCTTGGTGAATGTGAACGCTG | 578 | CGG |
| Asian | 1993 | −1 | TGTCCACCGCCATCTGAGCTGGAACCTTGC | 579 | AGG |
| Asian | 2390 | 1 | GGTTCTCACAAATTCTCATTGGAACGTTGC | 580 | TGG |
| Asian | 2537 | 1 | ACTTCTCAAAGAAGGAAACGAGATGCGGTA | 581 | CGG |
| Asian | 2644 | 1 | GCAGCAGCAGTCAAGCAAGCCTGGGRAGAT | 582 | GGG |
| Asian | 3140 | −1 | TTTGGCCATTCACATGTTTTCATCTCGATC | 583 | AGG |
| Asian | 3712 | −1 | CAAAGGTGGCACCCATCAAAATTGCAAGCT | 584 | TGG |
| Asian | 3872 | −1 | GAGATCGCAGTTTGCAGAAGACACGAAGCC | 585 | AGG |
| Asian | 4102 | 1 | TGCGGGGGGTTCATGCTTCTCTCTCTGAAG | 586 | GGG |
| Asian | 4106 | 1 | GGGGGTTCATGCTTCTCTCTCTGAAGGGGA | 587 | AGG |
| Asian | 4130 | −1 | GCGGTGAGTCCCAAGGCCATGACAAATGGT | 588 | AGG |
| Asian | 4292 | 1 | TATGCGCGTTGGCCGGAGGGTTCGCCAAGG | 589 | CGG |
| Asian | 4454 | 1 | CTGGAAACAGTCCCCGGCTCGATGTGGCAC | 590 | TGG |
| Asian | 4754 | −1 | GCGGATCCTTTTGTGACGTGCCACATAGTG | 591 | TGG |
| Asian | 5195 | −1 | ACTCTCCTGGTTTTCCCAGCTCCAGGATGC | 592 | AGG |
| Asian | 5371 | 1 | ATGACAACAGCAGTCAATGTCACCCATTCT | 593 | GGG |
| Asian | 5569 | −1 | AGTCCGGGAATGCGTCACGGGTTCCTGGTG | 594 | GGG |
| Asian | 5570 | −1 | GAGTCCGGGAATGCGTCACGGGTTCCTGGT | 595 | GGG |
| Asian | 5594 | −1 | TCGGTGTCCATAATTGGTGAGTTGGAGTCC | 596 | GGG |
| Asian | 6347 | 1 | ACACCATAATGGAAGACAGTGTGCCGGCAG | 597 | AGG |
| Asian | 6475 | −1 | GCAATGTTCCCAGGGCTTCCATCACTCCAA | 598 | AGG |
| Asian | 6503 | 1 | TTGGAGTGATGGAAGCCCTGGGAACATTGC | 599 | CGG |
| Asian | 6887 | 1 | ACCAAATGGCAATCATCATCATGATAGCAG | 600 | TGG |
| Asian | 7082 | 1 | CAACTTTCATCACCCCAGCCGTCCAACATG | 601 | CGG |
| Asian | 7206 | 1 | AT-GGGACTTTGGAGTCCCGCTGCTAATGA | 602 | TGG |
| Asian | 7571 | −1 | CCCTAAAAATGTTACACAGTGAAGTGGCTG | 603 | TGG |
| Asian | 7677 | 1 | CTGGCTTGGTCAAGAGACGTGGGGGTGGAA | 604 | CGG |
| Asian | 7716 | −1 | GAGTAGAACTCTAGGGCCGACATCTGGTTC | 605 | AGG |
| Asian | 8453 | −1 | CTTCAGCGCAGCTTACCACAGCCCGCGTGC | 606 | CGG |
| Asian | 9120 | 1 | TCTGGTATATGTGGCTAGGGGCTAGATTCC | 607 | TGG |
| Asian | 9123 | −1 | TCGTTCAAGAATCCAAGGGCTTCGAACTCC | 608 | AGG |
| Asian | 9516 | 1 | TCACTTACGCTCTTAATACATTCACCAACC | 609 | TGG |
| American | 650 | 1 | GCTATGAATGCCCTATGCTGGATGAGGGGG | 610 | TGG |
| American | 1507 | −1 | CTAGGCTTCCAAACCCCCCCAGGGTGGCTT | 611 | CGG |
| American | 1519 | 1 | AATTCACCAAGAGCCGAAGCCACCCTGGGG | 612 | GGG |
| American | 2068 | −1 | TCTTAGAGTTCTCAGTGCTTTCAGTGATTA | 613 | CGG |
| American | 2174 | −1 | TTTCCAATGGTGCTGCCACTCCTGTGCCAG | 614 | TGG |
| American | 2608 | −1 | AGGCTTGCTTGACTGCTGCTGCCAATCTAC | 615 | GGG |
| American | 2609 | −1 | CAGGCTTGCTTGACTGCTGCTGCCAATCTA | 616 | CGG |
| American | 2612 | 1 | ACAAGTACCATCCTGACTCCCCCCGTAGAT | 617 | TGG |
| American | 3277 | −1 | GCTCTTCACTGTGCCATGGCCCTTTCATTT | 618 | GGG |
| American | 3278 | −1 | AGCTCTTCACTGTGCCATGGCCCTTTCATT | 619 | TGG |
| American | 3569 | −1 | AGCAGAATCACAAGCACTCCAAGGGAGAAG | 620 | TGG |
| American | 3877 | −1 | AGGCGGAGATCGCAGTTTGCAAAAGACACG | 621 | AGG |
| American | 4007 | 1 | CACGCACTGATAACATCACCTTGGCAATCC | 622 | TGG |
| American | 4094 | −1 | TTCTTCACACTGCCTTTTCCCTTCAGAGAG | 623 | AGG |
| American | 4148 | −1 | GGGTCGACCAGCCTCACAGCGGTTAGTCCC | 624 | AGG |
| American | 4262 | −1 | TTGGCGAACCCTCCAGCCAATGCGCATATC | 625 | AGG |
| American | 4478 | 1 | TGGCGCTAGATGAGAGTGGTGATTTCTCCC | 626 | TGG |
| American | 4520 | 1 | ACGGTCCCCCCATGAGAGAGATCATACTCA | 627 | AGG |
| American | 4985 | 1 | TATTTAAGACAAAGGATGGGGACATTGGAG | 628 | CGG |
| American | 5312 | 1 | CAACCAGGGTTGTCGCTGCTGAAATGGAGG | 629 | AGG |
| American | 6095 | −1 | ATGAGGCCATCTTGGAGGTAAATATTGTCA | 630 | AGG |
| American | 6166 | −1 | GCTCCGTCCTAAGCTTGAACTCTCCCTCAA | 631 | TGG |
| American | 6440 | 1 | GTTCAGATCATGCGGCCCTGAAGTCATTCA | 632 | AGG |
| American | 6620 | −1 | CCCAGCAACCCCAAAAGCATAATGGTCTCT | 633 | AGG |
| American | 6878 | 1 | CCCAGGACAACCAAATGGCAATCATCATCA | 634 | TGG |
| American | 7039 | −1 | GGGTAATGAAAGTTGTCAAGGCAGCATAGA | 635 | TGG |
| American | 7051 | −1 | GTTGGACGGCTGGGGTAATGAAAGTTGTCA | 636 | AGG |
| American | 7096 | −1 | CCATCGCCATTAAGGAGTAGTTGTTGTATG | 637 | AGG |
| American | 7408 | −1 | TAGCACCTGTCCCATCTTTTTCTCCACTTG | 638 | GGG |
| American | 7409 | −1 | GTAGCACCTGTCCCATCTTTTTCTCCACTT | 639 | GGG |
| American | 7520 | −1 | TCGGAGAGCCTTCCCACAAAGTGGAAGTTG | 640 | CGG |
| American | 7731 | 1 | GGAAGGCCCGCTTGAACCAGATGTCGGCCC | 641 | TGG |
| American | 7815 | 1 | CCCGCCGCGCCCTCAAGGACGGTGTGGCAA | 642 | CGG |
| American | 7868 | 1 | GGAAGTGCAAAGCTGAGATGGTTGGTGGAG | 643 | CGG |
| American | 7869 | 1 | GAAGTGCAAAGCTGAGATGGTTGGTGGAGC | 644 | GGG |
| American | 8609 | −1 | CGTTTATTAGAGAGGACGCTGACCCTTGTG | 645 | TGG |
| American | 9015 | −1 | CTTTTTCCCATCATGTTGTACACACAACTC | 646 | TGG |
| American | 9881 | 1 | CCCTGCCGCCACCAAGATGAACTGATTGGC | 647 | CGG |
| American | 9882 | 1 | CCTGCCGCCACCAAGATGAACTGATTGGCC | 648 | GGG |
| American | 10278 | 1 | GGGCTGAGAACATTAAAAACACAGTCAACA | 649 | TGG |

TABLE 6
Sequences of Toehold Switch Sensors and Corresponding Target Sequences in
Zika Genome.

| Target | | | | | | Target RNA Fragment Used for | | | |
|---|---|---|---|---|---|---|---|---|---|
| sequence | | | | | | Initial Sensor Screening | | | |
| in Zika | Location | A Series Sensors | | B Series Sensors | | | Genome | Genome | Genome |
| virus | in | Sensor | Sensor | Sensor | Sensor | | Start | End | fragment |
| genome | genome | Name | sequence | Name | sequence | Sequence | site | site | length |
| UUGAGA | 3027 | 1A | UCUUCAGCC | 25B | UCUUCAGCC | GGGCAGUGAUC | 3007 | 3170 | 164 |
| GUGAGA | | | UCCAUGUGU | | UCCAUGUGU | UAGGCUACUGG | | | |
| AGAAUG | | | CAUUCUUCU | | CAUUCUUCU | AUUGAGAGUGA | | | |
| ACACAU | | | CACUCUCAA | | CACUCUCAA | GAAGAAUGACA | | | |
| GGAGGC | | | GUUAUAGUU | | GGACUUUAG | CAUGGAGGCUG | | | |
| UGAAGA | | | AUGAACAGA | | AACAGAGGA | AAGAGGGCCCAU | | | |
| (SEQ ID | | | GGAGACAUA | | GAUAAAGAU | CUGAUCGAGAU | | | |
| NO: 650) | | | ACAUGAACU | | GUUGAGAGU | GAAAACAUGUG | | | |
| | | | UGAGAAACC | | GAGUAACCU | AAUGGCCAAAG | | | |
| | | | AAGUUAACC | | GGCGGCAGC | UCCCACACAUUG | | | |
| | | | UGGCGGCAG | | GCAAAAG | UGGACAGAUGG | | | |
| | | | CGCAAAAG | | (SEQ ID NO: | AAUAGAAGAGA | | | |
| | | | (SEQ ID NO: | | 698) | GUGAUCUGAUC | | | |
| | | | 674) | | | AUACCCAAGUCU | | | |
| | | | | | | UUAGCUGGGC | | | |
| | | | | | | (SEQ ID NO: 722) | | | |
| AUGAUG | 8963 | 2A | AAAUUCCCC | 26B | AAAUUCCCC | GGGUGCCAGAG | 8941 | 9098 | 158 |
| GGAAAA | | | UUGUUUCUU | | UUGUUUCUU | UUGUGUGUACA | | | |
| AGAGAA | | | UUCUCUUUU | | UUCUCUUUU | ACAUGAUGGGA | | | |
| AAGAAA | | | UCCCAUCAU | | UCCCAUCAU | AAAAGAGAAAA | | | |
| CAAGGG | | | GUUAUAGUU | | GGACUUUAG | GAAACAAGGGG | | | |
| GAAUUU | | | AUGAACAGA | | AACAGAGGA | AAUUUGGAAAG | | | |
| (SEQ ID | | | GGAGACAUA | | GAUAAAGAU | GCCAAGGGCAGC | | | |
| NO: 651) | | | ACAUGAACA | | GAUGAUGGG | CGCGCCAUCUGG | | | |
| | | | UGAUGAACC | | AAAAAACCU | UAUAUGUGGCU | | | |
| | | | AUGUUAACC | | GGCGGCAGC | AGGGGCUAGAU | | | |
| | | | UGGCGGCAG | | GCAAAAG | UUCUAGAGUUC | | | |
| | | | CGCAAAAG | | (SEQ ID NO: | GAAGCCCUUGGA | | | |
| | | | (SEQ ID NO: | | 699) | UUCUUGAACGA | | | |
| | | | 675) | | | GGAUCACUGGA | | | |
| | | | | | | UGG (SEQ ID NO: | | | |
| | | | | | | 723) | | | |
| GACACAG | 1373 | 3A | UUUCGCUCU | 27B | UUUCGCUCU | GGGCCAGCACAG | 1348 | 1494 | 147 |
| GACAUG | | | AUUCUCAUC | | AUUCUCAUC | UGGGAUGAUCG | | | |
| AAACUG | | | AGUUUCAUG | | AGUUUCAUG | UUAAUGACACA | | | |
| AUGAGA | | | UCCUGUGUC | | UCCUGUGUC | GGACAUGAAAC | | | |
| AUAGAG | | | GUUAUAGUU | | GGACUUUAG | UGAUGAGAAUA | | | |
| CGAAA | | | AUGAACAGA | | AACAGAGGA | GAGCGAAAGUU | | | |
| (SEQ ID | | | GGAGACAUA | | GAUAAAGAU | GAGAUAACGCCC | | | |
| NO: 652) | | | ACAUGAACG | | GGACACAGG | AAUUCACCAAGA | | | |
| | | | ACACAAACG | | ACACAACCU | GCCGAAGCCACC | | | |
| | | | UCGUUAACC | | GGCGGCAGC | CUGGGGGGGUU | | | |
| | | | UGGCGGCAG | | GCAAAAG | UGGAAGCCUAG | | | |
| | | | CGCAAAAG | | (SEQ ID NO: | GACUUGAUUGU | | | |
| | | | (SEQ ID NO: | | 700) | GAACCGAGGACA | | | |
| | | | 676) | | | GG (SEQ ID NO: | | | |
| | | | | | | 724) | | | |
| UGGAAA | 2610 | 4A | GCUCCCCUU | 28B | GCUCCCCUU | GGGCGGGAUCUC | 2586 | 2733 | 148 |
| ACAUCA | | | CUACUGAUC | | CUACUGAUC | CUCUGUUUCAAG | | | |
| UGUGGA | | | UCCACAUGA | | UCCACAUGA | AAUGGAAAACA | | | |
| GAUCAG | | | UGUUUUCCA | | UGUUUUCCA | UCAUGUGGAGA | | | |
| UAGAAG | | | GUUAUAGUU | | GGACUUUAG | UCAGUAGAAGG | | | |
| GGGAGC | | | AUGAACAGA | | AACAGAGGA | GGAGCUCAACGC | | | |
| (SEQ ID | | | GGAGACAUA | | GAUAAAGAU | AAUCCUGGAAG | | | |
| NO: 653) | | | ACAUGAACU | | GUGGAAAAC | AGAAUGGAGUU | | | |
| | | | GGAAAAACC | | AUCAAACCU | CAACUGACGGUC | | | |
| | | | CAGUUAACC | | GGCGGCAGC | GUUGUGGGAUC | | | |
| | | | UGGCGGCAG | | GCAAAAG | UGUAAAAAACCC | | | |
| | | | CGCAAAAG | | (SEQ ID NO: | CAUGUGGAGAG | | | |
| | | | (SEQ ID NO: | | 701) | GUCCACAGAGAU | | | |
| | | | 677) | | | U (SEQ ID NO: | | | |
| | | | | | | 725) | | | |
| GGGGAA | 256 | 5A | AACUUCUUU | 29B | AACUUCUUU | GGGCCAUCACUG | 220 | 385 | 166 |
| AAAAGA | | | AUUAUUUCC | | AUUAUUUCC | GGUCUCAUCAAU | | | |
| GGCUAU | | | AUAGCCUCU | | AUAGCCUCU | AGAUGGGGUUC | | | |
| GGAAAU | | | UUUUUCCCC | | UUUUUCCCC | AGUGGGGAAAA | | | |
| AAUAAA | | | GUUAUAGUU | | GGACUUUAG | AAGAGGCUAUG | | | |
| GAAGUU | | | AUGAACAGA | | AACAGAGGA | GAAAUAAUAAA | | | |
| (SEQ ID | | | GGAGACAUA | | GAUAAAGAU | GAAGUUCAAGA | | | |
| NO: 654) | | | ACAUGAACG | | GGGGGAAAA | AAGAUCUGGCU | | | |
| | | | GGGAAAACC | | AAGAAACCU | GCCAUGCUGAGA | | | |
| | | | CCGUUAACC | | GGCGGCAGC | AUAAUCAAUGC | | | |
| | | | UGGCGGCAG | | GCAAAAG | UAGGAAGGAGA | | | |
| | | | CGCAAAAG | | (SEQ ID NO: | AGAAGAGACGA | | | |
| | | | (SEQ ID NO: | | 702) | GGCGCAGAUACU | | | |
| | | | 678) | | | AGUGUCGGAAU | | | |
| | | | | | | UGUUGGCCUC | | | |
| | | | | | | (SEQ ID NO: 726) | | | |
| GAUAAC | 1414 | 6A | AGGGUGGCU | 30B | AGGGUGGCU | GGGAAUGCUGU | 1327 | 1494 | 168 |
| GCCCAAU | | | UCGGCUCUU | | UCGGCUCUU | CAGUUCAUGGCU | | | |
| UCACCAA | | | GGUGAAUUG | | GGUGAAUUG | CCCAGCACAGUG | | | |
| GAGCCGA | | | GGCGUUAUC | | GGCGUUAUC | GGAUGAUCGUU | | | |
| AGCCACC | | | GUUAUAGUU | | GGACUUUAG | AAUGACACAGG | | | |
| CU (SEQ | | | AUGAACAGA | | AACAGAGGA | ACAUGAAACUG | | | |
| ID NO: | | | GGAGACAUA | | GAUAAAGAU | AUGAGAAUAGA | | | |
| 655) | | | ACAUGAACG | | GGAUAACGC | GCGAAAGUUGA | | | |
| | | | AUAACAACA | | CCAUAACCU | GAUAACGCCCAA | | | |
| | | | UCGUUAACC | | GGCGGCAGC | UUCACCAAGAGC | | | |
| | | | UGGCGGCAG | | GCAAAAG | CGAAGCCACCCU | | | |
| | | | CGCAAAAG | | (SEQ ID NO: | GGGGGGGUUUG | | | |
| | | | (SEQ ID NO: | | 703) | GAAGCCUAGGAC | | | |
| | | | 679) | | | UUGAUUGUGAA | | | |
| | | | | | | CCGAGGACAGG | | | |
| | | | | | | (SEQ ID NO: 727) | | | |
| UUGAAG | 8841 | 7A | CUUCCACUG | 31B | CUUCCACUG | GGGAGAAGGAU | 8729 | 8920 | 192 |
| AGGAAA | | | CAGUCUUCC | | CAGUCUUCC | GGUCUCUUCCUG | | | |
| AAGAGU | | | ACUCUUUUU | | ACUCUUUUU | GUUGUGGAAAG | | | |
| GGAAGA | | | CCUCUUCAA | | CCUCUUCAA | AGCUAGGCAAAC | | | |
| CUGCAG | | | GUUAUAGUU | | GGACUUUAG | ACAAACGGCCAC | | | |
| UGGAAG | | | AUGAACAGA | | AACAGAGGA | GAGUCUGUACCA | | | |
| (SEQ ID | | | GGAGACAUA | | GAUAAAGAU | AAGAAGAGUUC | | | |
| NO: 656) | | | ACAUGAACU | | GUUGAAGAG | AUCAACAAGGU | | | |
| | | | UGAAGAACC | | GAAAAACCU | UCGUAGCAAUGC | | | |
| | | | AAGUUAACC | | GGCGGCAGC | AGCAUUAGGGG | | | |
| | | | UGGCGGCAG | | GCAAAAG | CAAUAUUUGAA | | | |
| | | | CGCAAAAG | | (SEQ ID NO: | GAGGAAAAAGA | | | |
| | | | (SEQ ID NO: | | 704) | GUGGAAGACUG | | | |
| | | | 680) | | | CAGUGGAAGCU | | | |
| | | | | | | GUGAACGAUCCA | | | |
| | | | | | | AGGUUCUGGGC | | | |
| | | | | | | UCUAGUGGACA | | | |
| | | | | | | AGGAAAGA (SEQ | | | |
| | | | | | | ID NO: 728) | | | |
| UUUUGC | 7188 | 8A | CUGGGAUCA | 32B | CUGGGAUCA | GGGCUGACCCUA | 7166 | 7299 | 134 |
| UCGUGG | | | AGUACAUGU | | AGUACAUGU | AUAGUGGCCAUC | | | |
| CGCACUA | | | AGUGCGCCA | | AGUGCGCCA | AUUUUGCUCGU | | | |
| CAUGUA | | | CGAGCAAAA | | CGAGCAAAA | GGCGCACUACAU | | | |
| CUUGAU | | | GUUAUAGUU | | GGACUUUAG | GUACUUGAUCCC | | | |
| CCCAG | | | AUGAACAGA | | AACAGAGGA | AGGGCUGCAGGC | | | |
| (SEQ ID | | | GGAGACAUA | | GAUAAAGAU | AGCAGCUGCGCG | | | |
| NO: 657) | | | ACAUGAACU | | GUUUUGCUC | UGCUGCCCAGAA | | | |
| | | | UUUGCAACA | | GUGUAACCU | GAGAACGGCAGC | | | |
| | | | AAGUUAACC | | GGCGGCAGC | UGGCAUCAUGA | | | |
| | | | UGGCGGCAG | | GCAAAAG | AGAACCCUGUUG | | | |
| | | | CGCAAAAG | | (SEQ ID NO: | UGGAUGG (SEQ | | | |
| | | | (SEQ ID NO: | | 705) | ID NO: 729) | | | |
| | | | 681) | | | | | | |
| UUGCUA | 7144 | 9A | ACUAUUAGG | 33B | ACUAUUAGG | GGGUUUGGUAU | 7078 | 7228 | 151 |
| CUCACAA | | | GUCAGGGGU | | GUCAGGGGU | GGGCAAAGGGA | | | |
| UUAACA | | | GUUAAUUGU | | GUUAAUUGU | UGCCAUUCUACG | | | |
| CCCCUGA | | | GAGUAGCAA | | GAGUAGCAA | CAUGGGACUUU | | | |
| CCCUAAU | | | GUUAUAGUU | | GGACUUUAG | GGAGUCCCGCUG | | | |
| AGU (SEQ | | | AUGAACAGA | | AACAGAGGA | CUAAUGAUAGG | | | |
| ID NO: | | | GGAGACAUA | | GAUAAAGAU | UUGCUACUCACA | | | |
| 658) | | | ACAUGAACU | | GUUGCUACU | AUUAACACCCCU | | | |
| | | | UGCUAAACC | | CACUAACCU | GACCCUAAUAGU | | | |
| | | | AAGUUAACC | | GGCGGCAGC | GGCCAUCAUUUU | | | |
| | | | UGGCGGCAG | | GCAAAAG | GCUCGUGGCGCA | | | |
| | | | CGCAAAAG | | (SEQ ID NO: | CUACAUGUACUU | | | |
| | | | (SEQ ID NO: | | 706) | GAUCCCAGGGCU | | | |
| | | | 682) | | | G (SEQ ID NO: | | | |
| | | | | | | 730) | | | |
| ACCACCU | 7022 | 10A | CAUCGCCAU | 34B | CAUCGCCAU | GGGCCAUCUAUG | 6966 | 7141 | 176 |
| CAUACA | | | UAAGGAGUA | | UAAGGAGUA | CUGCCUUGACAA | | | |
| ACAACU | | | GUUGUUGUA | | GUUGUUGUA | CUUUCAUUACCC | | | |
| ACUCCUU | | | UGAGGUGGU | | UGAGGUGGU | CAGCCGUCCAAC | | | |
| AAUGGC | | | GUUAUAGUU | | GGACUUUAG | AUGCAGUGACCA | | | |
| GAUG | | | AUGAACAGA | | AACAGAGGA | CCUCAUACAACA | | | |
| (SEQ ID | | | GGAGACAUA | | GAUAAAGAU | ACUACUCCUUAA | | | |
| NO: 659) | | | ACAUGAACA | | GACCACCUC | UGGCGAUGGCCA | | | |
| | | | CCACCAACG | | AUAUAACCU | CGCAAGCUGGAG | | | |
| | | | GUGUUAACC | | GGCGGCAGC | UGUUGUUUGGU | | | |
| | | | UGGCGGCAG | | GCAAAAG | AUGGGCAAAGG | | | |
| | | | CGCAAAAG | | (SEQ ID NO: | GAUGCCAUUCUA | | | |
| | | | (SEQ ID NO: | | 707) | CGCAUGGGACUU | | | |
| | | | 683) | | | UGGAGUCCCGCU | | | |
| | | | | | | GCUAAUGAUA | | | |
| | | | | | | (SEQ ID NO: 731) | | | |
| ACCACAA | 3563 | 11A | CACUGCCAU | 35B | CACUGCCAU | GGGAGAAGGGU | 3521 | 3702 | 182 |
| AGAUCA | | | UGAUGUGCU | | UGAUGUGCU | GAUUCUGCUCAU | | | |
| UCAUAA | | | UAUGAUGAU | | UAUGAUGAU | GGUGCAGGAAG | | | |
| GCACAUC | | | CUUUGUGGU | | CUUUGUGGU | GGUUGAAGAAG | | | |
| AAUGGC | | | GUUAUAGUU | | GGACUUUAG | AGAAUGACCACA | | | |
| AGUG | | | AUGAACAGA | | AACAGAGGA | AAGAUCAUCAU | | | |
| (SEQ ID | | | GGAGACAUA | | GAUAAAGAU | AAGCACAUCAAU | | | |
| NO: 660) | | | ACAUGAACA | | GACCACAAA | GGCAGUGCUGG | | | |
| | | | CCACAAACG | | GAUAAACCU | UAGCUAUGAUCC | | | |
| | | | GUGUUAACC | | GGCGGCAGC | UGGGAGGAUUU | | | |
| | | | UGGCGGCAG | | GCAAAAG | UCAAUGAGUGA | | | |
| | | | CGCAAAAG | | (SEQ ID NO: | CCUGGCUAAGCU | | | |
| | | | (SEQ ID NO: | | 708) | UGCAAUUUUGA | | | |
| | | | 684) | | | UGGGUGCCACCU | | | |
| | | | | | | UCGCGGAAAUG | | | |
| | | | | | | AACACUGGAGG | | | |
| | | | | | | AGAUGUAGC | | | |
| | | | | | | (SEQ ID NO: 732) | | | |
| AGACAG | 5721 | 12A | CCCACUCUU | 36B | CCCACUCUU | GGGAGAAGGGU | 5628 | 5850 | 223 |
| AGUUCC | | | GAUGUUUUG | | GAUGUUUUG | UUGUUCCAAGCG | | | |
| AGAAAA | | | UUUUCUGGA | | UUUUCUGGA | UGAGGAACGGC | | | |
| CAAAACA | | | ACUCUGUCU | | ACUCUGUCU | AAUGAGAUCGC | | | |
| UCAAGA | | | GUUAUAGUU | | GGACUUUAG | AGCUUGUCUGAC | | | |
| GUGGG | | | AUGAACAGA | | AACAGAGGA | AAAGGCUGGAA | | | |
| (SEQ ID | | | GGAGACAUA | | GAUAAAGAU | AACGGGUCAUAC | | | |
| NO: 661) | | | ACAUGAACA | | GAGACAGAG | AGCUCAGCAGAA | | | |
| | | | GACAGAACU | | UUCCAACCU | AGACUUUUGAG | | | |
| | | | CUGUUAACC | | GGCGGCAGC | ACAGAGUUCCAG | | | |
| | | | UGGCGGCAG | | GCAAAAG | AAAACAAAACA | | | |
| | | | CGCAAAAG | | (SEQ ID NO: | UCAAGAGUGGG | | | |
| | | | (SEQ ID NO: | | 709) | ACUUUGUCGUG | | | |
| | | | 685) | | | ACAACUGACAUU | | | |
| | | | | | | UCAGAGAUGGG | | | |
| | | | | | | CGCCAACUUUAA | | | |
| | | | | | | AGCUGACCGUGU | | | |
| | | | | | | CAUAGAUUCCAG | | | |
| | | | | | | GAGAUGCCUAA | | | |
| | | | | | | AGCCGGUCAUAC | | | |
| | | | | | | U (SEQ ID NO: | | | |
| | | | | | | 733) | | | |
| UGCACA | 3368 | 13A | AUCUUUAGC | 37B | AUCUUUAGC | GGGAGAUCAACC | 3316 | 3445 | 130 |
| AUGCCCC | | | CCGGAACGA | | CCGGAACGA | ACUGCAAGCGGA | | | |
| CACUGUC | | | CAGUGGGGG | | CAGUGGGGG | AGGGUGAUCGA | | | |
| GUUCCG | | | CAUUGUGCA | | CAUUGUGCA | GGAAUGGUGCU | | | |
| GGCUAA | | | GUUAUAGUU | | GGACUUUAG | GCAGGGAGUGC | | | |
| AGAU | | | AUGAACAGA | | AACAGAGGA | ACAAUGCCCCCA | | | |
| (SEQ ID | | | GGAGACAUA | | GAUAAAGAU | CUGUCGUUCCGG | | | |
| NO: 662) | | | ACAUGAACU | | GUGCACAAU | GCUAAAGAUGG | | | |
| | | | GCACAAACG | | GCCGAACCU | CUGUUGGUAUG | | | |
| | | | CAGUUAACC | | GGCGGCAGC | GAAUGGAGAUA | | | |
| | | | UGGCGGCAG | | GCAAAAG | AGGCCCAGGAAA | | | |
| | | | CGCAAAAG | | (SEQ ID NO: | GAACCA (SEQ ID | | | |
| | | | (SEQ ID NO: | | 710) | NO: 734) | | | |
| | | | 686) | | | | | | |
| GACACCG | 1598 | 14A | UGCUUCUUU | 38B | UGCUUCUUU | GGGAGUGGUUC | 1555 | 1711 | 157 |
| GAACUCC | | | GUUGUUCCA | | GUUGUUCCA | CACGACAUUCCA | | | |
| ACACUG | | | GUGUGGAGU | | GUGUGGAGU | UUACCUUGGCAC | | | |
| GAACAAC | | | UCCGGUGUC | | UCCGGUGUC | GCUGGGGCAGAC | | | |
| AAAGAA | | | GUUAUAGUU | | GGACUUUAG | ACCGGAACUCCA | | | |
| GCA (SEQ | | | AUGAACAGA | | AACAGAGGA | CACUGGAACAAC | | | |
| ID NO: | | | GGAGACAUA | | GAUAAAGAU | AAAGAAGCACU | | | |
| 663) | | | ACAUGAACG | | GGACACCGG | GGUAGAGUUCA | | | |
| | | | ACACCAACG | | AACAAACCU | AGGACGCACAUG | | | |
| | | | UCGUUAACC | | GGCGGCAGC | CCAAAAGGCAAA | | | |
| | | | UGGCGGCAG | | GCAAAAG | CUGUCGUGGUUC | | | |
| | | | CGCAAAAG | | (SEQ ID NO: | UAGGGAGUCAA | | | |
| | | | (SEQ ID NO: | | 711) | GAAGGAGCAGU | | | |
| | | | 687) | | | UCACACG (SEQ | | | |
| | | | | | | ID NO: 735) | | | |
| GUUUGC | 6379 | 15A | ACUCCAAAA | 39B | ACUCCAAAA | GGGUGGACGCCA | 6330 | 6458 | 129 |
| CGCUGGG | | | GCCGCUCCU | | GCCGCUCCU | GAGUUUGUUCA | | | |
| AAAAGA | | | CUUUUCCCA | | CUUUUCCCA | GAUCAUGCGGCC | | | |
| GGAGCG | | | GCGGCAAAC | | GCGGCAAAC | CUGAAGUCAUUC | | | |
| GCUUUU | | | GUUAUAGUU | | GGACUUUAG | AAGGAGUUUGC | | | |
| GGAGU | | | AUGAACAGA | | AACAGAGGA | CGCUGGGAAAA | | | |
| (SEQ ID | | | GGAGACAUA | | GAUAAAGAU | GAGGAGCGGCU | | | |
| NO: 664) | | | ACAUGAACG | | GGUUUGCCG | UUUGGAGUGAU | | | |
| | | | UUUGCAACA | | CUGCAACCU | GGAAGCCCUGGG | | | |
| | | | ACGUUAACC | | GGCGGCAGC | AACACUGCCAGG | | | |
| | | | UGGCGGCAG | | GCAAAAG | ACACAUGACAGA | | | |
| | | | CGCAAAAG | | (SEQ ID NO: | GAGAU (SEQ ID | | | |
| | | | (SEQ ID NO: | | 712) | NO: 736) | | | |
| | | | 688) | | | | | | |
| GGCAGA | 6277 | 16A | ACUCUUUUC | 40B | ACUCUUUUC | GGGCUGCCGGAA | 6198 | 6351 | 154 |
| AGUGUG | | | UCUCCGUGU | | UCUCCGUGU | UAACCUACACAG | | | |
| GACCAGA | | | CUGGUCCAC | | CUGGUCCAC | AUAGAAGAUGG | | | |
| CACGGAG | | | ACUUCUGCC | | ACUUCUGCC | UGCUUUGAUGG | | | |
| AGAAAA | | | GUUAUAGUU | | GGACUUUAG | CACGACCAACAA | | | |
| GAGU | | | AUGAACAGA | | AACAGAGGA | CACCAUAAUGGA | | | |
| (SEQ ID | | | GGAGACAUA | | GAUAAAGAU | AGACAGUGUGCC | | | |
| NO: 665) | | | ACAUGAACG | | GGGCAGAAG | GGCAGAAGUGU | | | |
| | | | GCAGAAACG | | UGUAAACCU | GGACCAGACACG | | | |
| | | | CCGUUAACC | | GGCGGCAGC | GAGAGAAAAGA | | | |
| | | | UGGCGGCAG | | GCAAAAG | GUGCUCAAACCG | | | |
| | | | CGCAAAAG | | (SEQ ID NO: | AGGUGGAUGGA | | | |
| | | | (SEQ ID NO: | | 713) | CGCCAGAGUUUG | | | |
| | | | 689) | | | UUCAGA (SEQ ID | | | |
| | | | | | | NO: 737) | | | |
| CUGAUC | 3071 | 17A | GGACUUUGG | 41B | GGACUUUGG | GGGCUACUGGA | 3017 | 3170 | 154 |
| GAGAUG | | | CCAUUCACA | | CCAUUCACA | UUGAGAGUGAG | | | |
| AAAACA | | | UGUUUUCAU | | UGUUUUCAU | AAGAAUGACAC | | | |
| UGUGAA | | | CUCGAUCAG | | CUCGAUCAG | AUGGAGGCUGA | | | |
| UGGCCA | | | GUUAUAGUU | | GGACUUUAG | AGAGGGCCCAUC | | | |
| AAGUCC | | | AUGAACAGA | | AACAGAGGA | UGAUCGAGAUG | | | |
| (SEQ ID | | | GGAGACAUA | | GAUAAAGAU | AAAACAUGUGA | | | |
| NO: 666) | | | ACAUGAACC | | GCUGAUCGA | AUGGCCAAAGUC | | | |
| | | | UGAUCAACC | | GAUGAACCU | CCACACAUUGUG | | | |
| | | | AGGUUAACC | | GGCGGCAGC | GACAGAUGGAA | | | |
| | | | UGGCGGCAG | | GCAAAAG | UAGAAGAGAGU | | | |
| | | | CGCAAAAG | | (SEQ ID NO: | GAUCUGAUCAU | | | |
| | | | (SEQ ID NO: | | 714) | ACCCAAGUCUUU | | | |
| | | | 690) | | | AGCUGGGC (SEQ | | | |
| | | | | | | ID NO: 738) | | | |
| GAGCCAG | 6761 | 18A | UUGGUUGUC | 42B | UUGGUUGUC | GGGCAGCCAGAA | 6702 | 6874 | 173 |
| AAAAGC | | | CUGGGGAGA | | CUGGGGAGA | UUGCAUGUGUCC | | | |
| AAAGAU | | | UCUUUGCUU | | UCUUUGCUU | UCAUUGUUGUG | | | |
| CUCCCCA | | | UUCUGGCUC | | UUCUGGCUC | UUCCUAUUGCU | | | |
| GGACAAC | | | GUUAUAGUU | | GGACUUUAG | GGUGGUGCUCA | | | |
| CAA (SEQ | | | AUGAACAGA | | AACAGAGGA | UACCUGAGCCAG | | | |
| ID NO: | | | GGAGACAUA | | GAUAAAGAU | AAAAGCAAAGA | | | |
| 667) | | | ACAUGAACG | | GGAGCCAGA | UCUCCCCAGGAC | | | |
| | | | AGCCAAACC | | AAAGAACCU | AACCAAAUGGCA | | | |
| | | | UCGUUAACC | | GGCGGCAGC | AUCAUCAUCAUG | | | |
| | | | UGGCGGCAG | | GCAAAAG | GUAGCAGUAGG | | | |
| | | | CGCAAAAG | | (SEQ ID NO: | UCUUCUGGGCU | | | |
| | | | (SEQ ID NO: | | 715) | UGAUUACCGCCA | | | |
| | | | 691) | | | AUGAACUCGGA | | | |
| | | | | | | UGGUUGGAGAG | | | |
| | | | | | | AACA (SEQ ID | | | |
| | | | | | | NO: 739) | | | |
| CUUAAC | 9431 | 19A | AAUGAGUUG | 43B | AAUGAGUUG | GGGAGCGGACA | 9404 | 9581 | 178 |
| ACAUUU | | | CACCACUAG | | CACCACUAG | AGUUGUCACUU | | | |
| ACCAACC | | | GUUGGUAAA | | GUUGGUAAA | ACGCUCUUAACA | | | |
| UAGUGG | | | UGUGUUAAG | | UGUGUUAAG | CAUUUACCAACC | | | |
| UGCAAC | | | GUUAUAGUU | | GGACUUUAG | UAGUGGUGCAA | | | |
| UCAUU | | | AUGAACAGA | | AACAGAGGA | CUCAUUCGGAAU | | | |
| (SEQ ID | | | GGAGACAUA | | GAUAAAGAU | AUGGAGGCUGA | | | |
| NO: 668) | | | ACAUGAACC | | GCUUAACAC | GGAAGUUCUAG | | | |
| | | | UUAACAACA | | AUUGAACCU | AGAUGCAAGAC | | | |
| | | | AGGUUAACC | | GGCGGCAGC | UUGUGGCUGCU | | | |
| | | | UGGCGGCAG | | GCAAAAG | GCGGAGGUCAG | | | |
| | | | CGCAAAAG | | (SEQ ID NO: | AGAAAGUGACU | | | |
| | | | (SEQ ID NO: | | 716) | AACUGGUUGCA | | | |
| | | | 692) | | | GAGCAACGGAU | | | |
| | | | | | | GGGAUAGGCUC | | | |
| | | | | | | AAACGAAUGG | | | |
| | | | | | | (SEQ ID NO: 740) | | | |
| GCGGUA | 2466 | 20A | CUUCAACGU | 44B | CUUCAACGU | GGGUGCUCGGU | 2429 | 2578 | 150 |
| CAGGGG | | | CGUUAUAGA | | CGUUAUAGA | GGACUUCUCAAA | | | |
| UGUUCG | | | CGAACACCC | | CGAACACCC | GAAGGAGACGA | | | |
| UCUAUA | | | CUGUACCGC | | CUGUACCGC | GAUGCGGUACA | | | |
| ACGACG | | | GUUAUAGUU | | GGACUUUAG | GGGGUGUUCGU | | | |
| UUGAAG | | | AUGAACAGA | | AACAGAGGA | CUAUAACGACGU | | | |
| (SEQ ID | | | GGAGACAUA | | GAUAAAGAU | UGAAGCCUGGA | | | |
| NO: 669) | | | ACAUGAACG | | GGCGGUACA | GGGACAGGUAC | | | |
| | | | CGGUAAACC | | GGGAAACCU | AAGUACCAUCCU | | | |
| | | | GCGUUAACC | | GGCGGCAGC | GACUCCCCCCGU | | | |
| | | | UGGCGGCAG | | GCAAAAG | AGAUUGGCAGC | | | |
| | | | CGCAAAAG | | (SEQ ID NO: | AGCAGUCAAGCA | | | |
| | | | (SEQ ID NO: | | 717) | AGCCUGGGAAG | | | |
| | | | 693) | | | AU (SEQ ID NO: | | | |
| | | | | | | 741) | | | |
| GAGGCU | 9476 | 21A | CCACAAGUC | 45B | CCACAAGUC | GGGAGUGGUGC | 9451 | 9584 | 134 |
| GAGGAA | | | UUGCAUCUC | | UUGCAUCUC | AACUCAUUCGGA | | | |
| GUUCUA | | | UAGAACUUC | | UAGAACUUC | AUAUGGAGGCU | | | |
| GAGAUG | | | CUCAGCCUC | | CUCAGCCUC | GAGGAAGUUCU | | | |
| CAAGAC | | | GUUAUAGUU | | GGACUUUAG | AGAGAUGCAAG | | | |
| UUGUGG | | | AUGAACAGA | | AACAGAGGA | ACUUGUGGCUGC | | | |
| (SEQ ID | | | GGAGACAUA | | GAUAAAGAU | UGCGGAGGUCA | | | |
| NO: 670) | | | ACAUGAACG | | GGAGGCUGA | GAGAAAGUGAC | | | |
| | | | AGGCUAACC | | GGACAACCU | UAACUGGUUGC | | | |
| | | | UCGUUAACC | | GGCGGCAGC | AGAGCAACGGA | | | |
| | | | UGGCGGCAG | | GCAAAAG | UGGGAUAGGCU | | | |
| | | | CGCAAAAG | | (SEQ ID NO: | CAAACGAAUGGC | | | |
| | | | (SEQ ID NO: | | 718) | AG (SEQ ID NO: | | | |
| | | | 694) | | | 742) | | | |
| GGCACAG | 3225 | 22A | AUUCCUCAA | 46B | AUUCCUCAA | GGGCAAUACCAG | 3184 | 3334 | 151 |
| UGAAGA | | | ACCGAAUUU | | ACCGAAUUU | AGAGGGCUACA | | | |
| GCUUGA | | | CAAGCUCUU | | CAAGCUCUU | GGACCCAAAUGA | | | |
| AAUUCG | | | CACUGUGCC | | CACUGUGCC | AAGGGCCAUGGC | | | |
| GUUUGA | | | GUUAUAGUU | | GGACUUUAG | ACAGUGAAGAG | | | |
| GGAAU | | | AUGAACAGA | | AACAGAGGA | CUUGAAAUUCG | | | |
| | | | GGAGACAUA | | GAUAAAGAU | GUUUGAGGAAU | | | |
| (SEQ ID | | | ACAUGAACG | | GGGCACAGU | GCCCAGGCACUA | | | |
| NO: 671) | | | GCACAAACG | | GAAAAACCU | AGGUCCACGUGG | | | |
| | | | CCGUUAACC | | GGCGGCAGC | AGGAAACAUGU | | | |
| | | | UGGCGGCAG | | GCAAAAG | GGAACGAGAGG | | | |
| | | | CGCAAAAG | | (SEQ ID NO: | ACCAUCUCUGAG | | | |
| | | | (SEQ ID NO: | | 719) | AUCAACCACUGC | | | |
| | | | 695) | | | AAGC (SEQ ID | | | |
| | | | | | | NO: 743) | | | |
| CAUCUA | 6890 | 23A | UAUGGUUGC | 47B | UAUGGUUGC | GGGAGUAGGUC | 6820 | 6952 | 133 |
| AUGGGA | | | CCCCUCCUCU | | CCCCUCCUC | UUCUGGGCUUG | | | |
| AGGAGA | | | CUCCUUCCC | | UCUCCUUCC | AUUACCGCCAAU | | | |
| GAGGAG | | | AUUAGAUGG | | CAUUAGAUG | GAACUCGGAUG | | | |
| GGGGCA | | | UUAUAGUUA | | GGACUUUAG | GUUGGAGAGAA | | | |
| ACCAUA | | | UGAACAGAG | | AACAGAGGA | CAAAGAGUGACC | | | |
| (SEQ ID | | | GAGACAUAA | | GAUAAAGAU | UAAGCCAUCUAA | | | |
| NO: 672) | | | CAUGAACCA | | GCAUCUAAU | UGGGAAGGAGA | | | |
| | | | UCUAAACAU | | GGGAAACCU | GAGGAGGGGGC | | | |
| | | | GGUUAACCU | | GGCGGCAGC | AACCAUAGGAU | | | |
| | | | GGCGGCAGC | | GCAAAAG | UCUCAAUGGACA | | | |
| | | | GCAAAAG | | (SEQ ID NO: | UUGACCUGCGG | | | |
| | | | (SEQ ID NO: | | 720) | (SEQ ID NO: 744) | | | |
| | | | 696) | | | | | | |
| GAUAGG | 9620 | 24A | AUCAUUCAA | 48B | AUCAUUCAA | GGGAUAGGCUC | 9561 | 9688 | 128 |
| UUUGCA | | | GAACCUGAG | | GAACCUGAG | AAACGAAUGGC | | | |
| CAUGCCC | | | GGCAUGUGC | | GGCAUGUGC | AGUCAGUGGAG | | | |
| UCAGGU | | | AAACCUAUC | | AAACCUAUC | AUGAUUGCGUU | | | |
| UCUUGA | | | GUUAUAGUU | | GGACUUUAG | GUGAAGCCAAU | | | |
| AUGAU | | | AUGAACAGA | | AACAGAGGA | UGAUGAUAGGU | | | |
| (SEQ ID | | | GGAGACAUA | | GAUAAAGAU | UUGCACAUGCCC | | | |
| NO: 673) | | | ACAUGAACG | | GGAUAGGUU | UCAGGUUCUUG | | | |
| | | | AUAGGAACA | | UGCUAACCU | AAUGAUAUGGG | | | |
| | | | UCGUUAACC | | GGCGGCAGC | AAAAGUUAGGA | | | |
| | | | UGGCGGCAG | | GCAAAAG | AGGACACACAAG | | | |
| | | | CGCAAAAG | | (SEQ ID NO: | AGUGG (SEQ ID | | | |
| | | | (SEQ ID NO: | | 721) | NO: 745) | | | |
| | | | 697) | | | | | | |

NOTES:
1. The sequence GGG was added to the 5′ end of all sensor RNA and target RNA fragment sequences for efficient expression by T7 RNA polymerase. If the RNA sequence began with G or GG, only GG or G, respectively, was added to the 5′ end of the sequence. 2. The GGG prefix is not shown in the sensor sequences in the table so that the target RNA binding site can be readily identified. 3. The coding sequence of the reporter protein lacZ was added immediately after the switch RNA sequences in the tables. 4. Two Zika virus strains (KU312312, AY632535) have sufficient sequence homology to be detected using the same toehold switch sensors (27B, 32B). 5. Target RNAs fragments for sensors 7A/31B and 11A/35B have a GGGAGAAGG sequence added at the 5′ end. 6. Target RNA fragments for sensors 12A/36B have a GGGAG A AG sequence added at the 5′ end.

TABLE 7

| | |
|---|---|
| Zika virus | ACAGGUUUUAUUUUGGAUUUGGAAACGAGAGUUUCUGGUCAUGAAAAACCCAA |
| from the | AAAAGAAAUCCGGAGGAUUCCGGAUUGUCAAUAUGCUAAAACGCGGAGUAGCC |
| Americas | CGUGUGAGCCCCUUUGGGGGCUUGAAGAGGCUGCCAGCCGGACUUCUGCUGGG |
| genome | UCAUGGGCCCAUCAGGAUGGUCUUGGCGAUUCUAGCCUUUUUGAGAUUCACGG |
| sequence | CAAUCAAGCCAUCACUGGGUCUCAUCAAUAGAUGGGGUUCAGUGGGGAAAAAA |
| (Accession | GAGGCUAUGGAAAUAAUAAAGAAGUUCAAGAAAGAUCUGGCUGCCAUGCUGA |
| number: | GAAUAAUCAAUGCUAGGAAGGAGAAGAAGAGACGAGGCGCAGAUACUAGUGU |
| KU312312; | CGGAAUUGUUGGCCUCCUGCUGACCACAGCUAUGGCAGCGGAGGUCACUAGAC |
| 10,374-nts) | GUGGGAGUGCAUACUAUAUGUACUUGGACAGAAACGAUGCUGGGGAGGCCAUA |
| | UCUUUUCCAACCACAUUGGGGAUGAAUAAGUGUUAUAUACAGAUCAUGGAUCU |
| | UGGACACACGUGUGAUGCCACCAUGAGCUAUGAAUGCCCUAUGCUGGAUGAGG |
| | GGGUGGAACCAGAUGACGUCGAUUGUUGGUGCAACACGACGUCAACUUGGGUU |
| | GUGUACGGAACCUGCCAUCACAAAAAAGGUGAAGCACGGAGAUCUAGAAGAGC |
| | UGUGACGCUCCCCUCCCAUUCCACUAGGAAGCUGCAAACGCGGUCGCAAACCU |
| | GGUUGGAAUCAAGAGAAUACACAAAGCACUUGAUUAGAGUCGAAAAUUGGAU |
| | AUUCAGGAACCCUGGCUUCGCGUUAGCAGCAGCUGCCAUCGCUUGGCUUUUGG |
| | GAAGCUCAACGAGCCAAAAAGUCAUAUACUUGGUCAUGAUACUGCUGAUUGCC |
| | CCGGCAUACAGCAUCAGGUGCAUAGGAGUCAGCAAUAGGGACUUUGUGGAAGG |
| | UAUGUCAGGUGGGACUUGGGUUGAUGUUGUCUUGGAACAUGGAGGUUGUGUC |
| | ACUGUAAUGGCACAGGACAAACCGACUGUCGACAUAGAGCUGGUUACAACAAC |
| | AGUCAGCAACAUGGCGGAGGUAAGAUCCUACUGCUAUGAGGCAUCAAUAUCAG |
| | ACAUGGCUUCGGACAGCCGCUGCCCAACACAAGGUGAAGCCUACCUUGACAAG |
| | CAAUCAGACACUCAAUAUGUCUGCAAAAGAACGUUAGUGGACAGAGGCUGGGG |
| | AAAUGGAUGUGGACUUUUUGGCAAAGGGAGCCUGGUGACAUGCGCUAAGUUU |
| | GCAUGCUCCAAGAAAAUGACCGGGAAGAGCAUCCAGCCAGAGAAUCUGGAGUA |
| | CCGGAUAAUGCUGUCAGUUCAUGGCUCCCAGCACAGUGGGAUGAUCGUUAAUG |
| | ACACAGGACAUGAAACUGAUGAGAAUAGAGCGAAAGUUGAGAUAACGCCCAAU |
| | UCACCAAGAGCCGAAGCCACCCUGGGGGGGUUUGGAAGCCUAGGACUUGAUUG |
| | UGAACCGAGGACAGGCCUUGACUUUUCAGAUUUGUAUUACUUGACUAUGAAUA |
| | ACAAGCACUGGCUGGUUCACAAGGAGUGGUUCCACGACAUUCCAUUACCUUGG |
| | CACGCUGGGGCAGACACCGGAACUCCACACUGGAACAACAAAGAAGCACUGGU |
| | AGAGUUCAAGGACGCACAUGCCAAAAGGCAAACUGUCGUGGUUCUAGGGAGUC |
| | AAGAAGGAGCAGUUCACACGGCCCUUGCUGGAGCUCUGGAGGCUGAGAUGGAU |
| | GGUGCAAAGGGAAGGCUGUCCUCUGGCCACUUGAAAUGUCGCCUGAAAAUGGA |
| | UAAACUUAGAUUGAAGGGCGUGUCAUACUCCUUGUGUACUGCAGCGUUCACAU |
| | UCACCAAGAUCCCGGCUGAAACACUGCACGGGACAGUCACAGUGGAGGUACAG |
| | UACGCAGGGACAGAUGGACCUUGCAAGGUUCCAGCUCAGAUGGCGGUGGACAU |
| | GCAAACUCUGACCCCAGUUGGGAGGUUGAUAACCGCUAACCCCGUAAUCACUG |
| | AAAGCACUGAGAACUCUAAGAUGAUGCUGGAACUUGAUCCACCAUUUGGGGAC |
| | UCUUACAUUGUCAUAGGAGUCGGGGAGAAGAAGAUCACCCACCACUGGCACAG |
| | GAGUGGCAGCACCAUUGGAAAAGCAUUUGAAGCCACUGUGAGAGGUGCCAAGA |
| | GAAUGGCAGUCUUGGGAGACACAGCCUGGGACUUUGGAUCAGUUGGAGGCGCU |
| | CUCAACUCAUUGGGCAAGGGCAUCCAUCAAAUCUUUGGAGCAGCUUUCAAAUC |
| | AUUGUUUGGAGGAAUGUCCUGGUUCUCACAAAUUCUCAUUGGAACGUUGCUGA |
| | UGUGGUUGGGUCUGAACGCAAAGAAUGGAUCUAUUUCCCUUAUGUGCUUGGCC |
| | UUAGGGGGAGUGUUGAUCUUCUUAUCCACAGCCGUCUCUGCUGAUGUGGGGUG |
| | CUCGGUGGACUUCUCAAAGAAGGAGACGAGAUGCGGUACAGGGGUGUUCGUCU |
| | AUAACGACGUUGAAGCCUGGAGGGACAGGUACAAGUACCAUCCUGACUCCCCC |
| | CGUAGAUUGGCAGCAGCAGUCAAGCAAGCCUGGGAAGAUGGUAUCUGCGGGAU |
| | CUCCUCUGUUUCAAGAAUGGAAAACAUCAUGUGGAGAUCAGUAGAAGGGGAGC |
| | UCAACGCAAUCCUGGAAGAGAAUGGAGUUCAACUGACGGUCGUUGUGGGAUCU |
| | GUAAAAAACCCCAUGUGGAGAGGUCCACAGAGAUUGCCCGUGCCUGUGAACGA |
| | GCUGCCCCACGGCUGGAAGGCUUGGGGGAAAUCGUACUUCGUCAGAGCAGCAA |
| | AGACAAAUAACAGCUUUGUCGUGGAUGGUGACACACUGAAGGAAUGCCCACUC |
| | AAACAUAGAGCAUGGAACAGCUUUCUUGUGGAGGAUCAUGGGUUCGGGGUAU |
| | UUCACACUAGUGUCUGGCUCAAGGUUAGAGAAGAUUAUUCAUUAGAGUGUGA |
| | UCCAGCCGUUAUUGGAACAGCUGUUAAGGGAAAGGAGGCUGUACACAGUGAUC |
| | UAGGCUACUGGAUUGAGAGUGAGAAGAAUGACACAUGGAGGCUGAAGAGGGC |
| | CCAUCUGAUCGAGAUGAAAACAUGUGAAUGGCCAAAGUCCCACACAUUGUGGA |
| | CAGAUGGAAUAGAAGAGAGUGAUCUGAUCAUACCCAAGUCUUUAGCUGGGCCA |
| | CUCAGCCAUCACAAUACCAGAGAGGGCUACAGGACCCAAAUGAAAGGGCCAUG |
| | GCACAGUGAAGAGCUUGAAAUUCGGUUUGAGGAAUGCCCAGGCACUAAGGUCC |
| | ACGUGGAGGAAACAUGUGGAACGAGAGGACCAUCUCUGAGAUCAACCACUGCA |
| | AGCGGAAGGGUGAUCGAGGAAUGGUGCUGCAGGGAGUGCACAAUGCCCCCACU |
| | GUCGUUCCGGGCUAAAGAUGGCUGUUGGUAUGGAAUGGAGAUAAGGCCCAGGA |
| | AAGAACCAGAAAGCAACUUAGUAAGGUCAAUGGUGACUGCAGGAUCAACUGAU |
| | CACAUGGACCACUUCUCCCUUGGAGUGCUUGUGAUUCUGCUCAUGGUGCAGGA |
| | AGGGUUGAAGAAGAGAAUGACCACAAAGAUCAUCAUAAGCACAUCAAUGGCAG |
| | UGCUGGUAGCUAUGAUCCUGGGAGGAUUUUCAAUGAGUGACCUGGCUAAGCUU |
| | GCAAUUUUGAUGGGUGCCACCUUCGCGGAAAUGAACACUGGAGGAGAUGUAGC |
| | UCAUCUGGCGCUGAUAGCGGCAUUCAAAGUCAGACCAGCGUUGCUGGUAUCUU |
| | UCAUCUUCAGAGCUAAUUGGACACCCCGUGAAAGCAUGCUGCUGGCCUUGGCC |
| | UCGUGUCUUUUGCAAACUGCGAUCUCCGCCUUGGAAGGCGACCUGAUGGUUCU |
| | CAUCAAUGGUUUUGCUUUGGCCUGGUUGGCAAUACGAGCGAUGGUUGUUCCAC |
| | GCACUGAUAACAUCACCUUGGCAAUCCUGGCUGCUCUGACACCACUGGCCCGG |
| | GGCACACUGCUUGUGGCGUGGAGAGCAGGCCUUGCUACUUGCGGGGGGUUUAU |
| | GCUCCUCUCUCUGAAGGGAAAAGGCAGUGUGAAGAAGAACUUACCAUUUGUCA |
| | UGGCCCUGGGACUAACCGCUGUGAGGCUGGUCGACCCCAUCAACGUGGUGGGA |
| | CUGCUGUUGCUCACAAGGAGUGGGAAGCGGAGCUGGCCCCCUAGCGAAGUACU |
| | CACAGCUGUUGGCCUGAUAUGCGCAUUGGCUGGAGGGUUCGCCAAGGCAGAUA |
| | UAGAGAUGGCUGGGCCCAUGGCCGCGGUCGGUCUGCUAAUUGUCAGUUACGUG |
| | GUCUCAGGAAAGAGUGUGGACAUGUACAUUGAAAGAGCAGGUGACAUCACAUG |
| | GGAAAAAGAUGCGGAAGUCACUGGAAACAGUCCCCGGCUCGAUGUGGCGCUAG |
| | AUGAGAGUGGUGAUUUCUCCCUGGUGGAGGAUGACGGUCCCCCCAUGAGAGAG |
| | AUCAUACUCAAGGUGGUCCUGAUGACCAUCUGUGGCAUGAACCCAAUAGCCAU |
| | ACCCUUUGCAGCUGGAGCGUGGUACGUAUACGUGAAGACUGGAAAAAGGAGUG |
| | GUGCUCUAUGGGAUGUGCCUGCUCCCAAGGAAGUAAAAAAGGGGGAGACCACA |
| | GAUGGAGUGUACAGAGUAAUGACUCGUAGACUGCUAGGUUCAACACAAGUUGG |
| | AGUGGGAGUUAUGCAAGAGGGGGUCUUUCACACUAUGUGGCACGUCACAAAAG |
| | GAUCCGCGCUGAGAAGCGGUGAAGGGAGACUUGAUCCAUACUGGGGAGAUGUC |
| | AAGCAGGAUCUGGUGUCAUACUGUGGUCCAUGGAAGCUAGAUGCCGCCUGGGA |
| | CGGGCACAGCGAGGUGCAGCUCUUGGCCGUGCCCCCCGGAGAGAGAGCGAGGA |
| | ACAUCCAGACUCUGCCCGGAAUAUUUAAGACAAAGGAUGGGGACAUUGGAGCG |
| | GUUGCGCUGGAUUACCCAGCAGGAACUUCAGGAUCUCCUAUCCUAGACAAGUG |
| | UGGGAGAGUGAUAGGACUUUAUGGCAAUGGGGUCGUGAUCAAAAAUGGGAGU |
| | UAUGUUAGUGCCAUCACCCAAGGGAGGAGGGAGGAAGAGACUCCUGUUGAGUG |
| | CUUCGAGCCUUCGAUGCUGAAGAAGAAGCAGCUAACUGUCUUAGACUUGCAUC |
| | CUGGAGCUGGGAAAACCAGGAGAGUUCUUCCUGAAAUAGUCCGUGAAGCCAUA |
| | AAAACAAGACUCCGUACUGUGAUCUUAGCUCCAACCAGGGUUGUCGCUGCUGA |
| | AAUGGAGGAGGCCCUUAGAGGGCUUCCAGUGCGUUAUAUGACAACAGCAGUCA |
| | AUGUCACCCACUCUGGAACAGAAAUCGUCGACUUAAUGUGCCAUGCCACCUUC |
| | ACUUCGCGUCUACUACAGCCAAUCAGAGUCCCCAACUAUAAUCUGUAUAUUAU |
| | GGAUGAGGCCCACUUCACAGAUCCCUCAAGUAUAGCAGCAAGAGGAUACAUUU |
| | CAACAAGGGUUGAGAUGGGCGAGGCGGCCGCCAUCUUCAUGACCGCCACGCCA |
| | CCAGGAACCCGUGACGCAUUUCCGGACUCCAACUCACCAAUUAUGGACACCGA |
| | AGUGGAAGUCCCAGAGAGAGCCUGGAGCUCAGGCUUUGAUUGGGUGACGGAUC |
| | AUUCUGGAAAAACAGUUUGGUUUGUUCCAAGCGUGAGGAACGGCAAUGAGAUC |
| | GCAGCUUGUCUGACAAAGGCUGGAAAACGGGUCAUACAGCUCAGCAGAAAGAC |
| | UUUUGAGACAGAGUUCCAGAAAACAAAACAUCAAGAGUGGGACUUUGUCGUGA |
| | CAACUGACAUUUCAGAGAUGGGCGCCAACUUUAAAGCUGACCGUGUCAUAGAU |
| | UCCAGGAGAUGCCUAAAGCCGGUCAUACUUGAUGGCGAGAGAGUCAUUCUGGC |
| | UGGACCCAUGCCUGUCACACAUGCCAGCGCUGCCCAGAGGAGGGGGCGCAUAG |
| | GCAGGAAUCCCAACAAACCUGGAGAUGAGUAUCUGUAUGGAGGUGGGUGCGCA |
| | GAGACUGACGAAGACCAUGCACACUGGCUUGAAGCAAGAAUGCUCCUUGACAA |
| | UAUUUACCUCCAAGAUGGCCUCAUAGCCUCGCUCUAUCGACCUGAGGCCGACA |
| | AAGUAGCAGCCAUUGAGGGAGAGUUCAAGCUUAGGACGGAGCAAAGGAAGACC |
| | UUUGUGGAACUCAUGAAAAGAGGAGAUCUUCCUGUUUGGCUGGCCUAUCAGGU |
| | UGCAUCUGCCGGAAUAACCUACACAGAUAGAAGAUGGUGCUUUGAUGGCACGA |
| | CCAACAACACCAUAAUGGAAGACAGUGUGCCGGCAGAAGUGUGGACCAGACAC |
| | GGAGAGAAAAGAGUGCUCAAACCGAGGUGGAUGGACGCCAGAGUUUGUUCAGA |
| | UCAUGCGGCCCUGAAGUCAUUCAAGGAGUUUGCCGCUGGGAAAAGAGGAGCGG |
| | CUUUUGGAGUGAUGGAAGCCCUGGGAACACUGCCAGGACACAUGACAGAGAGA |
| | UUCCAGGAAGCCAUUGACAACCUCGCUGUGCUCAUGCGGGCAGAGACUGGAAG |
| | CAGGCCUUACAAAGCCGCGGCGGCCCAAUUGCCGGAGACCCUAGAGACCAUUA |
| | UGCUUUUGGGGUUGCUGGGAACAGUCUCGCUGGGAAUCUUCUUCGUCUUGAUG |
| | AGGAACAAGGGCAUAGGGAAGAUGGGCUUUGGAAUGGUGACUCUUGGGGCCA |
| | GCGCAUGGCUCAUGUGGCUCUCGGAAAUUGAGCCAGCCAGAAUUGCAUGUGUC |
| | CUCAUUGUUGUGUUCCUAUUGCUGGUGGUGCUCAUACCUGAGCCAGAAAAGCA |
| | AAGAUCUCCCCAGGACAACCAAAUGGCAAUCAUCAUCAUGGUAGCAGUAGGUC |
| | UUCUGGGCUUGAUUACCGCCAAUGAACUCGGAUGGUUGGAGAGAACAAAGAGU |
| | GACCUAAGCCAUCUAAUGGGAAGGAGAGAGGAGGGGGCAACCAUAGGAUUCUC |
| | AAUGGACAUUGACCUGCGGCCAGCCUCAGCUUGGGCCAUCUAUGCUGCCUUGA |
| | CAACUUUCAUUACCCCAGCCGUCCAACAUGCAGUGACCACCUCAUACAACAACU |
| | ACUCCUUAAUGGCGAUGGCCACGCAAGCUGGAGUGUUGUUUGGUAUGGGCAAA |
| | GGGAUGCCAUUCUACGCAUGGGACUUUGGAGUCCCGCUGCUAAUGAUAGGUUG |
| | CUACUCACAAUUAACACCCCUGACCCUAAUAGUGGCCAUCAUUUUGCUCGUGG |
| | CGCACUACAUGUACUUGAUCCCAGGGCUGCAGGCAGCAGCUGCGCGUGCUGCC |
| | CAGAAGAGAACGGCAGCUGGCAUCAUGAAGAACCCUGUUGUGGAUGGAAUAGU |
| | GGUGACUGACAUUGACACAAUGACAAUUGACCCCCAAGUGGAGAAAAAGAUGG |
| | GACAGGUGCUACUCAUAGCAGUAGCCGUCUCCAGCGCCAUACUGUCGCGGACC |
| | GCCUGGGGGUGGGGGGAGGCUGGGGCCCUGAUCACAGCCGCAACUUCCACUUU |
| | GUGGGAAGGCUCUCCGAACAAGUACUGGAACUCCUCUACAGCCACUUCACUGU |
| | GUAACAUUUUUAGGGGAAGUUACUUGGCUGGAGCUUCUCUAAUCUACACAGUA |
| | ACAAGAAACGCUGGCUUGGUCAAGAGACGUGGGGGUGGAACAGGAGAGACCCU |
| | GGGAGAGAAAUGGAAGGCCCGCUUGAACCAGAUGUCGGCCCUGGAGUUCUACU |
| | CCUACAAAAAGUCAGGCAUCACCGAGGUGUGCAGAGAAGAGGCCCGCCGCGCC |
| | CUCAAGGACGGUGUGGCAACGGGAGGCCAUGCUGUGUCCCGAGGAAGUGCAAA |
| | GCUGAGAUGGUUGGUGGAGCGGGGAUACCUGCAGCCCUAUGGAAAGGUCAUUG |
| | AUCUUGGAUGUGGCAGAGGGGGCUGGAGUUACUACGCCGCCACCAUCCGCAAA |
| | GUUCAAGAAGUGAAAGGAUACACAAAAGGAGGCCCUGGUCAUGAAGAACCCGU |
| | GUUGGUGCAAAGCUAUGGGUGGAACAUAGUCCGUCUUAAGAGUGGGGUGGAC |
| | GUCUUUCAUAUGGCGGCUGAGCCGUGUGACACGUUGCUGUGUGACAUAGGUGA |
| | GUCAUCAUCUAGUCCUGAAGUGGAAGAAGCACGGACGCUCAGAGUCCUCUCCA |
| | UGGUGGGGGAUUGGCUUGAAAAAAGACCAGGAGCCUUUUGUAUAAAAGUGUU |
| | GUGCCCAUACACCAGCACUAUGAUGGAAACCCUGGAGCGACUGCAGCGUAGGU |
| | AUGGGGGAGGACUGGUCAGAGUGCCACUCUCCCGCAACUCUACACAUGAGAUG |
| | UACUGGGUCUCUGGAGCGAAAAGCAACACCAUAAAAAGUGUGUCCACCACGAG |
| | CCAGCUCCUCUUGGGGCGCAUGGACGGGCCUAGGAGGCCAGUGAAAUAUGAGG |
| | AGGAUGUGAAUCUCGGCUCUGGCACGCGGGCUGUGGUAAGCUGCGCUGAAGCU |
| | CCCAACAUGAAGAUCAUUGGUAACCGCAUUGAAAGGAUCCGCAGUGAGCACGC |
| | GGAAACGUGGUUCUUUGACGAGAACCACCCAUAUAGGACAUGGGCUUACCAUG |
| | GAAGCUAUGAGGCCCCCACACAAGGGUCAGCGUCCUCUCUAAUAAACGGGGUU |
| | GUCAGGCUCCUGUCAAAACCCUGGGAUGUGGUGACUGGAGUCACAGGAAUAGC |
| | CAUGACCGACACCACACCGUAUGGUCAGCAAAGAGUUUUCAAGGAAAAAGUGG |
| | ACACUAGGGUGCCAGACCCCCAAGAAGGCACUCGUCAGGUUAUGAGCAUGGUC |
| | UCUUCCUGGUUGUGGAAAGAGCUAGGCAAACACAAACGGCCACGAGUCUGUAC |
| | CAAAGAAGAGUUCAUCAACAAGGUUCGUAGCAAUGCAGCAUUAGGGGCAAUAU |
| | UUGAAGAGGAAAAAGAGUGGAAGACUGCAGUGGAAGCUGUGAACGAUCCAAG |
| | GUUCUGGGCUCUAGUGGACAAGGAAAGAGAGCACCACCUGAGAGGAGAGUGCC |
| | AGAGUUGUGUGUACAACAUGAUGGGAAAAAGAGAAAAGAAACAAGGGGAAUU |
| | UGGAAAGGCCAAGGGCAGCCGCGCCAUCUGGUAUAUGUGGCUAGGGGCUAGAU |
| | UUCUAGAGUUCGAAGCCCUUGGAUUCUUGAACGAGGAUCACUGGAUGGGGAGA |
| | GAGAACUCAGGAGGUGGUGUUGAAGGGCUGGGAUUACAAAGACUCGGAUAUG |
| | UCCUAGAAGAGAUGAGUCGUAUACCAGGAGGAAGGAUGUAUGCAGAUGACACU |
| | GCUGGCUGGGACACCCGCAUUAGCAGGUUUGAUCUGGAGAAUGAAGCUCUAAU |
| | CACCAACCAAAUGGAGAAAGGGCACAGGGCCUUGGCAUUGGCCAUAAUCAAGU |
| | ACACAUACCAAAACAAAGUGGUAAAGGUCCUUAGACCAGCUGAAAAAGGGAAA |
| | ACAGUUAUGGACAUUAUUUCGAGACAAGACCAAAGGGGGAGCGGACAAGUUGU |
| | CACUUACGCUCUUAACACAUUUACCAACCUAGUGGUGCAACUCAUUCGGAAUA |
| | UGGAGGCUGAGGAAGUUCUAGAGAUGCAAGACUUGUGGCUGCUGCGGAGGUCA |
| | GAGAAAGUGACUAACUGGUUGCAGAGCAACGGAUGGGAUAGGCUCAAACGAAU |
| | GGCAGUCAGUGGAGAUGAUUGCGUUGUGAAGCCAAUUGAUGAUAGGUUUGCA |
| | CAUGCCCUCAGGUUCUUGAAUGAUAUGGGAAAAGUUAGGAAGGACACACAAGA |
| | GUGGAAACCCUCAACUGGAUGGGACAACUGGGAAGAAGUUCCGUUUUGCUCCC |
| | ACCACUUCAACAAGCUCCAUCUCAAGGACGGGAGGUCCAUUGUGGUUCCCUGC |
| | CGCCACCAAGAUGAACUGAUUGGCCGGGCCCGCGUCUCUCCAGGGGCGGGAUG |
| | GAGCAUCCGGGAGACUGCUUGCCUAGCAAAAUCAUAUGCGCAAAUGUGGCAGC |
| | UCCUUUAUUUCCACAGAAGGGACCUCCGACUGAUGGCCAAUGCCAUUUGUUCA |
| | UCUGUGCCAGUUGACUGGGUUCCAACUGGGAGAACUACCUGGUCAAUCCAUGG |
| | AAAGGGAGAAUGGAUGACCACUGAAGACAUGCUUGUGGUGUGGAACAGAGUG |
| | UGGAUUGAGGAGAACGACCACAUGGAAGACAAGACCCCAGUUACGAAAUGGAC |
| | AGACAUUCCCUAUUUGGGAAAAAGGGAAGACUUGUGGUGUGGAUCUCUCAUAG |
| | GGCACAGACCGCGCACCACCUGGGCUGAGAACAUUAAAAACACAGUCAACAUG |
| | GUGCGCAGGAUCAUAGGUGAUGAAGAAAAGUACAUGGACUACCUAUCCACCCA |
| | AGUUCGCUACUUGGGUGAAGAAGGGUCUACACCUGGAGUGCUGUAAGCACCAA |
| | UCUUAAUGUUGUCAGGCCUGCUAGUCAGCCACAGCUUGGGGAAAGCUGUGCAG |
| | CC (SEQ ID NO: 746) |
| MR 766 | AGUUGUUGAUCUGUGUGAGUCAGACUGCGACAGUUCGAGUCUGAAGCGAGAGC |
| Zika virus | UAACAACAGUAUCAACAGGUUUAAUUUGGAUUUGGAAACGAGAGUUUCUGGU |
| genome, | CAUGAAAAACCCCAAAGAAGAAAUCCGGAGGAUCCGGAUUGUCAAUAUGCUAA |
| Uganda | AACGCGGAGUAGCCCGUGUAAACCCCUUGGGAGGUUUGAAGAGGUUGCCAGCC |
| 1947 | GGACUUCUGCUGGGUCAUGGACCCAUCAGAAUGGUUUUGGCGAUACUAGCCUU |
| (Accession | UUUGAGAUUUACAGCAAUCAAGCCAUCACUGGGCCUUAUCAACAGAUGGGGUU |
| number: | CCGUGGGGAAAAAAGAGGCUAUGGAAAUAAUAAAGAAGUUCAAGAAAGAUCU |
| AY632535; | UGCUGCCAUGUUGAGAAUAAUCAAUGCUAGGAAAGAGAGGAAGAGACGUGGC |
| 10,794-nts) | GCAGACACCAGCAUCGGAAUCAUUGGCCUCCUGCUGACUACAGCCAUGGCAGC |
| | AGAGAUCACUAGACGCGGGAGUGCAUACUACAUGUACUUGGAUAGGAGCGAUG |
| | CCGGGAAGGCCAUUUCGUUUGCUACCACAUUGGGAGUGAACAAGUGCCACGUA |
| | CAGAUCAUGGACCUCGGGCACAUGUGUGACGCCACCAUGAGUUAUGAGUGCCC |
| | UAUGCUGGAUGAGGGAGUGGAACCAGAUGAUGUCGAUUGCUGGUGCAACACGA |
| | CAUCAACUUGGGUUGUGUACGGAACCUGUCAUCACAAAAAAGGUGAGGCACGG |
| | CGAUCUAGAAGAGCCGUGACGCUCCCUUCUCACUCUACAAGGAAGUUGCAAAC |
| | GCGGUCGCAGACCUGGUUAGAAUCAAGAGAAUACACGAAGCACUUGAUCAAGG |
| | UUGAAAACUGGAUAUUCAGGAACCCCGGGUUUGCGCUAGUGGCCGUUGCCAUU |
| | GCCUGGCUUUUGGGAAGCUCGACGAGCCAAAAAGUCAUAUACUUGGUCAUGAU |
| | ACUGCUGAUUGCCCCGGCAUACAGUAUCAGGUGCAUUGGAGUCAGCAAUAGAG |
| | ACUUCGUGGAGGGCAUGUCAGGUGGGACCUGGGUUGAUGUUGUCUUGGAACAU |
| | GGAGGCUGCGUUACCGUGAUGGCACAGGACAAGCCAACAGUCGACAUAGAGUU |
| | GGUCACGACGACGGUUAGUAACAUGGCCGAGGUAAGAUCCUAUUGCUACGAGG |
| | CAUCGAUAUCGGACAUGGCUUCGGACAGUCGUUGCCCAACACAAGGUGAAGCC |
| | UACCUUGACAAGCAAUCAGACACUCAAUAUGUCUGCAAAAGAACAUUAGUGGA |
| | CAGAGGUUGGGGAAACGGUUGUGGACUUUUUGGCAAAGGGAGCUUGGUGACA |
| | UGUGCCAAGUUUACGUGUUCUAAGAAGAUGACCGGGAAGAGCAUUCAACCGGA |
| | AAAUCUGGAGUAUCGGAUAAUGCUAUCAGUGCAUGGCUCCCAGCAUAGCGGGA |
| | UGAUUGGAUAUGAAACUGACGAAGAUAGAGCGAAAGUCGAGGUUACGCCUAA |
| | UUCACCAAGAGCGGAAGCAACCUUGGGAGGCUUUGGAAGCUUAGGACUUGACU |
| | GUGAACCAAGGACAGGCCUUGACUUUUCAGAUCUGUAUUACCUGACCAUGAAC |
| | AAUAAGCAUUGGUUGGUGCACAAAGAGUGGUUUCAUGACAUCCCAUUGCCUUG |
| | GCAUGCUGGGGCAGACACCGGAACUCCACACUGGAACAACAAAGAGGCAUUGG |
| | UAGAAUUCAAGGAUGCCCACGCCAAGAGGCAAACCGUCGUCGUUCUGGGGAGC |
| | CAGGAAGGAGCCGUUCACACGGCUCUCGCUGGAGCUCUAGAGGCUGAGAUGGA |
| | UGGUGCAAAGGGAAGGCUGUUCUCUGGCCAUUUGAAAUGCCGCCUAAAAAUGG |
| | ACAAGCUUAGAUUGAAGGGCGUGUCAUAUUCCUUGUGCACUGCGGCAUUCACA |
| | UUCACCAAGGUCCCAGCUGAAACACUGCAUGGAACAGUCACAGUGGAGGUGCA |
| | GUAUGCAGGGACAGAUGGACCCUGCAAGAUCCCAGUCCAGAUGGCGGUGGACA |
| | UGCAGACCCUGACCCCAGUUGGAAGGCUGAUAACCGCCAACCCCGUGAUUACU |
| | GAAAGCACUGAGAACUCAAAGAUGAUGUUGGAGCUUGACCCACCAUUUGGGGA |
| | UUCUUACAUUGUCAUAGGAGUUGGGGACAAGAAAAUCACCCACCACUGGCAUA |
| | GGAGUGGUAGCACCAUCGGAAAGGCAUUUGAGGCCACUGUGAGAGGCGCCAAG |
| | AGAAUGGCAGUCCUGGGGGAUACAGCCUGGGACUUCGGAUCAGUCGGGGGUGU |
| | GUUCAACUCACUGGGUAAGGGCAUUCACCAGAUUUUUGGAGCAGCCUUCAAAU |
| | CACUGUUUGGAGGAAUGUCCUGGUUCUCACAGAUCCUCAUAGGCACGCUGCUA |
| | GUGUGGUUAGGUUUGAACACAAAGAAUGGAUCUAUCUCCCUCACAUGCUUGGC |
| | CCUGGGGGGAGUGAUGAUCUUCCUCUCCACGGCUGUUUCUGCUGACGUGGGGU |
| | GCUCAGUGGACUUCUCAAAAAAGGAAACGAGAUGUGGCACGGGGGUAUUCAUC |
| | UAUAAUGAUGUUGAAGCCUGGAGGGACCGGUACAAGUACCAUCCUGACUCCCC |
| | CCGCAGAUUGGCAGCAGCAGUCAAGCAGGCCUGGGAAGAGGGGAUCUGUGGGA |
| | UCUCAUCCGUUUCAAGAAUGGAAAACAUCAUGUGGAAAUCAGUAGAAGGGGAG |
| | CUCAAUGCUAUCCUAGAGGAGAAUGGAGUUCAACUGACAGUUGUUGUGGGAUC |
| | UGUAAAAAACCCCAUGUGGAGAGGUCCACAAAGAUUGCCAGUGCCUGUGAAUG |
| | AGCUGCCCCAUGGCUGGAAAGCCUGGGGGAAAUCGUAUUUUGUUAGGGCGGCA |
| | AAGACCAACAACAGUUUUGUUGUCGACGGUGACACACUGAAGGAAUGUCCGCU |
| | UGAGCACAGAGCAUGGAAUAGUUUUCUUGUGGAGGAUCACGGGUUUGGAGUC |
| | UUCCACACCAGUGUCUGGCUUAAGGUCAGAGAAGAUUACUCAUUAGAAUGUGA |
| | CCCAGCCGUCAUAGGAACAGCUGUUAAGGGAAGGGAGGCCGCGCACAGUGAUC |
| | UGGGCUAUUGGAUUGAAAGUGAAAAGAAUGACACAUGGAGGCUGAAGAGGGC |
| | CCACCUGAUUGAGAUGAAAACAUGUGAAUGGCCAAAGUCUCACACAUUGUGGA |
| | CAGAUGGAGUAGAAGAAAGUGAUCUUAUCAUACCCAAGUCUUUAGCUGGUCCA |
| | CUCAGCCACCACAACACCAGAGAGGGUUACAGAACCCAAGUGAAAGGGCCAUG |
| | GCACAGUGAAGAGCUUGAAAUCCGGUUUGAGGAAUGUCCAGGCACCAAGGUUU |
| | ACGUGGAGGAGACAUGCGGAACUAGAGGACCAUCUCUGAGAUCAACUACUGCA |
| | AGUGGAAGGGUCAUUGAGGAAUGGUGCUGUAGGGAAUGCACAAUGCCCCCACU |
| | AUCGUUUCGAGCAAAAGACGGCUGCUGGUAUGGAAUGGAGAUAAGGCCCAGGA |
| | AAGAACCAGAGAGCAACUUAGUGAGGUCAAUGGUGACAGCGGGGUCAACCGAU |
| | CAUAUGGACCACUUCUCUCUUGGAGUGCUUGUGAUUCUACUCAUGGUGCAGGA |
| | GGGGUUGAAGAAGAGAAUGACCACAAAGAUCAUCAUGAGCACAUCAAUGGCAG |
| | UGCUGGUAGUCAUGAUCUUGGGAGGAUUUUCAAUGAGUGACCUGGCCAAGCUU |
| | GUGAUCCUGAUGGGUGCUACUUUCGCAGAAAUGAACACUGGAGGAGAUGUAGC |
| | UCACUUGGCAUUGGUAGCGGCAUUUAAAGUCAGACCAGCCUUGCUGGUCUCCU |
| | UCAUUUUCAGAGCCAAUUGGACACCCCGUGAGAGCAUGCUGCUAGCCCUGGCU |
| | UCGUGUCUUCUGCAAACUGCGAUCUCUGCUCUUGAAGGUGACUUGAUGGUCCU |
| | CAUUAAUGGAUUUGCUUUGGCCUGGUUGGCAAUUCGAGCAAUGGCCGUGCCAC |
| | GCACUGACAACAUCGCUCUACCAAUCUUGGCUGCUCUAACACCACUAGCUCGA |
| | GGCACACUGCUCGUGGCAUGGAGAGCGGGCCUGGCUACUUGUGGAGGGAUCAU |
| | GCUCCUCUCCCUGAAAGGGAAAGGUAGUGUGAAGAAGAACCUGCCAUUUGUCA |
| | UGGCCCUGGGAUUGACAGCUGUGAGGGUAGUAGACCCUAUUAAUGUGGUAGGA |
| | CUACUGUUACUCACAAGGAGUGGGAAGCGGAGCUGGCCCCCUAGUGAAGUUCU |
| | CACAGCCGUUGGCCUGAUAUGUGCACUGGCCGGAGGGUUUGCCAAGGCAGACA |
| | UUGAGAUGGCUGGACCCAUGGCUGCAGUAGGCUUGCUAAUUGUCAGCUAUGUG |
| | GUCUCGGGAAAGAGUGUGGACAUGUACAUUGAAAGAGCAGGUGACAUCACAUG |
| | GGAAAAGGACGCGGAAGUCACUGGAAACAGUCCUCGGCUUGACGUGGCACUGG |
| | AUGAGAGUGGUGACUUCUCCUUGGUAGAGGAAGAUGGUCCACCCAUGAGAGAG |
| | AUCAUACUCAAGGUGGUCCUGAUGGCCAUCUGUGGCAUGAACCCAAUAGCUAU |
| | ACCUUUUGCUGCAGGAGCGUGGUAUGUGUAUGUGAAGACUGGGAAAAGGAGU |
| | GGCGCCCUCUGGGACGUGCCUGCUCCCAAAGAAGUGAAGAAAGGAGAGACCAC |
| | AGAUGGAGUGUACAGAGUGAUGACUCGCAGACUGCUAGGUUCAACACAGGUUG |
| | GAGUGGGAGUCAUGCAAGAGGGAGUCUUCCACACCAUGUGGCACGUUACAAAA |
| | GGAGCCGCACUGAGGAGCGGUGAGGGAAGACUUGAUCCAUACUGGGGGGAUGU |
| | CAAGCAGGACUUGGUGUCAUACUGUGGGCCUUGGAAGUUGGAUGCAGCUUGGG |
| | AUGGACUCAGCGAGGUACAGCUUUUGGCCGUACCUCCCGGAGAGAGGGCCAGA |
| | AACAUUCAGACCCUGCCUGGAAUAUUCAAGACAAAGGACGGGGACAUCGGAGC |
| | AGUUGCUCUGGACUACCCUGCAGGGACCUCAGGAUCUCCGAUCCUAGACAAAU |
| | GUGGAAGAGUGAUAGGACUCUAUGGCAAUGGGGUUGUGAUCAAGAAUGGAAG |
| | CUAUGUUAGUGCUAUAACCCAGGGAAAGAGGGAGGAGGAGACUCCGGUUGAAU |
| | GUUUCGAACCCUCGAUGCUGAAGAAGAAGCAGCUAACUGUCUUGGAUCUGCAU |
| | CCAGGAGCCGGAAAAACCAGGAGAGUUCUUCCUGAAAUAGUCCGUGAAGCCAU |
| | AAAAAAGAGACUCCGGACAGUGAUCUUGGCACCAACUAGGGUUGUCGCUGCUG |
| | AGAUGGAGGAGGCCUUGAGAGGACUUCCGGUGCGUUACAUGACAACAGCAGUC |
| | AACGUCACCCAUUCUGGGACAGAAAUCGUUGAUUUGAUGUGCCAUGCCACUUU |
| | CACUUCACGCUUACUACAACCCAUCAGAGUCCCUAAUUACAAUCUCAACAUCA |
| | UGGAUGAAGCCCACUUCACAGACCCCUCAAGUAUAGCUGCAAGAGGAUACAUA |
| | UCAACAAGGGUUGAAAUGGGCGAGGCGGCUGCCAUUUUUAUGACUGCCACACC |
| | ACCAGGAACCCGUGAUGCGUUUCCUGACUCUAACUCACCAAUCAUGGACACAG |
| | AAGUGGAAGUCCCAGAGAGAGCCUGGAGCUCAGGCUUUGAUUGGGUGACAGAC |
| | CAUUCUGGGAAAACAGUUUGGUUCGUUCCAAGCGUGAGAAACGGAAAUGAAAU |
| | CGCAGCCUGUCUGACAAAGGCUGGAAAGCGGGUCAUACAGCUCAGCAGGAAGA |
| | CUUUUGAGACAGAAUUUCAGAAAACAAAAAAUCAAGAGUGGGACUUUGUCAU |
| | AACAACUGACAUCUCAGAGAUGGGCGCCAACUUCAAGGCUGACCGGGUCAUAG |
| | ACUCUAGGAGAUGCCUAAAACCAGUCAUACUUGAUGGUGAGAGAGUCAUCUUG |
| | GCUGGGCCCAUGCCUGUCACGCAUGCUAGUGCUGCUCAGAGGAGAGGACGUAU |
| | AGGCAGGAACCCUAACAAACCUGGAGAUGAGUACAUGUAUGGAGGUGGGUGUG |
| | CAGAGACUGAUGAAGGCCAUGCACACUGGCUUGAAGCAAGAAUGCUUCUUGAC |
| | AACAUCUACCUCCAGGAUGGCCUCAUAGCCUCGCUCUAUCGGCCUGAGGCCGA |
| | UAAGGUAGCCGCCAUUGAGGGAGAGUUUAAGCUGAGGACAGAGCAAAGGAAG |
| | ACCUUCGUGGAACUCAUGAAGAGAGGAGACCUUCCCGUCUGGCUAGCCUAUCA |
| | GGUUGCAUCUGCCGGAAUAACUUACACAGACAGAAGAUGGUGCUUUGAUGGCA |
| | CAACCAACAACACCAUAAUGGAAGACAGUGUACCAGCAGAGGUUUGGACAAAG |
| | UAUGGAGAGAAGAGAGUGCUCAAACCGAGAUGGAUGGAUGCUAGGGUCUGUU |
| | CAGACCAUGCGGCCCUGAAGUCGUUCAAAGAAUUCGCCGCUGGAAAAAGAGGA |
| | GCGGCUUUGGGAGUAAUGGAGGCCCUGGGAACACUGCCAGGACACAUGACAGA |
| | GAGGUUUCAGGAAGCCAUUGACAACCUCGCCGUGCUCAUGCGAGCAGAGACUG |
| | GAAGCAGGCCUUAUAAGGCAGCGGCAGCCCAACUGCCGGAGACCCUAGAGACC |
| | AUUAUGCUCUUAGGUUUGCUGGGAACAGUUUCACUGGGGAUCUUCUUCGUCUU |
| | GAUGCGGAAUAAGGGCAUCGGGAAGAUGGGCUUUGGAAUGGUAACCCUUGGG |
| | GCCAGUGCAUGGCUCAUGUGGCUUUCGGAAAUUGAACCAGCCAGAAUUGCAUG |
| | UGUCCUCAUUGUUGUGUUUUUAUUACUGGUGGUGCUCAUACCCGAGCCAGAGA |
| | AGCAAAGAUCUCCCCAAGAUAACCAGAUGGCAAUUAUCAUCAUGGUGGCAGUG |
| | GGCCUUCUAGGUUUGAUAACUGCAAACGAACUUGGAUGGCUGGAAAGAACAAA |
| | AAAUGACAUAGCUCAUCUAAUGGGAAGGAGAGAAGAAGGAGCAACCAUGGGA |
| | UUCUCAAUGGACAUUGAUCUGCGGCCAGCCUCCGCCUGGGCUAUCUAUGCCGC |
| | AUUGACAACUCUCAUCACCCCAGCUGUCCAACAUGCGGUAACCACUUCAUACA |
| | ACAACUACUCCUUAAUGGCGAUGGCCACACAAGCUGGAGUGCUGUUUGGCAUG |
| | GGCAAAGGGAUGCCAUUUAUGCAUGGGGACCUUGGAGUCCCGCUGCUAAUGAU |
| | GGGUUGCUAUUCACAAUUAACACCCCUGACUCUGAUAGUAGCUAUCAUUCUGC |
| | UUGUGGCGCACUACAUGUACUUGAUCCCAGGCCUACAAGCGGCAGCAGCGCGU |
| | GCUGCCCAGAAAAGGACAGCAGCUGGCAUCAUGAAGAAUCCCGUUGUGGAUGG |
| | AAUAGUGGUAACUGACAUUGACACAAUGACAAUAGACCCCCAGGUGGAGAAGA |
| | AGAUGGGACAAGUGUUACUCAUAGCAGUAGCCAUCUCCAGUGCUGUGCUGCUG |
| | CGGACCGCCUGGGGAUGGGGGGAGGCUGGAGCUCUGAUCACAGCAGCGACCUC |
| | CACCUUGUGGGAAGGCUCUCCAAACAAAUACUGGAACUCCUCUACAGCCACCU |
| | CACUGUGCAACAUCUUCAGAGGAAGCUAUCUGGCAGGAGCUUCCCUUAUCUAU |
| | ACAGUGACGAGAAACGCUGGCCUGGUUAAGAGACGUGGAGGUGGGACGGGAGA |
| | GACUCUGGGAGAGAAGUGGAAAGCUCGUCUGAAUCAGAUGUCGGCCCUGGAGU |
| | UCUACUCUUAUAAAAAGUCAGGUAUCACUGAAGUGUGUAGAGAGGAGGCUCGC |
| | CGUGCCCUCAAGGAUGGAGUGGCCACAGGAGGACAUGCCGUAUCCCGGGGAAG |
| | UGCAAAGAUCAGAUGGUUGGAGGAGAGAGGAUAUCUGCAGCCCUAUGGGAAG |
| | GUUGUUGACCUCGGAUGUGGCAGAGGGGGCUGGAGCUAUUAUGCCGCCACCAU |
| | CCGCAAAGUGCAGGAGGUGAGAGGAUACACAAAGGGAGGUCCCGGUCAUGAAG |
| | AACCCAUGCUGGUGCAAAGCUAUGGGUGGAACAUAGUUCGUCUCAAGAGUGGA |
| | GUGGACGUCUUCCACAUGGCGGCUGAGCCGUGUGACACUCUGCUGUGUGACAU |
| | AGGUGAGUCAUCAUCUAGUCCUGAAGUGGAAGAGACACGAACACUCAGAGUGC |
| | UCUCUAUGGUGGGGGACUGGCUUGAAAAAAGACCAGGGGCCUUCUGUAUAAAG |
| | GUGCUGUGCCCAUACACCAGCACUAUGAUGGAAACCAUGGAGCGACUGCAACG |
| | UAGGCAUGGGGGAGGAUUAGUCAGAGUGCCAUUGUGUCGCAACUCCACACAUG |
| | AGAUGUACUGGGUCUCUGGGGCAAAGAGCAACAUCAUAAAAAGUGUGUCCACC |
| | ACAAGUCAGCUCCUCCUGGGACGCAUGGAUGGCCCCAGGAGGCCAGUGAAAUA |
| | UGAGGAGGAUGUGAACCUCGGCUCGGGUACACGAGCUGUGGCAAGCUGUGCUG |
| | AGGCUCCUAACAUGAAAAUCAUCGGCAGGCGCAUUGAGAGAAUCCGCAAUGAA |
| | CAUGCAGAAACAUGGUUUCUUGAUGAAAACCACCCAUACAGGACAUGGGCCUA |
| | CCAUGGGAGCUACGAAGCCCCCACGCAAGGAUCAGCGUCUUCCCUCGUGAACG |
| | GGGUUGUUAGACUCCUGUCAAAGCCUUGGGACGUGGUGACUGGAGUUACAGGA |
| | AUAGCCAUGACUGACACCACACCAUACGGCCAACAAAGAGUCUUCAAAGAAAA |
| | AGUGGACACCAGGGUGCCAGAUCCCCAAGAAGGCACUCGCCAGGUAAUGAACA |
| | UAGUCUCUUCCUGGCUGUGGAAGGAGCUGGGGAAACGCAAGCGGCCACGCGUC |
| | UGCACCAAAGAAGAGUUUAUCAACAAGGUGCGCAGCAAUGCAGCACUGGGAGC |
| | AAUAUUUGAAGAGGAAAAAGAAUGGAAGACGGCUGUGGAAGCUGUGAAUGAU |
| | CCAAGGUUUUGGGCCCUAGUGGAUAGGGAGAGAGAACACCACCUGAGAGGAGA |
| | GUGUCACAGCUGUGUGUACAACAUGAUGGGAAAAAGAGAAAAGAAGCAAGGA |
| | GAGUUCGGGAAAGCAAAAGGUAGCCGCGCCAUCUGGUACAUGUGGUUGGGAGC |
| | CAGAUUCUUGGAGUUUGAAGCCCUUGGAUUCUUGAACGAGGACCAUUGGAUGG |
| | GAAGAGAAAACUCAGGAGGUGGAGUCGAAGGGUUAGGAUUGCAAAGACUUGG |
| | AUACAUUCUAGAAGAAAUGAAUCGGGCACCAGGAGGAAAGAUGUACGCAGAUG |
| | ACACUGCUGGCUGGGACACCCGCAUUAGUAAGUUUGAUCUGGAGAAUGAAGCU |
| | CUGAUUACCAACCAAAUGGAGGAAGGGCACAGAACUCUGGCGUUGGCCGUGAU |
| | UAAAUACACAUACCAAAACAAAGUGGUGAAGGUUCUCAGACCAGCUGAAGGAG |
| | GAAAAACAGUUAUGGACAUCAUUUCAAGACAAGACCAGAGAGGGAGUGGACAA |
| | GUUGUCACUUAUGCUCUCAACACAUUCACCAACUUGGUGGUGCAGCUUAUCCG |
| | GAACAUGGAAGCUGAGGAAGUGUUAGAGAUGCAAGACUUAUGGUUGUUGAGG |
| | AAGCCAGAGAAAGUGACCAGAUGGUUGCAGAGCAAUGGAUGGGAUAGACUCAA |
| | ACGAAUGGCGGUCAGUGGAGAUGACUGCGUUGUGAAGCCAAUCGAUGAUAGGU |
| | UUGCACAUGCCCUCAGGUUCUUGAAUGACAUGGGAAAAGUUAGGAAAGACACA |
| | CAGGAGUGGAAACCCUCGACUGGAUGGAGCAAUUGGGAAGAAGUCCCGUUCUG |
| | CUCCCACCACUUCAACAAGCUGUACCUCAAGGAUGGGAGAUCCAUUGUGGUCC |
| | CUUGCCGCCACCAAGAUGAACUGAUUGGCCGAGCUCGCGUCUCACCAGGGGCA |
| | GGAUGGAGCAUCCGGGAGACUGCCUGUCUUGCAAAAUCAUAUGCGCAGAUGUG |
| | GCAGCUCCUUUAUUUCCACAGAAGAGACCUUCGACUGAUGGCUAAUGCCAUUU |
| | GCUCGGCUGUGCCAGUUGACUGGGUACCAACUGGGAGAACCACCUGGUCAAUC |
| | CAUGGAAAGGGAGAAUGGAUGACCACUGAGGACAUGCUCAUGGUGUGGAAUA |
| | GAGUGUGGAUUGAGGAGAACGACCAUAUGGAGGACAAGACUCCUGUAACAAAA |
| | UGGACAGACAUUCCCUAUCUAGGAAAAAGGGAGGACUUAUGGUGUGGAUCCCU |
| | UAUAGGGCACAGACCCCGCACCACUUGGGCUGAAAACAUCAAAGACACAGUCA |
| | ACAUGGUGCGCAGGAUCAUAGGUGAUGAAGAAAAGUACAUGGACUAUCUAUCC |
| | ACCCAAGUCCGCUACUUGGGUGAGGAAGGGUCCACACCCGGAGUGUUGUAAGC |
| | ACCAAUUUUAGUGUUGUCAGGCCUGCUAGUCAGCCACAGUUUGGGGAAAGCUG |
| | UGCAGCCUGUAACCCCCCCAGGAGAAGCUGGGAAACCAAGCUCAUAGUCAGGC |
| | CGAGAACGCCAUGGCACGGAAGAAGCCAUGCUGCCUGUGAGCCCCUCAGAGGA |
| | CACUGAGUCAAAAAACCCCACGCGCUUGGAAGCGCAGGAUGGGAAAAGAAGGU |
| | GGCGACCUUCCCCACCCUUCAAUCUGGGGCCUGAACUGGAGACUAGCUGUGAA |
| | UCUCCAGCAGAGGGACUAGUGGUUAGAGGAGACCCCCCGGAAAACGCAAAACA |
| | GCAUAUUGACGUGGGAAAGACCAGAGACUCCAUGAGUUUCCACCACGCUGGCC |
| | GCCAGGCACAGAUCGCCGAACUUCGGCGGCCGGUGUGGGGAAAUCCAUGGUUU |
| | CU (SEQ ID NO: 747) |

TABLE 8
Sequences of Toehold Switch Sensors Used for Zika RNA Detection

| Toehold | Toehold Switch RNA Sequences for | |
|---|---|---|
| Switch | Detection of Zika Virus from the | Target Sequence in Zika Virus from |
| Name | Americas (KU312312) | the Americas (KU312312) |
| 27B_N1 | GGGUUUCGCUCUAUUCUCAUCAGU | GACACAGGACAUGAAACUGAUGAGA |
| | UUCAUGUCCUGUGUCGGACUUUAG | AUAGAGCGAAA (SEQ ID NO: 758) |
| | AACAGAGGAGAUAAAGAUGGACAC | |
| | AGGACACAACCUGGCGGCAGCGCA | |
| | AAAG (SEQ ID NO: 748) | |
| 27B_N2 | GGGCUCAACUUUCGCUCUAUUCUC | GGACAUGAAACUGAUGAGAAUAGA |
| | AUCAGUUUCAUGUCCGGACUUUAG | GCGAAAGUUGAG (SEQ ID NO: 759) |
| | AACAGAGGAGAUAAAGAUGGGACA | |
| | UGAAACAACCUGGCGGCAGCGCAA | |
| | GAAG (SEQ ID NO: 749) | |
| 27B_N3 | GGGUUAUCUCAACUUUCGCUCUAU | AUGAAACUGAUGAGAAUAGAGCGA |
| | UCUCAUCAGUUUCAUGGACUUUAG | AAGUUGAGAUAA (SEQ ID NO: 760) |
| | AACAGAGGAGAUAAAGAUGAUGAA | |
| | ACUGAUAACCUGGCGGCAGCGCAA | |
| | GAAG (SEQ ID NO: 750) | |
| 27B_N4 | GGGUCGCUCUAUUCUCAUCAGUUU | AUGACACAGGACAUGAAACUGAUGA |
| | CAUGUCCUGUGUCAUGGACUUUAG | GAAUAGAGCGA (SEQ ID NO: 761) |
| | AACAGAGGAGAUAAAGAUGAUGAC | |
| | ACAGGAAACCUGGCGGCAGCGCAA | |
| | GAAG (SEQ ID NO: 751) | |
| 27B_N5 | GGGUGGCUUCGGCUCUUGGUGAAU | GAGAUAACGCCCAAUUCACCAAGAG |
| | UGGGCGUUAUCUCGGACUUUAGAA | CCGAAGCCACC (SEQ ID NO: 762) |
| | CAGAGGAGAUAAAGAUGGAGAUAA | |
| | CGCCAACCUGGCGGCAGCGCAAGA | |
| | AG (SEQ ID NO: 752) | |
| 32B_N1 | GGGCUGGGAUCAAGUACAUGUAGU | UUUUGCUCGUGGCGCACUACAUGUA |
| | GCGCCACGAGCAAAAGGACUUUAG | CUUGAUCCCAG (SEQ ID NO: 763) |
| | AACAGAGGAGAUAAAGAUGUUUUG | |
| | CUCGUGUAACCUGGCGGCAGCGCA | |
| | AAAG (SEQ ID NO: 753) | |
| 32B_N2 | GGGCCUGCAGCCCUGGGAUCAAGU | GGCGCACUACAUGUACUUGAUCCCA |
| | ACAUGUAGUGCGCCGGACUUUAGA | GGGCUGCAGGC (SEQ ID NO: 764) |
| | ACAGAGGAGAUAAAGAUGGGCGCA | |
| | CUACAAACCUGGCGGCAGCGCAAG | |
| | AAG (SEQ ID NO: 754) | |
| 32B_N3 | GGGCUGCCGUUCUCUUCUGGGCAG | CAGCAGCUGCGCGUGCUGCCCAGAA |
| | CACGCGCAGCUGCUGGGACUUUAG | GAGAACGGCAG (SEQ ID NO: 765) |
| | AACAGAGGAGAUAAAGAUGCAGCA | |
| | GCUGCGAACCUGGCGGCAGCGCAA | |
| | GAAG (SEQ ID NO: 755) | |
| 32B_N4 | GGGCAGCCCUGGGAUCAAGUACAU | CUCGUGGCGCACUACAUGUACUUGA |
| | GUAGUGCGCCACGAGGGACUUUAG | UCCCAGGGCUG (SEQ ID NO: 766) |
| | AACAGAGGAGAUAAAGAUGCUCGU | |
| | GGCGCAAACCUGGCGGCAGCGCAA | |
| | GAAG (SEQ ID NO: 756) | |
| 32B_N5 | GGGAUGCCAGCUGCCGUUCUCUUC | GCGCGUGCUGCCCAGAAGAGAACGG |
| | UGGGCAGCACGCGCGGACUUUAGA | CAGCUGGCAUC (SEQ ID NO: 767) |
| | ACAGAGGAGAUAAAGAUGGCGCGU | |
| | GCUGCAACCUGGCGGCAGCGCAAG | |
| | AAG (SEQ ID NO: 757) | |

1.-62. (canceled)

63. A composition comprising a sgRNA selected from SEQ ID NOs: 60-649.

64. The composition of claim 63, further comprising a Cas nuclease.

65. The composition of claim 64, wherein the Cas nuclease is Cas9 or Cas13.

66. The composition of claim 63, further comprising a CRISPR targeting RNA (crRNA) and a trans-activating crRNA (tracrRNA).


SEQUENCE LISTING (789 sequences)

SEQ ID NO: 1 - DNA, 100 bp, synthetic construct
  misc_feature 1..100: note = Description of Artificial Sequence: Synthetic polynucleotide
  source 1..100: mol_type = other DNA
tggagtcccgctgctaatgagttttagagctagaaatagcaagttaaaataaggctagtccgttatcaacttgaaaaagtggcaccgagtcggtgctttt

SEQ ID NO: 2 - protein, 9 aa, unidentified
  REGION 1..9: note = Description of Unknown: 'LAGLIDADG' homing endonuclease motif sequence
LAGLIDADG

SEQ ID NO: 3 - DNA, 36 bp, synthetic construct
  misc_feature 1..36: note = Description of Artificial Sequence: Synthetic oligonucleotide
  source 1..36: mol_type = other DNA
gtttgaatgaattgtaggcttgttatagttatgttt

SEQ ID NO: 4 - DNA, 20 bp, synthetic construct
  misc_feature 1..20: note = Description of Artificial Sequence: Synthetic oligonucleotide
  source 1..20: mol_type = other DNA
ctaatacgactcactatagg

SEQ ID NO: 5 - DNA, 31 bp, synthetic construct
  misc_feature 1..31: note = Description of Artificial Sequence: Synthetic oligonucleotide
  source 1..31: mol_type = other DNA
aattctaatacgactcactatagggagaagg

SEQ ID NO: 6 - RNA, 35 nt, synthetic construct
  misc_feature 1..35: note = Description of Artificial Sequence: Synthetic oligonucleotide
  source 1..35: mol_type = other RNA
gttatagttatgaacagaggagacataacatgaac

SEQ ID NO: 7 - RNA, 28 nt, synthetic construct
  misc_feature 1..28: note = Description of Artificial Sequence: Synthetic oligonucleotide
  source 1..28: mol_type = other RNA
ggactttagaacagaggagataaagatg

SEQ ID NO: 8 - DNA, 51 bp, synthetic construct
  misc_feature 1..51: note = Description of Artificial Sequence: Synthetic primer
  source 1..51: mol_type = other DNA
aattctaatacgactcactatagggagaaggcagtgatctaggctactgga

SEQ ID NO: 9 - DNA, 51 bp, synthetic construct
  misc_feature 1..51: note = Description of Artificial Sequence: Synthetic primer
  source 1..51: mol_type = other DNA
aattctaatacgactcactatagggagaagggtgccagagttgtgtgtaca

SEQ ID NO: 10 - DNA, 51 bp, synthetic construct
  misc_feature 1..51: note = Description of Artificial Sequence: Synthetic primer
  source 1..51: mol_type = other DNA
aattctaatacgactcactatagggagaagggcacagtgggatgatcgtta

SEQ ID NO: 11 - DNA, 51 bp, synthetic construct
  misc_feature 1..51: note = Description of Artificial Sequence: Synthetic primer
  source 1..51: mol_type = other DNA
aattctaatacgactcactatagggagaaggcgggatctcctctgtttcaa

SEQ ID NO: 12 - DNA, 51 bp, synthetic construct
  misc_feature 1..51: note = Description of Artificial Sequence: Synthetic primer
  source 1..51: mol_type = other DNA
aattctaatacgactcactatagggagaaggccatcactgggtctcatcaa

SEQ ID NO: 13 - DNA, 55 bp, synthetic construct
  misc_feature 1..55: note = Description of Artificial Sequence: Synthetic primer
  source 1..55: mol_type = other DNA
aattctaatacgactcactatagggagaaggaatgctgtcagttcatggctccca

SEQ ID NO: 14 - DNA, 53 bp, synthetic construct
  misc_feature 1..53: note = Description of Artificial Sequence: Synthetic primer
  source 1..53: mol_type = other DNA
aattctaatacgactcactatagggagaaggatggtctcttcctggttgtgga

SEQ ID NO: 15 - DNA, 51 bp, synthetic construct
  misc_feature 1..51: note = Description of Artificial Sequence: Synthetic primer
  source 1..51: mol_type = other DNA
aattctaatacgactcactatagggagaagggaccctaatagtggccatca

SEQ ID NO: 16 - DNA, 51 bp, synthetic construct
  misc_feature 1..51: note = Description of Artificial Sequence: Synthetic primer
  source 1..51: mol_type = other DNA
aattctaatacgactcactatagggagaagggtttggtatgggcaaaggga

SEQ ID NO: 17 - DNA, 53 bp, synthetic construct
  misc_feature 1..53: note = Description of Artificial Sequence: Synthetic primer
  source 1..53: mol_type = other DNA
aattctaatacgactcactatagggagaagggccatctatgctgccttgacaa

SEQ ID NO: 18 - DNA, 51 bp, synthetic construct
  misc_feature 1..51: note = Description of Artificial Sequence: Synthetic primer
  source 1..51: mol_type = other DNA
aattctaatacgactcactatagggagaagggtgattctgctcatggtgca

SEQ ID NO: 19 - DNA, 51 bp, synthetic construct
  misc_feature 1..51: note = Description of Artificial Sequence: Synthetic primer
  source 1..51: mol_type = other DNA
aattctaatacgactcactatagggagaagggtttgttccaagcgtgagga

SEQ ID NO: 20 - DNA, 53 bp, synthetic construct
  misc_feature 1..53: note = Description of Artificial Sequence: Synthetic primer
  source 1..53: mol_type = other DNA
aattctaatacgactcactatagggagaaggagatcaaccactgcaagcggaa

SEQ ID NO: 21 - DNA, 51 bp, synthetic construct
  misc_feature 1..51: note = Description of Artificial Sequence: Synthetic primer
  source 1..51: mol_type = other DNA
aattctaatacgactcactatagggagaaggagtggttccacgacattcca

SEQ ID NO: 22 - DNA, 53 bp, synthetic construct
  misc_feature 1..53: note = Description of Artificial Sequence: Synthetic primer
  source 1..53: mol_type = other DNA
aattctaatacgactcactatagggagaaggtggacgccagagtttgttcaga

SEQ ID NO: 23 - DNA, 51 bp, synthetic construct
  misc_feature 1..51: note = Description of Artificial Sequence: Synthetic primer
  source 1..51: mol_type = other DNA
aattctaatacgactcactatagggagaagggccggaataacctacacaga

SEQ ID NO: 24 - DNA, 53 bp, synthetic construct
  misc_feature 1..53: note = Description of Artificial Sequence: Synthetic primer
  source 1..53: mol_type = other DNA
aattctaatacgactcactatagggagaaggggctactggattgagagtgaga

SEQ ID NO: 25 - DNA, 55 bp, synthetic construct
  misc_feature 1..55: note = Description of Artificial Sequence: Synthetic primer
  source 1..55: mol_type = other DNA
aattctaatacgactcactatagggagaaggcagccagaattgcatgtgtcctca

SEQ ID NO: 26 - DNA, 53 bp, synthetic construct
  misc_feature 1..53: note = Description of Artificial Sequence: Synthetic primer
  source 1..53: mol_type = other DNA
aattctaatacgactcactatagggagaaggggagcggacaagttgtcactta

SEQ ID NO: 27 - DNA, 55 bp, synthetic construct
  misc_feature 1..55: note = Description of Artificial Sequence: Synthetic primer
  source 1..55: mol_type = other DNA
aattctaatacgactcactatagggagaagggtgctcggtggacttctcaaagaa

SEQ ID NO: 28 - DNA, 51 bp, synthetic construct
  misc_feature 1..51: note = Description of Artificial Sequence: Synthetic primer
  source 1..51: mol_type = other DNA
aattctaatacgactcactatagggagaaggagtggtgcaactcattcgga

SEQ ID NO: 29 - DNA, 51 bp, synthetic construct
  misc_feature 1..51: note = Description of Artificial Sequence: Synthetic primer
  source 1..51: mol_type = other DNA
aattctaatacgactcactatagggagaaggcaataccagagagggctaca

SEQ ID NO: 30 - DNA, 51 bp, synthetic construct
  misc_feature 1..51: note = Description of Artificial Sequence: Synthetic primer
  source 1..51: mol_type = other DNA
aattctaatacgactcactatagggagaaggagtaggtcttctgggcttga

SEQ ID NO: 31 - DNA, 51 bp, synthetic construct
  misc_feature 1..51: note = Description of Artificial Sequence: Synthetic primer
  source 1..51: mol_type = other DNA
aattctaatacgactcactatagggagaagggctcaaacgaatggcagtca

SEQ ID NO: 32 - DNA, 24 bp, synthetic construct
  misc_feature 1..24: note = Description of Artificial Sequence: Synthetic primer
  source 1..24: mol_type = other DNA
gcccagctaaagacttgggtatga

SEQ ID NO: 33 - DNA, 20 bp, synthetic construct
  misc_feature 1..20: note = Description of Artificial Sequence: Synthetic primer
  source 1..20: mol_type = other DNA
catccagtgatcctcgttca

SEQ ID NO: 34 - DNA, 22 bp, synthetic construct
  misc_feature 1..22: note = Description of Artificial Sequence: Synthetic primer
  source 1..22: mol_type = other DNA
cctgtcctcggttcacaatcaa

SEQ ID NO: 35 - DNA, 20 bp, synthetic construct
  misc_feature 1..20: note = Description of Artificial Sequence: Synthetic primer
  source 1..20: mol_type = other DNA
aatctctgtggacctctcca

SEQ ID NO: 36 - DNA, 22 bp, synthetic construct
  misc_feature 1..22: note = Description of Artificial Sequence: Synthetic primer
  source 1..22: mol_type = other DNA
gaggccaacaattccgacacta

SEQ ID NO: 37 - DNA, 20 bp, synthetic construct
  misc_feature 1..20: note = Description of Artificial Sequence: Synthetic primer
  source 1..20: mol_type = other DNA
ctgtcctcggttcacaatca

SEQ ID NO: 38 - DNA, 20 bp, synthetic construct
  misc_feature 1..20: note = Description of Artificial Sequence: Synthetic primer
  source 1..20: mol_type = other DNA
ccagaaccttggatcgttca

SEQ ID NO: 39 - DNA, 22 bp, synthetic construct
  misc_feature 1..22: note = Description of Artificial Sequence: Synthetic primer
  source 1..22: mol_type = other DNA
ccatccacaacagggttcttca

SEQ ID NO: 40 - DNA, 24 bp, synthetic construct
  misc_feature 1..24: note = Description of Artificial Sequence: Synthetic primer
  source 1..24: mol_type = other DNA
cagccctgggatcaagtacatgta

SEQ ID NO: 41 - DNA, 22 bp, synthetic construct
  misc_feature 1..22: note = Description of Artificial Sequence: Synthetic primer
  source 1..22: mol_type = other DNA
gcccataccaaacaacactcca

SEQ ID NO: 42 - DNA, 22 bp, synthetic construct
  misc_feature 1..22: note = Description of Artificial Sequence: Synthetic primer
  source 1..22: mol_type = other DNA
gcttagccaggtcactcattga

SEQ ID NO: 43 - DNA, 22 bp, synthetic construct
  misc_feature 1..22: note = Description of Artificial Sequence: Synthetic primer
  source 1..22: mol_type = other DNA
aagttggcgcccatctctgaaa

SEQ ID NO: 44 - DNA, 24 bp, synthetic construct
  misc_feature 1..24: note = Description of Artificial Sequence: Synthetic primer
  source 1..24: mol_type = other DNA
gttctttcctgggccttatctcca

SEQ ID NO: 45 - DNA, 22 bp, synthetic construct
  misc_feature 1..22: note = Description of Artificial Sequence: Synthetic primer
  source 1..22: mol_type = other DNA
ccttcttgactccctagaacca

SEQ ID NO: 46 - DNA, 24 bp, synthetic construct
  misc_feature 1..24: note = Description of Artificial Sequence: Synthetic primer
  source 1..24: mol_type = other DNA
atctctctgtcatgtgtcctggca

SEQ ID NO: 47 - DNA, 22 bp, synthetic construct
  misc_feature 1..22: note = Description of Artificial Sequence: Synthetic primer
  source 1..22: mol_type = other DNA
tctgaacaaactctggcgtcca

SEQ ID NO: 48 - DNA, 24 bp, synthetic construct
  misc_feature 1..24: note = Description of Artificial Sequence: Synthetic primer
  source 1..24: mol_type = other DNA
gcccagctaaagacttgggtatga

SEQ ID NO: 49 - DNA, 20 bp, synthetic construct
  misc_feature 1..20: note = Description of Artificial Sequence: Synthetic primer
  source 1..20: mol_type = other DNA
tgttctctccaaccatccga

SEQ ID NO: 50 - DNA, 20 bp, synthetic construct
  misc_feature 1..20: note = Description of Artificial Sequence: Synthetic primer
  source 1..20: mol_type = other DNA
gctctgcaaccagttagtca

SEQ ID NO: 51 - DNA, 20 bp, synthetic construct
  misc_feature 1..20: note = Description of Artificial Sequence: Synthetic primer
  source 1..20: mol_type = other DNA
atcttcccaggcttgcttga

SEQ ID NO: 52 - DNA, 24 bp, synthetic construct
  misc_feature 1..24: note = Description of Artificial Sequence: Synthetic primer
  source 1..24: mol_type = other DNA
tgccattcgtttgagcctatccca

SEQ ID NO: 53 - DNA, 20 bp, synthetic construct
  misc_feature 1..20: note = Description of Artificial Sequence: Synthetic primer
  source 1..20: mol_type = other DNA
gcagtggttgatctcagaga

SEQ ID NO: 54 - DNA, 22 bp, synthetic construct
  misc_feature 1..22: note = Description of Artificial Sequence: Synthetic primer
  source 1..22: mol_type = other DNA
cgcaggtcaatgtccattgaga

SEQ ID NO: 55 - DNA, 22 bp, synthetic construct
  misc_feature 1..22: note = Description of Artificial Sequence: Synthetic primer
  source 1..22: mol_type = other DNA
ccactcttgtgtgtccttccta

SEQ ID NO: 56 - DNA, 51 bp, synthetic construct; identical to SEQ ID NO: 10
  misc_feature 1..51: note = Description of Artificial Sequence: Synthetic primer
  source 1..51: mol_type = other DNA

SEQ ID NO: 57 - DNA, 50 bp, synthetic construct
  misc_feature 1..50: note = Description of Artificial Sequence: Synthetic primer
  source 1..50: mol_type = other DNA
aattctaatacgactcactatagggagaaggactctgatagtagctatca

SEQ ID NO: 58 - DNA, 22 bp, synthetic construct
  misc_feature 1..22: note = Description of Artificial Sequence: Synthetic primer
  source 1..22: mol_type = other DNA
cctgtcctcggttcacaatcaa

SEQ ID NO: 59 - DNA, 22 bp, synthetic construct
  misc_feature 1..22: note = Description of Artificial Sequence: Synthetic primer
  source 1..22: mol_type = other DNA
ccatccacaacgggattcttca

SEQ ID NO: 60 - DNA, 30 bp, synthetic construct
  misc_feature 1..30: note = Description of Artificial Sequence: Synthetic oligonucleotide
  source 1..30: mol_type = other DNA
tttttccccacagaaccccatctgttgatg

SEQ ID NO: 61 - DNA, 30 bp, synthetic construct
  misc_feature 1..30: note = Description of Artificial Sequence: Synthetic oligonucleotide
  source 1..30: mol_type = other DNA
atgttgagaataatcaatgctaggaaggag

SEQ ID NO: 62 - DNA, 30 bp, synthetic construct
  misc_feature 1..30: note = Description of Artificial Sequence: Synthetic oligonucleotide
  source 1..30: mol_type = other DNA
tagtcagcaggagaccaatgattccgatgc

SEQ ID NO: 63 - DNA, 30 bp, synthetic construct
  misc_feature 1..30: note = Description of Artificial Sequence: Synthetic oligonucleotide
  source 1..30: mol_type = other DNA
cacttccacgtctggtaatctccgctgcca

SEQ ID NO: 64 - DNA, 30 bp, synthetic construct
  misc_feature 1..30: note = Description of Artificial Sequence: Synthetic oligonucleotide
  source 1..30: mol_type = other DNA
ccaagtacatgtagtatgcacttccacgtc

SEQ ID NO: 65 - DNA, 30 bp, synthetic construct
  misc_feature 1..30: note = Description of Artificial Sequence: Synthetic oligonucleotide
  source 1..30: mol_type = other DNA
cgtggaagtgcatactacatgtacttggac

SEQ ID NO: 66 - DNA, 30 bp, synthetic construct
  misc_feature 1..30: note = Description of Artificial Sequence: Synthetic oligonucleotide
  source 1..30: mol_type = other DNA
ggcatttgttaactcccaagttggtagcaa

SEQ ID NO: 67 - DNA, 30 bp, synthetic construct
  misc_feature 1..30: note = Description of Artificial Sequence: Synthetic oligonucleotide
  source 1..30: mol_type = other DNA
cacatgtgcccgagatccatgatctgtaca

SEQ ID NO: 68 - DNA, 30 bp, synthetic construct
  misc_feature 1..30: note = Description of Artificial Sequence: Synthetic oligonucleotide
  source 1..30: mol_type = other DNA
aacaaatgccatgtacagatcatggatctc

SEQ ID NO: 69 - DNA, 30 bp, synthetic construct
  misc_feature 1..30: note = Description of Artificial Sequence: Synthetic oligonucleotide
  source 1..30: mol_type = other DNA
ttcctaaatatccagttttcaaccttgatc

SEQ ID NO: 70 - DNA, 30 bp, synthetic construct
  misc_feature 1..30: note = Description of Artificial Sequence: Synthetic oligonucleotide
  source 1..30: mol_type = other DNA
aatcaagagaatacacgaagcacctgatca

SEQ ID NO: 71 - DNA, 30 bp, synthetic construct
  misc_feature 1..30: note = Description of Artificial Sequence: Synthetic oligonucleotide
  source 1..30: mol_type = other DNA
aaggttgaaaactggatatttaggaacccc

SEQ ID NO: 72 - DNA, 30 bp, synthetic construct
  misc_feature 1..30: note = Description of Artificial Sequence: Synthetic oligonucleotide
  source 1..30: mol_type = other DNA
aggcaatggcaacagctgcgagcgcaaacc

SEQ ID NO: 73 - DNA, 30 bp, synthetic construct
  misc_feature 1..30: note = Description of Artificial Sequence: Synthetic oligonucleotide
  source 1..30: mol_type = other DNA
ctttttggctcgtcgagcttcccaaaagcc

SEQ ID NO: 74 - DNA, 30 bp, synthetic construct
  misc_feature 1..30: note = Description of Artificial Sequence: Synthetic oligonucleotide
  source 1..30: mol_type = other DNA
aacctccatgttccaagacgacatcaaccc

SEQ ID NO: 75 - DNA, 30 bp, synthetic construct
  misc_feature 1..30: note = Description of Artificial Sequence: Synthetic oligonucleotide
  source 1..30: mol_type = other DNA
tgtcaactgttggcttgtcctgtgccataa

SEQ ID NO: 76 - DNA, 30 bp, synthetic construct
  misc_feature 1..30: note = Description of Artificial Sequence: Synthetic oligonucleotide
  source 1..30: mol_type = other DNA
caacagttgacatagagttggtcacgacaa

SEQ ID NO: 77 - DNA, 30 bp, synthetic construct
  misc_feature 1..30: note = Description of Artificial Sequence: Synthetic oligonucleotide
  source 1..30: mol_type = other DNA
ttgatgcctcataacagtaggatcttacct

SEQ ID NO: 78 - DNA, 30 bp, synthetic construct
  misc_feature 1..30: note = Description of Artificial Sequence: Synthetic oligonucleotide
  source 1..30: mol_type = other DNA
tgtccaccaatgttcttttgcacacatatt

SEQ ID NO: 79 - DNA, 30 bp, synthetic construct
  misc_feature 1..30: note = Description of Artificial Sequence: Synthetic oligonucleotide
  source 1..30: mol_type = other DNA
ctgtccaccaatgttcttttgcacacatat

SEQ ID NO: 80 - DNA, 30 bp, synthetic construct
  misc_feature 1..30: note = Description of Artificial Sequence: Synthetic oligonucleotide
  source 1..30: mol_type = other DNA
cagacacccaatatgtgtgcaaaagaacat

SEQ ID NO: 81 - DNA, 30 bp, synthetic construct
  misc_feature 1..30: note = Description of Artificial Sequence: Synthetic oligonucleotide
  source 1..30: mol_type = other DNA
agaacattggtggacagaggttggggaaat

SEQ ID NO: 82 - DNA, 30 bp, synthetic construct
  misc_feature 1..30: note = Description of Artificial Sequence: Synthetic oligonucleotide
  source 1..30: mol_type = other DNA
tcccagtcatcttcttggaacacgtgaact

SEQ ID NO: 83 - DNA, 30 bp, synthetic construct
  misc_feature 1..30: note = Description of Artificial Sequence: Synthetic oligonucleotide
  source 1..30: mol_type = other DNA
atgaaactgacgaaaacagagcgaaagtcg

SEQ ID NO: 84 - DNA, 30 bp, synthetic construct
  misc_feature 1..30: note = Description of Artificial Sequence: Synthetic oligonucleotide
  source 1..30: mol_type = other DNA
taggacttgattgtgaaccaaggacaggcc

SEQ ID NO: 85 - DNA, 30 bp, synthetic construct
  misc_feature 1..30: note = Description of Artificial Sequence: Synthetic oligonucleotide
  source 1..30: mol_type = other DNA
ctttgtgcaccaaccagtgcttgttgttca

SEQ ID NO: 86 - DNA, 30 bp, synthetic construct
  misc_feature 1..30: note = Description of Artificial Sequence: Synthetic oligonucleotide
  source 1..30: mol_type = other DNA
ccggtatctgccccagcatgccaaggcaat

SEQ ID NO: 87 - DNA, 30 bp, synthetic construct
  misc_feature 1..30: note = Description of Artificial Sequence: Synthetic oligonucleotide
  source 1..30: mol_type = other DNA
cagataccggaactccacactggaacaaca

SEQ ID NO: 88 - DNA, 30 bp, synthetic construct
  misc_feature 1..30: note = Description of Artificial Sequence: Synthetic oligonucleotide
  source 1..30: mol_type = other DNA
ataccggaactccacactggaacaacaagg

SEQ ID NO: 89 - DNA, 30 bp, synthetic construct
  misc_feature 1..30: note = Description of Artificial Sequence: Synthetic oligonucleotide
  source 1..30: mol_type = other DNA
ctagaacaacgacggtttgcctcttggcgt

SEQ ID NO: 90 - DNA, 30 bp, synthetic construct
  misc_feature 1..30: note = Description of Artificial Sequence: Synthetic oligonucleotide
  source 1..30: mol_type = other DNA
cctagaacaacgacggtttgcctcttggcg

SEQ ID NO: 91 - DNA, 30 bp, synthetic construct
  misc_feature 1..30: note = Description of Artificial Sequence: Synthetic oligonucleotide
  source 1..30: mol_type = other DNA
cggctccttcctggctccctagaacaacga

SEQ ID NO: 92 - DNA, 30 bp, synthetic construct
  misc_feature 1..30: note = Description of Artificial Sequence: Synthetic oligonucleotide
  source 1..30: mol_type = other DNA
agaggcaaaccgtcgttgttctagggagcc

SEQ ID NO: 93 - DNA, 30 bp, synthetic construct
  misc_feature 1..30: note = Description of Artificial Sequence: Synthetic oligonucleotide
  source 1..30: mol_type = other DNA
ccagcaagagccgtgtgaacggctccttcc

SEQ ID NO: 94 - DNA, 30 bp, synthetic construct
  misc_feature 1..30: note = Description of Artificial Sequence: Synthetic oligonucleotide
  source 1..30: mol_type = other DNA
cctccagagctccagcaagagccgtgtgaa

SEQ ID NO: 95 - DNA, 30 bp, synthetic construct
  misc_feature 1..30: note = Description of Artificial Sequence: Synthetic oligonucleotide
  source 1..30: mol_type = other DNA
tgtccatttttaagcggcatttcaaatggc

SEQ ID NO: 96 - DNA, 30 bp, synthetic construct
  misc_feature 1..30: note = Description of Artificial Sequence: Synthetic oligonucleotide
  source 1..30: mol_type = other DNA
cccttcaatctaagcttgtccatttttaag

SEQ ID NO: 97 - DNA, 30 bp, synthetic construct
  misc_feature 1..30: note = Description of Artificial Sequence: Synthetic oligonucleotide
  source 1..30: mol_type = other DNA
cagctgggaccttggtaaatgtgaacgctg

SEQ ID NO: 98 - DNA, 30 bp, synthetic construct
  misc_feature 1..30: note = Description of Artificial Sequence: Synthetic oligonucleotide
  source 1..30: mol_type = other DNA
ccttgtgcaccgcagcgttcacatttacca

SEQ ID NO: 99 - DNA, 30 bp, synthetic construct
  misc_feature 1..30: note = Description of Artificial Sequence: Synthetic oligonucleotide
  source 1..30: mol_type = other DNA
tgtccaccgccatctgggctgggaccttgc

SEQ ID NO: 100 - DNA, 30 bp, synthetic construct
  misc_feature 1..30: note = Description of Artificial Sequence: Synthetic oligonucleotide
  source 1..30: mol_type = other DNA
agggtctgcatgtccaccgccatctgggct

SEQ ID NO: 101 - DNA, 30 bp, synthetic construct
  misc_feature 1..30: note = Description of Artificial Sequence: Synthetic oligonucleotide
  source 1..30: mol_type = other DNA
gggtcagggtctgcatgtccaccgccatct

SEQ ID NO: 102 - DNA, 30 bp, synthetic construct
  misc_feature 1..30: note = Description of Artificial Sequence: Synthetic oligonucleotide
  source 1..30: mol_type = other DNA
tggcggttatcagcctcccgactggggtca

SEQ ID NO: 103 - DNA, 30 bp, synthetic construct
  misc_feature 1..30: note = Description of Artificial Sequence: Synthetic oligonucleotide
  source 1..30: mol_type = other DNA
ttggcggttatcagcctcccgactggggtc

SEQ ID NO: 104 - DNA, 30 bp, synthetic construct
  misc_feature 1..30: note = Description of Artificial Sequence: Synthetic oligonucleotide
  source 1..30: mol_type = other DNA
aattctcagtgctttcagtaatcacagggt

SEQ ID NO: 105 - DNA, 30 bp, synthetic construct
  misc_feature 1..30: note = Description of Artificial Sequence: Synthetic oligonucleotide
  source 1..30: mol_type = other DNA
cctatgacaatgtaagaatccccaaatggt

SEQ ID NO: 106 - DNA, 30 bp, synthetic construct
  misc_feature 1..30: note = Description of Artificial Sequence: Synthetic oligonucleotide
  source 1..30: mol_type = other DNA
ggagtggtagcaccatcggaaaagcatttg

SEQ ID NO: 107 - DNA, 30 bp, synthetic construct
  misc_feature 1..30: note = Description of Artificial Sequence: Synthetic oligonucleotide
  source 1..30: mol_type = other DNA
gtgagaggtgccaagagaatggcagttctg

SEQ ID NO: 108 - DNA, 30 bp, synthetic construct
  misc_feature 1..30: note = Description of Artificial Sequence: Synthetic oligonucleotide
  source 1..30: mol_type = other DNA
tgagaggtgccaagagaatggcagttctgg

SEQ ID NO: 109 - DNA, 30 bp, synthetic construct
  misc_feature 1..30: note = Description of Artificial Sequence: Synthetic oligonucleotide
  source 1..30: mol_type = other DNA
ggggatacagcctgggacttcggatcagtc

SEQ ID NO: 110 - DNA, 30 bp, synthetic construct
  misc_feature 1..30: note = Description of Artificial Sequence: Synthetic oligonucleotide
  source 1..30: mol_type = other DNA
gggatacagcctgggacttcggatcagtcg

SEQ ID NO: 111 - DNA, 30 bp, synthetic construct
  misc_feature 1..30: note = Description of Artificial Sequence: Synthetic oligonucleotide
  source 1..30: mol_type = other DNA
aacagtgatttgaaagctgctccaaaaatc

SEQ ID NO: 112 - DNA, 30 bp, synthetic construct
  misc_feature 1..30: note = Description of Artificial Sequence: Synthetic oligonucleotide
  source 1..30: mol_type = other DNA
aaacccaaccacaccagcagcgtgcctatg

SEQ ID NO: 113 - DNA, 30 bp, synthetic construct
  misc_feature 1..30: note = Description of Artificial Sequence: Synthetic oligonucleotide
  source 1..30: mol_type = other DNA
ggttctcacagatcctcataggcacgctgc

SEQ ID NO: 114 - DNA, 30 bp, synthetic construct
  misc_feature 1..30: note = Description of Artificial Sequence: Synthetic oligonucleotide
  source 1..30: mol_type = other DNA
ctccccccagggccaagcatgtgagggaga

SEQ ID NO: 115 - DNA, 30 bp, synthetic construct
  misc_feature 1..30: note = Description of Artificial Sequence: Synthetic oligonucleotide
  source 1..30: mol_type = other DNA
atggatccatctccctcacatgcttggccc

SEQ ID NO: 116 - DNA, 30 bp, synthetic construct
  misc_feature 1..30: note = Description of Artificial Sequence: Synthetic oligonucleotide
  source 1..30: mol_type = other DNA
caccccacgtcagcagagacagccgtggag

SEQ ID NO: 117 - DNA, 30 bp, synthetic construct
  misc_feature 1..30: note = Description of Artificial Sequence: Synthetic oligonucleotide
  source 1..30: mol_type = other DNA
ccctggggggagtgatgatcttcctctcca

SEQ ID NO: 118 - DNA, 30 bp, synthetic construct
  misc_feature 1..30: note = Description of Artificial Sequence: Synthetic oligonucleotide
  source 1..30: mol_type = other DNA
acttctcaaaaagagaaacgagatgtggca

SEQ ID NO: 119 - DNA, 30 bp, synthetic construct
  misc_feature 1..30: note = Description of Artificial Sequence: Synthetic oligonucleotide
  source 1..30: mol_type = other DNA
ccccccgcagattggcagcagcagtcaagc

SEQ ID NO: 120 - DNA, 30 bp, synthetic construct
  misc_feature 1..30: note = Description of Artificial Sequence: Synthetic oligonucleotide
  source 1..30: mol_type = other DNA
tggcagcagcagtcaagcaggcttgggaag

SEQ ID NO: 121 - DNA, 30 bp, synthetic construct
  misc_feature 1..30: note = Description of Artificial Sequence: Synthetic oligonucleotide
  source 1..30: mol_type = other DNA
gcagcagcagtcaagcaggcttgggaagag

SEQ ID NO: 122 - DNA, 30 bp, synthetic construct
  misc_feature 1..30: note = Description of Artificial Sequence: Synthetic oligonucleotide
  source 1..30: mol_type = other DNA
atttccacatgatgttttccattcttgaaa

SEQ ID NO: 123 - DNA, 30 bp, synthetic construct
  misc_feature 1..30: note = Description of Artificial Sequence: Synthetic oligonucleotide
  source 1..30: mol_type = other DNA
caagaatggaaaacatcatgtggaaatcag

SEQ ID NO: 124 - DNA, 30 bp, synthetic construct
  misc_feature 1..30: note = Description of Artificial Sequence: Synthetic oligonucleotide
  source 1..30: mol_type = other DNA
agctcattcacaggcactggcaatctttgt

SEQ ID NO: 125 - DNA, 30 bp, synthetic construct
  misc_feature 1..30: note = Description of Artificial Sequence: Synthetic oligonucleotide
  source 1..30: mol_type = other DNA
aagcctgggggaaatcatattttgtcagag

SEQ ID NO: 126 - DNA, 30 bp, synthetic construct
  misc_feature 1..30: note = Description of Artificial Sequence: Synthetic oligonucleotide
  source 1..30: mol_type = other DNA
gtgtgtcaccatcgacaacaaaactgttgt

SEQ ID NO: 127 - DNA, 30 bp, synthetic construct
  misc_feature 1..30: note = Description of Artificial Sequence: Synthetic oligonucleotide
  source 1..30: mol_type = other DNA
tcttctctaaccttgagccaaacactggtg

SEQ ID NO: 128 - DNA, 30 bp, synthetic construct
  misc_feature 1..30: note = Description of Artificial Sequence: Synthetic oligonucleotide
  source 1..30: mol_type = other DNA
agtagtcttctctaaccttgagccaaacac

SEQ ID NO: 129 - DNA, 30 bp, synthetic construct
  misc_feature 1..30: note = Description of Artificial Sequence: Synthetic oligonucleotide
  source 1..30: mol_type = other DNA
tttcccttaacagctgttcctatgacggct

SEQ ID NO: 130 - DNA, 30 bp, synthetic construct
  misc_feature 1..30: note = Description of Artificial Sequence: Synthetic oligonucleotide
  source 1..30: mol_type = other DNA
tttcaatccagtagcccaggtcactgtggg

SEQ ID NO: 131 - DNA, 30 bp, synthetic construct
  misc_feature 1..30: note = Description of Artificial Sequence: Synthetic oligonucleotide
  source 1..30: mol_type = other DNA
cactttcaatccagtagcccaggtcactgt

SEQ ID NO: 132 - DNA, 30 bp, synthetic construct
  misc_feature 1..30: note = Description of Artificial Sequence: Synthetic oligonucleotide
  source 1..30: mol_type = other DNA
tcactttcaatccagtagcccaggtcactg

SEQ ID NO: 133 - DNA, 30 bp, synthetic construct
  misc_feature 1..30: note = Description of Artificial Sequence: Synthetic oligonucleotide
  source 1..30: mol_type = other DNA
ttaagggaaaggaggccgcccacagtgacc

SEQ ID NO: 134 - DNA, 30 bp, synthetic construct
  misc_feature 1..30: note = Description of Artificial Sequence: Synthetic oligonucleotide
  source 1..30: mol_type = other DNA
tcattcttttcactttcaatccagtagccc

SEQ ID NO: 135 - DNA, 30 bp, synthetic construct
  misc_feature 1..30: note = Description of Artificial Sequence: Synthetic oligonucleotide
  source 1..30: mol_type = other DNA
gcttgagttctgtaaccctctctagtgttg

SEQ ID NO: 136 - DNA, 30 bp, synthetic construct
  misc_feature 1..30: note = Description of Artificial Sequence: Synthetic oligonucleotide
  source 1..30: mol_type = other DNA
tgaaccttggtgcctggacattcctcaaac

SEQ ID NO: 137 - DNA, 30 bp, synthetic construct
  misc_feature 1..30: note = Description of Artificial Sequence: Synthetic oligonucleotide
  source 1..30: mol_type = other DNA
tagttccgcatgtctcctccacgtgaacct

SEQ ID NO: 138 - DNA, 30 bp, synthetic construct
  misc_feature 1..30: note = Description of Artificial Sequence: Synthetic oligonucleotide
  source 1..30: mol_type = other DNA
agagcaacttagtgaggtcaatggtgacag

SEQ ID NO: 139 - DNA, 30 bp, synthetic construct
  misc_feature 1..30: note = Description of Artificial Sequence: Synthetic oligonucleotide
  source 1..30: mol_type = other DNA
gcactccaagagagaagtggtccatgtgat

SEQ ID NO: 140 - DNA, 30 bp, synthetic construct
  misc_feature 1..30: note = Description of Artificial Sequence: Synthetic oligonucleotide
  source 1..30: mol_type = other DNA
tcattatgagcacatcaatggcagtgctgg

SEQ ID NO: 141 - DNA, 30 bp, synthetic construct
  misc_feature 1..30: note = Description of Artificial Sequence: Synthetic oligonucleotide
  source 1..30: mol_type = other DNA
agtcactcattaaaaatcctcccaggatca

SEQ ID NO: 142 - DNA, 30 bp, synthetic construct
  misc_feature 1..30: note = Description of Artificial Sequence: Synthetic oligonucleotide
  source 1..30: mol_type = other DNA
ccttcgcagaaatgaacactggaggagatg

SEQ ID NO: 143 - DNA, 30 bp, synthetic construct
  misc_feature 1..30: note = Description of Artificial Sequence: Synthetic oligonucleotide
  source 1..30: mol_type = other DNA
acactggaggagatgtggctcacttggcat

SEQ ID NO: 144 - DNA, 30 bp, synthetic construct
  misc_feature 1..30: note = Description of Artificial Sequence: Synthetic oligonucleotide
  source 1..30: mol_type = other DNA
aattagctctgaaaataaaggagaccagca

SEQ ID NO: 145 - DNA, 30 bp, synthetic construct
  misc_feature 1..30: note = Description of Artificial Sequence: Synthetic oligonucleotide
  source 1..30: mol_type = other DNA
cacgaggtgtccaattagctctgaaaataa

SEQ ID NO: 146 - DNA, 30 bp, synthetic construct
  misc_feature 1..30: note = Description of Artificial Sequence: Synthetic oligonucleotide
  source 1..30: mol_type = other DNA
gagattgcagtttgcaaaagacacgaagcc

SEQ ID NO: 147 - DNA, 30 bp, synthetic construct
  misc_feature 1..30: note = Description of Artificial Sequence: Synthetic oligonucleotide
  source 1..30: mol_type = other DNA
gccaaccaggccaaagcaaatccattgacg

SEQ ID NO: 148 - DNA, 30 bp, synthetic construct
  misc_feature 1..30: note = Description of Artificial Sequence: Synthetic oligonucleotide
  source 1..30: mol_type = other DNA
ttgccagagcaatgttgtcagtgcgtggca

SEQ ID NO: 149 - DNA, 30 bp, synthetic construct
  misc_feature 1..30: note = Description of Artificial Sequence: Synthetic oligonucleotide
  source 1..30: mol_type = other DNA
cccgaggtacactgctcgtggcatggagag

SEQ ID NO: 150 - DNA, 30 bp, synthetic construct
  misc_feature 1..30: note = Description of Artificial Sequence: Synthetic oligonucleotide
  source 1..30: mol_type = other DNA
ggttcttcttcacactacctttccctttca

SEQ ID NO: 151 - DNA, 30 bp, synthetic construct
  misc_feature 1..30: note = Description of Artificial Sequence: Synthetic oligonucleotide
  source 1..30: mol_type = other DNA
tgtggagggtttatgctcctctccctgaaa

SEQ ID NO: 152 - DNA, 30 bp, synthetic construct
  misc_feature 1..30: note = Description of Artificial Sequence: Synthetic oligonucleotide
  source 1..30: mol_type = other DNA
gcagtcaatcccaaggccatgacaaatggc

SEQ ID NO: 153 - DNA, 30 bp, synthetic construct
  misc_feature 1..30: note = Description of Artificial Sequence: Synthetic oligonucleotide
  source 1..30: mol_type = other DNA
ttgaaagagcaggtgacatcacatgggaaa

SEQ ID NO: 154 - DNA, 30 bp, synthetic construct
  misc_feature 1..30: note = Description of Artificial Sequence: Synthetic oligonucleotide
  source 1..30: mol_type = other DNA
tggtctctccttttttcacttctttgggag

SEQ ID NO: 155 - DNA, 30 bp, synthetic construct
  misc_feature 1..30: note = Description of Artificial Sequence: Synthetic oligonucleotide
  source 1..30: mol_type = other DNA
aaagaagtgaaaaaaggagagaccacagat

SEQ ID NO: 156 - DNA, 30 bp, synthetic construct
  misc_feature 1..30: note = Description of Artificial Sequence: Synthetic oligonucleotide
  source 1..30: mol_type = other DNA
aagaagtgaaaaaaggagagaccacagatg

SEQ ID NO: 157 - DNA, 30 bp, synthetic construct
  misc_feature 1..30: note = Description of Artificial Sequence: Synthetic oligonucleotide
  source 1..30: mol_type = other DNA
ctccaacctgtgttgaacccagcagtctgc

SEQ ID NO: 158 - DNA, 30 bp, synthetic construct
  misc_feature 1..30: note = Description of Artificial Sequence: Synthetic oligonucleotide
  source 1..30: mol_type = other DNA
actccaacctgtgttgaacccagcagtctg

SEQ ID NO: 159 - DNA, 30 bp, synthetic construct
  misc_feature 1..30: note = Description of Artificial Sequence: Synthetic oligonucleotide
  source 1..30: mol_type = other DNA
gggtatacagagtgatgacccgcagactgc

SEQ ID NO: 160 - DNA, 30 bp, synthetic construct
  misc_feature 1..30: note = Description of Artificial Sequence: Synthetic oligonucleotide
  source 1..30: mol_type = other DNA
tgatgacccgcagactgctgggttcaacac

SEQ ID NO: 161 - DNA, 30 bp, synthetic construct
  misc_feature 1..30: note = Description of Artificial Sequence: Synthetic oligonucleotide
  source 1..30: mol_type = other DNA
gcagctccttttgtgacgtgccacatggtg

SEQ ID NO: 162 - DNA, 30 bp, synthetic construct
  misc_feature 1..30: note = Description of Artificial Sequence: Synthetic oligonucleotide
  source 1..30: mol_type = other DNA
atgtggcacgtcacaaaaggagctgcactg

SEQ ID NO: 163 - DNA, 30 bp, synthetic construct
  misc_feature 1..30: note = Description of Artificial Sequence: Synthetic oligonucleotide
  source 1..30: mol_type = other DNA
agcggtgaagggagacttgatccatactgg

SEQ ID NO: 164 - DNA, 30 bp, synthetic construct
  misc_feature 1..30: note = Description of Artificial Sequence: Synthetic oligonucleotide
  source 1..30: mol_type = other DNA
gcggtgaagggagacttgatccatactggg

SEQ ID NO: 165 - DNA, 30 bp, synthetic construct
  misc_feature 1..30: note = Description of Artificial Sequence: Synthetic oligonucleotide
  source 1..30: mol_type = other DNA
gatgtcaagcaggacttagtgtcatactgt

SEQ ID NO: 166 - DNA, 30 bp, synthetic construct
  misc_feature 1..30: note = Description of Artificial Sequence: Synthetic oligonucleotide
  source 1..30: mol_type = other DNA
acttagtgtcatactgtgggccttggaagt

SEQ ID NO: 167 - DNA, 30 bp, synthetic construct
  misc_feature 1..30: note = Description of Artificial Sequence: Synthetic oligonucleotide
  source 1..30: mol_type = other DNA
gtgcagctcttggcagtaccccccggagag

SEQ ID NO: 168 - DNA, 30 bp, synthetic construct
  misc_feature 1..30: note = Description of Artificial Sequence: Synthetic oligonucleotide
  source 1..30: mol_type = other DNA
tgcagctcttggcagtaccccccggagaga

SEQ ID NO: 169 - DNA, 30 bp, synthetic construct
  misc_feature 1..30: note = Description of Artificial Sequence: Synthetic oligonucleotide
  source 1..30: mol_type = other DNA
tgaatattccaggcagagtctgaatgtttc

SEQ ID NO: 170 - DNA, 30 bp, synthetic construct
  misc_feature 1..30: note = Description of Artificial Sequence: Synthetic oligonucleotide
  source 1..30: mol_type = other DNA
tgtctaggatcggggatcctgaagttcctg

SEQ ID NO: 171 - DNA, 30 bp, synthetic construct
  misc_feature 1..30: note = Description of Artificial Sequence: Synthetic oligonucleotide
  source 1..30: mol_type = other DNA
ctatcactcttccgcatttgtctaggatcg

SEQ ID NO: 172 - DNA, 30 bp, synthetic construct
  misc_feature 1..30: note = Description of Artificial Sequence: Synthetic oligonucleotide
  source 1..30: mol_type = other DNA
cctatcactcttccgcatttgtctaggatc

SEQ ID NO: 173 - DNA, 30 bp, synthetic construct
  misc_feature 1..30: note = Description of Artificial Sequence: Synthetic oligonucleotide
  source 1..30: mol_type = other DNA
agaatggaagctatgttagtgccataaccc

SEQ ID NO: 174 - DNA, 30 bp, synthetic construct
  misc_feature 1..30: note = Description of Artificial Sequence: Synthetic oligonucleotide
  source 1..30: mol_type = other DNA
ttagtgccataacccagggaaagagggagg

SEQ ID NO: 175 - DNA, 30 bp, synthetic construct
  misc_feature 1..30: note = Description of Artificial Sequence: Synthetic oligonucleotide
  source 1..30: mol_type = other DNA
taacccagggaaagagggaggaggagactc

SEQ ID NO: 176 - DNA, 30 bp, synthetic construct
  misc_feature 1..30: note = Description of Artificial Sequence: Synthetic oligonucleotide
  source 1..30: mol_type = other DNA
ggacagttagctgcttcttcttcagcatcg

SEQ ID NO: 177 - DNA, 30 bp, synthetic construct
  misc_feature 1..30: note = Description of Artificial Sequence: Synthetic oligonucleotide
  source 1..30: mol_type = other DNA
cgatgctgaagaagaagcagctaactgtcc

SEQ ID NO: 178 - DNA, 30 bp, synthetic construct
  misc_feature 1..30: note = Description of Artificial Sequence: Synthetic oligonucleotide
  source 1..30: mol_type = other DNA
ctagtctttccggctcctggatgcagatcc

SEQ ID NO: 179 - DNA, 30 bp, synthetic construct
  misc_feature 1..30: note = Description of Artificial Sequence: Synthetic oligonucleotide
  source 1..30: mol_type = other DNA
ctatttcaggaagaactctcctagtctttc

SEQ ID NO: 180 - DNA, 30 bp, synthetic construct
  misc_feature 1..30: note = Description of Artificial Sequence: Synthetic oligonucleotide
  source 1..30: mol_type = other DNA
ccataaaaaagagactccgcacagtgattt

SEQ ID NO: 181 - DNA, 30 bp, synthetic construct
  misc_feature 1..30: note = Description of Artificial Sequence: Synthetic oligonucleotide
  source 1..30: mol_type = other DNA
ctgagatggaggaagccttgagaggacttc

SEQ ID NO: 182 - DNA, 30 bp, synthetic construct
  misc_feature 1..30: note = Description of Artificial Sequence: Synthetic oligonucleotide
  source 1..30: mol_type = other DNA
atgacaacagcagttaacgtcacccactct

SEQ ID NO: 183 - DNA, 30 bp, synthetic construct
  misc_feature 1..30: note = Description of Artificial Sequence: Synthetic oligonucleotide
  source 1..30: mol_type = other DNA
tagggactctgatgggttgtaataggcgtg

SEQ ID NO: 184 - DNA, 30 bp, synthetic construct
  misc_feature 1..30: note = Description of Artificial Sequence: Synthetic oligonucleotide
  source 1..30: mol_type = other DNA
ttgtaattagggactctgatgggttgtaat

SEQ ID NO: 185 - DNA, 30 bp, synthetic construct
  misc_feature 1..30: note = Description of Artificial Sequence: Synthetic oligonucleotide
  source 1..30: mol_type = other DNA
tgatgtagagattgtaattagggactctga

SEQ ID NO: 186 - DNA, 30 bp, synthetic construct
  misc_feature 1..30: note = Description of Artificial Sequence: Synthetic oligonucleotide
  source 1..30: mol_type = other DNA
gatatgtatcctcttgcagctatacttgag

SEQ ID NO: 187 - DNA, 30 bp, synthetic construct
  misc_feature 1..30: note = Description of Artificial Sequence: Synthetic oligonucleotide
  source 1..30: mol_type = other DNA
cttggaacgaaccaaactgttttcccagaa

SEQ ID NO: 188 - DNA, 30 bp, synthetic construct
  misc_feature 1..30: note = Description of Artificial Sequence: Synthetic oligonucleotide
  source 1..30: mol_type = other DNA
tcaggctttgattgggtgacagaccattct

SEQ ID NO: 189 - DNA, 30 bp, synthetic construct
  misc_feature 1..30: note = Description of Artificial Sequence: Synthetic oligonucleotide
  source 1..30: mol_type = other DNA
gtatgacccgctttccagcctttgtcagac

SEQ ID NO: 190 - DNA, 30 bp, synthetic construct
  misc_feature 1..30: note = Description of Artificial Sequence: Synthetic oligonucleotide
  source 1..30: mol_type = other DNA
aaggctggaaagcgggtcatacaactcagc

SEQ ID NO: 191 - DNA, 30 bp, synthetic construct
  misc_feature 1..30: note = Description of Artificial Sequence: Synthetic oligonucleotide
  source 1..30: mol_type = other DNA
tcagagatgggcgcgaatttcaaagctgac

SEQ ID NO: 192 - DNA, 30 bp, synthetic construct
  misc_feature 1..30: note = Description of Artificial Sequence: Synthetic oligonucleotide
  source 1..30: mol_type = other DNA
cagagatgggcgcgaatttcaaagctgacc

SEQ ID NO: 193 - DNA, 30 bp, synthetic construct
  misc_feature 1..30: note = Description of Artificial Sequence: Synthetic oligonucleotide
  source 1..30: mol_type = other DNA
atacttgatggtgagagagtcatcttggct

SEQ ID NO: 194 - DNA, 30 bp, synthetic construct
  misc_feature 1..30: note = Description of Artificial Sequence: Synthetic oligonucleotide
  source 1..30: mol_type = other DNA
ccatacatgtactcatctccaggtttgtta

SEQ ID NO: 195 - DNA, 30 bp, synthetic construct
  misc_feature 1..30: note = Description of Artificial Sequence: Synthetic oligonucleotide
  source 1..30: mol_type = other DNA
ttgcttcaagccagtgtgcatggtcttcat

SEQ ID NO: 196 - DNA, 30 bp, synthetic construct
  misc_feature 1..30: note = Description of Artificial Sequence: Synthetic oligonucleotide
  source 1..30: mol_type = other DNA
ctccaggatggcctcatagcctcgctctac

SEQ ID NO: 197 - DNA, 30 bp, synthetic construct
  misc_feature 1..30: note = Description of Artificial Sequence: Synthetic oligonucleotide
  source 1..30: mol_type = other DNA
tcaatggcagctaccttatcggcctcaggc

SEQ ID NO: 198 - DNA, 30 bp, synthetic construct
  misc_feature 1..30: note = Description of Artificial Sequence: Synthetic oligonucleotide
  source 1..30: mol_type = other DNA
tagcctcgctctaccggcctgaggccgata

SEQ ID NO: 199 - DNA, 30 bp, synthetic construct
  misc_feature 1..30: note = Description of Artificial Sequence: Synthetic oligonucleotide
  source 1..30: mol_type = other DNA
gatgcaacctgataggctagccaaacggga

SEQ ID NO: 200 - DNA, 30 bp, synthetic construct
  misc_feature 1..30: note = Description of Artificial Sequence: Synthetic oligonucleotide
  source 1..30: mol_type = other DNA
agagtgctcaaaccaagatggatggatgcg

SEQ ID NO: 201 - DNA, 30 bp, synthetic construct
  misc_feature 1..30: note = Description of Artificial Sequence: Synthetic oligonucleotide
  source 1..30: mol_type = other DNA
gagtgctcaaaccaagatggatggatgcga

SEQ ID NO: 202 - DNA, 30 bp, synthetic construct
  misc_feature 1..30: note = Description of Artificial Sequence: Synthetic oligonucleotide
  source 1..30: mol_type = other DNA
attctttgaacgacttcagggcagcatgat

SEQ ID NO: 203 - DNA, 30 bp, synthetic construct
  misc_feature 1..30: note = Description of Artificial Sequence: Synthetic oligonucleotide
  source 1..30: mol_type = other DNA
ccattactcctaaagccactcctcttttcc

SEQ ID NO: 204 - DNA, 30 bp, synthetic construct
  misc_feature 1..30: note = Description of Artificial Sequence: Synthetic oligonucleotide
  source 1..30: mol_type = other DNA
ggaaaagaggagtggctttaggagtaatgg

SEQ ID NO: 205 - DNA, 30 bp, synthetic construct
  misc_feature 1..30: note = Description of Artificial Sequence: Synthetic oligonucleotide
  source 1..30: mol_type = other DNA
ctgggaacattgccaggacacatgacagag

SEQ ID NO: 206 - DNA, 30 bp, synthetic construct
  misc_feature 1..30: note = Description of Artificial Sequence: Synthetic oligonucleotide
  source 1..30: mol_type = other DNA
tgcgagcagagactggaagcaggccttaca

SEQ ID NO: 207 - DNA, 30 bp, synthetic construct
  misc_feature 1..30: note = Description of Artificial Sequence: Synthetic oligonucleotide
  source 1..30: mol_type = other DNA
ctcttaggcttgttgggaacagtttcgttg

SEQ ID NO: 208 - DNA, 30 bp, synthetic construct
  misc_feature 1..30: note = Description of Artificial Sequence: Synthetic oligonucleotide
  source 1..30: mol_type = other DNA
tgtcctggggagatctttgcttctctggct

SEQ ID NO: 209 - DNA, 30 bp, synthetic construct
  misc_feature 1..30: note = Description of Artificial Sequence: Synthetic oligonucleotide
  source 1..30: mol_type = other DNA
aggacaaccagatggcaatcatcatcatgg

SEQ ID NO: 210 - DNA, 30 bp, synthetic construct
  misc_feature 1..30: note = Description of Artificial Sequence: Synthetic oligonucleotide
  source 1..30: mol_type = other DNA
accagatggcaatcatcatcatggtggcag

SEQ ID NO: 211 - DNA, 30 bp, synthetic construct
  misc_feature 1..30: note = Description of Artificial Sequence: Synthetic oligonucleotide
  source 1..30: mol_type = other DNA
taatgggaaggagagaagaaggagtaacta

SEQ ID NO: 212 - DNA, 30 bp, synthetic construct
  misc_feature 1..30: note = Description of Artificial Sequence: Synthetic oligonucleotide
  source 1..30: mol_type = other DNA
tgagagttgtcagtgcggcatagatagccc

SEQ ID NO: 213 - DNA, 30 bp, synthetic construct
  misc_feature 1..30: note = Description of Artificial Sequence: Synthetic oligonucleotide
  source 1..30: mol_type = other DNA
ggacggctggggtgatgagagttgtcagtg

SEQ ID NO: 214 - DNA, 30 bp, synthetic construct
  misc_feature 1..30: note = Description of Artificial Sequence: Synthetic oligonucleotide
  source 1..30: mol_type = other DNA
caactctcatcaccccagccgtccaacacg

SEQ ID NO: 215 - DNA, 29 bp, synthetic construct
  misc_feature 1..29: note = Description of Artificial Sequence: Synthetic oligonucleotide
  source 1..29: mol_type = other DNA
atgggactttggagtcccgctgctaatga

SEQ ID NO: 216 - DNA, 30 bp, synthetic construct
  misc_feature 1..30: note = Description of Artificial Sequence: Synthetic oligonucleotide
  source 1..30: mol_type = other DNA
caatgtcagttaccactattccatccacaa

SEQ ID NO: 217 - DNA, 30 bp, synthetic construct
  misc_feature 1..30: note = Description of Artificial Sequence: Synthetic oligonucleotide
  source 1..30: mol_type = other DNA
atttgtttggagaaccttcccacaaggtgg

SEQ ID NO: 218 - DNA, 30 bp, synthetic construct
  misc_feature 1..30: note = Description of Artificial Sequence: Synthetic oligonucleotide
  source 1..30: mol_type = other DNA
ggtaacttcctctgaagatgttgcacagtg

SEQ ID NO: 219 - DNA, 30 bp, synthetic construct
  misc_feature 1..30: note = Description of Artificial Sequence: Synthetic oligonucleotide
  source 1..30: mol_type = other DNA
gtcactgtataaataagagaagcgcctgcc

SEQ ID NO: 220 - DNA, 30 bp, synthetic construct
  misc_feature 1..30: note = Description of Artificial Sequence: Synthetic oligonucleotide
  source 1..30: mol_type = other DNA
tctcccgttccacctccacgtctcttgact

SEQ ID NO: 221 - DNA, 30 bp, synthetic construct
  misc_feature 1..30: note = Description of Artificial Sequence: Synthetic oligonucleotide
  source 1..30: mol_type = other DNA
ctggcctagtcaagagacgtggaggtggaa

SEQ ID NO: 222 - DNA, 30 bp, synthetic construct
  misc_feature 1..30: note = Description of Artificial Sequence: Synthetic oligonucleotide
  source 1..30: mol_type = other DNA
gagtagaactccagggccgacatctgattc

SEQ ID NO: 223 - DNA, 30 bp, synthetic construct
  misc_feature 1..30: note = Description of Artificial Sequence: Synthetic oligonucleotide
  source 1..30: mol_type = other DNA
aaaagtcaggcatcactgaagtgtgtagag

SEQ ID NO: 224 - DNA, 30 bp, synthetic construct
  misc_feature 1..30: note = Description of Artificial Sequence: Synthetic oligonucleotide
  source 1..30: mol_type = other DNA
cgcttccccgggatacagcatgtcctcctg

SEQ ID NO: 225 - DNA, 30 bp, synthetic construct
  misc_feature 1..30: note = Description of Artificial Sequence: Synthetic oligonucleotide
  source 1..30: mol_type = other DNA
ggagtggccacaggaggacatgctgtatcc

SEQ ID NO: 226 - DNA, 30 bp, synthetic construct
  misc_feature 1..30: note = Description of Artificial Sequence: Synthetic oligonucleotide
  source 1..30: mol_type = other DNA
gagtggccacaggaggacatgctgtatccc

SEQ ID NO: 227 - DNA, 30 bp, synthetic construct
  misc_feature 1..30: note = Description of Artificial Sequence: Synthetic oligonucleotide
  source 1..30: mol_type = other DNA
ctgccacatccgaggtcaacaacctttcca

SEQ ID NO: 228 - DNA, 30 bp, synthetic construct
  misc_feature 1..30: note = Description of Artificial Sequence: Synthetic oligonucleotide
  source 1..30: mol_type = other DNA
taatagctccaaccccctctgccacatccg

SEQ ID NO: 229 - DNA, 30 bp, synthetic construct
  misc_feature 1..30: note = Description of Artificial Sequence: Synthetic oligonucleotide
  source 1..30: mol_type = other DNA
gctattatgccgccaccatccggaaagtgc

SEQ ID NO: 230 - DNA, 30 bp, synthetic construct
  misc_feature 1..30: note = Description of Artificial Sequence: Synthetic oligonucleotide
  source 1..30: mol_type = other DNA
attatgccgccaccatccggaaagtgcagg

SEQ ID NO: 231 - DNA, 30 bp, synthetic construct
  misc_feature 1..30: note = Description of Artificial Sequence: Synthetic oligonucleotide
  source 1..30: mol_type = other DNA
ggaaagtgcaggaggtgaaaggatacacaa

SEQ ID NO: 232 - DNA, 30 bp, synthetic construct
  misc_feature 1..30: note = Description of Artificial Sequence: Synthetic oligonucleotide
  source 1..30: mol_type = other DNA
agctttgcaccagcatgggttcttcatgac

SEQ ID NO: 233 - DNA, 30 bp, synthetic construct
  misc_feature 1..30: note = Description of Artificial Sequence: Synthetic oligonucleotide
  source 1..30: mol_type = other DNA
ctatgtcacacagcaaggtatcacacggct

SEQ ID NO: 234 - DNA, 30 bp, synthetic construct
  misc_feature 1..30: note = Description of Artificial Sequence: Synthetic oligonucleotide
  source 1..30: mol_type = other DNA
tagatgatgactcacctatgtcacacagca

SEQ ID NO: 235 - DNA, 30 bp, synthetic construct
  misc_feature 1..30: note = Description of Artificial Sequence: Synthetic oligonucleotide
  source 1..30: mol_type = other DNA
tcaagccagtcccccaccatagagagcact

SEQ ID NO: 236 - DNA, 30 bp, synthetic construct
  misc_feature 1..30: note = Description of Artificial Sequence: Synthetic oligonucleotide
  source 1..30: mol_type = other DNA
ctatggtgggggactggcttgagaaaagac

SEQ ID NO: 237 - DNA, 30 bp, synthetic construct
  misc_feature 1..30: note = Description of Artificial Sequence: Synthetic oligonucleotide
  source 1..30: mol_type = other DNA
atggtgggggactggcttgagaaaagaccg

SEQ ID NO: 238 - DNA, 30 bp, synthetic construct
  misc_feature 1..30: note = Description of Artificial Sequence: Synthetic oligonucleotide
  source 1..30: mol_type = other DNA
tggtgggggactggcttgagaaaagaccgg

SEQ ID NO: 239 - DNA, 30 bp, synthetic construct
  misc_feature 1..30: note = Description of Artificial Sequence: Synthetic oligonucleotide
  source 1..30: mol_type = other DNA
ttgagaaaagaccgggggccttctgtataa

SEQ ID NO: 240 - DNA, 30 bp, synthetic construct
  misc_feature 1..30: note = Description of Artificial Sequence: Synthetic oligonucleotide
  source 1..30: mol_type = other DNA
ctgactaatcctcccccatacctacgttgc

SEQ ID NO: 241 - DNA, 30 bp, synthetic construct
  misc_feature 1..30: note = Description of Artificial Sequence: Synthetic oligonucleotide
  source 1..30: mol_type = other DNA
ttgctccagagacccaatacatctcatgtg

SEQ ID NO: 242 - DNA, 30 bp, synthetic construct
  misc_feature 1..30: note = Description of Artificial Sequence: Synthetic oligonucleotide
  source 1..30: mol_type = other DNA
actggcctcctgggaccatccatgcgtccc

SEQ ID NO: 243 - DNA, 30 bp, synthetic construct
  misc_feature 1..30: note = Description of Artificial Sequence: Synthetic oligonucleotide
  source 1..30: mol_type = other DNA
cagcttgccacagctcgtgtgcccgagccg

SEQ ID NO: 244 - DNA, 30 bp, synthetic construct
  misc_feature 1..30: note = Description of Artificial Sequence: Synthetic oligonucleotide
  source 1..30: mol_type = other DNA
tgaaatatgaggaagatgtgaacctcggct

SEQ ID NO: 245 - DNA, 30 bp, synthetic construct
  misc_feature 1..30: note = Description of Artificial Sequence: Synthetic oligonucleotide
  source 1..30: mol_type = other DNA
tggcaagctgtgctgaagctcccaacatga

SEQ ID NO: 246 - DNA, 30 bp, synthetic construct
  misc_feature 1..30: note = Description of Artificial Sequence: Synthetic oligonucleotide
  source 1..30: mol_type = other DNA
cttgtgtgggggcttcgtagctcccatggt

SEQ ID NO: 247 - DNA, 30 bp, synthetic construct
  misc_feature 1..30: note = Description of Artificial Sequence: Synthetic oligonucleotide
  source 1..30: mol_type = other DNA
aaccatccatacaggacatgggcctaccat

SEQ ID NO: 248 - DNA, 30 bp, synthetic construct
  misc_feature 1..30: note = Description of Artificial Sequence: Synthetic oligonucleotide
  source 1..30: mol_type = other DNA
ttgacaggagtctaacaaccccattcacga

SEQ ID NO: 249 - DNA, 30 bp, synthetic construct
  misc_feature 1..30: note = Description of Artificial Sequence: Synthetic oligonucleotide
  source 1..30: mol_type = other DNA
tttgacaggagtctaacaaccccattcacg

SEQ ID NO: 250 - DNA, 30 bp, synthetic construct
  misc_feature 1..30: note = Description of Artificial Sequence: Synthetic oligonucleotide
  source 1..30: mol_type = other DNA
gtgtccactttttctttgaagactctttgt

SEQ ID NO: 251 - DNA, 30 bp, synthetic construct
  misc_feature 1..30: note = Description of Artificial Sequence: Synthetic oligonucleotide
  source 1..30: mol_type = other DNA
ggcgagtgccttcttggggatctggcaccc

SEQ ID NO: 252 - DNA, 30 bp, synthetic construct
  misc_feature 1..30: note = Description of Artificial Sequence: Synthetic oligonucleotide
  source 1..30: mol_type = other DNA
cacagccaggaagagaccatgttcattgcc

SEQ ID NO: 253 - DNA, 30 bp, synthetic construct
  misc_feature 1..30: note = Description of Artificial Sequence: Synthetic oligonucleotide
  source 1..30: mol_type = other DNA
caatgaacatggtctcttcctggctgtgga

SEQ ID NO: 254 - DNA, 30 bp, synthetic construct
  misc_feature 1..30: note = Description of Artificial Sequence: Synthetic oligonucleotide
  source 1..30: mol_type = other DNA
acatggtctcttcctggctgtggaaggagt

SEQ ID NO: 255 - DNA, 30 bp, synthetic construct
  misc_feature 1..30: note = Description of Artificial Sequence: Synthetic oligonucleotide
  source 1..30: mol_type = other DNA
atggtctcttcctggctgtggaaggagttg

SEQ ID NO: 256 - DNA, 30 bp, synthetic construct
  misc_feature 1..30: note = Description of Artificial Sequence: Synthetic oligonucleotide
  source 1..30: mol_type = other DNA
tcatcaacaaggtgcgcagcaatgcagcac

SEQ ID NO: 257 - DNA, 30 bp, synthetic construct
  misc_feature 1..30: note = Description of Artificial Sequence: Synthetic oligonucleotide
  source 1..30: mol_type = other DNA
caatatttgaagaggaaaaagaatggaaga

SEQ ID NO: 258 - DNA, 30 bp, synthetic construct
  misc_feature 1..30: note = Description of Artificial Sequence: Synthetic oligonucleotide
  source 1..30: mol_type = other DNA
cccaaaaccttggatcattcacagcctcca

SEQ ID NO: 259 - DNA, 30 bp, synthetic construct
  misc_feature 1..30: note = Description of Artificial Sequence: Synthetic oligonucleotide
  source 1..30: mol_type = other DNA
aagaggaaaaagaatggaagacggccgtgg

SEQ ID NO: 260 - DNA, 30 bp, synthetic construct
  misc_feature 1..30: note = Description of Artificial Sequence: Synthetic oligonucleotide
  source 1..30: mol_type = other DNA
aaggcagccgcgccatctggtacatgtggt

SEQ ID NO: 261 - DNA, 30 bp, synthetic construct
  misc_feature 1..30: note = Description of Artificial Sequence: Synthetic oligonucleotide
  source 1..30: mol_type = other DNA
agaatccaagggcttcaaactccaagaatc

SEQ ID NO: 262 - DNA, 30 bp, synthetic construct
  misc_feature 1..30: note = Description of Artificial Sequence: Synthetic oligonucleotide
  source 1..30: mol_type = other DNA
tctggtacatgtggttgggagccagattct

SEQ ID NO: 263 - DNA, 30 bp, synthetic construct
  misc_feature 1..30: note = Description of Artificial Sequence: Synthetic oligonucleotide
  source 1..30: mol_type = other DNA
ccacctcctgagttttctcttcccatccaa

SEQ ID NO: 264 - DNA, 30 bp, synthetic construct
  misc_feature 1..30: note = Description of Artificial Sequence: Synthetic oligonucleotide
  source 1..30: mol_type = other DNA
agacttggatacattctagaagaaatgaat

SEQ ID NO: 265 - DNA, 30 bp, synthetic construct
  misc_feature 1..30: note = Description of Artificial Sequence: Synthetic oligonucleotide
  source 1..30: mol_type = other DNA
gacttggatacattctagaagaaatgaatc

SEQ ID NO: 266 - DNA, 30 bp, synthetic construct
  misc_feature 1..30: note = Description of Artificial Sequence: Synthetic oligonucleotide
  source 1..30: mol_type = other DNA
caaacttactaatgcgggtgtcccagccag

SEQ ID NO: 267 - DNA, 30 bp, synthetic construct
  misc_feature 1..30: note = Description of Artificial Sequence: Synthetic oligonucleotide
  source 1..30: mol_type = other DNA
tgtgcccttcctccatttggttggtaatta

SEQ ID NO: 268 - DNA, 30 bp, synthetic construct
  misc_feature 1..30: note = Description of Artificial Sequence: Synthetic oligonucleotide
  source 1..30: mol_type = other DNA
agaatgaagccttaattaccaaccaaatgg

SEQ ID NO: 269 - DNA, 30 bp, synthetic construct
  misc_feature 1..30: note = Description of Artificial Sequence: Synthetic oligonucleotide
  source 1..30: mol_type = other DNA
caaagtggtgaaggtcctcagaccagctga

SEQ ID NO: 270 - DNA, 30 bp, synthetic construct
  misc_feature 1..30: note = Description of Artificial Sequence: Synthetic oligonucleotide
  source 1..30: mol_type = other DNA
ggaaaacagttatggacatcatttcaagac

SEQ ID NO: 271 - DNA, 30 bp, synthetic construct
  misc_feature 1..30: note = Description of Artificial Sequence: Synthetic oligonucleotide
  source 1..30: mol_type = other DNA
ccactaagttggtaaatgtgttgagagcat

SEQ ID NO: 272 - DNA, 30 bp, synthetic construct
  misc_feature 1..30: note = Description of Artificial Sequence: Synthetic oligonucleotide
  source 1..30: mol_type = other DNA
atctctaacacttcctcagcctccatattc

SEQ ID NO: 273 - DNA, 30 bp, synthetic construct
  misc_feature 1..30: note = Description of Artificial Sequence: Synthetic oligonucleotide
  source 1..30: mol_type = other DNA
attgcactgcaaccatctggtcactttctc

SEQ ID NO: 274 - DNA, 30 bp, synthetic construct
  misc_feature 1..30: note = Description of Artificial Sequence: Synthetic oligonucleotide
  source 1..30: mol_type = other DNA
atgacatgggaaaagttaggaaagacacac

SEQ ID NO: 275 - DNA, 30 bp, synthetic construct
  misc_feature 1..30: note = Description of Artificial Sequence: Synthetic oligonucleotide
  source 1..30: mol_type = other DNA
tgcagcttgttgaaatggtgggagcagaac

SEQ ID NO: 276 - DNA, 30 bp, synthetic construct
  misc_feature 1..30: note = Description of Artificial Sequence: Synthetic oligonucleotide
  source 1..30: mol_type = other DNA
cggccaatcaattcatcttggtggcggcaa

SEQ ID NO: 277 - DNA, 30 bp, synthetic construct
  misc_feature 1..30: note = Description of Artificial Sequence: Synthetic oligonucleotide
  source 1..30: mol_type = other DNA
gccacatctgtgcatatgattttgctagac

SEQ ID NO: 278 - DNA, 30 bp, synthetic construct
  misc_feature 1..30: note = Description of Artificial Sequence: Synthetic oligonucleotide
  source 1..30: mol_type = other DNA
accttcgactgatggccaatgctatttgtt

SEQ ID NO: 279 - DNA, 30 bp, synthetic construct
  misc_feature 1..30: note = Description of Artificial Sequence: Synthetic oligonucleotide
  source 1..30: mol_type = other DNA
cctttccgtggattgaccaggtggttctcc

SEQ ID NO: 280 - DNA, 30 bp, synthetic construct
  misc_feature 1..30: note = Description of Artificial Sequence: Synthetic oligonucleotide
  source 1..30: mol_type = other DNA
tccacggaaagggagaatggatgactactg

SEQ ID NO: 281 - DNA, 30 bp, synthetic construct
  misc_feature 1..30: note = Description of Artificial Sequence: Synthetic oligonucleotide
  source 1..30: mol_type = other DNA
gagtgtggattgaggagaacgaccatatgg

SEQ ID NO: 282 - DNA, 30 bp, synthetic construct
  misc_feature 1..30: note = Description of Artificial Sequence: Synthetic oligonucleotide
  source 1..30: mol_type = other DNA
caccataagtcctccctttttcccagatag

SEQ ID NO: 283 - DNA, 30 bp, synthetic construct
  misc_feature 1..30: note = Description of Artificial Sequence: Synthetic oligonucleotide
  source 1..30: mol_type = other DNA
ggacagacatcccctatctgggaaaaaggg

SEQ ID NO: 284 - DNA, 30 bp, synthetic construct
  misc_feature 1..30: note = Description of Artificial Sequence: Synthetic oligonucleotide
  source 1..30: mol_type = other DNA
cccaagtggtgcggggcctgtgccctataa

SEQ ID NO: 285 - DNA, 30 bp, synthetic construct
  misc_feature 1..30: note = Description of Artificial Sequence: Synthetic oligonucleotide
  source 1..30: mol_type = other DNA
gcccaagtggtgcggggcctgtgccctata

SEQ ID NO: 286 - DNA, 30 bp, synthetic construct
  misc_feature 1..30: note = Description of Artificial Sequence: Synthetic oligonucleotide
  source 1..30: mol_type = other DNA
gacttatggtgtggatcccttatagggcac

SEQ ID NO: 287 - DNA, 30 bp, synthetic construct
  misc_feature 1..30: note = Description of Artificial Sequence: Synthetic oligonucleotide
  source 1..30: mol_type = other DNA
tgtctttgatgttctcagcccaagtggtgc

SEQ ID NO: 288 - DNA, 30 bp, synthetic construct
  misc_feature 1..30: note = Description of Artificial Sequence: Synthetic oligonucleotide
  source 1..30: mol_type = other DNA
gtgtctttgatgttctcagcccaagtggtg

SEQ ID NO: 289 - DNA, 30 bp, synthetic construct
  misc_feature 1..30: note = Description of Artificial Sequence: Synthetic oligonucleotide
  source 1..30: mol_type = other DNA
aagaaaaatacatggactacttatccaccc

SEQ ID NO: 290 - DNA, 30 bp, synthetic construct
  misc_feature 1..30: note = Description of Artificial Sequence: Synthetic oligonucleotide
  source 1..30: mol_type = other DNA
ccaggtgtggacccttcctcacccaagtag

SEQ ID NO: 291 - DNA, 30 bp, synthetic construct
  misc_feature 1..30: note = Description of Artificial Sequence: Synthetic oligonucleotide
  source 1..30: mol_type = other DNA
acttatccacccaggtccgctacttgggtg

SEQ ID NO: 292 - DNA, 30 bp, synthetic construct
  misc_feature 1..30: note = Description of Artificial Sequence: Synthetic oligonucleotide
  source 1..30: mol_type = other DNA
aacgcggagtagcccgtgtgagcccctttg

SEQ ID NO: 293 - DNA, 30 bp, synthetic construct
  misc_feature 1..30: note = Description of Artificial Sequence: Synthetic oligonucleotide
  source 1..30: mol_type = other DNA
aggctgccagccggacttctgctgggtcat

SEQ ID NO: 294 - DNA, 30 bp, synthetic construct
  misc_feature 1..30: note = Description of Artificial Sequence: Synthetic oligonucleotide
  source 1..30: mol_type = other DNA
tcttggcgattctagcctttttgagattca

SEQ ID NO: 295 - DNA, 30 bp, synthetic construct
  misc_feature 1..30: note = Description of Artificial Sequence: Synthetic oligonucleotide
  source 1..30: mol_type = other DNA
tggaaataataaagaagttcaagaaagatc

SEQ ID NO: 296 - DNA, 30 bp, synthetic construct
  misc_feature 1..30: note = Description of Artificial Sequence: Synthetic oligonucleotide
  source 1..30: mol_type = other DNA
acctccgctgccatagctgtggtcagcagg

SEQ ID NO: 297 - DNA, 30 bp, synthetic construct
  misc_feature 1..30: note = Description of Artificial Sequence: Synthetic oligonucleotide
  source 1..30: mol_type = other DNA
cacgtctagtgacctccgctgccatagctg

SEQ ID NO: 298 - DNA, 30 bp, synthetic construct
  misc_feature 1..30: note = Description of Artificial Sequence: Synthetic oligonucleotide
  source 1..30: mol_type = other DNA
gcctcctgctgaccacagctatggcagcgg

SEQ ID NO: 299 - DNA, 30 bp, synthetic construct
  misc_feature 1..30: note = Description of Artificial Sequence: Synthetic oligonucleotide
  source 1..30: mol_type = other DNA
acagctatggcagcggaggtcactagacgt

SEQ ID NO: 300 - DNA, 30 bp, synthetic construct
  misc_feature 1..30: note = Description of Artificial Sequence: Synthetic oligonucleotide
  source 1..30: mol_type = other DNA
actatatgtacttggacagaaacgatgctg

SEQ ID NO: 301 - DNA, 30 bp, synthetic construct
  misc_feature 1..30: note = Description of Artificial Sequence: Synthetic oligonucleotide
  source 1..30: mol_type = other DNA
tatataacacttattcatccccaatgtggt

SEQ ID NO: 302 - DNA, 30 bp, synthetic construct
  misc_feature 1..30: note = Description of Artificial Sequence: Synthetic oligonucleotide
  source 1..30: mol_type = other DNA
ggggaggccatatcttttccaaccacattg

SEQ ID NO: 303 - DNA, 30 bp, synthetic construct
  misc_feature 1..30: note = Description of Artificial Sequence: Synthetic oligonucleotide
  source 1..30: mol_type = other DNA
atgagctatgaatgccctatgctggatgag

SEQ ID NO: 304 - DNA, 30 bp, synthetic construct
  misc_feature 1..30: note = Description of Artificial Sequence: Synthetic oligonucleotide
  source 1..30: mol_type = other DNA
tgagctatgaatgccctatgctggatgagg

SEQ ID NO: 305 - DNA, 30 bp, synthetic construct
  misc_feature 1..30: note = Description of Artificial Sequence: Synthetic oligonucleotide
  source 1..30: mol_type = other DNA
ctagatctccgtgcttcaccttttttgtga

SEQ ID NO: 306 - DNA, 30 bp, synthetic construct
  misc_feature 1..30: note = Description of Artificial Sequence: Synthetic oligonucleotide
  source 1..30: mol_type = other DNA
accgcgtttgcagcttcctagtggaatggg

SEQ ID NO: 307 - DNA, 30 bp, synthetic construct
  misc_feature 1..30: note = Description of Artificial Sequence: Synthetic oligonucleotide
  source 1..30: mol_type = other DNA
gcgaccgcgtttgcagcttcctagtggaat

SEQ ID NO: 308 - DNA, 30 bp, synthetic construct
  misc_feature 1..30: note = Description of Artificial Sequence: Synthetic oligonucleotide
  source 1..30: mol_type = other DNA
tgcgaccgcgtttgcagcttcctagtggaa

SEQ ID NO: 309 - DNA, 30 bp, synthetic construct
  misc_feature 1..30: note = Description of Artificial Sequence: Synthetic oligonucleotide
  source 1..30: mol_type = other DNA
ggaagctgcaaacgcggtcgcaaacctggt

SEQ ID NO: 310 - DNA, 30 bp, synthetic construct
  misc_feature 1..30: note = Description of Artificial Sequence: Synthetic oligonucleotide
  source 1..30: mol_type = other DNA
tacagcatcaggtgcataggagtcagcaat

SEQ ID NO: 311 - DNA, 30 bp, synthetic construct
  misc_feature 1..30: note = Description of Artificial Sequence: Synthetic oligonucleotide
  source 1..30: mol_type = other DNA
acagcatcaggtgcataggagtcagcaata

SEQ ID NO: 312 - DNA, 30 bp, synthetic construct
  misc_feature 1..30: note = Description of Artificial Sequence: Synthetic oligonucleotide
  source 1..30: mol_type = other DNA
agctggttacaacaacagtcagcaacatgg

SEQ ID NO: 313 - DNA, 30 bp, synthetic construct
  misc_feature 1..30: note = Description of Artificial Sequence: Synthetic oligonucleotide
  source 1..30: mol_type = other DNA
tcaaggtaggcttcaccttgtgttgggcag

SEQ ID NO: 314 - DNA, 30 bp, synthetic construct
  misc_feature 1..30: note = Description of Artificial Sequence: Synthetic oligonucleotide
  source 1..30: mol_type = other DNA
actccagattctctggctggatgctcttcc

SEQ ID NO: 315 - DNA, 30 bp, synthetic construct
  misc_feature 1..30: note = Description of Artificial Sequence: Synthetic oligonucleotide
  source 1..30: mol_type = other DNA
aagagcatccagccagagaatctggagtac

SEQ ID NO: 316 - DNA, 30 bp, synthetic construct
  misc_feature 1..30: note = Description of Artificial Sequence: Synthetic oligonucleotide
  source 1..30: mol_type = other DNA
tgctgggagccatgaactgacagcattatc

SEQ ID NO: 317 - DNA, 30 bp, synthetic construct
  misc_feature 1..30: note = Description of Artificial Sequence: Synthetic oligonucleotide
  source 1..30: mol_type = other DNA
ctaggcttccaaacccccccagggtggctt

SEQ ID NO: 318 - DNA, 30 bp, synthetic construct
  misc_feature 1..30: note = Description of Artificial Sequence: Synthetic oligonucleotide
  source 1..30: mol_type = other DNA
caagtcctaggcttccaaacccccccaggg

SEQ ID NO: 319 - DNA, 30 bp, synthetic construct
  misc_feature 1..30: note = Description of Artificial Sequence: Synthetic oligonucleotide
  source 1..30: mol_type = other DNA
caatcaagtcctaggcttccaaaccccccc

SEQ ID NO: 320 - DNA, 30 bp, synthetic construct
  misc_feature 1..30: note = Description of Artificial Sequence: Synthetic oligonucleotide
  source 1..30: mol_type = other DNA
aattcaccaagagccgaagccaccctgggg

SEQ ID NO: 321 - DNA, 30 bp, synthetic construct
  misc_feature 1..30: note = Description of Artificial Sequence: Synthetic oligonucleotide
  source 1..30: mol_type = other DNA
aggcctgtcctcggttcacaatcaagtcct

SEQ ID NO: 322 - DNA, 30 bp, synthetic construct
  misc_feature 1..30: note = Description of Artificial Sequence: Synthetic oligonucleotide
  source 1..30: mol_type = other DNA
ctatgaataacaagcactggctggttcaca

SEQ ID NO: 323 - DNA, 30 bp, synthetic construct
  misc_feature 1..30: note = Description of Artificial Sequence: Synthetic oligonucleotide
  source 1..30: mol_type = other DNA
gccccagcgtgccaaggtaatggaatgtcg

SEQ ID NO: 324 - DNA, 30 bp, synthetic construct
  misc_feature 1..30: note = Description of Artificial Sequence: Synthetic oligonucleotide
  source 1..30: mol_type = other DNA
ggaacaacaaagaagcactggtagagttca

SEQ ID NO: 325 - DNA, 30 bp, synthetic construct
  misc_feature 1..30: note = Description of Artificial Sequence: Synthetic oligonucleotide
  source 1..30: mol_type = other DNA
aggacgcacatgccaaaaggcaaactgtcg

SEQ ID NO: 326 - DNA, 30 bp, synthetic construct
  misc_feature 1..30: note = Description of Artificial Sequence: Synthetic oligonucleotide
  source 1..30: mol_type = other DNA
catccatctcagcctccagagctccagcaa

SEQ ID NO: 327 - DNA, 30 bp, synthetic construct
  misc_feature 1..30: note = Description of Artificial Sequence: Synthetic oligonucleotide
  source 1..30: mol_type = other DNA
ccatccatctcagcctccagagctccagca

SEQ ID NO: 328 - DNA, 30 bp, synthetic construct
  misc_feature 1..30: note = Description of Artificial Sequence: Synthetic oligonucleotide
  source 1..30: mol_type = other DNA
gagctctggaggctgagatggatggtgcaa

SEQ ID NO: 329 - DNA, 30 bp, synthetic construct
  misc_feature 1..30: note = Description of Artificial Sequence: Synthetic oligonucleotide
  source 1..30: mol_type = other DNA
ccattttcaggcgacatttcaagtggccag

SEQ ID NO: 330 - DNA, 30 bp, synthetic construct
  misc_feature 1..30: note = Description of Artificial Sequence: Synthetic oligonucleotide
  source 1..30: mol_type = other DNA
acgcccttcaatctaagtttatccattttc

SEQ ID NO: 331 - DNA, 30 bp, synthetic construct
  misc_feature 1..30: note = Description of Artificial Sequence: Synthetic oligonucleotide
  source 1..30: mol_type = other DNA
gtactgcagcgttcacattcaccaagatcc

SEQ ID NO: 332 - DNA, 30 bp, synthetic construct
  misc_feature 1..30: note = Description of Artificial Sequence: Synthetic oligonucleotide
  source 1..30: mol_type = other DNA
ttcaccaagatcccggctgaaacactgcac

SEQ ID NO: 333 - DNA, 30 bp, synthetic construct
  misc_feature 1..30: note = Description of Artificial Sequence: Synthetic oligonucleotide
  source 1..30: mol_type = other DNA
tcttagagttctcagtgctttcagtgatta

SEQ ID NO: 334 - DNA, 30 bp, synthetic construct
  misc_feature 1..30: note = Description of Artificial Sequence: Synthetic oligonucleotide
  source 1..30: mol_type = other DNA
tttccaatggtgctgccactcctgtgccag

SEQ ID NO: 335 - DNA, 30 bp, synthetic construct
  misc_feature 1..30: note = Description of Artificial Sequence: Synthetic oligonucleotide
  source 1..30: mol_type = other DNA
aatgatttgaaagctgctccaaagatttga

SEQ ID NO: 336 - DNA, 30 bp, synthetic construct
  misc_feature 1..30: note = Description of Artificial Sequence: Synthetic oligonucleotide
  source 1..30: mol_type = other DNA
agtccaccgagcaccccacatcagcagaga

SEQ ID NO: 337 - DNA, 30 bp, synthetic construct
  misc_feature 1..30: note = Description of Artificial Sequence: Synthetic oligonucleotide
  source 1..30: mol_type = other DNA
atgtggggtgctcggtggacttctcaaaga

SEQ ID NO: 338 - DNA, 30 bp, synthetic construct
  misc_feature 1..30: note = Description of Artificial Sequence: Synthetic oligonucleotide
  source 1..30: mol_type = other DNA
cagaggagatcccgcagataccatcttccc

SEQ ID NO: 339 - DNA, 30 bp, synthetic construct
  misc_feature 1..30: note = Description of Artificial Sequence: Synthetic oligonucleotide
  source 1..30: mol_type = other DNA
gatcagtagaaggggagctcaacgcaatcc

SEQ ID NO: 340 - DNA, 30 bp, synthetic construct
  misc_feature 1..30: note = Description of Artificial Sequence: Synthetic oligonucleotide
  source 1..30: mol_type = other DNA
caatcctggaagagaatggagttcaactga

SEQ ID NO: 341 - DNA, 30 bp, synthetic construct
  misc_feature 1..30: note = Description of Artificial Sequence: Synthetic oligonucleotide
  source 1..30: mol_type = other DNA
aagagaatggagttcaactgacggtcgttg

SEQ ID NO: 342 - DNA, 30 bp, synthetic construct
  misc_feature 1..30: note = Description of Artificial Sequence: Synthetic oligonucleotide
  source 1..30: mol_type = other DNA
tccagccgtggggcagctcgttcacaggca

SEQ ID NO: 343 - DNA, 30 bp, synthetic construct
  misc_feature 1..30: note = Description of Artificial Sequence: Synthetic oligonucleotide
  source 1..30: mol_type = other DNA
tgcctgtgaacgagctgccccacggctgga

SEQ ID NO: 344 - DNA, 30 bp, synthetic construct
  misc_feature 1..30: note = Description of Artificial Sequence: Synthetic oligonucleotide
  source 1..30: mol_type = other DNA
gagcagcaaagacaaataacagctttgtcg

SEQ ID NO: 345 - DNA, 30 bp, synthetic construct
  misc_feature 1..30: note = Description of Artificial Sequence: Synthetic oligonucleotide
  source 1..30: mol_type = other DNA
agaaagctgttccatgctctatgtttgagt

SEQ ID NO: 346 - DNA, 30 bp, synthetic construct
  misc_feature 1..30: note = Description of Artificial Sequence: Synthetic oligonucleotide
  source 1..30: mol_type = other DNA
tctcttctattccatctgtccacaatgtgt

SEQ ID NO: 347 - DNA, 30 bp, synthetic construct
  misc_feature 1..30: note = Description of Artificial Sequence: Synthetic oligonucleotide
  source 1..30: mol_type = other DNA
ctctcttctattccatctgtccacaatgtg

SEQ ID NO: 348 - DNA, 30 bp, synthetic construct
  misc_feature 1..30: note = Description of Artificial Sequence: Synthetic oligonucleotide
  source 1..30: mol_type = other DNA
tgtgatggctgagtggcccagctaaagact

SEQ ID NO: 349 - DNA, 30 bp, synthetic construct
  misc_feature 1..30: note = Description of Artificial Sequence: Synthetic oligonucleotide
  source 1..30: mol_type = other DNA
agtgatctgatcatacccaagtctttagct

SEQ ID NO: 350 - DNA, 30 bp, synthetic construct
  misc_feature 1..30: note = Description of Artificial Sequence: Synthetic oligonucleotide
  source 1..30: mol_type = other DNA
gccctttcatttgggtcctgtagccctctc

SEQ ID NO: 351 - DNA, 30 bp, synthetic construct
  misc_feature 1..30: note = Description of Artificial Sequence: Synthetic oligonucleotide
  source 1..30: mol_type = other DNA
ctcagccatcacaataccagagagggctac

SEQ ID NO: 352 - DNA, 30 bp, synthetic construct
  misc_feature 1..30: note = Description of Artificial Sequence: Synthetic oligonucleotide
  source 1..30: mol_type = other DNA
gctcttcactgtgccatggccctttcattt

SEQ ID NO: 353 - DNA, 30 bp, synthetic construct
  misc_feature 1..30: note = Description of Artificial Sequence: Synthetic oligonucleotide
  source 1..30: mol_type = other DNA
agctcttcactgtgccatggccctttcatt

SEQ ID NO: 354 - DNA, 30 bp, synthetic construct
  misc_feature 1..30: note = Description of Artificial Sequence: Synthetic oligonucleotide
  source 1..30: mol_type = other DNA
catgtttcctccacgtggaccttagtgcct

SEQ ID NO: 355 - DNA, 30 bp, synthetic construct
  misc_feature 1..30: note = Description of Artificial Sequence: Synthetic oligonucleotide
  source 1..30: mol_type = other DNA
agcggaagggtgatcgaggaatggtgctgc

SEQ ID NO: 356 - DNA, 30 bp, synthetic construct
  misc_feature 1..30: note = Description of Artificial Sequence: Synthetic oligonucleotide
  source 1..30: mol_type = other DNA
gcggaagggtgatcgaggaatggtgctgca

SEQ ID NO: 357 - DNA, 30 bp, synthetic construct
  misc_feature 1..30: note = Description of Artificial Sequence: Synthetic oligonucleotide
  source 1..30: mol_type = other DNA
agggagtgcacaatgcccccactgtcgttc

SEQ ID NO: 358 - DNA, 30 bp, synthetic construct
  misc_feature 1..30: note = Description of Artificial Sequence: Synthetic oligonucleotide
  source 1..30: mol_type = other DNA
gggagtgcacaatgcccccactgtcgttcc

SEQ ID NO: 359 - DNA, 30 bp, synthetic construct
  misc_feature 1..30: note = Description of Artificial Sequence: Synthetic oligonucleotide
  source 1..30: mol_type = other DNA
tccattccataccaacagccatctttagcc

SEQ ID NO: 360 - DNA, 30 bp, synthetic construct
  misc_feature 1..30: note = Description of Artificial Sequence: Synthetic oligonucleotide
  source 1..30: mol_type = other DNA
gcaccatgagcagaatcacaagcactccaa

SEQ ID NO: 361 - DNA, 30 bp, synthetic construct
  misc_feature 1..30: note = Description of Artificial Sequence: Synthetic oligonucleotide
  source 1..30: mol_type = other DNA
tgcaccatgagcagaatcacaagcactcca

SEQ ID NO: 362 - DNA, 30 bp, synthetic construct
  misc_feature 1..30: note = Description of Artificial Sequence: Synthetic oligonucleotide
  source 1..30: mol_type = other DNA
ttggagtgcttgtgattctgctcatggtgc

SEQ ID NO: 363 - DNA, 30 bp, synthetic construct
  misc_feature 1..30: note = Description of Artificial Sequence: Synthetic oligonucleotide
  source 1..30: mol_type = other DNA
gtggcacccatcaaaattgcaagcttagcc

SEQ ID NO: 364 - DNA, 30 bp, synthetic construct
  misc_feature 1..30: note = Description of Artificial Sequence: Synthetic oligonucleotide
  source 1..30: mol_type = other DNA
agcttgcaattttgatgggtgccaccttcg

SEQ ID NO: 365 - DNA, 30 bp, synthetic construct
  misc_feature 1..30: note = Description of Artificial Sequence: Synthetic oligonucleotide
  source 1..30: mol_type = other DNA
acgaggccaaggccagcagcatgctttcac

SEQ ID NO: 366 - DNA, 30 bp, synthetic construct
  misc_feature 1..30: note = Description of Artificial Sequence: Synthetic oligonucleotide
  source 1..30: mol_type = other DNA
cacgaggccaaggccagcagcatgctttca

SEQ ID NO: 367 - DNA, 30 bp, synthetic construct
  misc_feature 1..30: note = Description of Artificial Sequence: Synthetic oligonucleotide
  source 1..30: mol_type = other DNA
ctaattggacaccccgtgaaagcatgctgc

SEQ ID NO: 368 - DNA, 30 bp, synthetic construct
  misc_feature 1..30: note = Description of Artificial Sequence: Synthetic oligonucleotide
  source 1..30: mol_type = other DNA
aggcggagatcgcagtttgcaaaagacacg

SEQ ID NO: 369 - DNA, 30 bp, synthetic construct
  misc_feature 1..30: note = Description of Artificial Sequence: Synthetic oligonucleotide
  source 1..30: mol_type = other DNA
tgatgagaaccatcaggtcgccttccaagg

SEQ ID NO: 370 - DNA, 30 bp, synthetic construct
  misc_feature 1..30: note = Description of Artificial Sequence: Synthetic oligonucleotide
  source 1..30: mol_type = other DNA
cgtgtcttttgcaaactgcgatctccgcct

SEQ ID NO: 371 - DNA, 30 bp, synthetic construct
  misc_feature 1..30: note = Description of Artificial Sequence: Synthetic oligonucleotide
  source 1..30: mol_type = other DNA
cattgatgagaaccatcaggtcgccttcca

SEQ ID NO: 372 - DNA, 30 bp, synthetic construct
  misc_feature 1..30: note = Description of Artificial Sequence: Synthetic oligonucleotide
  source 1..30: mol_type = other DNA
ccagtggtgtcagagcagccaggattgcca

SEQ ID NO: 373 - DNA, 30 bp, synthetic construct
  misc_feature 1..30: note = Description of Artificial Sequence: Synthetic oligonucleotide
  source 1..30: mol_type = other DNA
ccttggcaatcctggctgctctgacaccac

SEQ ID NO: 374 - DNA, 30 bp, synthetic construct
  misc_feature 1..30: note = Description of Artificial Sequence: Synthetic oligonucleotide
  source 1..30: mol_type = other DNA
gcaatcctggctgctctgacaccactggcc

SEQ ID NO: 375 - DNA, 30 bp, synthetic construct
  misc_feature 1..30: note = Description of Artificial Sequence: Synthetic oligonucleotide
  source 1..30: mol_type = other DNA
caatcctggctgctctgacaccactggccc

SEQ ID NO: 376 - DNA, 30 bp, synthetic construct
  misc_feature 1..30: note = Description of Artificial Sequence: Synthetic oligonucleotide
  source 1..30: mol_type = other DNA
gtggcgtggagagcaggccttgctacttgc

SEQ ID NO: 377 - DNA, 30 bp, synthetic construct
  misc_feature 1..30: note = Description of Artificial Sequence: Synthetic oligonucleotide
  source 1..30: mol_type = other DNA
tggcgtggagagcaggccttgctacttgcg

SEQ ID NO: 378 - DNA, 30 bp, synthetic construct
  misc_feature 1..30: note = Description of Artificial Sequence: Synthetic oligonucleotide
  source 1..30: mol_type = other DNA
gggtcgaccagcctcacagcggttagtccc

SEQ ID NO: 379 - DNA, 30 bp, synthetic construct
  misc_feature 1..30: note = Description of Artificial Sequence: Synthetic oligonucleotide
  source 1..30: mol_type = other DNA
ccacgttgatggggtcgaccagcctcacag

SEQ ID NO: 380 - DNA, 30 bp, synthetic construct
  misc_feature 1..30: note = Description of Artificial Sequence: Synthetic oligonucleotide
  source 1..30: mol_type = other DNA
tcatggccctgggactaaccgctgtgaggc

SEQ ID NO: 381 - DNA, 30 bp, synthetic construct
  misc_feature 1..30: note = Description of Artificial Sequence: Synthetic oligonucleotide
  source 1..30: mol_type = other DNA
cagatatagagatggctgggcccatggccg

SEQ ID NO: 382 - DNA, 30 bp, synthetic construct
  misc_feature 1..30: note = Description of Artificial Sequence: Synthetic oligonucleotide
  source 1..30: mol_type = other DNA
ccacgtaactgacaattagcagaccgaccg

SEQ ID NO: 383 - DNA, 30 bp, synthetic construct
  misc_feature 1..30: note = Description of Artificial Sequence: Synthetic oligonucleotide
  source 1..30: mol_type = other DNA
caccactctcatctagcgccacatcgagcc

SEQ ID NO: 384 - DNA, 30 bp, synthetic construct
  misc_feature 1..30: note = Description of Artificial Sequence: Synthetic oligonucleotide
  source 1..30: mol_type = other DNA
tcaccactctcatctagcgccacatcgagc

SEQ ID NO: 385 - DNA, 30 bp, synthetic construct
  misc_feature 1..30: note = Description of Artificial Sequence: Synthetic oligonucleotide
  source 1..30: mol_type = other DNA
ctctcatggggggaccgtcatcctccacca

SEQ ID NO: 386 - DNA, 30 bp, synthetic construct
  misc_feature 1..30: note = Description of Artificial Sequence: Synthetic oligonucleotide
  source 1..30: mol_type = other DNA
ggaccaccttgagtatgatctctctcatgg

SEQ ID NO: 387 - DNA, 30 bp, synthetic construct
  misc_feature 1..30: note = Description of Artificial Sequence: Synthetic oligonucleotide
  source 1..30: mol_type = other DNA
aggaccaccttgagtatgatctctctcatg

SEQ ID NO: 388 - DNA, 30 bp, synthetic construct
  misc_feature 1..30: note = Description of Artificial Sequence: Synthetic oligonucleotide
  source 1..30: mol_type = other DNA
gtggtgctctatgggatgtgcctgctccca

SEQ ID NO: 389 - DNA, 30 bp, synthetic construct
  misc_feature 1..30: note = Description of Artificial Sequence: Synthetic oligonucleotide
  source 1..30: mol_type = other DNA
gggatgtgcctgctcccaaggaagtaaaaa

SEQ ID NO: 390 - DNA, 30 bp, synthetic construct
  misc_feature 1..30: note = Description of Artificial Sequence: Synthetic oligonucleotide
  source 1..30: mol_type = other DNA
gatgtgcctgctcccaaggaagtaaaaaag

SEQ ID NO: 391 - DNA, 30 bp, synthetic construct
  misc_feature 1..30: note = Description of Artificial Sequence: Synthetic oligonucleotide
  source 1..30: mol_type = other DNA
atgtgcctgctcccaaggaagtaaaaaagg

SEQ ID NO: 392 - DNA, 30 bp, synthetic construct
  misc_feature 1..30: note = Description of Artificial Sequence: Synthetic oligonucleotide
  source 1..30: mol_type = other DNA
acacaagttggagtgggagttatgcaagag

SEQ ID NO: 393 - DNA, 30 bp, synthetic construct
  misc_feature 1..30: note = Description of Artificial Sequence: Synthetic oligonucleotide
  source 1..30: mol_type = other DNA
cacaagttggagtgggagttatgcaagagg

SEQ ID NO: 394 - DNA, 30 bp, synthetic construct
  misc_feature 1..30: note = Description of Artificial Sequence: Synthetic oligonucleotide
  source 1..30: mol_type = other DNA
gatcaagtctcccttcaccgcttctcagcg

SEQ ID NO: 395 - DNA, 30 bp, synthetic construct
  misc_feature 1..30: note = Description of Artificial Sequence: Synthetic oligonucleotide
  source 1..30: mol_type = other DNA
atccatactggggagatgtcaagcaggatc

SEQ ID NO: 396 - DNA, 30 bp, synthetic construct
  misc_feature 1..30: note = Description of Artificial Sequence: Synthetic oligonucleotide
  source 1..30: mol_type = other DNA
ccaagagctgcacctcgctgtgcccgtccc

SEQ ID NO: 397 - DNA, 30 bp, synthetic construct
  misc_feature 1..30: note = Description of Artificial Sequence: Synthetic oligonucleotide
  source 1..30: mol_type = other DNA
ggtccatggaagctagatgccgcctgggac

SEQ ID NO: 398 - DNA, 30 bp, synthetic construct
  misc_feature 1..30: note = Description of Artificial Sequence: Synthetic oligonucleotide
  source 1..30: mol_type = other DNA
ggatgttcctcgctctctctccggggggca

SEQ ID NO: 399 - DNA, 30 bp, synthetic construct
  misc_feature 1..30: note = Description of Artificial Sequence: Synthetic oligonucleotide
  source 1..30: mol_type = other DNA
ctcttggccgtgccccccggagagagagcg

SEQ ID NO: 400 - DNA, 30 bp, synthetic construct
  misc_feature 1..30: note = Description of Artificial Sequence: Synthetic oligonucleotide
  source 1..30: mol_type = other DNA
tcctttgtcttaaatattccgggcagagtc

SEQ ID NO: 401 - DNA, 30 bp, synthetic construct
  misc_feature 1..30: note = Description of Artificial Sequence: Synthetic oligonucleotide
  source 1..30: mol_type = other DNA
caatgtccccatcctttgtcttaaatattc

SEQ ID NO: 402 - DNA, 30 bp, synthetic construct
  misc_feature 1..30: note = Description of Artificial Sequence: Synthetic oligonucleotide
  source 1..30: mol_type = other DNA
tatttaagacaaaggatggggacattggag

SEQ ID NO: 403 - DNA, 30 bp, synthetic construct
  misc_feature 1..30: note = Description of Artificial Sequence: Synthetic oligonucleotide
  source 1..30: mol_type = other DNA
acttcaggatctcctatcctagacaagtgt

SEQ ID NO: 404 - DNA, 30 bp, synthetic construct
  misc_feature 1..30: note = Description of Artificial Sequence: Synthetic oligonucleotide
  source 1..30: mol_type = other DNA
ctttatggcaatggggtcgtgatcaaaaat

SEQ ID NO: 405 - DNA, 30 bp, synthetic construct
  misc_feature 1..30: note = Description of Artificial Sequence: Synthetic oligonucleotide
  source 1..30: mol_type = other DNA
gggagttatgttagtgccatcacccaaggg

SEQ ID NO: 406 - DNA, 30 bp, synthetic construct
  misc_feature 1..30: note = Description of Artificial Sequence: Synthetic oligonucleotide
  source 1..30: mol_type = other DNA
ctaactgtcttagacttgcatcctggagct

SEQ ID NO: 407 - DNA, 30 bp, synthetic construct
  misc_feature 1..30: note = Description of Artificial Sequence: Synthetic oligonucleotide
  source 1..30: mol_type = other DNA
cttcacggactatttcaggaagaactctcc

SEQ ID NO: 408 - DNA, 30 bp, synthetic construct
  misc_feature 1..30: note = Description of Artificial Sequence: Synthetic oligonucleotide
  source 1..30: mol_type = other DNA
caaccagggttgtcgctgctgaaatggagg

SEQ ID NO: 409 - DNA, 30 bp, synthetic construct
  misc_feature 1..30: note = Description of Artificial Sequence: Synthetic oligonucleotide
  source 1..30: mol_type = other DNA
gttgtcatataacgcactggaagccctcta

SEQ ID NO: 410 - DNA, 30 bp, synthetic construct
  misc_feature 1..30: note = Description of Artificial Sequence: Synthetic oligonucleotide
  source 1..30: mol_type = other DNA
gtcgctgctgaaatggaggaggcccttaga

SEQ ID NO: 411 - DNA, 30 bp, synthetic construct
  misc_feature 1..30: note = Description of Artificial Sequence: Synthetic oligonucleotide
  source 1..30: mol_type = other DNA
gggcctcatccataatatacagattatagt

SEQ ID NO: 412 - DNA, 30 bp, synthetic construct
  misc_feature 1..30: note = Description of Artificial Sequence: Synthetic oligonucleotide
  source 1..30: mol_type = other DNA
tccccaactataatctgtatattatggatg

SEQ ID NO: 413 - DNA, 30 bp, synthetic construct
  misc_feature 1..30: note = Description of Artificial Sequence: Synthetic oligonucleotide
  source 1..30: mol_type = other DNA
ttcctggtggcgtggcggtcatgaagatgg

SEQ ID NO: 414 - DNA, 30 bp, synthetic construct
  misc_feature 1..30: note = Description of Artificial Sequence: Synthetic oligonucleotide
  source 1..30: mol_type = other DNA
gaaatgcgtcacgggttcctggtggcgtgg

SEQ ID NO: 415 - DNA, 30 bp, synthetic construct
  misc_feature 1..30: note = Description of Artificial Sequence: Synthetic oligonucleotide
  source 1..30: mol_type = other DNA
ccacgccaccaggaacccgtgacgcatttc

SEQ ID NO: 416 - DNA, 30 bp, synthetic construct
  misc_feature 1..30: note = Description of Artificial Sequence: Synthetic oligonucleotide
  source 1..30: mol_type = other DNA
agctccaggctctctctgggacttccactt

SEQ ID NO: 417 - DNA, 30 bp, synthetic construct
  misc_feature 1..30: note = Description of Artificial Sequence: Synthetic oligonucleotide
  source 1..30: mol_type = other DNA
gagcctggagctcaggctttgattgggtga

SEQ ID NO: 418 - DNA, 30 bp, synthetic construct
  misc_feature 1..30: note = Description of Artificial Sequence: Synthetic oligonucleotide
  source 1..30: mol_type = other DNA
ggaaaaacagtttggtttgttccaagcgtg

SEQ ID NO: 419 - DNA, 30 bp, synthetic construct
  misc_feature 1..30: note = Description of Artificial Sequence: Synthetic oligonucleotide
  source 1..30: mol_type = other DNA
acaaagtcccactcttgatgttttgttttc

SEQ ID NO: 420 - DNA, 30 bp, synthetic construct
  misc_feature 1..30: note = Description of Artificial Sequence: Synthetic oligonucleotide
  source 1..30: mol_type = other DNA
tggaatctatgacacggtcagctttaaagt

SEQ ID NO: 421 - DNA, 30 bp, synthetic construct
  misc_feature 1..30: note = Description of Artificial Sequence: Synthetic oligonucleotide
  source 1..30: mol_type = other DNA
catcaagtatgaccggctttaggcatctcc

SEQ ID NO: 422 - DNA, 30 bp, synthetic construct
  misc_feature 1..30: note = Description of Artificial Sequence: Synthetic oligonucleotide
  source 1..30: mol_type = other DNA
gtgtcatagattccaggagatgcctaaagc

SEQ ID NO: 423 - DNA, 30 bp, synthetic construct
  misc_feature 1..30: note = Description of Artificial Sequence: Synthetic oligonucleotide
  source 1..30: mol_type = other DNA
tgcctatgcgccccctcctctgggcagcgc

SEQ ID NO: 424 - DNA, 30 bp, synthetic construct
  misc_feature 1..30: note = Description of Artificial Sequence: Synthetic oligonucleotide
  source 1..30: mol_type = other DNA
ttgggattcctgcctatgcgccccctcctc

SEQ ID NO: 425 - DNA, 30 bp, synthetic construct
  misc_feature 1..30: note = Description of Artificial Sequence: Synthetic oligonucleotide
  source 1..30: mol_type = other DNA
cctgtcacacatgccagcgctgcccagagg

SEQ ID NO: 426 - DNA, 30 bp, synthetic construct
  misc_feature 1..30: note = Description of Artificial Sequence: Synthetic oligonucleotide
  source 1..30: mol_type = other DNA
ctgtcacacatgccagcgctgcccagagga

SEQ ID NO: 427 - DNA, 30 bp, synthetic construct
  misc_feature 1..30: note = Description of Artificial Sequence: Synthetic oligonucleotide
  source 1..30: mol_type = other DNA
ctccatacagatactcatctccaggtttgt

SEQ ID NO: 428 - DNA, 30 bp, synthetic construct
  misc_feature 1..30: note = Description of Artificial Sequence: Synthetic oligonucleotide
  source 1..30: mol_type = other DNA
aaacctggagatgagtatctgtatggaggt

SEQ ID NO: 429 - DNA, 30 bp, synthetic construct
  misc_feature 1..30: note = Description of Artificial Sequence: Synthetic oligonucleotide
  source 1..30: mol_type = other DNA
atgaggccatcttggaggtaaatattgtca

SEQ ID NO: 430 - DNA, 30 bp, synthetic construct
  misc_feature 1..30: note = Description of Artificial Sequence: Synthetic oligonucleotide
  source 1..30: mol_type = other DNA
cagccattgagggagagttcaagcttagga

SEQ ID NO: 431 - DNA, 30 bp, synthetic construct
  misc_feature 1..30: note = Description of Artificial Sequence: Synthetic oligonucleotide
  source 1..30: mol_type = other DNA
tcatgaaaagaggagatcttcctgtttggc

SEQ ID NO: 432 - DNA, 30 bp, synthetic construct
  misc_feature 1..30: note = Description of Artificial Sequence: Synthetic oligonucleotide
  source 1..30: mol_type = other DNA
tgccatcaaagcaccatcttctatctgtgt

SEQ ID NO: 433 - DNA, 30 bp, synthetic construct
  misc_feature 1..30: note = Description of Artificial Sequence: Synthetic oligonucleotide
  source 1..30: mol_type = other DNA
ccaacaacaccataatggaagacagtgtgc

SEQ ID NO: 434 - DNA, 30 bp, synthetic construct
  misc_feature 1..30: note = Description of Artificial Sequence: Synthetic oligonucleotide
  source 1..30: mol_type = other DNA
tcggtttgagcactcttttctctccgtgtc

SEQ ID NO: 435 - DNA, 30 bp, synthetic construct
  misc_feature 1..30: note = Description of Artificial Sequence: Synthetic oligonucleotide
  source 1..30: mol_type = other DNA
agacacggagagaaaagagtgctcaaaccg

SEQ ID NO: 436 - DNA, 30 bp, synthetic construct
  misc_feature 1..30: note = Description of Artificial Sequence: Synthetic oligonucleotide
  source 1..30: mol_type = other DNA
acttcagggccgcatgatctgaacaaactc

SEQ ID NO: 437 - DNA, 30 bp, synthetic construct
  misc_feature 1..30: note = Description of Artificial Sequence: Synthetic oligonucleotide
  source 1..30: mol_type = other DNA
ggatggacgccagagtttgttcagatcatg

SEQ ID NO: 438 - DNA, 30 bp, synthetic construct
  misc_feature 1..30: note = Description of Artificial Sequence: Synthetic oligonucleotide
  source 1..30: mol_type = other DNA
gttcagatcatgcggccctgaagtcattca

SEQ ID NO: 439 - DNA, 30 bp, synthetic construct
  misc_feature 1..30: note = Description of Artificial Sequence: Synthetic oligonucleotide
  source 1..30: mol_type = other DNA
cttccagtctctgcccgcatgagcacagcg

SEQ ID NO: 440 - DNA, 30 bp, synthetic construct
  misc_feature 1..30: note = Description of Artificial Sequence: Synthetic oligonucleotide
  source 1..30: mol_type = other DNA
gaagccattgacaacctcgctgtgctcatg

SEQ ID NO: 441 - DNA, 30 bp, synthetic construct
  misc_feature 1..30: note = Description of Artificial Sequence: Synthetic oligonucleotide
  source 1..30: mol_type = other DNA
aagccattgacaacctcgctgtgctcatgc

SEQ ID NO: 442 - DNA, 30 bp, synthetic construct
  misc_feature 1..30: note = Description of Artificial Sequence: Synthetic oligonucleotide
  source 1..30: mol_type = other DNA
tctctagggtctccggcaattgggccgccg

SEQ ID NO: 443 - DNA, 30 bp, synthetic construct
  misc_feature 1..30: note = Description of Artificial Sequence: Synthetic oligonucleotide
  source 1..30: mol_type = other DNA
agactggaagcaggccttacaaagccgcgg

SEQ ID NO: 444 - DNA, 30 bp, synthetic construct
  misc_feature 1..30: note = Description of Artificial Sequence: Synthetic oligonucleotide
  source 1..30: mol_type = other DNA
tgccggagaccctagagaccattatgcttt

SEQ ID NO: 445 - DNA, 30 bp, synthetic construct
  misc_feature 1..30: note = Description of Artificial Sequence: Synthetic oligonucleotide
  source 1..30: mol_type = other DNA
ccggagaccctagagaccattatgcttttg

SEQ ID NO: 446 - DNA, 30 bp, synthetic construct
  misc_feature 1..30: note = Description of Artificial Sequence: Synthetic oligonucleotide
  source 1..30: mol_type = other DNA
tctggctcaggtatgagcaccaccagcaat

SEQ ID NO: 447 - DNA, 30 bp, synthetic construct
  misc_feature 1..30: note = Description of Artificial Sequence: Synthetic oligonucleotide
  source 1..30: mol_type = other DNA
ttgttctctccaaccatccgagttcattgg

SEQ ID NO: 448 - DNA, 30 bp, synthetic construct
  misc_feature 1..30: note = Description of Artificial Sequence: Synthetic oligonucleotide
  source 1..30: mol_type = other DNA
tctttgttctctccaaccatccgagttcat

SEQ ID NO: 449 - DNA, 30 bp, synthetic construct
  misc_feature 1..30: note = Description of Artificial Sequence: Synthetic oligonucleotide
  source 1..30: mol_type = other DNA
ccctcctctctccttcccattagatggctt

SEQ ID NO: 450 - DNA, 30 bp, synthetic construct
  misc_feature 1..30: note = Description of Artificial Sequence: Synthetic oligonucleotide
  source 1..30: mol_type = other DNA
gttgccccctcctctctccttcccattaga

SEQ ID NO: 451 - DNA, 30 bp, synthetic construct
  misc_feature 1..30: note = Description of Artificial Sequence: Synthetic oligonucleotide
  source 1..30: mol_type = other DNA
gtgacctaagccatctaatgggaaggagag

SEQ ID NO: 452 - DNA, 30 bp, synthetic construct
  misc_feature 1..30: note = Description of Artificial Sequence: Synthetic oligonucleotide
  source 1..30: mol_type = other DNA
acctaagccatctaatgggaaggagagagg

SEQ ID NO: 453 - DNA, 30 bp, synthetic construct
  misc_feature 1..30: note = Description of Artificial Sequence: Synthetic oligonucleotide
  source 1..30: mol_type = other DNA
ctaagccatctaatgggaaggagagaggag

SEQ ID NO: 454 - DNA, 30 bp, synthetic construct
  misc_feature 1..30: note = Description of Artificial Sequence: Synthetic oligonucleotide
  source 1..30: mol_type = other DNA
taagccatctaatgggaaggagagaggagg

SEQ ID NO: 455 - DNA, 30 bp, synthetic construct
  misc_feature 1..30: note = Description of Artificial Sequence: Synthetic oligonucleotide
  source 1..30: mol_type = other DNA
gccgcaggtcaatgtccattgagaatccta

SEQ ID NO: 456 - DNA, 30 bp, synthetic construct
  misc_feature 1..30: note = Description of Artificial Sequence: Synthetic oligonucleotide
  source 1..30: mol_type = other DNA
gcatagatggcccaagctgaggctggccgc

SEQ ID NO: 457 - DNA, 30 bp, synthetic construct
  misc_feature 1..30: note = Description of Artificial Sequence: Synthetic oligonucleotide
  source 1..30: mol_type = other DNA
gggtaatgaaagttgtcaaggcagcataga

SEQ ID NO: 458 - DNA, 30 bp, synthetic construct
  misc_feature 1..30: note = Description of Artificial Sequence: Synthetic oligonucleotide
  source 1..30: mol_type = other DNA
gttggacggctggggtaatgaaagttgtca

SEQ ID NO: 459 - DNA, 30 bp, synthetic construct
  misc_feature 1..30: note = Description of Artificial Sequence: Synthetic oligonucleotide
  source 1..30: mol_type = other DNA
ccatcgccattaaggagtagttgttgtatg

SEQ ID NO: 460 - DNA, 30 bp, synthetic construct
  misc_feature 1..30: note = Description of Artificial Sequence: Synthetic oligonucleotide
  source 1..30: mol_type = other DNA
tgcgccacgagcaaaatgatggccactatt

SEQ ID NO: 461 - DNA, 30 bp, synthetic construct
  misc_feature 1..30: note = Description of Artificial Sequence: Synthetic oligonucleotide
  source 1..30: mol_type = other DNA
agtacatgtagtgcgccacgagcaaaatga

SEQ ID NO: 462 - DNA, 30 bp, synthetic construct
  misc_feature 1..30: note = Description of Artificial Sequence: Synthetic oligonucleotide
  source 1..30: mol_type = other DNA
ctcgtggcgcactacatgtacttgatccca

SEQ ID NO: 463 - DNA, 30 bp, synthetic construct
  misc_feature 1..30: note = Description of Artificial Sequence: Synthetic oligonucleotide
  source 1..30: mol_type = other DNA
cagcagctgcgcgtgctgcccagaagagaa

SEQ ID NO: 464 - DNA, 30 bp, synthetic construct
  misc_feature 1..30: note = Description of Artificial Sequence: Synthetic oligonucleotide
  source 1..30: mol_type = other DNA
atgtcagtcaccactattccatccacaaca

SEQ ID NO: 465 - DNA, 30 bp, synthetic construct
  misc_feature 1..30: note = Description of Artificial Sequence: Synthetic oligonucleotide
  source 1..30: mol_type = other DNA
ttgacccccaagtggagaaaaagatgggac

SEQ ID NO: 466 - DNA, 30 bp, synthetic construct
  misc_feature 1..30: note = Description of Artificial Sequence: Synthetic oligonucleotide
  source 1..30: mol_type = other DNA
ccccccacccccaggcggtccgcgacagta

SEQ ID NO: 467 - DNA, 30 bp, synthetic construct
  misc_feature 1..30: note = Description of Artificial Sequence: Synthetic oligonucleotide
  source 1..30: mol_type = other DNA
tgatcagggccccagcctccccccaccccc

SEQ ID NO: 468 - DNA, 30 bp, synthetic construct
  misc_feature 1..30: note = Description of Artificial Sequence: Synthetic oligonucleotide
  source 1..30: mol_type = other DNA
tcgcggaccgcctgggggtggggggaggct

SEQ ID NO: 469 - DNA, 30 bp, synthetic construct
  misc_feature 1..30: note = Description of Artificial Sequence: Synthetic oligonucleotide
  source 1..30: mol_type = other DNA
cgcggaccgcctgggggtggggggaggctg

SEQ ID NO: 470 - DNA, 30 bp, synthetic construct
  misc_feature 1..30: note = Description of Artificial Sequence: Synthetic oligonucleotide
  source 1..30: mol_type = other DNA
cccacaaagtggaagttgcggctgtgatca

SEQ ID NO: 471 - DNA, 30 bp, synthetic construct
  misc_feature 1..30: note = Description of Artificial Sequence: Synthetic oligonucleotide
  source 1..30: mol_type = other DNA
tcccacaaagtggaagttgcggctgtgatc

SEQ ID NO: 472 - DNA, 30 bp, synthetic construct
  misc_feature 1..30: note = Description of Artificial Sequence: Synthetic oligonucleotide
  source 1..30: mol_type = other DNA
tcggagagccttcccacaaagtggaagttg

SEQ ID NO: 473 - DNA, 30 bp, synthetic construct
  misc_feature 1..30: note = Description of Artificial Sequence: Synthetic oligonucleotide
  source 1..30: mol_type = other DNA
tctacagccacttcactgtgtaacattttt

SEQ ID NO: 474 - DNA, 30 bp, synthetic construct
  misc_feature 1..30: note = Description of Artificial Sequence: Synthetic oligonucleotide
  source 1..30: mol_type = other DNA
ctacagccacttcactgtgtaacattttta

SEQ ID NO: 475 - DNA, 30 bp, synthetic construct
  misc_feature 1..30: note = Description of Artificial Sequence: Synthetic oligonucleotide
  source 1..30: mol_type = other DNA
taatctacacagtaacaagaaacgctggct

SEQ ID NO: 476 - DNA, 30 bp, synthetic construct
  misc_feature 1..30: note = Description of Artificial Sequence: Synthetic oligonucleotide
  source 1..30: mol_type = other DNA
acaagaaacgctggcttggtcaagagacgt

SEQ ID NO: 477 - DNA, 30 bp, synthetic construct
  misc_feature 1..30: note = Description of Artificial Sequence: Synthetic oligonucleotide
  source 1..30: mol_type = other DNA
caagaaacgctggcttggtcaagagacgtg

SEQ ID NO: 478 - DNA, 30 bp, synthetic construct
  misc_feature 1..30: note = Description of Artificial Sequence: Synthetic oligonucleotide
  source 1..30: mol_type = other DNA
agagacgtgggggtggaacaggagagaccc

SEQ ID NO: 479 - DNA, 30 bp, synthetic construct
  misc_feature 1..30: note = Description of Artificial Sequence: Synthetic oligonucleotide
  source 1..30: mol_type = other DNA
ggttcaagcgggccttccatttctctccca

SEQ ID NO: 480 - DNA, 30 bp, synthetic construct
  misc_feature 1..30: note = Description of Artificial Sequence: Synthetic oligonucleotide
  source 1..30: mol_type = other DNA
tggttcaagcgggccttccatttctctccc

SEQ ID NO: 481 - DNA, 30 bp, synthetic construct
  misc_feature 1..30: note = Description of Artificial Sequence: Synthetic oligonucleotide
  source 1..30: mol_type = other DNA
gaacaggagagaccctgggagagaaatgga

SEQ ID NO: 482 - DNA, 30 bp, synthetic construct
  misc_feature 1..30: note = Description of Artificial Sequence: Synthetic oligonucleotide
  source 1..30: mol_type = other DNA
agaactccagggccgacatctggttcaagc

SEQ ID NO: 483 - DNA, 30 bp, synthetic construct
  misc_feature 1..30: note = Description of Artificial Sequence: Synthetic oligonucleotide
  source 1..30: mol_type = other DNA
tagaactccagggccgacatctggttcaag

SEQ ID NO: 484 - DNA, 30 bp, synthetic construct
  misc_feature 1..30: note = Description of Artificial Sequence: Synthetic oligonucleotide
  source 1..30: mol_type = other DNA
ttgtaggagtagaactccagggccgacatc

SEQ ID NO: 485 - DNA, 30 bp, synthetic construct
  misc_feature 1..30: note = Description of Artificial Sequence: Synthetic oligonucleotide
  source 1..30: mol_type = other DNA
tgcacacctcggtgatgcctgactttttgt

SEQ ID NO: 486 - DNA, 30 bp, synthetic construct
  misc_feature 1..30: note = Description of Artificial Sequence: Synthetic oligonucleotide
  source 1..30: mol_type = other DNA
tctactcctacaaaaagtcaggcatcaccg

SEQ ID NO: 487 - DNA, 30 bp, synthetic construct
  misc_feature 1..30: note = Description of Artificial Sequence: Synthetic oligonucleotide
  source 1..30: mol_type = other DNA
gggcgcggcgggcctcttctctgcacacct

SEQ ID NO: 488 - DNA, 30 bp, synthetic construct
  misc_feature 1..30: note = Description of Artificial Sequence: Synthetic oligonucleotide
  source 1..30: mol_type = other DNA
ccgttgccacaccgtccttgagggcgcggc

SEQ ID NO: 489 - DNA, 30 bp, synthetic construct
  misc_feature 1..30: note = Description of Artificial Sequence: Synthetic oligonucleotide
  source 1..30: mol_type = other DNA
cccgttgccacaccgtccttgagggcgcgg

SEQ ID NO: 490 - DNA, 30 bp, synthetic construct
  misc_feature 1..30: note = Description of Artificial Sequence: Synthetic oligonucleotide
  source 1..30: mol_type = other DNA
cccgccgcgccctcaaggacggtgtggcaa

SEQ ID NO: 491 - DNA, 30 bp, synthetic construct
  misc_feature 1..30: note = Description of Artificial Sequence: Synthetic oligonucleotide
  source 1..30: mol_type = other DNA
ctcagctttgcacttcctcgggacacagca

SEQ ID NO: 492 - DNA, 30 bp, synthetic construct
  misc_feature 1..30: note = Description of Artificial Sequence: Synthetic oligonucleotide
  source 1..30: mol_type = other DNA
ggaagtgcaaagctgagatggttggtggag

SEQ ID NO: 493 - DNA, 30 bp, synthetic construct
  misc_feature 1..30: note = Description of Artificial Sequence: Synthetic oligonucleotide
  source 1..30: mol_type = other DNA
gaagtgcaaagctgagatggttggtggagc

SEQ ID NO: 494 - DNA, 30 bp, synthetic construct
  misc_feature 1..30: note = Description of Artificial Sequence: Synthetic oligonucleotide
  source 1..30: mol_type = other DNA
ctttgcaccaacacgggttcttcatgacca

SEQ ID NO: 495 - DNA, 30 bp, synthetic construct
  misc_feature 1..30: note = Description of Artificial Sequence: Synthetic oligonucleotide
  source 1..30: mol_type = other DNA
atatgaaagacgtccaccccactcttaaga

SEQ ID NO: 496 - DNA, 30 bp, synthetic construct
  misc_feature 1..30: note = Description of Artificial Sequence: Synthetic oligonucleotide
  source 1..30: mol_type = other DNA
tatgggtggaacatagtccgtcttaagagt

SEQ ID NO: 497 - DNA, 30 bp, synthetic construct
  misc_feature 1..30: note = Description of Artificial Sequence: Synthetic oligonucleotide
  source 1..30: mol_type = other DNA
atgggtggaacatagtccgtcttaagagtg

SEQ ID NO: 498 - DNA, 30 bp, synthetic construct
  misc_feature 1..30: note = Description of Artificial Sequence: Synthetic oligonucleotide
  source 1..30: mol_type = other DNA
tcatcatctagtcctgaagtggaagaagca

SEQ ID NO: 499 - DNA, 30 bp, synthetic construct
  misc_feature 1..30: note = Description of Artificial Sequence: Synthetic oligonucleotide
  source 1..30: mol_type = other DNA
cttttttcaagccaatcccccaccatggag

SEQ ID NO: 500 - DNA, 30 bp, synthetic construct
  misc_feature 1..30: note = Description of Artificial Sequence: Synthetic oligonucleotide
  source 1..30: mol_type = other DNA
ctggtcttttttcaagccaatcccccacca

SEQ ID NO: 501 - DNA, 30 bp, synthetic construct
  misc_feature 1..30: note = Description of Artificial Sequence: Synthetic oligonucleotide
  source 1..30: mol_type = other DNA
tccagggtttccatcatagtgctggtgtat

SEQ ID NO: 502 - DNA, 30 bp, synthetic construct
  misc_feature 1..30: note = Description of Artificial Sequence: Synthetic oligonucleotide
  source 1..30: mol_type = other DNA
cctcccccatacctacgctgcagtcgctcc

SEQ ID NO: 503 - DNA, 30 bp, synthetic construct
  misc_feature 1..30: note = Description of Artificial Sequence: Synthetic oligonucleotide
  source 1..30: mol_type = other DNA
agcgactgcagcgtaggtatgggggaggac

SEQ ID NO: 504 - DNA, 30 bp, synthetic construct
  misc_feature 1..30: note = Description of Artificial Sequence: Synthetic oligonucleotide
  source 1..30: mol_type = other DNA
agacccagtacatctcatgtgtagagttgc

SEQ ID NO: 505 - DNA, 30 bp, synthetic construct
  misc_feature 1..30: note = Description of Artificial Sequence: Synthetic oligonucleotide
  source 1..30: mol_type = other DNA
gagacccagtacatctcatgtgtagagttg

SEQ ID NO: 506 - DNA, 30 bp, synthetic construct
  misc_feature 1..30: note = Description of Artificial Sequence: Synthetic oligonucleotide
  source 1..30: mol_type = other DNA
ggagctggctcgtggtggacacacttttta

SEQ ID NO: 507 - DNA, 30 bp, synthetic construct
  misc_feature 1..30: note = Description of Artificial Sequence: Synthetic oligonucleotide
  source 1..30: mol_type = other DNA
ctaggcccgtccatgcgccccaagaggagc

SEQ ID NO: 508 - DNA, 30 bp, synthetic construct
  misc_feature 1..30: note = Description of Artificial Sequence: Synthetic oligonucleotide
  source 1..30: mol_type = other DNA
agtgtgtccaccacgagccagctcctcttg

SEQ ID NO: 509 - DNA, 30 bp, synthetic construct
  misc_feature 1..30: note = Description of Artificial Sequence: Synthetic oligonucleotide
  source 1..30: mol_type = other DNA
acgagccagctcctcttggggcgcatggac

SEQ ID NO: 510 - DNA, 30 bp, synthetic construct
  misc_feature 1..30: note = Description of Artificial Sequence: Synthetic oligonucleotide
  source 1..30: mol_type = other DNA
acgggcctaggaggccagtgaaatatgagg

SEQ ID NO: 511 - DNA, 30 bp, synthetic construct
  misc_feature 1..30: note = Description of Artificial Sequence: Synthetic oligonucleotide
  source 1..30: mol_type = other DNA
gaggaggatgtgaatctcggctctggcacg

SEQ ID NO: 512 - DNA, 30 bp, synthetic construct
  misc_feature 1..30: note = Description of Artificial Sequence: Synthetic oligonucleotide
  source 1..30: mol_type = other DNA
aggaggatgtgaatctcggctctggcacgc

SEQ ID NO: 513 - DNA, 30 bp, synthetic construct
  misc_feature 1..30: note = Description of Artificial Sequence: Synthetic oligonucleotide
  source 1..30: mol_type = other DNA
tccgcgtgctcactgcggatcctttcaatg

SEQ ID NO: 514 - DNA, 30 bp, synthetic construct
  misc_feature 1..30: note = Description of Artificial Sequence: Synthetic oligonucleotide
  source 1..30: mol_type = other DNA
aacatgaagatcattggtaaccgcattgaa

SEQ ID NO: 515 - DNA, 30 bp, synthetic construct
  misc_feature 1..30: note = Description of Artificial Sequence: Synthetic oligonucleotide
  source 1..30: mol_type = other DNA
accgcattgaaaggatccgcagtgagcacg

SEQ ID NO: 516 - DNA, 30 bp, synthetic construct
  misc_feature 1..30: note = Description of Artificial Sequence: Synthetic oligonucleotide
  source 1..30: mol_type = other DNA
tagcttccatggtaagcccatgtcctatat

SEQ ID NO: 517 - DNA, 30 bp, synthetic construct
  misc_feature 1..30: note = Description of Artificial Sequence: Synthetic oligonucleotide
  source 1..30: mol_type = other DNA
ataggacatgggcttaccatggaagctatg

SEQ ID NO: 518 - DNA, 30 bp, synthetic construct
  misc_feature 1..30: note = Description of Artificial Sequence: Synthetic oligonucleotide
  source 1..30: mol_type = other DNA
acaggagcctgacaaccccgtttattagag

SEQ ID NO: 519 - DNA, 30 bp, synthetic construct
  misc_feature 1..30: note = Description of Artificial Sequence: Synthetic oligonucleotide
  source 1..30: mol_type = other DNA
tcagcgtcctctctaataaacggggttgtc

SEQ ID NO: 520 - DNA, 30 bp, synthetic construct
  misc_feature 1..30: note = Description of Artificial Sequence: Synthetic oligonucleotide
  source 1..30: mol_type = other DNA
ttgtcaggctcctgtcaaaaccctgggatg

SEQ ID NO: 521 - DNA, 30 bp, synthetic construct
  misc_feature 1..30: note = Description of Artificial Sequence: Synthetic oligonucleotide
  source 1..30: mol_type = other DNA
tttgctgaccatacggtgtggtgtcggtca

SEQ ID NO: 522 - DNA, 30 bp, synthetic construct
  misc_feature 1..30: note = Description of Artificial Sequence: Synthetic oligonucleotide
  source 1..30: mol_type = other DNA
aaactctttgctgaccatacggtgtggtgt

SEQ ID NO: 523 - DNA, 30 bp, synthetic construct
  misc_feature 1..30: note = Description of Artificial Sequence: Synthetic oligonucleotide
  source 1..30: mol_type = other DNA
ccttgaaaactctttgctgaccatacggtg

SEQ ID NO: 524 - DNA, 30 bp, synthetic construct
  misc_feature 1..30: note = Description of Artificial Sequence: Synthetic oligonucleotide
  source 1..30: mol_type = other DNA
ccacaccgtatggtcagcaaagagttttca

SEQ ID NO: 525 - DNA, 30 bp, synthetic construct
  misc_feature 1..30: note = Description of Artificial Sequence: Synthetic oligonucleotide
  source 1..30: mol_type = other DNA
atgctcataacctgacgagtgccttcttgg

SEQ ID NO: 526 - DNA, 30 bp, synthetic construct
  misc_feature 1..30: note = Description of Artificial Sequence: Synthetic oligonucleotide
  source 1..30: mol_type = other DNA
atcaacaaggttcgtagcaatgcagcatta

SEQ ID NO: 527 - DNA, 30 bp, synthetic construct
  misc_feature 1..30: note = Description of Artificial Sequence: Synthetic oligonucleotide
  source 1..30: mol_type = other DNA
tcaacaaggttcgtagcaatgcagcattag

SEQ ID NO: 528 - DNA, 30 bp, synthetic construct
  misc_feature 1..30: note = Description of Artificial Sequence: Synthetic oligonucleotide
  source 1..30: mol_type = other DNA
tacacacaactctggcactctcctctcagg

SEQ ID NO: 529 - DNA, 30 bp, synthetic construct
  misc_feature 1..30: note = Description of Artificial Sequence: Synthetic oligonucleotide
  source 1..30: mol_type = other DNA
aacatgatgggaaaaagagaaaagaaacaa

SEQ ID NO: 530 - DNA, 30 bp, synthetic construct
  misc_feature 1..30: note = Description of Artificial Sequence: Synthetic oligonucleotide
  source 1..30: mol_type = other DNA
acatgatgggaaaaagagaaaagaaacaag

SEQ ID NO: 531 - DNA, 30 bp, synthetic construct
  misc_feature 1..30: note = Description of Artificial Sequence: Synthetic oligonucleotide
  source 1..30: mol_type = other DNA
aaagagaaaagaaacaaggggaatttggaa

SEQ ID NO: 532 - DNA, 30 bp, synthetic construct
  misc_feature 1..30: note = Description of Artificial Sequence: Synthetic oligonucleotide
  source 1..30: mol_type = other DNA
aaaagaaacaaggggaatttggaaaggcca

SEQ ID NO: 533 - DNA, 30 bp, synthetic construct
  misc_feature 1..30: note = Description of Artificial Sequence: Synthetic oligonucleotide
  source 1..30: mol_type = other DNA
gccacatataccagatggcgcggctgccct

SEQ ID NO: 534 - DNA, 30 bp, synthetic construct
  misc_feature 1..30: note = Description of Artificial Sequence: Synthetic oligonucleotide
  source 1..30: mol_type = other DNA
ggcagccgcgccatctggtatatgtggcta

SEQ ID NO: 535 - DNA, 30 bp, synthetic construct
  misc_feature 1..30: note = Description of Artificial Sequence: Synthetic oligonucleotide
  source 1..30: mol_type = other DNA
gcagccgcgccatctggtatatgtggctag

SEQ ID NO: 536 - DNA, 30 bp, synthetic construct
  misc_feature 1..30: note = Description of Artificial Sequence: Synthetic oligonucleotide
  source 1..30: mol_type = other DNA
cttggattcttgaacgaggatcactggatg

SEQ ID NO: 537 - DNA, 30 bp, synthetic construct
  misc_feature 1..30: note = Description of Artificial Sequence: Synthetic oligonucleotide
  source 1..30: mol_type = other DNA
gagagaactcaggaggtggtgttgaagggc

SEQ ID NO: 538 - DNA, 30 bp, synthetic construct
  misc_feature 1..30: note = Description of Artificial Sequence: Synthetic oligonucleotide
  source 1..30: mol_type = other DNA
cttcctcctggtatacgactcatctcttct

SEQ ID NO: 539 - DNA, 30 bp, synthetic construct
  misc_feature 1..30: note = Description of Artificial Sequence: Synthetic oligonucleotide
  source 1..30: mol_type = other DNA
ctagaagagatgagtcgtataccaggagga

SEQ ID NO: 540 - DNA, 30 bp, synthetic construct
  misc_feature 1..30: note = Description of Artificial Sequence: Synthetic oligonucleotide
  source 1..30: mol_type = other DNA
gacactgctggctgggacacccgcattagc

SEQ ID NO: 541 - DNA, 30 bp, synthetic construct
  misc_feature 1..30: note = Description of Artificial Sequence: Synthetic oligonucleotide
  source 1..30: mol_type = other DNA
ctaatcaccaaccaaatggagaaagggcac

SEQ ID NO: 542 - DNA, 30 bp, synthetic construct
  misc_feature 1..30: note = Description of Artificial Sequence: Synthetic oligonucleotide
  source 1..30: mol_type = other DNA
taatcaccaaccaaatggagaaagggcaca

SEQ ID NO: 543 - DNA, 30 bp, synthetic construct
  misc_feature 1..30: note = Description of Artificial Sequence: Synthetic oligonucleotide
  source 1..30: mol_type = other DNA
ggtatgtgtacttgattatggccaatgcca

SEQ ID NO: 544 - DNA, 30 bp, synthetic construct
  misc_feature 1..30: note = Description of Artificial Sequence: Synthetic oligonucleotide
  source 1..30: mol_type = other DNA
ccactttgttttggtatgtgtacttgatta

SEQ ID NO: 545 - DNA, 30 bp, synthetic construct
  misc_feature 1..30: note = Description of Artificial Sequence: Synthetic oligonucleotide
  source 1..30: mol_type = other DNA
gctggtctaaggacctttaccactttgttt

SEQ ID NO: 546 - DNA, 30 bp, synthetic construct
  misc_feature 1..30: note = Description of Artificial Sequence: Synthetic oligonucleotide
  source 1..30: mol_type = other DNA
gttatggacattatttcgagacaagaccaa

SEQ ID NO: 547 - DNA, 30 bp, synthetic construct
  misc_feature 1..30: note = Description of Artificial Sequence: Synthetic oligonucleotide
  source 1..30: mol_type = other DNA
ctagagatgcaagacttgtggctgctgcgg

SEQ ID NO: 548 - DNA, 30 bp, synthetic construct
  misc_feature 1..30: note = Description of Artificial Sequence: Synthetic oligonucleotide
  source 1..30: mol_type = other DNA
actaactggttgcagagcaacggatgggat

SEQ ID NO: 549 - DNA, 30 bp, synthetic construct
  misc_feature 1..30: note = Description of Artificial Sequence: Synthetic oligonucleotide
  source 1..30: mol_type = other DNA
ggttcttgaatgatatgggaaaagttagga

SEQ ID NO: 550 - DNA, 30 bp, synthetic construct
  misc_feature 1..30: note = Description of Artificial Sequence: Synthetic oligonucleotide
  source 1..30: mol_type = other DNA
acacacaagagtggaaaccctcaactggat

SEQ ID NO: 551 - DNA, 30 bp, synthetic construct
  misc_feature 1..30: note = Description of Artificial Sequence: Synthetic oligonucleotide
  source 1..30: mol_type = other DNA
ggaaccacaatggacctcccgtccttgaga

SEQ ID NO: 552 - DNA, 30 bp, synthetic construct
  misc_feature 1..30: note = Description of Artificial Sequence: Synthetic oligonucleotide
  source 1..30: mol_type = other DNA
caccacttcaacaagctccatctcaaggac

SEQ ID NO: 553 - DNA, 30 bp, synthetic construct
  misc_feature 1..30: note = Description of Artificial Sequence: Synthetic oligonucleotide
  source 1..30: mol_type = other DNA
cacttcaacaagctccatctcaaggacggg

SEQ ID NO: 554 - DNA, 30 bp, synthetic construct
  misc_feature 1..30: note = Description of Artificial Sequence: Synthetic oligonucleotide
  source 1..30: mol_type = other DNA
cccggccaatcagttcatcttggtggcggc

SEQ ID NO: 555 - DNA, 30 bp, synthetic construct
  misc_feature 1..30: note = Description of Artificial Sequence: Synthetic oligonucleotide
  source 1..30: mol_type = other DNA
ccctgccgccaccaagatgaactgattggc

SEQ ID NO: 556 - DNA, 30 bp, synthetic construct
  misc_feature 1..30: note = Description of Artificial Sequence: Synthetic oligonucleotide
  source 1..30: mol_type = other DNA
cctgccgccaccaagatgaactgattggcc

SEQ ID NO: 557 - DNA, 30 bp, synthetic construct
  misc_feature 1..30: note = Description of Artificial Sequence: Synthetic oligonucleotide
  source 1..30: mol_type = other DNA
tgattggccgggcccgcgtctctccagggg

SEQ ID NO: 558 - DNA, 30 bp, synthetic construct
  misc_feature 1..30: note = Description of Artificial Sequence: Synthetic oligonucleotide
  source 1..30: mol_type = other DNA
gcatatgattttgctaggcaagcagtctcc

SEQ ID NO: 559 - DNA, 30 bp, synthetic construct
  misc_feature 1..30: note = Description of Artificial Sequence: Synthetic oligonucleotide
  source 1..30: mol_type = other DNA
agctgccacatttgcgcatatgattttgct

SEQ ID NO: 560 - DNA, 30 bp, synthetic construct
  misc_feature 1..30: note = Description of Artificial Sequence: Synthetic oligonucleotide
  source 1..30: mol_type = other DNA
atcagtcggaggtcccttctgtggaaataa

SEQ ID NO: 561 - DNA, 30 bp, synthetic construct
  misc_feature 1..30: note = Description of Artificial Sequence: Synthetic oligonucleotide
  source 1..30: mol_type = other DNA
caaatgtggcagctcctttatttccacaga

SEQ ID NO: 562 - DNA, 30 bp, synthetic construct
  misc_feature 1..30: note = Description of Artificial Sequence: Synthetic oligonucleotide
  source 1..30: mol_type = other DNA
aaatgtggcagctcctttatttccacagaa

SEQ ID NO: 563 - DNA, 30 bp, synthetic construct
  misc_feature 1..30: note = Description of Artificial Sequence: Synthetic oligonucleotide
  source 1..30: mol_type = other DNA
acagatgaacaaatggcattggccatcagt

SEQ ID NO: 564 - DNA, 30 bp, synthetic construct
  misc_feature 1..30: note = Description of Artificial Sequence: Synthetic oligonucleotide
  source 1..30: mol_type = other DNA
gaacccagtcaactggcacagatgaacaaa

SEQ ID NO: 565 - DNA, 30 bp, synthetic construct
  misc_feature 1..30: note = Description of Artificial Sequence: Synthetic oligonucleotide
  source 1..30: mol_type = other DNA
ctctgttccacaccacaagcatgtcttcag

SEQ ID NO: 566 - DNA, 30 bp, synthetic construct
  misc_feature 1..30: note = Description of Artificial Sequence: Synthetic oligonucleotide
  source 1..30: mol_type = other DNA
aatagggaatgtctgtccatttcgtaactg

SEQ ID NO: 567 - DNA, 30 bp, synthetic construct
  misc_feature 1..30: note = Description of Artificial Sequence: Synthetic oligonucleotide
  source 1..30: mol_type = other DNA
aaatagggaatgtctgtccatttcgtaact

SEQ ID NO: 568 - DNA, 30 bp, synthetic construct
  misc_feature 1..30: note = Description of Artificial Sequence: Synthetic oligonucleotide
  source 1..30: mol_type = other DNA
tgttgactgtgtttttaatgttctcagccc

SEQ ID NO: 569 - DNA, 30 bp, synthetic construct
  misc_feature 1..30: note = Description of Artificial Sequence: Synthetic oligonucleotide
  source 1..30: mol_type = other DNA
tcttcacccaagtagcgaacttgggtggat

SEQ ID NO: 570 - DNA, 30 bp, synthetic construct
  misc_feature 1..30: note = Description of Artificial Sequence: Synthetic oligonucleotide
  source 1..30: mol_type = other DNA
cactcccacgtctagtgacctccactgcca

SEQ ID NO: 571 - DNA, 30 bp, synthetic construct
  misc_feature 1..30: note = Description of Artificial Sequence: Synthetic oligonucleotide
  source 1..30: mol_type = other DNA
cttctagatctccgtgcttcaccttttttg

SEQ ID NO: 572 - DNA, 30 bp, synthetic construct
  misc_feature 1..30: note = Description of Artificial Sequence: Synthetic oligonucleotide
  source 1..30: mol_type = other DNA
ttcctgaatatccaattttcgactctaatc

SEQ ID NO: 573 - DNA, 30 bp, synthetic construct
  misc_feature 1..30: note = Description of Artificial Sequence: Synthetic oligonucleotide
  source 1..30: mol_type = other DNA
ctttttggctcgttgaacttcccaaaagcc

SEQ ID NO: 574 - DNA, 30 bp, synthetic construct
  misc_feature 1..30: note = Description of Artificial Sequence: Synthetic oligonucleotide
  source 1..30: mol_type = other DNA
tgtcgacagccggtttgtcctgtgccatta

SEQ ID NO: 575 - DNA, 30 bp, synthetic construct
  misc_feature 1..30: note = Description of Artificial Sequence: Synthetic oligonucleotide
  source 1..30: mol_type = other DNA
gttgtgttaccgtaatggcacaggacaaac

SEQ ID NO: 576 - DNA, 30 bp, synthetic construct
  misc_feature 1..30: note = Description of Artificial Sequence: Synthetic oligonucleotide
  source 1..30: mol_type = other DNA
taagatcctattgctatgaggcatcaatat

SEQ ID NO: 577 - DNA, 30 bp, synthetic construct
  misc_feature 1..30: note = Description of Artificial Sequence: Synthetic oligonucleotide
  source 1..30: mol_type = other DNA
caggacatgaaactgatgagaatagagcga

SEQ ID NO: 578 - DNA, 30 bp, synthetic construct
  misc_feature 1..30: note = Description of Artificial Sequence: Synthetic oligonucleotide
  source 1..30: mol_type = other DNA
cagccgggatcttggtgaatgtgaacgctg

SEQ ID NO: 579 - DNA, 30 bp, synthetic construct
  misc_feature 1..30: note = Description of Artificial Sequence: Synthetic oligonucleotide
  source 1..30: mol_type = other DNA
tgtccaccgccatctgagctggaaccttgc

SEQ ID NO: 580 - DNA, 30 bp, synthetic construct
  misc_feature 1..30: note = Description of Artificial Sequence: Synthetic oligonucleotide
  source 1..30: mol_type = other DNA
ggttctcacaaattctcattggaacgttgc

SEQ ID NO: 581 - DNA, 30 bp, synthetic construct
  misc_feature 1..30: note = Description of Artificial Sequence: Synthetic oligonucleotide
  source 1..30: mol_type = other DNA
acttctcaaagaaggaaacgagatgcggta

SEQ ID NO: 582 - DNA, 30 bp, synthetic construct
  misc_feature 1..30: note = Description of Artificial Sequence: Synthetic oligonucleotide
  source 1..30: mol_type = other DNA
gcagcagcagtcaagcaagcctgggragat

SEQ ID NO: 583 - DNA, 30 bp, synthetic construct
  misc_feature 1..30: note = Description of Artificial Sequence: Synthetic oligonucleotide
  source 1..30: mol_type = other DNA
tttggccattcacatgttttcatctcgatc

SEQ ID NO: 584 - DNA, 30 bp, synthetic construct
  misc_feature 1..30: note = Description of Artificial Sequence: Synthetic oligonucleotide
  source 1..30: mol_type = other DNA
caaaggtggcacccatcaaaattgcaagct

SEQ ID NO: 585 - DNA, 30 bp, synthetic construct
  misc_feature 1..30: note = Description of Artificial Sequence: Synthetic oligonucleotide
  source 1..30: mol_type = other DNA
gagatcgcagtttgcagaagacacgaagcc

SEQ ID NO: 586 - DNA, 30 bp, synthetic construct
  misc_feature 1..30: note = Description of Artificial Sequence: Synthetic oligonucleotide
  source 1..30: mol_type = other DNA
tgcggggggttcatgcttctctctctgaag

SEQ ID NO: 587 - DNA, 30 bp, synthetic construct
  misc_feature 1..30: note = Description of Artificial Sequence: Synthetic oligonucleotide
  source 1..30: mol_type = other DNA
gggggttcatgcttctctctctgaagggga

SEQ ID NO: 588 - DNA, 30 bp, synthetic construct
  misc_feature 1..30: note = Description of Artificial Sequence: Synthetic oligonucleotide
  source 1..30: mol_type = other DNA
gcggtgagtcccaaggccatgacaaatggt

SEQ ID NO: 589 - DNA, 30 bp, synthetic construct
  misc_feature 1..30: note = Description of Artificial Sequence: Synthetic oligonucleotide
  source 1..30: mol_type = other DNA
tatgcgcgttggccggagggttcgccaagg

SEQ ID NO: 590 - DNA, 30 bp, synthetic construct
  misc_feature 1..30: note = Description of Artificial Sequence: Synthetic oligonucleotide
  source 1..30: mol_type = other DNA
ctggaaacagtccccggctcgatgtggcac

SEQ ID NO: 591 - DNA, 30 bp, synthetic construct
  misc_feature 1..30: note = Description of Artificial Sequence: Synthetic oligonucleotide
  source 1..30: mol_type = other DNA
gcggatccttttgtgacgtgccacatagtg

SEQ ID NO: 592 - DNA, 30 bp, synthetic construct
  misc_feature 1..30: note = Description of Artificial Sequence: Synthetic oligonucleotide
  source 1..30: mol_type = other DNA
actctcctggttttcccagctccaggatgc

SEQ ID NO: 593 - DNA, 30 bp, synthetic construct
  misc_feature 1..30: note = Description of Artificial Sequence: Synthetic oligonucleotide
  source 1..30: mol_type = other DNA
atgacaacagcagtcaatgtcacccattct

SEQ ID NO: 594 - DNA, 30 bp, synthetic construct
  misc_feature 1..30: note = Description of Artificial Sequence: Synthetic oligonucleotide
  source 1..30: mol_type = other DNA
agtccgggaatgcgtcacgggttcctggtg

SEQ ID NO: 595 - DNA, 30 bp, synthetic construct
  misc_feature 1..30: note = Description of Artificial Sequence: Synthetic oligonucleotide
  source 1..30: mol_type = other DNA
gagtccgggaatgcgtcacgggttcctggt

SEQ ID NO: 596 - DNA, 30 bp, synthetic construct
  misc_feature 1..30: note = Description of Artificial Sequence: Synthetic oligonucleotide
  source 1..30: mol_type = other DNA
tcggtgtccataattggtgagttggagtcc

SEQ ID NO: 597 - DNA, 30 bp, synthetic construct
  misc_feature 1..30: note = Description of Artificial Sequence: Synthetic oligonucleotide
  source 1..30: mol_type = other DNA
acaccataatggaagacagtgtgccggcag

SEQ ID NO: 598 - DNA, 30 bp, synthetic construct
  misc_feature 1..30: note = Description of Artificial Sequence: Synthetic oligonucleotide
  source 1..30: mol_type = other DNA
gcaatgttcccagggcttccatcactccaa

SEQ ID NO: 599 - DNA, 30 bp, synthetic construct
  misc_feature 1..30: note = Description of Artificial Sequence: Synthetic oligonucleotide
  source 1..30: mol_type = other DNA
ttggagtgatggaagccctgggaacattgc

SEQ ID NO: 600 - DNA, 30 bp, synthetic construct
  misc_feature 1..30: note = Description of Artificial Sequence: Synthetic oligonucleotide
  source 1..30: mol_type = other DNA
accaaatggcaatcatcatcatgatagcag

SEQ ID NO: 601 - DNA, 30 bp, synthetic construct
  misc_feature 1..30: note = Description of Artificial Sequence: Synthetic oligonucleotide
  source 1..30: mol_type = other DNA
caactttcatcaccccagccgtccaacatg

SEQ ID NO: 602 - DNA, 29 bp, synthetic construct
  misc_feature 1..29: note = Description of Artificial Sequence: Synthetic oligonucleotide
  source 1..29: mol_type = other DNA
atgggactttggagtcccgctgctaatga

SEQ ID NO: 603 - DNA, 30 bp, synthetic construct
  misc_feature 1..30: note = Description of Artificial Sequence: Synthetic oligonucleotide
  source 1..30: mol_type = other DNA
ccctaaaaatgttacacagtgaagtggctg

SEQ ID NO: 604 - DNA, 30 bp, synthetic construct
  misc_feature 1..30: note = Description of Artificial Sequence: Synthetic oligonucleotide
  source 1..30: mol_type = other DNA
ctggcttggtcaagagacgtgggggtggaa

SEQ ID NO: 605 - DNA, 30 bp, synthetic construct
  misc_feature 1..30: note = Description of Artificial Sequence: Synthetic oligonucleotide
  source 1..30: mol_type = other DNA
gagtagaactctagggccgacatctggttc

SEQ ID NO: 606 - DNA, 30 bp, synthetic construct
  misc_feature 1..30: note = Description of Artificial Sequence: Synthetic oligonucleotide
  source 1..30: mol_type = other DNA
cttcagcgcagcttaccacagcccgcgtgc

SEQ ID NO: 607 - DNA, 30 bp, synthetic construct
  misc_feature 1..30: note = Description of Artificial Sequence: Synthetic oligonucleotide
  source 1..30: mol_type = other DNA
tctggtatatgtggctaggggctagattcc

SEQ ID NO: 608 - DNA, 30 bp, synthetic construct
  misc_feature 1..30: note = Description of Artificial Sequence: Synthetic oligonucleotide
  source 1..30: mol_type = other DNA
tcgttcaagaatccaagggcttcgaactcc

SEQ ID NO: 609 - DNA, 30 bp, synthetic construct
  misc_feature 1..30: note = Description of Artificial Sequence: Synthetic oligonucleotide
  source 1..30: mol_type = other DNA
tcacttacgctcttaatacattcaccaacc

SEQ ID NO: 610 - DNA, 30 bp, synthetic construct
  misc_feature 1..30: note = Description of Artificial Sequence: Synthetic oligonucleotide
  source 1..30: mol_type = other DNA
gctatgaatgccctatgctggatgaggggg

SEQ ID NO: 611 - DNA, 30 bp, synthetic construct; identical to SEQ ID NO: 317
  misc_feature 1..30: note = Description of Artificial Sequence: Synthetic oligonucleotide
  source 1..30: mol_type = other DNA

SEQ ID NO: 612 - DNA, 30 bp, synthetic construct; identical to SEQ ID NO: 320
  misc_feature 1..30: note = Description of Artificial Sequence: Synthetic oligonucleotide
  source 1..30: mol_type = other DNA

SEQ ID NO: 613 - DNA, 30 bp, synthetic construct; identical to SEQ ID NO: 333
  misc_feature 1..30: note = Description of Artificial Sequence: Synthetic oligonucleotide
  source 1..30: mol_type = other DNA

SEQ ID NO: 614 - DNA, 30 bp, synthetic construct; identical to SEQ ID NO: 334
  misc_feature 1..30: note = Description of Artificial Sequence: Synthetic oligonucleotide
  source 1..30: mol_type = other DNA

SEQ ID NO: 615 - DNA, 30 bp, synthetic construct
  misc_feature 1..30: note = Description of Artificial Sequence: Synthetic oligonucleotide
  source 1..30: mol_type = other DNA
aggcttgcttgactgctgctgccaatctac

SEQ ID NO: 616 - DNA, 30 bp, synthetic construct
  misc_feature 1..30: note = Description of Artificial Sequence: Synthetic oligonucleotide
  source 1..30: mol_type = other DNA
caggcttgcttgactgctgctgccaatcta

SEQ ID NO: 617 - DNA, 30 bp, synthetic construct
  misc_feature 1..30: note = Description of Artificial Sequence: Synthetic oligonucleotide
  source 1..30: mol_type = other DNA
acaagtaccatcctgactccccccgtagat

SEQ ID NO: 618 - DNA, 30 bp, synthetic construct; identical to SEQ ID NO: 352
  misc_feature 1..30: note = Description of Artificial Sequence: Synthetic oligonucleotide
  source 1..30: mol_type = other DNA

SEQ ID NO: 619 - DNA, 30 bp, synthetic construct; identical to SEQ ID NO: 353
  misc_feature 1..30: note = Description of Artificial Sequence: Synthetic oligonucleotide
  source 1..30: mol_type = other DNA

SEQ ID NO: 620 - DNA, 30 bp, synthetic construct
  misc_feature 1..30: note = Description of Artificial Sequence: Synthetic oligonucleotide
  source 1..30: mol_type = other DNA
agcagaatcacaagcactccaagggagaag

SEQ ID NO: 621 - DNA, 30 bp, synthetic construct; identical to SEQ ID NO: 368
  misc_feature 1..30: note = Description of Artificial Sequence: Synthetic oligonucleotide
  source 1..30: mol_type = other DNA

SEQ ID NO: 622 - DNA, 30 bp, synthetic construct
  misc_feature 1..30: note = Description of Artificial Sequence: Synthetic oligonucleotide
  source 1..30: mol_type = other DNA
cacgcactgataacatcaccttggcaatcc

SEQ ID NO: 623 - DNA, 30 bp, synthetic construct
  misc_feature 1..30: note = Description of Artificial Sequence: Synthetic oligonucleotide
  source 1..30: mol_type = other DNA
ttcttcacactgccttttcccttcagagag

SEQ ID NO: 624 - DNA, 30 bp, synthetic construct; identical to SEQ ID NO: 378
  misc_feature 1..30: note = Description of Artificial Sequence: Synthetic oligonucleotide
  source 1..30: mol_type = other DNA

SEQ ID NO: 625 - DNA, 30 bp, synthetic construct
  misc_feature 1..30: note = Description of Artificial Sequence: Synthetic oligonucleotide
  source 1..30: mol_type = other DNA
ttggcgaaccctccagccaatgcgcatatc

SEQ ID NO: 626 - DNA, 30 bp, synthetic construct
  misc_feature 1..30: note = Description of Artificial Sequence: Synthetic oligonucleotide
  source 1..30: mol_type = other DNA
tggcgctagatgagagtggtgatttctccc

SEQ ID NO: 627 - DNA, 30 bp, synthetic construct
  misc_feature 1..30: note = Description of Artificial Sequence: Synthetic oligonucleotide
  source 1..30: mol_type = other DNA
acggtccccccatgagagagatcatactca

SEQ ID NO: 628 - DNA, 30 bp, synthetic construct; identical to SEQ ID NO: 402
  misc_feature 1..30: note = Description of Artificial Sequence: Synthetic oligonucleotide
  source 1..30: mol_type = other DNA

SEQ ID NO: 629 - DNA, 30 bp, synthetic construct; identical to SEQ ID NO: 408
  misc_feature 1..30: note = Description of Artificial Sequence: Synthetic oligonucleotide
  source 1..30: mol_type = other DNA

SEQ ID NO: 630 - DNA, 30 bp, synthetic construct; identical to SEQ ID NO: 429
  misc_feature 1..30: note = Description of Artificial Sequence: Synthetic oligonucleotide
  source 1..30: mol_type = other DNA

SEQ ID NO: 631 - DNA, 30 bp, synthetic construct
  misc_feature 1..30: note = Description of Artificial Sequence: Synthetic oligonucleotide
  source 1..30: mol_type = other DNA
gctccgtcctaagcttgaactctccctcaa

SEQ ID NO: 632 - DNA, 30 bp, synthetic construct; identical to SEQ ID NO: 438
  misc_feature 1..30: note = Description of Artificial Sequence: Synthetic oligonucleotide
  source 1..30: mol_type = other DNA

SEQ ID NO: 633 - DNA, 30 bp, synthetic construct
  misc_feature 1..30: note = Description of Artificial Sequence: Synthetic oligonucleotide
  source 1..30: mol_type = other DNA
cccagcaaccccaaaagcataatggtctct

SEQ ID NO: 634 - DNA, 30 bp, synthetic construct
  misc_feature 1..30: note = Description of Artificial Sequence: Synthetic oligonucleotide
  source 1..30: mol_type = other DNA
cccaggacaaccaaatggcaatcatcatca

SEQ ID NO: 635 - DNA, 30 bp, synthetic construct; identical to SEQ ID NO: 457
  misc_feature 1..30: note = Description of Artificial Sequence: Synthetic oligonucleotide
  source 1..30: mol_type = other DNA

SEQ ID NO: 636 - DNA, 30 bp, synthetic construct; identical to SEQ ID NO: 458
  misc_feature 1..30: note = Description of Artificial Sequence: Synthetic oligonucleotide
  source 1..30: mol_type = other DNA

SEQ ID NO: 637 - DNA, 30 bp, synthetic construct; identical to SEQ ID NO: 459
  misc_feature 1..30: note = Description of Artificial Sequence: Synthetic oligonucleotide
  source 1..30: mol_type = other DNA

SEQ ID NO: 638 - DNA, 30 bp, synthetic construct
  misc_feature 1..30: note = Description of Artificial Sequence: Synthetic oligonucleotide
  source 1..30: mol_type = other DNA
tagcacctgtcccatctttttctccacttg

SEQ ID NO: 639 - DNA, 30 bp, synthetic construct
  misc_feature 1..30: note = Description of Artificial Sequence: Synthetic oligonucleotide
  source 1..30: mol_type = other DNA
gtagcacctgtcccatctttttctccactt

SEQ ID NO: 640 - DNA, 30 bp, synthetic construct; identical to SEQ ID NO: 472
  misc_feature 1..30: note = Description of Artificial Sequence: Synthetic oligonucleotide
  source 1..30: mol_type = other DNA

SEQ ID NO: 641 - DNA, 30 bp, synthetic construct
  misc_feature 1..30: note = Description of Artificial Sequence: Synthetic oligonucleotide
  source 1..30: mol_type = other DNA
ggaaggcccgcttgaaccagatgtcggccc

SEQ ID NO: 642 - DNA, 30 bp, synthetic construct; identical to SEQ ID NO: 490
  misc_feature 1..30: note = Description of Artificial Sequence: Synthetic oligonucleotide
  source 1..30: mol_type = other DNA

SEQ ID NO: 643 - DNA, 30 bp, synthetic construct; identical to SEQ ID NO: 492
  misc_feature 1..30: note = Description of Artificial Sequence: Synthetic oligonucleotide
  source 1..30: mol_type = other DNA

SEQ ID NO: 644 - DNA, 30 bp, synthetic construct; identical to SEQ ID NO: 493
  misc_feature 1..30: note = Description of Artificial Sequence: Synthetic oligonucleotide
  source 1..30: mol_type = other DNA

SEQ ID NO: 645 - DNA, 30 bp, synthetic construct
  misc_feature 1..30: note = Description of Artificial Sequence: Synthetic oligonucleotide
  source 1..30: mol_type = other DNA
cgtttattagagaggacgctgacccttgtg

SEQ ID NO: 646 - DNA, 30 bp, synthetic construct
  misc_feature 1..30: note = Description of Artificial Sequence: Synthetic oligonucleotide
  source 1..30: mol_type = other DNA
ctttttcccatcatgttgtacacacaactc

SEQ ID NO: 647 - DNA, 30 bp, synthetic construct; identical to SEQ ID NO: 555
  misc_feature 1..30: note = Description of Artificial Sequence: Synthetic oligonucleotide
  source 1..30: mol_type = other DNA

SEQ ID NO: 648 - DNA, 30 bp, synthetic construct; identical to SEQ ID NO: 556
  misc_feature 1..30: note = Description of Artificial Sequence: Synthetic oligonucleotide
  source 1..30: mol_type = other DNA

SEQ ID NO: 649 - DNA, 30 bp, synthetic construct
  misc_feature 1..30: note = Description of Artificial Sequence: Synthetic oligonucleotide
  source 1..30: mol_type = other DNA
gggctgagaacattaaaaacacagtcaaca

SEQ ID NO: 650 - RNA, 36 nt, Zika virus
  source 1..36: mol_type = genomic RNA
ttgagagtgagaagaatgacacatggaggctgaaga

SEQ ID NO: 651 - RNA, 36 nt, Zika virus
  source 1..36: mol_type = genomic RNA
atgatgggaaaaagagaaaagaaacaaggggaattt

SEQ ID NO: 652 - RNA, 36 nt, Zika virus
  source 1..36: mol_type = genomic RNA
gacacaggacatgaaactgatgagaatagagcgaaa

SEQ ID NO: 653 - RNA, 36 nt, Zika virus
  source 1..36: mol_type = genomic RNA
tggaaaacatcatgtggagatcagtagaaggggagc

SEQ ID NO: 654 - RNA, 36 nt, Zika virus
  source 1..36: mol_type = genomic RNA
ggggaaaaaagaggctatggaaataataaagaagtt

SEQ ID NO: 655 - RNA, 36 nt, Zika virus
  source 1..36: mol_type = genomic RNA
gataacgcccaattcaccaagagccgaagccaccct

SEQ ID NO: 656 - RNA, 36 nt, Zika virus
  source 1..36: mol_type = genomic RNA
ttgaagaggaaaaagagtggaagactgcagtggaag

SEQ ID NO: 657 - RNA, 36 nt, Zika virus
  source 1..36: mol_type = genomic RNA
ttttgctcgtggcgcactacatgtacttgatcccag

SEQ ID NO: 658 - RNA, 36 nt, Zika virus
  source 1..36: mol_type = genomic RNA
ttgctactcacaattaacacccctgaccctaatagt

SEQ ID NO: 659 - RNA, 36 nt, Zika virus
  source 1..36: mol_type = genomic RNA
accacctcatacaacaactactccttaatggcgatg

SEQ ID NO: 660 - RNA, 36 nt, Zika virus
  source 1..36: mol_type = genomic RNA
accacaaagatcatcataagcacatcaatggcagtg

SEQ ID NO: 661 - RNA, 36 nt, Zika virus
  source 1..36: mol_type = genomic RNA
agacagagttccagaaaacaaaacatcaagagtggg

SEQ ID NO: 662 - RNA, 36 nt, Zika virus
  source 1..36: mol_type = genomic RNA
tgcacaatgcccccactgtcgttccgggctaaagat

SEQ ID NO: 663 - RNA, 36 nt, Zika virus
  source 1..36: mol_type = genomic RNA
gacaccggaactccacactggaacaacaaagaagca

SEQ ID NO: 664 - RNA, 36 nt, Zika virus
  source 1..36: mol_type = genomic RNA
gtttgccgctgggaaaagaggagcggcttttggagt

SEQ ID NO: 665 - RNA, 36 nt, Zika virus
  source 1..36: mol_type = genomic RNA
ggcagaagtgtggaccagacacggagagaaaagagt

SEQ ID NO: 666 - RNA, 36 nt, Zika virus
  source 1..36: mol_type = genomic RNA
ctgatcgagatgaaaacatgtgaatggccaaagtcc

SEQ ID NO: 667 - RNA, 36 nt, Zika virus
  source 1..36: mol_type = genomic RNA
gagccagaaaagcaaagatctccccaggacaaccaa

SEQ ID NO: 668 - RNA, 36 nt, Zika virus
  source 1..36: mol_type = genomic RNA
cttaacacatttaccaacctagtggtgcaactcatt

SEQ ID NO: 669 - RNA, 36 nt, Zika virus
  source 1..36: mol_type = genomic RNA
gcggtacaggggtgttcgtctataacgacgttgaag

SEQ ID NO: 670 - RNA, 36 nt, Zika virus
  source 1..36: mol_type = genomic RNA
gaggctgaggaagttctagagatgcaagacttgtgg

SEQ ID NO: 671 - RNA, 36 nt, Zika virus
  source 1..36: mol_type = genomic RNA
ggcacagtgaagagcttgaaattcggtttgaggaat

SEQ ID NO: 672 - RNA, 36 nt, Zika virus
  source 1..36: mol_type = genomic RNA
catctaatgggaaggagagaggagggggcaaccata

SEQ ID NO: 673 - RNA, 36 nt, Zika virus
  source 1..36: mol_type = genomic RNA
gataggtttgcacatgccctcaggttcttgaatgat

SEQ ID NO: 674 - RNA, 107 nt, synthetic construct
  misc_feature 1..107: note = Description of Artificial Sequence: Synthetic polynucleotide
  source 1..107: mol_type = other RNA
tcttcagcctccatgtgtcattcttctcactctcaagttatagttatgaacagaggagacataacatgaacttgagaaaccaagttaacctggcggcagcgcaaaag

SEQ ID NO: 675 - RNA, 107 nt, synthetic construct
  misc_feature 1..107: note = Description of Artificial Sequence: Synthetic polynucleotide
  source 1..107: mol_type = other RNA
aaattccccttgtttcttttctctttttcccatcatgttatagttatgaacagaggagacataacatgaacatgatgaaccatgttaacctggcggcagcgcaaaag

SEQ ID NO: 676 - RNA, 107 nt, synthetic construct
  misc_feature 1..107: note = Description of Artificial Sequence: Synthetic polynucleotide
  source 1..107: mol_type = other RNA
tttcgctctattctcatcagtttcatgtcctgtgtcgttatagttatgaacagaggagacataacatgaacgacacaaacgtcgttaacctggcggcagcgcaaaag

SEQ ID NO: 677 - RNA, 107 nt, synthetic construct
  misc_feature 1..107: note = Description of Artificial Sequence: Synthetic polynucleotide
  source 1..107: mol_type = other RNA
gctccccttctactgatctccacatgatgttttccagttatagttatgaacagaggagacataacatgaactggaaaaacccagttaacctggcggcagcgcaaaag

SEQ ID NO: 678 - RNA, 107 nt, synthetic construct
  misc_feature 1..107: note = Description of Artificial Sequence: Synthetic polynucleotide
  source 1..107: mol_type = other RNA
aacttctttattatttccatagcctcttttttccccgttatagttatgaacagaggagacataacatgaacggggaaaaccccgttaacctggcggcagcgcaaaag

SEQ ID NO: 679 - RNA, 107 nt, synthetic construct
  misc_feature 1..107: note = Description of Artificial Sequence: Synthetic polynucleotide
  source 1..107: mol_type = other RNA
agggtggcttcggctcttggtgaattgggcgttatcgttatagttatgaacagaggagacataacatgaacgataacaacatcgttaacctggcggcagcgcaaaag

SEQ ID NO: 680 - RNA, 107 nt, synthetic construct
  misc_feature 1..107: note = Description of Artificial Sequence: Synthetic polynucleotide
  source 1..107: mol_type = other RNA
cttccactgcagtcttccactctttttcctcttcaagttatagttatgaacagaggagacataacatgaacttgaagaaccaagttaacctggcggcagcgcaaaag

SEQ ID NO: 681 - RNA, 107 nt, synthetic construct
  misc_feature 1..107: note = Description of Artificial Sequence: Synthetic polynucleotide
  source 1..107: mol_type = other RNA
ctgggatcaagtacatgtagtgcgccacgagcaaaagttatagttatgaacagaggagacataacatgaacttttgcaacaaagttaacctggcggcagcgcaaaag

SEQ ID NO: 682 - RNA, 107 nt, synthetic construct
  misc_feature 1..107: note = Description of Artificial Sequence: Synthetic polynucleotide
  source 1..107: mol_type = other RNA
actattagggtcaggggtgttaattgtgagtagcaagttatagttatgaacagaggagacataacatgaacttgctaaaccaagttaacctggcggcagcgcaaaag

SEQ ID NO: 683 - RNA, 107 nt, synthetic construct
  misc_feature 1..107: note = Description of Artificial Sequence: Synthetic polynucleotide
  source 1..107: mol_type = other RNA
catcgccattaaggagtagttgttgtatgaggtggtgttatagttatgaacagaggagacataacatgaacaccaccaacggtgttaacctggcggcagcgcaaaag

SEQ ID NO: 684 - RNA, 107 nt, synthetic construct
  misc_feature 1..107: note = Description of Artificial Sequence: Synthetic polynucleotide
  source 1..107: mol_type = other RNA
cactgccattgatgtgcttatgatgatctttgtggtgttatagttatgaacagaggagacataacatgaacaccacaaacggtgttaacctggcggcagcgcaaaag

SEQ ID NO: 685 - RNA, 107 nt, synthetic construct
  misc_feature 1..107: note = Description of Artificial Sequence: Synthetic polynucleotide
  source 1..107: mol_type = other RNA
cccactcttgatgttttgttttctggaactctgtctgttatagttatgaacagaggagacataacatgaacagacagaactctgttaacctggcggcagcgcaaaag

SEQ ID NO: 686 - RNA, 107 nt, synthetic construct
  misc_feature 1..107: note = Description of Artificial Sequence: Synthetic polynucleotide
  source 1..107: mol_type = other RNA
atctttagcccggaacgacagtgggggcattgtgcagttatagttatgaacagaggagacataacatgaactgcacaaacgcagttaacctggcggcagcgcaaaag

SEQ ID NO: 687 - RNA, 107 nt, synthetic construct
  misc_feature 1..107: note = Description of Artificial Sequence: Synthetic polynucleotide
  source 1..107: mol_type = other RNA
tgcttctttgttgttccagtgtggagttccggtgtcgttatagttatgaacagaggagacataacatgaacgacaccaacgtcgttaacctggcggcagcgcaaaag

SEQ ID NO: 688 - RNA, 107 nt, synthetic construct
  misc_feature 1..107: note = Description of Artificial Sequence: Synthetic polynucleotide
  source 1..107: mol_type = other RNA
actccaaaagccgctcctcttttcccagcggcaaacgttatagttatgaacagaggagacataacatgaacgtttgcaacaacgttaacctggcggcagcgcaaaag

SEQ ID NO: 689 - RNA, 107 nt, synthetic construct
  misc_feature 1..107: note = Description of Artificial Sequence: Synthetic polynucleotide
  source 1..107: mol_type = other RNA
actcttttctctccgtgtctggtccacacttctgccgttatagttatgaacagaggagacataacatgaacggcagaaacgccgttaacctggcggcagcgcaaaag

SEQ ID NO: 690 - RNA, 107 nt, synthetic construct
  misc_feature 1..107: note = Description of Artificial Sequence: Synthetic polynucleotide
  source 1..107: mol_type = other RNA
ggactttggccattcacatgttttcatctcgatcaggttatagttatgaacagaggagacataacatgaacctgatcaaccaggttaacctggcggcagcgcaaaag

SEQ ID NO: 691 - RNA, 107 nt, synthetic construct
  misc_feature 1..107: note = Description of Artificial Sequence: Synthetic polynucleotide
  source 1..107: mol_type = other RNA
ttggttgtcctggggagatctttgcttttctggctcgttatagttatgaacagaggagacataacatgaacgagccaaacctcgttaacctggcggcagcgcaaaag

SEQ ID NO: 692 - RNA, 107 nt, synthetic construct
  misc_feature 1..107: note = Description of Artificial Sequence: Synthetic polynucleotide
  source 1..107: mol_type = other RNA
aatgagttgcaccactaggttggtaaatgtgttaaggttatagttatgaacagaggagacataacatgaaccttaacaacaaggttaacctggcggcagcgcaaaag

SEQ ID NO: 693 - RNA, 107 nt, synthetic construct
  misc_feature 1..107: note = Description of Artificial Sequence: Synthetic polynucleotide
  source 1..107: mol_type = other RNA
cttcaacgtcgttatagacgaacacccctgtaccgcgttatagttatgaacagaggagacataacatgaacgcggtaaaccgcgttaacctggcggcagcgcaaaag

SEQ ID NO: 694 - RNA, 107 nt, synthetic construct
  misc_feature 1..107: note = Description of Artificial Sequence: Synthetic polynucleotide
  source 1..107: mol_type = other RNA
ccacaagtcttgcatctctagaacttcctcagcctcgttatagttatgaacagaggagacataacatgaacgaggctaacctcgttaacctggcggcagcgcaaaag

SEQ ID NO: 695 - RNA, 107 nt, synthetic construct
  misc_feature 1..107: note = Description of Artificial Sequence: Synthetic polynucleotide
  source 1..107: mol_type = other RNA
attcctcaaaccgaatttcaagctcttcactgtgccgttatagttatgaacagaggagacataacatgaacggcacaaacgccgttaacctggcggcagcgcaaaag

SEQ ID NO: 696 - RNA, 107 nt, synthetic construct
  misc_feature 1..107: note = Description of Artificial Sequence: Synthetic polynucleotide
  source 1..107: mol_type = other RNA
tatggttgccccctcctctctccttcccattagatggttatagttatgaacagaggagacataacatgaaccatctaaacatggttaacctggcggcagcgcaaaag

SEQ ID NO: 697 - RNA, 107 nt, synthetic construct
  misc_feature 1..107: note = Description of Artificial Sequence: Synthetic polynucleotide
  source 1..107: mol_type = other RNA
atcattcaagaacctgagggcatgtgcaaacctatcgttatagttatgaacagaggagacataacatgaacgataggaacatcgttaacctggcggcagcgcaaaag

SEQ ID NO: 698 - RNA, 97 nt, synthetic construct
  misc_feature 1..97: note = Description of Artificial Sequence: Synthetic oligonucleotide
  source 1..97: mol_type = other RNA
tcttcagcctccatgtgtcattcttctcactctcaaggactttagaacagaggagataaagatgttgagagtgagtaacctggcggcagcgcaaaag

SEQ ID NO: 699 - RNA, 97 nt, synthetic construct
  misc_feature 1..97: note = Description of Artificial Sequence: Synthetic oligonucleotide
  source 1..97: mol_type = other RNA
aaattccccttgtttcttttctctttttcccatcatggactttagaacagaggagataaagatgatgatgggaaaaaacctggcggcagcgcaaaag

SEQ ID NO: 700 - RNA, 97 nt, synthetic construct
  misc_feature 1..97: note = Description of Artificial Sequence: Synthetic oligonucleotide
  source 1..97: mol_type = other RNA
tttcgctctattctcatcagtttcatgtcctgtgtcggactttagaacagaggagataaagatggacacaggacacaacctggcggcagcgcaaaag

SEQ ID NO: 701 - RNA, 97 nt, synthetic construct
  misc_feature 1..97: note = Description of Artificial Sequence: Synthetic oligonucleotide
  source 1..97: mol_type = other RNA
gctccccttctactgatctccacatgatgttttccaggactttagaacagaggagataaagatgtggaaaacatcaaacctggcggcagcgcaaaag

SEQ ID NO: 702 - RNA, 97 nt, synthetic construct
  misc_feature 1..97: note = Description of Artificial Sequence: Synthetic oligonucleotide
  source 1..97: mol_type = other RNA
aacttctttattatttccatagcctcttttttccccggactttagaacagaggagataaagatgggggaaaaaagaaacctggcggcagcgcaaaag

SEQ ID NO: 703 - RNA, 97 nt, synthetic construct
  misc_feature 1..97: note = Description of Artificial Sequence: Synthetic oligonucleotide
  source 1..97: mol_type = other RNA
agggtggcttcggctcttggtgaattgggcgttatcggactttagaacagaggagataaagatggataacgcccataacctggcggcagcgcaaaag

SEQ ID NO: 704 - RNA, 97 nt, synthetic construct
  misc_feature 1..97: note = Description of Artificial Sequence: Synthetic oligonucleotide
  source 1..97: mol_type = other RNA
cttccactgcagtcttccactctttttcctcttcaaggactttagaacagaggagataaagatgttgaagaggaaaaacctggcggcagcgcaaaag

SEQ ID NO: 705 - RNA, 97 nt, synthetic construct
  misc_feature 1..97: note = Description of Artificial Sequence: Synthetic oligonucleotide
  source 1..97: mol_type = other RNA
ctgggatcaagtacatgtagtgcgccacgagcaaaaggactttagaacagaggagataaagatgttttgctcgtgtaacctggcggcagcgcaaaag

SEQ ID NO: 706 - RNA, 97 nt, synthetic construct
  misc_feature 1..97: note = Description of Artificial Sequence: Synthetic oligonucleotide
  source 1..97: mol_type = other RNA
actattagggtcaggggtgttaattgtgagtagcaaggactttagaacagaggagataaagatgttgctactcactaacctggcggcagcgcaaaag

SEQ ID NO: 707 - RNA, 97 nt, synthetic construct
  misc_feature 1..97: note = Description of Artificial Sequence: Synthetic oligonucleotide
  source 1..97: mol_type = other RNA
catcgccattaaggagtagttgttgtatgaggtggtggactttagaacagaggagataaagatgaccacctcatataacctggcggcagcgcaaaag

SEQ ID NO: 708 - RNA, 97 nt, synthetic construct
  misc_feature 1..97: note = Description of Artificial Sequence: Synthetic oligonucleotide
  source 1..97: mol_type = other RNA
cactgccattgatgtgcttatgatgatctttgtggtggactttagaacagaggagataaagatgaccacaaagataaacctggcggcagcgcaaaag

SEQ ID NO: 709 - RNA, 97 nt, synthetic construct
  misc_feature 1..97: note = Description of Artificial Sequence: Synthetic oligonucleotide
  source 1..97: mol_type = other RNA
cccactcttgatgttttgttttctggaactctgtctggactttagaacagaggagataaagatgagacagagttccaacctggcggcagcgcaaaag

SEQ ID NO: 710 - RNA, 97 nt, synthetic construct
  misc_feature 1..97: note = Description of Artificial Sequence: Synthetic oligonucleotide
  source 1..97: mol_type = other RNA
atctttagcccggaacgacagtgggggcattgtgcaggactttagaacagaggagataaagatgtgcacaatgccgaacctggcggcagcgcaaaag

SEQ ID NO: 711 - RNA, 97 nt, synthetic construct
  misc_feature 1..97: note = Description of Artificial Sequence: Synthetic oligonucleotide
  source 1..97: mol_type = other RNA
tgcttctttgttgttccagtgtggagttccggtgtcggactttagaacagaggagataaagatggacaccggaacaaacctggcggcagcgcaaaag

SEQ ID NO: 712 - RNA, 97 nt, synthetic construct
  misc_feature 1..97: note = Description of Artificial Sequence: Synthetic oligonucleotide
  source 1..97: mol_type = other RNA
actccaaaagccgctcctcttttcccagcggcaaacggactttagaacagaggagataaagatggtttgccgctgcaacctggcggcagcgcaaaag

SEQ ID NO: 713 - RNA, 97 nt, synthetic construct
  misc_feature 1..97: note = Description of Artificial Sequence: Synthetic oligonucleotide
  source 1..97: mol_type = other RNA
actcttttctctccgtgtctggtccacacttctgccggactttagaacagaggagataaagatgggcagaagtgtaaacctggcggcagcgcaaaag

SEQ ID NO: 714 - RNA, 97 nt, synthetic construct
  misc_feature 1..97: note = Description of Artificial Sequence: Synthetic oligonucleotide
  source 1..97: mol_type = other RNA
ggactttggccattcacatgttttcatctcgatcagggactttagaacagaggagataaagatgctgatcgagatgaacctggcggcagcgcaaaag

SEQ ID NO: 715 - RNA, 97 nt, synthetic construct
  misc_feature 1..97: note = Description of Artificial Sequence: Synthetic oligonucleotide
  source 1..97: mol_type = other RNA
ttggttgtcctggggagatctttgcttttctggctcggactttagaacagaggagataaagatggagccagaaaagaacctggcggcagcgcaaaag

SEQ ID NO: 716 - RNA, 97 nt, synthetic construct
  misc_feature 1..97: note = Description of Artificial Sequence: Synthetic oligonucleotide
  source 1..97: mol_type = other RNA
aatgagttgcaccactaggttggtaaatgtgttaagggactttagaacagaggagataaagatgcttaacacattgaacctggcggcagcgcaaaag

SEQ ID NO: 717 - RNA, 97 nt, synthetic construct
  misc_feature 1..97: note = Description of Artificial Sequence: Synthetic oligonucleotide
  source 1..97: mol_type = other RNA
cttcaacgtcgttatagacgaacacccctgtaccgcggactttagaacagaggagataaagatggcggtacagggaaacctggcggcagcgcaaaag

SEQ ID NO: 718 - RNA, 97 nt, synthetic construct
  misc_feature 1..97: note = Description of Artificial Sequence: Synthetic oligonucleotide
  source 1..97: mol_type = other RNA
ccacaagtcttgcatctctagaacttcctcagcctcggactttagaacagaggagataaagatggaggctgaggacaacctggcggcagcgcaaaag

SEQ ID NO: 719 - RNA, 97 nt, synthetic construct
  misc_feature 1..97: note = Description of Artificial Sequence: Synthetic oligonucleotide
  source 1..97: mol_type = other RNA
attcctcaaaccgaatttcaagctcttcactgtgccggactttagaacagaggagataaagatgggcacagtgaaaaacctggcggcagcgcaaaag

SEQ ID NO: 720 - RNA, 97 nt, synthetic construct
  misc_feature 1..97: note = Description of Artificial Sequence: Synthetic oligonucleotide
  source 1..97: mol_type = other RNA
tatggttgccccctcctctctccttcccattagatgggactttagaacagaggagataaagatgcatctaatgggaaacctggcggcagcgcaaaag

SEQ ID NO: 721 - RNA, 97 nt, synthetic construct
  misc_feature 1..97: note = Description of Artificial Sequence: Synthetic oligonucleotide
  source 1..97: mol_type = other RNA
atcattcaagaacctgagggcatgtgcaaacctatcggactttagaacagaggagataaagatggataggtttgctaacctggcggcagcgcaaaag

SEQ ID NO: 722 - RNA, 167 nt, Zika virus
  source 1..167: mol_type = genomic RNA
gggcagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggc

SEQ ID NO: 723 - RNA, 160 nt, Zika virus
  source 1..160: mol_type = genomic RNA
gggtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatgg

SEQ ID NO: 724 - RNA, 150 nt, Zika virus
  source 1..150: mol_type = genomic RNA
gggccagcacagtgggatgatcgttaatgacacaggacatgaaactgatgagaatagagcgaaagttgagataacgcccaattcaccaagagccgaagccaccctgggggggtttggaagcctaggacttgattgtgaaccgaggacagg

SEQ ID NO: 725 - RNA, 150 nt, Zika virus
  source 1..150: mol_type = genomic RNA
gggcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagatt

SEQ ID NO: 726 - RNA, 168 nt, Zika virus
  source 1..168: mol_type = genomic RNA
gggccatcactgggtctcatcaatagatggggttcagtggggaaaaaagaggctatggaaataataaagaagttcaagaaagatctggctgccatgctgagaataatcaatgctaggaaggagaagaagagacgaggcgcagatactagtgtcggaattgttggcctc

SEQ ID NO: 727 - RNA, 171 nt, Zika virus
  source 1..171: mol_type = genomic RNA
gggaatgctgtcagttcatggctcccagcacagtgggatgatcgttaatgacacaggacatgaaactgatgagaatagagcgaaagttgagataacgcccaattcaccaagagccgaagccaccctgggggggtttggaagcctaggacttgattgtgaaccgaggacagg

SEQ ID NO: 728 - RNA, 201 nt, Zika virus
  source 1..201: mol_type = genomic RNA
gggagaaggatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaaga

SEQ ID NO: 729 - RNA, 137 nt, Zika virus
  source 1..137: mol_type = genomic RNA
gggctgaccctaatagtggccatcattttgctcgtggcgcactacatgtacttgatcccagggctgcaggcagcagctgcgcgtgctgcccagaagagaacggcagctggcatcatgaagaaccctgttgtggatgg

SEQ ID NO: 730 - RNA, 153 nt, Zika virus
  source 1..153: mol_type = genomic RNA
gggtttggtatgggcaaagggatgccattctacgcatgggactttggagtcccgctgctaatgataggttgctactcacaattaacacccctgaccctaatagtggccatcattttgctcgtggcgcactacatgtacttgatcccagggctg

SEQ ID NO: 731 - RNA, 176 nt, Zika virus
  source 1..176: mol_type = genomic RNA
gggccatctatgctgccttgacaactttcattaccccagccgtccaacatgcagtgaccacctcatacaacaactactccttaatggcgatggccacgcaagctggagtgttgtttggtatgggcaaagggatgccattctacgcatgggactttggagtcccgctgctaatgata

SEQ ID NO: 732 - RNA, 191 nt, Zika virus
  source 1..191: mol_type = genomic RNA
gggagaagggtgattctgctcatggtgcaggaagggttgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagc

SEQ ID NO: 733 - RNA, 231 nt, Zika virus
  source 1..231: mol_type = genomic RNA
gggagaagggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatact

SEQ ID NO: 734 - RNA, 132 nt, Zika virus
  source 1..132: mol_type = genomic RNA
gggagatcaaccactgcaagcggaagggtgatcgaggaatggtgctgcagggagtgcacaatgcccccactgtcgttccgggctaaagatggctgttggtatggaatggagataaggcccaggaaagaacca

SEQ ID NO: 735 - RNA, 158 nt, Zika virus
  source 1..158: mol_type = genomic RNA
gggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacg

SEQ ID NO: 736 - RNA, 132 nt, Zika virus
  source 1..132: mol_type = genomic RNA
gggtggacgccagagtttgttcagatcatgcggccctgaagtcattcaaggagtttgccgctgggaaaagaggagcggcttttggagtgatggaagccctgggaacactgccaggacacatgacagagagat

SEQ ID NO: 737 - RNA, 157 nt, Zika virus
  source 1..157: mol_type = genomic RNA
gggctgccggaataacctacacagatagaagatggtgctttgatggcacgaccaacaacaccataatggaagacagtgtgccggcagaagtgtggaccagacacggagagaaaagagtgctcaaaccgaggtggatggacgccagagtttgttcaga

SEQ ID NO: 738 - RNA, 155 nt, Zika virus
  source 1..155: mol_type = genomic RNA
gggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggc

SEQ ID NO: 739 - RNA, 176 nt, Zika virus
  source 1..176: mol_type = genomic RNA
gggcagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaaca

SEQ ID NO: 740 - RNA, 178 nt, Zika virus
  source 1..178: mol_type = genomic RNA
gggagcggacaagttgtcacttacgctcttaacacatttaccaacctagtggtgcaactcattcggaatatggaggctgaggaagttctagagatgcaagacttgtggctgctgcggaggtcagagaaagtgactaactggttgcagagcaacggatgggataggctcaaacgaatgg

SEQ ID NO: 741 - RNA, 150 nt, Zika virus
  source 1..150: mol_type = genomic RNA
gggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagat

SEQ ID NO: 742 - RNA, 137 nt, Zika virus
  source 1..137: mol_type = genomic RNA
gggagtggtgcaactcattcggaatatggaggctgaggaagttctagagatgcaagacttgtggctgctgcggaggtcagagaaagtgactaactggttgcagagcaacggatgggataggctcaaacgaatggcag

SEQ ID NO: 743 - RNA, 154 nt, Zika virus
  source 1..154: mol_type = genomic RNA
gggcaataccagagagggctacaggacccaaatgaaagggccatggcacagtgaagagcttgaaattcggtttgaggaatgcccaggcactaaggtccacgtggaggaaacatgtggaacgagaggaccatctctgagatcaaccactgcaagc

SEQ ID NO: 744 - RNA, 136 nt, Zika virus
  source 1..136: mol_type = genomic RNA
gggagtaggtcttctgggcttgattaccgccaatgaactcggatggttggagagaacaaagagtgacctaagccatctaatgggaaggagagaggagggggcaaccataggattctcaatggacattgacctgcgg

SEQ ID NO: 745 - RNA, 128 nt, Zika virus
  source 1..128: mol_type = genomic RNA
gggataggctcaaacgaatggcagtcagtggagatgattgcgttgtgaagccaattgatgataggtttgcacatgccctcaggttcttgaatgatatgggaaaagttaggaaggacacacaagagtgg

SEQ ID NO: 746 - RNA, 10374 nt, Zika virus
  source 1..10374: mol_type = genomic RNA
acaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacacgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcactgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctgggggggtttggaagcctaggacttgattgtgaaccgaggacaggccttgacttttcagatttgtattacttgactatgaataacaagcactggctggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatctttggagcagctttcaaatcattgtttggaggaatgtcctggttctcacaaattctcattggaacgttgctgatgtggttgggtctgaacgcaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacgagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgattctgctcatggtgcaggaagggttgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctcctatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcgcgtctactacagccaatcagagtccccaactataatctgtatattatggatgaggcccacttcacagatccctcaagtatagcagcaagaggatacatttcaacaagggttgagatgggcgaggcggccgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaagtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgactaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcc

SEQ ID NO: 747 - RNA, 10794 nt, Zika virus
  source 1..10794: mol_type = genomic RNA
agttgttgatctgtgtgagtcagactgcgacagttcgagtctgaagcgagagctaacaacagtatcaacaggtttaatttggatttggaaacgagagtttctggtcatgaaaaaccccaaagaagaaatccggaggatccggattgtcaatatgctaaaacgcggagtagcccgtgtaaaccccttgggaggtttgaagaggttgccagccggacttctgctgggtcatggacccatcagaatggttttggcgatactagcctttttgagatttacagcaatcaagccatcactgggccttatcaacagatggggttccgtggggaaaaaagaggctatggaaataataaagaagttcaagaaagatcttgctgccatgttgagaataatcaatgctaggaaagagaggaagagacgtggcgcagacaccagcatcggaatcattggcctcctgctgactacagccatggcagcagagatcactagacgcgggagtgcatactacatgtacttggataggagcgatgccgggaaggccatttcgtttgctaccacattgggagtgaacaagtgccacgtacagatcatggacctcgggcacatgtgtgacgccaccatgagttatgagtgccctatgctggatgagggagtggaaccagatgatgtcgattgctggtgcaacacgacatcaacttgggttgtgtacggaacctgtcatcacaaaaaaggtgaggcacggcgatctagaagagccgtgacgctcccttctcactctacaaggaagttgcaaacgcggtcgcagacctggttagaatcaagagaatacacgaagcacttgatcaaggttgaaaactggatattcaggaaccccgggtttgcgctagtggccgttgccattgcctggcttttgggaagctcgacgagccaaaaagtcatatacttggtcatgatactgctgattgccccggcatacagtatcaggtgcattggagtcagcaatagagacttcgtggagggcatgtcaggtgggacctgggttgatgttgtcttggaacatggaggctgcgttaccgtgatggcacaggacaagccaacagtcgacatagagttggtcacgacgacggttagtaacatggccgaggtaagatcctattgctacgaggcatcgatatcggacatggcttcggacagtcgttgcccaacacaaggtgaagcctaccttgacaagcaatcagacactcaatatgtctgcaaaagaacattagtggacagaggttggggaaacggttgtggactttttggcaaagggagcttggtgacatgtgccaagtttacgtgttctaagaagatgaccgggaagagcattcaaccggaaaatctggagtatcggataatgctatcagtgcatggctcccagcatagcgggatgattggatatgaaactgacgaagatagagcgaaagtcgaggttacgcctaattcaccaagagcggaagcaaccttgggaggctttggaagcttaggacttgactgtgaaccaaggacaggccttgacttttcagatctgtattacctgaccatgaacaataagcattggttggtgcacaaagagtggtttcatgacatcccattgccttggcatgctggggcagacaccggaactccacactggaacaacaaagaggcattggtagaattcaaggatgcccacgccaagaggcaaaccgtcgtcgttctggggagccaggaaggagccgttcacacggctctcgctggagctctagaggctgagatggatggtgcaaagggaaggctgttctctggccatttgaaatgccgcctaaaaatggacaagcttagattgaagggcgtgtcatattccttgtgcactgcggcattcacattcaccaaggtcccagctgaaacactgcatggaacagtcacagtggaggtgcagtatgcagggacagatggaccctgcaagatcccagtccagatggcggtggacatgcagaccctgaccccagttggaaggctgataaccgccaaccccgtgattactgaaagcactgagaactcaaagatgatgttggagcttgacccaccatttggggattcttacattgtcataggagttggggacaagaaaatcacccaccactggcataggagtggtagcaccatcggaaaggcatttgaggccactgtgagaggcgccaagagaatggcagtcctgggggatacagcctgggacttcggatcagtcgggggtgtgttcaactcactgggtaagggcattcaccagatttttggagcagccttcaaatcactgtttggaggaatgtcctggttctcacagatcctcataggcacgctgctagtgtggttaggtttgaacacaaagaatggatctatctccctcacatgcttggccctggggggagtgatgatcttcctctccacggctgtttctgctgacgtggggtgctcagtggacttctcaaaaaaggaaacgagatgtggcacgggggtattcatctataatgatgttgaagcctggagggaccggtacaagtaccatcctgactccccccgcagattggcagcagcagtcaagcaggcctgggaagaggggatctgtgggatctcatccgtttcaagaatggaaaacatcatgtggaaatcagtagaaggggagctcaatgctatcctagaggagaatggagttcaactgacagttgttgtgggatctgtaaaaaaccccatgtggagaggtccacaaagattgccagtgcctgtgaatgagctgccccatggctggaaagcctgggggaaatcgtattttgttagggcggcaaagaccaacaacagttttgttgtcgacggtgacacactgaaggaatgtccgcttgagcacagagcatggaatagttttcttgtggaggatcacgggtttggagtcttccacaccagtgtctggcttaaggtcagagaagattactcattagaatgtgacccagccgtcataggaacagctgttaagggaagggaggccgcgcacagtgatctgggctattggattgaaagtgaaaagaatgacacatggaggctgaagagggcccacctgattgagatgaaaacatgtgaatggccaaagtctcacacattgtggacagatggagtagaagaaagtgatcttatcatacccaagtctttagctggtccactcagccaccacaacaccagagagggttacagaacccaagtgaaagggccatggcacagtgaagagcttgaaatccggtttgaggaatgtccaggcaccaaggtttacgtggaggagacatgcggaactagaggaccatctctgagatcaactactgcaagtggaagggtcattgaggaatggtgctgtagggaatgcacaatgcccccactatcgtttcgagcaaaagacggctgctggtatggaatggagataaggcccaggaaagaaccagagagcaacttagtgaggtcaatggtgacagcggggtcaaccgatcatatggaccacttctctcttggagtgcttgtgattctactcatggtgcaggaggggttgaagaagagaatgaccacaaagatcatcatgagcacatcaatggcagtgctggtagtcatgatcttgggaggattttcaatgagtgacctggccaagcttgtgatcctgatgggtgctactttcgcagaaatgaacactggaggagatgtagctcacttggcattggtagcggcatttaaagtcagaccagccttgctggtctccttcattttcagagccaattggacaccccgtgagagcatgctgctagccctggcttcgtgtcttctgcaaactgcgatctctgctcttgaaggtgacttgatggtcctcattaatggatttgctttggcctggttggcaattcgagcaatggccgtgccacgcactgacaacatcgctctaccaatcttggctgctctaacaccactagctcgaggcacactgctcgtggcatggagagcgggcctggctacttgtggagggatcatgctcctctccctgaaagggaaaggtagtgtgaagaagaacctgccatttgtcatggccctgggattgacagctgtgagggtagtagaccctattaatgtggtaggactactgttactcacaaggagtgggaagcggagctggccccctagtgaagttctcacagccgttggcctgatatgtgcactggccggagggtttgccaaggcagacattgagatggctggacccatggctgcagtaggcttgctaattgtcagctatgtggtctcgggaaagagtgtggacatgtacattgaaagagcaggtgacatcacatgggaaaaggacgcggaagtcactggaaacagtcctcggcttgacgtggcactggatgagagtggtgacttctccttggtagaggaagatggtccacccatgagagagatcatactcaaggtggtcctgatggccatctgtggcatgaacccaatagctataccttttgctgcaggagcgtggtatgtgtatgtgaagactgggaaaaggagtggcgccctctgggacgtgcctgctcccaaagaagtgaagaaaggagagaccacagatggagtgtacagagtgatgactcgcagactgctaggttcaacacaggttggagtgggagtcatgcaagagggagtcttccacaccatgtggcacgttacaaaaggagccgcactgaggagcggtgagggaagacttgatccatactggggggatgtcaagcaggacttggtgtcatactgtgggccttggaagttggatgcagcttgggatggactcagcgaggtacagcttttggccgtacctcccggagagagggccagaaacattcagaccctgcctggaatattcaagacaaaggacggggacatcggagcagttgctctggactaccctgcagggacctcaggatctccgatcctagacaaatgtggaagagtgataggactctatggcaatggggttgtgatcaagaatggaagctatgttagtgctataacccagggaaagagggaggaggagactccggttgaatgtttcgaaccctcgatgctgaagaagaagcagctaactgtcttggatctgcatccaggagccggaaaaaccaggagagttcttcctgaaatagtccgtgaagccataaaaaagagactccggacagtgatcttggcaccaactagggttgtcgctgctgagatggaggaggccttgagaggacttccggtgcgttacatgacaacagcagtcaacgtcacccattctgggacagaaatcgttgatttgatgtgccatgccactttcacttcacgcttactacaacccatcagagtccctaattacaatctcaacatcatggatgaagcccacttcacagacccctcaagtatagctgcaagaggatacatatcaacaagggttgaaatgggcgaggcggctgccatttttatgactgccacaccaccaggaacccgtgatgcgtttcctgactctaactcaccaatcatggacacagaagtggaagtcccagagagagcctggagctcaggctttgattgggtgacagaccattctgggaaaacagtttggttcgttccaagcgtgagaaacggaaatgaaatcgcagcctgtctgacaaaggctggaaagcgggtcatacagctcagcaggaagacttttgagacagaatttcagaaaacaaaaaatcaagagtgggactttgtcataacaactgacatctcagagatgggcgccaacttcaaggctgaccgggtcatagactctaggagatgcctaaaaccagtcatacttgatggtgagagagtcatcttggctgggcccatgcctgtcacgcatgctagtgctgctcagaggagaggacgtataggcaggaaccctaacaaacctggagatgagtacatgtatggaggtgggtgtgcagagactgatgaaggccatgcacactggcttgaagcaagaatgcttcttgacaacatctacctccaggatggcctcatagcctcgctctatcggcctgaggccgataaggtagccgccattgagggagagtttaagctgaggacagagcaaaggaagaccttcgtggaactcatgaagagaggagaccttcccgtctggctagcctatcaggttgcatctgccggaataacttacacagacagaagatggtgctttgatggcacaaccaacaacaccataatggaagacagtgtaccagcagaggtttggacaaagtatggagagaagagagtgctcaaaccgagatggatggatgctagggtctgttcagaccatgcggccctgaagtcgttcaaagaattcgccgctggaaaaagaggagcggctttgggagtaatggaggccctgggaacactgccaggacacatgacagagaggtttcaggaagccattgacaacctcgccgtgctcatgcgagcagagactggaagcaggccttataaggcagcggcagcccaactgccggagaccctagagaccattatgctcttaggtttgctgggaacagtttcactggggatcttcttcgtcttgatgcggaataagggcatcgggaagatgggctttggaatggtaacccttggggccagtgcatggctcatgtggctttcggaaattgaaccagccagaattgcatgtgtcctcattgttgtgtttttattactggtggtgctcatacccgagccagagaagcaaagatctccccaagataaccagatggcaattatcatcatggtggcagtgggccttctaggtttgataactgcaaacgaacttggatggctggaaagaacaaaaaatgacatagctcatctaatgggaaggagagaagaaggagcaaccatgggattctcaatggacattgatctgcggccagcctccgcctgggctatctatgccgcattgacaactctcatcaccccagctgtccaacatgcggtaaccacttcatacaacaactactccttaatggcgatggccacacaagctggagtgctgtttggcatgggcaaagggatgccatttatgcatggggaccttggagtcccgctgctaatgatgggttgctattcacaattaacacccctgactctgatagtagctatcattctgcttgtggcgcactacatgtacttgatcccaggcctacaagcggcagcagcgcgtgctgcccagaaaaggacagcagctggcatcatgaagaatcccgttgtggatggaatagtggtaactgacattgacacaatgacaatagacccccaggtggagaagaagatgggacaagtgttactcatagcagtagccatctccagtgctgtgctgctgcggaccgcctggggatggggggaggctggagctctgatcacagcagcgacctccaccttgtgggaaggctctccaaacaaatactggaactcctctacagccacctcactgtgcaacatcttcagaggaagctatctggcaggagcttcccttatctatacagtgacgagaaacgctggcctggttaagagacgtggaggtgggacgggagagactctgggagagaagtggaaagctcgtctgaatcagatgtcggccctggagttctactcttataaaaagtcaggtatcactgaagtgtgtagagaggaggctcgccgtgccctcaaggatggagtggccacaggaggacatgccgtatcccggggaagtgcaaagatcagatggttggaggagagaggatatctgcagccctatgggaaggttgttgacctcggatgtggcagagggggctggagctattatgccgccaccatccgcaaagtgcaggaggtgagaggatacacaaagggaggtcccggtcatgaagaacccatgctggtgcaaagctatgggtggaacatagttcgtctcaagagtggagtggacgtcttccacatggcggctgagccgtgtgacactctgctgtgtgacataggtgagtcatcatctagtcctgaagtggaagagacacgaacactcagagtgctctctatggtgggggactggcttgaaaaaagaccaggggccttctgtataaaggtgctgtgcccatacaccagcactatgatggaaaccatggagcgactgcaacgtaggcatgggggaggattagtcagagtgccattgtgtcgcaactccacacatgagatgtactgggtctctggggcaaagagcaacatcataaaaagtgtgtccaccacaagtcagctcctcctgggacgcatggatggccccaggaggccagtgaaatatgaggaggatgtgaacctcggctcgggtacacgagctgtggcaagctgtgctgaggctcctaacatgaaaatcatcggcaggcgcattgagagaatccgcaatgaacatgcagaaacatggtttcttgatgaaaaccacccatacaggacatgggcctaccatgggagctacgaagcccccacgcaaggatcagcgtcttccctcgtgaacggggttgttagactcctgtcaaagccttgggacgtggtgactggagttacaggaatagccatgactgacaccacaccatacggccaacaaagagtcttcaaagaaaaagtggacaccagggtgccagatccccaagaaggcactcgccaggtaatgaacatagtctcttcctggctgtggaaggagctggggaaacgcaagcggccacgcgtctgcaccaaagaagagtttatcaacaaggtgcgcagcaatgcagcactgggagcaatatttgaagaggaaaaagaatggaagacggctgtggaagctgtgaatgatccaaggttttgggccctagtggatagggagagagaacaccacctgagaggagagtgtcacagctgtgtgtacaacatgatgggaaaaagagaaaagaagcaaggagagttcgggaaagcaaaaggtagccgcgccatctggtacatgtggttgggagccagattcttggagtttgaagcccttggattcttgaacgaggaccattggatgggaagagaaaactcaggaggtggagtcgaagggttaggattgcaaagacttggatacattctagaagaaatgaatcgggcaccaggaggaaagatgtacgcagatgacactgctggctgggacacccgcattagtaagtttgatctggagaatgaagctctgattaccaaccaaatggaggaagggcacagaactctggcgttggccgtgattaaatacacataccaaaacaaagtggtgaaggttctcagaccagctgaaggaggaaaaacagttatggacatcatttcaagacaagaccagagagggagtggacaagttgtcacttatgctctcaacacattcaccaacttggtggtgcagcttatccggaacatggaagctgaggaagtgttagagatgcaagacttatggttgttgaggaagccagagaaagtgaccagatggttgcagagcaatggatgggatagactcaaacgaatggcggtcagtggagatgactgcgttgtgaagccaatcgatgataggtttgcacatgccctcaggttcttgaatgacatgggaaaagttaggaaagacacacaggagtggaaaccctcgactggatggagcaattgggaagaagtcccgttctgctcccaccacttcaacaagctgtacctcaaggatgggagatccattgtggtcccttgccgccaccaagatgaactgattggccgagctcgcgtctcaccaggggcaggatggagcatccgggagactgcctgtcttgcaaaatcatatgcgcagatgtggcagctcctttatttccacagaagagaccttcgactgatggctaatgccatttgctcggctgtgccagttgactgggtaccaactgggagaaccacctggtcaatccatggaaagggagaatggatgaccactgaggacatgctcatggtgtggaatagagtgtggattgaggagaacgaccatatggaggacaagactcctgtaacaaaatggacagacattccctatctaggaaaaagggaggacttatggtgtggatcccttatagggcacagaccccgcaccacttgggctgaaaacatcaaagacacagtcaacatggtgcgcaggatcataggtgatgaagaaaagtacatggactatctatccacccaagtccgctacttgggtgaggaagggtccacacccggagtgttgtaagcaccaattttagtgttgtcaggcctgctagtcagccacagtttggggaaagctgtgcagcctgtaacccccccaggagaagctgggaaaccaagctcatagtcaggccgagaacgccatggcacggaagaagccatgctgcctgtgagcccctcagaggacactgagtcaaaaaaccccacgcgcttggaagcgcaggatgggaaaagaaggtggcgaccttccccacccttcaatctggggcctgaactggagactagctgtgaatctccagcagagggactagtggttagaggagaccccccggaaaacgcaaaacagcatattgacgtgggaaagaccagagactccatgagtttccaccacgctggccgccaggcacagatcgccgaacttcggcggccggtgtggggaaatccatggtttct

SEQ ID NO: 748 - RNA, 100 nt, synthetic construct
  misc_feature 1..100: note = Description of Artificial Sequence: Synthetic polynucleotide
  source 1..100: mol_type = other RNA
gggtttcgctctattctcatcagtttcatgtcctgtgtcggactttagaacagaggagataaagatggacacaggacacaacctggcggcagcgcaaaag

SEQ ID NO: 749 - RNA, 100 nt, synthetic construct
  misc_feature 1..100: note = Description of Artificial Sequence: Synthetic polynucleotide
  source 1..100: mol_type = other RNA
gggctcaactttcgctctattctcatcagtttcatgtccggactttagaacagaggagataaagatgggacatgaaacaacctggcggcagcgcaagaag

SEQ ID NO: 750 - RNA, 100 nt, synthetic construct
  misc_feature 1..100: note = Description of Artificial Sequence: Synthetic polynucleotide
  source 1..100: mol_type = other RNA
gggttatctcaactttcgctctattctcatcagtttcatggactttagaacagaggagataaagatgatgaaactgataacctggcggcagcgcaagaag

SEQ ID NO: 751 - RNA, 100 nt, synthetic construct
  misc_feature 1..100: note = Description of Artificial Sequence: Synthetic polynucleotide
  source 1..100: mol_type = other RNA
gggtcgctctattctcatcagtttcatgtcctgtgtcatggactttagaacagaggagataaagatgatgacacaggaaacctggcggcagcgcaagaag

SEQ ID NO: 752 - RNA, 98 nt, synthetic construct
  misc_feature 1..98: note = Description of Artificial Sequence: Synthetic oligonucleotide
  source 1..98: mol_type = other RNA
gggtggcttcggctcttggtgaattgggcgttatctcggactttagaacagaggagataaagatggagataacgccaacctggcggcagcgcaagaag

SEQ ID NO: 753 - RNA, 100 nt, synthetic construct
  misc_feature 1..100: note = Description of Artificial Sequence: Synthetic polynucleotide
  source 1..100: mol_type = other RNA
gggctgggatcaagtacatgtagtgcgccacgagcaaaaggactttagaacagaggagataaagatgttttgctcgtgtaacctggcggcagcgcaaaag

SEQ ID NO: 754 - RNA, 99 nt, synthetic construct
  misc_feature 1..99: note = Description of Artificial Sequence: Synthetic oligonucleotide
  source 1..99: mol_type = other RNA
gggcctgcagccctgggatcaagtacatgtagtgcgccggactttagaacagaggagataaagatgggcgcactacaaacctggcggcagcgcaagaag

SEQ ID NO: 755 - RNA, 100 nt, synthetic construct
  misc_feature 1..100: note = Description of Artificial Sequence: Synthetic polynucleotide
  source 1..100: mol_type = other RNA
gggctgccgttctcttctgggcagcacgcgcagctgctgggactttagaacagaggagataaagatgcagcagctgcgaacctggcggcagcgcaagaag

SEQ ID NO: 756 - RNA, 100 nt, synthetic construct
  misc_feature 1..100: note = Description of Artificial Sequence: Synthetic polynucleotide
  source 1..100: mol_type = other RNA
gggcagccctgggatcaagtacatgtagtgcgccacgagggactttagaacagaggagataaagatgctcgtggcgcaaacctggcggcagcgcaagaag

SEQ ID NO: 757 - RNA, 99 nt, synthetic construct
  misc_feature 1..99: note = Description of Artificial Sequence: Synthetic oligonucleotide
  source 1..99: mol_type = other RNA
gggatgccagctgccgttctcttctgggcagcacgcgcggactttagaacagaggagataaagatggcgcgtgctgcaacctggcggcagcgcaagaag

SEQ ID NO: 758 - RNA, 36 nt, Zika virus; identical to SEQ ID NO: 652
  source 1..36: mol_type = genomic RNA

SEQ ID NO: 759 - RNA, 36 nt, Zika virus
  source 1..36: mol_type = genomic RNA
ggacatgaaactgatgagaatagagcgaaagttgag

SEQ ID NO: 760 - RNA, 36 nt, Zika virus
  source 1..36: mol_type = genomic RNA
atgaaactgatgagaatagagcgaaagttgagataa

SEQ ID NO: 761 - RNA, 36 nt, Zika virus
  source 1..36: mol_type = genomic RNA
atgacacaggacatgaaactgatgagaatagagcga

SEQ ID NO: 762 - RNA, 36 nt, Zika virus
  source 1..36: mol_type = genomic RNA
gagataacgcccaattcaccaagagccgaagccacc

SEQ ID NO: 763 - RNA, 36 nt, Zika virus; identical to SEQ ID NO: 657
  source 1..36: mol_type = genomic RNA

SEQ ID NO: 764 - RNA, 36 nt, Zika virus
  source 1..36: mol_type = genomic RNA
ggcgcactacatgtacttgatcccagggctgcaggc

SEQ ID NO: 765 - RNA, 36 nt, Zika virus
  source 1..36: mol_type = genomic RNA
cagcagctgcgcgtgctgcccagaagagaacggcag

SEQ ID NO: 766 - RNA, 36 nt, Zika virus
  source 1..36: mol_type = genomic RNA
ctcgtggcgcactacatgtacttgatcccagggctg

SEQ ID NO: 767 - RNA, 36 nt, Zika virus
  source 1..36: mol_type = genomic RNA
gcgcgtgctgcccagaagagaacggcagctggcatc

SEQ ID NO: 768 - DNA, 20 bp, synthetic construct
  misc_feature 1..20: note = Description of Artificial Sequence: Synthetic oligonucleotide
  source 1..20: mol_type = other DNA
taatacgactcactataggg

SEQ ID NO: 769 - DNA, 20 bp, synthetic construct
  misc_feature 1..20: note = Description of Artificial Sequence: Synthetic oligonucleotide
  source 1..20: mol_type = other DNA
cttcagcctccagtacagcg

SEQ ID NO: 770 - DNA, 20 bp, synthetic construct
  misc_feature 1..20: note = Description of Artificial Sequence: Synthetic oligonucleotide
  source 1..20: mol_type = other DNA
gttcccacggagaatccgac

SEQ ID NO: 771 - DNA, 20 bp, synthetic construct
  misc_feature 1..20: note = Description of Artificial Sequence: Synthetic oligonucleotide
  source 1..20: mol_type = other DNA
tttcaagaatggaaaacatc

SEQ ID NO: 772 - DNA, 53 bp, Zika virus
  source 1..53: mol_type = genomic DNA
gcgggatctcctctgtttcaagaatggaaaacatcatgtggagatcagtagaa

SEQ ID NO: 773 - DNA, 20 bp, synthetic construct
  misc_feature 1..20: note = Description of Artificial Sequence: Synthetic oligonucleotide
  source 1..20: mol_type = other DNA
gatctcctctgtttcaagaa

SEQ ID NO: 774 - DNA, 53 bp, Zika virus
  source 1..53: mol_type = genomic DNA
agatggtatctgcgggatctcctctgtttcaagaatggaaaacatcatgtgga

SEQ ID NO: 775 - DNA, 20 bp, synthetic construct
  misc_feature 1..20: note = Description of Artificial Sequence: Synthetic oligonucleotide
  source 1..20: mol_type = other DNA
tggagtcccgctgctaatga

SEQ ID NO: 776 - DNA, 53 bp, Zika virus
  source 1..53: mol_type = genomic DNA
ttatgcatgggactttggagtcccgctgctaatgatgggttgctactcacaat

SEQ ID NO: 777 - DNA, 347 bp, Zika virus
  source 1..347: mol_type = genomic DNA
tggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctgggggggtttggaagcctaggacttgattgtgaaccgaggacaggccttgacttttcagatttgtattacttgactatgaataacaagcactggctggttcacaaggagtggttccacgacattccattaccttggcacgctggg

SEQ ID NO: 778 - DNA, 232 bp, Dengue virus
  source 1..232: mol_type = genomic DNA
tattgacgtgtgccaagttcaagtgtgtgacaaaactagaaggaaagatagttcaatatgaaaacctaaaatattcagtgatagtcactgtccacactggggaccagcaccaggtgggaaacgagactacagaacatggaacaattgcgaccataacacctcaagctcccacgtcggaaatacagctgaccgactacggagccctcacattggactgctcacctagaacagg

SEQ ID NO: 779 - DNA, 334 bp, Zika virus
  source 1..334: mol_type = genomic DNA
agctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccata

SEQ ID NO: 780 - DNA, 334 bp, Dengue virus
  source 1..334: mol_type = genomic DNA
ggcagctatattgatgggacttgacaagggatggccaatatcgaagatggacataggagttccacttctcgccttagggtgctattcccaggtgaacccattgacactgacagcggcggtgttgatgttagtggctcattatgccataattggaccaggactgcaagcaaaggccactagagaagctcaaaaaaggacagcggccggaataatgaaaaatccaaccgtagacgggattgttgcaatagacttggatcctgtggtttatgatacaaaatttgaaaaacagctaggccaaataatgttactgatactttgtacatcacagatcctc

SEQ ID NO: 781 - DNA, 32 bp, Zika virus
  source 1..32: mol_type = genomic DNA
ctgcgcgtgctgcccagaagagaacggcagct

SEQ ID NO: 782 - DNA, 32 bp, Zika virus
  source 1..32: mol_type = genomic DNA
cagcgcgtgctgcccagaagagaacagcagct

SEQ ID NO: 783 - DNA, 23 bp, synthetic construct
  misc_feature 1..23: note = Description of Artificial Sequence: Synthetic oligonucleotide
  source 1..23: mol_type = other DNA
cttcagcctccagtacagcgcgg

SEQ ID NO: 784 - DNA, 23 bp, synthetic construct
  misc_feature 1..23: note = Description of Artificial Sequence: Synthetic oligonucleotide
  source 1..23: mol_type = other DNA
gttcccacggagaatccgacggg

SEQ ID NO: 785 - DNA, 23 bp, synthetic construct
  misc_feature 1..23: note = Description of Artificial Sequence: Synthetic oligonucleotide
  source 1..23: mol_type = other DNA
ctgcgatgtcggtttccgcgagg

SEQ ID NO: 786 - DNA, 23 bp, synthetic construct
  misc_feature 1..23: note = Description of Artificial Sequence: Synthetic oligonucleotide
  source 1..23: mol_type = other DNA
tggaagatcaggatatgtggcgg

SEQ ID NO: 787 - DNA, 23 bp, synthetic construct
  misc_feature 1..23: note = Description of Artificial Sequence: Synthetic oligonucleotide
  source 1..23: mol_type = other DNA
ttaacgcctcgaatcagcaacgg

SEQ ID NO: 788 - DNA, 23 bp, synthetic construct
  misc_feature 1..23: note = Description of Artificial Sequence: Synthetic oligonucleotide
  source 1..23: mol_type = other DNA
gcgcgtcgtgattagcgccgtgg

SEQ ID NO: 789 - DNA, 80 bp, synthetic construct
  misc_feature 1..80: note = Description of Artificial Sequence: Synthetic oligonucleotide
  source 1..80: mol_type = other DNA
gttttagagctagaaatagcaagttaaaataaggctagtccgttatcaacttgaaaaagtggcaccgagtcggtgctttt